SEQUENCE LISTING (168 sequences)

SEQ ID NO: 1 - DNA, 345 bp, Homo sapiens
  source 1..345: mol_type = other DNA; note = Isolate AA12 immunoglobulin heavy chain variable region mRNA, partial CDS
gaggtgcagctggtggagtccggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagatcgaagggggtttgactactggggccagggaacaatggtcaccgtctcttca

SEQ ID NO: 2 - DNA, 321 bp, Homo sapiens
  source 1..321: mol_type = other DNA; note = Isolate AA12 immunoglobulin light chain variable region mRNA, partial CDS
gacatccagatgacccagtctccactctccctgtctgcatctgtaggagacagagtcaccatcacttgccggacaagtcagagcattagcagctatttaaattggtatcagcagaaaccagggaaagcccctaagctcctgatctatgctgcatccagtttgcaaagtggggtcccatcaaggttcagtggcagtggatctgggacagatttcactcttaccatcagcagtctgcaacctgaagattttgcaacttactactgtcaacagacttacagtacccctctcactttcggcggagggaccaaggtggaaatcaaa

SEQ ID NO: 3 - DNA, 357 bp, Homo sapiens
  source 1..357: mol_type = other DNA; note = Isolate EB9 immunoglobulin heavy chain variable region mRNA, partial CDS
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagatggggagggaaacgggggggggcttttgatatctggggccaagggacaatggtcaccgtctcttca

SEQ ID NO: 4 - DNA, 322 bp, Homo sapiens
  source 1..322: mol_type = other DNA; note = Isolate EB9 immunoglobulin light chain variable region mRNA, partial CDS
gacatccagatgacccagtctccattctccctgtctgcatctgtaggagacagagtcaccatcacttgccgggcaagtcagagcattagcagccatttaaattggtatcagcagaaaccagggaaagcccctaagttcctgatctatgctgcatccagtttgcaaagtggggtcccatcaaggttcagtggcagtggatctgggacagacttcactctcaccatcagcagtctgcaacctgaagattttgcaacttactactgtcaacagagttacagtactccgtacacttttggccaggggaccaaggtggaaatcaaac

SEQ ID NO: 5 - DNA, 349 bp, Homo sapiens
  source 1..349: mol_type = other DNA; note = Isolate GB5 immunoglobulin heavy chain variable region mRNA, partial CDS
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgagcagcctgagagccgaggacacggccgtgtattactgtgcgagactcatagcagcagctggtgactactggggccagggaacaatggtcaccgtctcttcag

SEQ ID NO: 6 - DNA, 322 bp, Homo sapiens
  source 1..322: mol_type = other DNA; note = Isolate GB5 immunoglobulin light chain variable region mRNA, partial CDS
gacatccagatgacccagtctccattcaccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtgaatctgggacagatttcactctcaccatcagcagtctgcaacctgaagattttgcaacttactactgtcaacagagttacagtaccccctggacgttcggccaagggaccaaggtggagatcaaac

SEQ ID NO: 7 - DNA, 369 bp, Homo sapiens
  source 1..369: mol_type = other DNA; note = Isolate FC12 immunoglobulin heavy chain variable region mRNA, partial CDS
gaggtgcagctggtggagtctggaggaggcttgatccagcctggggggtccctgagactctcctgtgcagcctctgggttcaccgtcagtagcaactacatgagctgggtccgccagactccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagagggcccgtacaactggaacgacggcctctgggtgcttttgatatctggggccaagggacaatggtcaccgtctcttca

SEQ ID NO: 8 - DNA, 319 bp, Homo sapiens
  source 1..319: mol_type = other DNA; note = Isolate FC12 immunoglobulin light chain variable region mRNA, partial CDS
tcctatgagctgactcagccaccctcagtgtccgtgtccccaggacagacagccagcatcacctgctctggagataaattgggggataaatatgcttgctggtatcagcagaagccaggccagtcccctgtgctggtcatctatcaagatagcaagcggccctcagggatccctgagcgattctctggctccaactctgggaacacagccactctgaccatcagcgggacccaggctatggatgaggctgactattactgtcaggcgtgggacagcagcaccgtggtattcggcggagggaccaagctgaccgtcctag

SEQ ID NO: 9 - protein, 115 aa, Homo sapiens
  source 1..115: note = Isolate AA12 immunoglobulin heavy chain variable region amino acid sequence
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARDRRGFDYWGQGTMVTVSS

SEQ ID NO: 10 - protein, 107 aa, Homo sapiens
  source 1..107: note = Isolate AA12 immunoglobulin light chain variable region amino acid sequence
DIQMTQSPLSLSASVGDRVTITCRTSQSISSYLNWYQQKPGKAPKLLIYAASSLQSGVPSRFSGSGSGTDFTLTISSLQPEDFATYYCQQTYSTPLTFGGGTKVEIK

SEQ ID NO: 11 - protein, 119 aa, Homo sapiens
  source 1..119: note = Isolate EB9 immunoglobulin heavy chain variable region amino acid sequence
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARWGGKRGGAFDIWGQGTMVTVSS

SEQ ID NO: 12 - protein, 107 aa, Homo sapiens
  source 1..107: note = Isolate EB9 immunoglobulin light chain variable region amino acid sequence
DIQMTQSPFSLSASVGDRVTITCRASQSISSHLNWYQQKPGKAPKFLIYAASSLQSGVPSRFSGSGSGTDFTLTISSLQPEDFATYYCQQSYSTPYTFGQGTKVEIK

SEQ ID NO: 13 - protein, 116 aa, Homo sapiens
  source 1..116: note = Isolate GB5 immunoglobulin heavy chain variable region amino acid sequence
EVQLVESGGGLIQPGGSLRLSCAASGFTVSSNYMSWVRQAPGKGLEWVSVIYSGGSTYYADSVKGRFTISRDNSKNTLYLQMSSLRAEDTAVYYCARLIAAAGDYWGQGTMVTVSS

SEQ ID NO: 14 - protein, 107 aa, Homo sapiens
  source 1..107: note = Isolate GB5 immunoglobulin light chain variable region amino acid sequence
DIQMTQSPFTLSASVGDRVTITCRASQSISSYLNWYQQKPGKAPKLLIYAASSLQSGVPSRFSGSESGTDFTLTISSLQPEDFATYYCQQSYSTPWTFGQGTKVEIK

SEQ ID NO: 15 - protein, 123 aa, Homo sapiens
  source 1..123: note = Isolate FC12 immunoglobulin heavy chain variable region amino acid sequence
EVQLVESGGGLIQPGGSLRLSCAASGFTVSSNYMSWVRQTPGKGLEWVSVIYSGGSTYYADSVKGRFTISRDNSKNTLYLQMNSLRAEDTAVYYCARGPVQLERRPLGAFDIWGQGTMVTVSS

SEQ ID NO: 16 - protein, 106 aa, Homo sapiens
  source 1..106: note = Isolate FC12 immunoglobulin light chain variable region amino acid sequence
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 17 - protein, 8 aa, Homo sapiens
  source 1..8: note = Isolate AA12 immunoglobulin heavy chain variable region CDR1 amino acid sequence (IMGT)
GFTVSSNY

SEQ ID NO: 18 - protein, 7 aa, Homo sapiens
  source 1..7: note = Isolate AA12 immunoglobulin heavy chain variable region CDR2 amino acid sequence (IMGT)
IYSGGST

SEQ ID NO: 19 - protein, 9 aa, Homo sapiens
  source 1..9: note = Isolate AA12 immunoglobulin heavy chain variable region CDR3 amino acid sequence (IMGT)
ARDRRGFDY

SEQ ID NO: 20 - protein, 6 aa, Homo sapiens
  source 1..6: note = Isolate AA12 immunoglobulin light chain variable region CDR1 amino acid sequence (IMGT)
QSISSY

SEQ ID NO: 21 - skipped

SEQ ID NO: 22 - protein, 9 aa, Homo sapiens
  source 1..9: note = Isolate AA12 immunoglobulin light chain variable region CDR3 amino acid sequence (IMGT)
QQTYSTPLT

SEQ ID NO: 23 - protein, 25 aa, Homo sapiens
  source 1..25: note = Isolate AA12 immunoglobulin heavy chain variable region framework region 1 amino acid sequence (IMGT)
EVQLVESGGGLIQPGGSLRLSCAAS

SEQ ID NO: 24 - protein, 17 aa, Homo sapiens
  source 1..17: note = Isolate AA12 immunoglobulin heavy chain variable region framework region 2 amino acid sequence (IMGT)
MSWVRQAPGKGLEWVSV

SEQ ID NO: 25 - protein, 38 aa, Homo sapiens
  source 1..38: note = Isolate AA12 immunoglobulin heavy chain variable region framework region 3 amino acid sequence (IMGT)
YYADSVKGRFTISRDNSKNTLYLQMNSLRAEDTAVYYC

SEQ ID NO: 26 - protein, 11 aa, Homo sapiens
  source 1..11: note = Isolate AA12 immunoglobulin heavy chain variable region framework region 4 amino acid sequence (IMGT)
WGQGTMVTVSS

SEQ ID NO: 27 - protein, 26 aa, Homo sapiens
  source 1..26: note = Isolate AA12 immunoglobulin light chain variable region framework region 1 amino acid sequence (IMGT)
DIQMTQSPLSLSASVGDRVTITCRTS

SEQ ID NO: 28 - protein, 17 aa, Homo sapiens
  source 1..17: note = Isolate AA12 immunoglobulin light chain variable region framework region 2 amino acid sequence (IMGT)
LNWYQQKPGKAPKLLIY

SEQ ID NO: 29 - protein, 36 aa, Homo sapiens
  source 1..36: note = Isolate AA12 immunoglobulin light chain variable region framework region 3 amino acid sequence (IMGT)
SLQSGVPSRFSGSGSGTDFTLTISSLQPEDFATYYC

SEQ ID NO: 30 - protein, 10 aa, Homo sapiens
  source 1..10: note = Isolate AA12 immunoglobulin light chain variable region framework region 4 amino acid sequence (IMGT)
FGGGTKVEIK

SEQ ID NO: 31 - protein, 9 aa, Homo sapiens
  source 1..9: note = Isolate AA12 immunoglobulin heavy chain variable region ABR1 amino acid sequence (Paratome)
FTVSSNYMS

SEQ ID NO: 32 - protein, 14 aa, Homo sapiens
  source 1..14: note = Isolate AA12 immunoglobulin heavy chain variable region ABR2 amino acid sequence (Paratome)
WVSVIYSGGSTYYA

SEQ ID NO: 33 - protein, 9 aa, Homo sapiens
  source 1..9: note = Isolate AA12 immunoglobulin heavy chain variable region ABR3 amino acid sequence (Paratome)
ARDRRGFDY

SEQ ID NO: 34 - protein, 8 aa, Homo sapiens
  source 1..8: note = Isolate AA12 immunoglobulin light chain variable region ABR1 amino acid sequence (Paratome)
QSISSYLN

SEQ ID NO: 35 - protein, 11 aa, Homo sapiens
  source 1..11: note = Isolate AA12 immunoglobulin light chain variable region ABR2 amino acid sequence (Paratome)
LLIYAASSLQS

SEQ ID NO: 36 - protein, 8 aa, Homo sapiens
  source 1..8: note = Isolate AA12 immunoglobulin light chain variable region ABR3 amino acid sequence (Paratome)
QQTYSTPL

SEQ ID NO: 37 - protein, 26 aa, Homo sapiens
  source 1..26: note = Isolate AA12 immunoglobulin heavy chain variable region framework region 1 amino acid sequence (Paratome)
EVQLVESGGGLIQPGGSLRLSCAASG

SEQ ID NO: 38 - protein, 11 aa, Homo sapiens
  source 1..11: note = Isolate AA12 immunoglobulin heavy chain variable region framework region 2 amino acid sequence (Paratome)
WVRQAPGKGLE

SEQ ID NO: 39 - protein, 35 aa, Homo sapiens
  source 1..35: note = Isolate AA12 immunoglobulin heavy chain variable region framework region 3 amino acid sequence (Paratome)
DSVKGRFTISRDNSKNTLYLQMNSLRAEDTAVYYC

SEQ ID NO: 40 - protein, 11 aa, Homo sapiens
  source 1..11: note = Isolate AA12 immunoglobulin heavy chain variable region framework region 4 amino acid sequence (Paratome)
WGQGTMVTVSS

SEQ ID NO: 41 - protein, 26 aa, Homo sapiens
  source 1..26: note = Isolate AA12 immunoglobulin light chain variable region framework region 1 amino acid sequence (Paratome)
DIQMTQSPLSLSASVGDRVTITCRTS

SEQ ID NO: 42 - protein, 11 aa, Homo sapiens
  source 1..11: note = Isolate AA12 immunoglobulin light chain variable region framework region 2 amino acid sequence (Paratome)
WYQQKPGKAPK

SEQ ID NO: 43 - protein, 32 aa, Homo sapiens
  source 1..32: note = Isolate AA12 immunoglobulin light chain variable region framework region 3 amino acid sequence (Paratome)
GVPSRFSGSGSGTDFTLTISSLQPEDFATYYC

SEQ ID NO: 44 - protein, 11 aa, Homo sapiens
  source 1..11: note = Isolate AA12 immunoglobulin light chain variable region framework region 4 amino acid sequence (Paratome)
TFGGGTKVEIK

SEQ ID NO: 45 - protein, 8 aa, Homo sapiens
  source 1..8: note = Isolate EB9 immunoglobulin heavy chain variable region CDR1 amino acid sequence (IMGT)
GFTVSSNY

SEQ ID NO: 46 - protein, 7 aa, Homo sapiens
  source 1..7: note = Isolate EB9 immunoglobulin heavy chain variable region CDR2 amino acid sequence (IMGT)
IYSGGST

SEQ ID NO: 47 - protein, 13 aa, Homo sapiens
  source 1..13: note = Isolate EB9 immunoglobulin heavy chain variable region CDR3 amino acid sequence (IMGT)
ARWGGKRGGAFDI

SEQ ID NO: 48 - protein, 6 aa, Homo sapiens
  source 1..6: note = Isolate EB9 immunoglobulin light chain variable region CDR1 amino acid sequence (IMGT)
QSISSH

SEQ ID NO: 49 - skipped

SEQ ID NO: 50 - protein, 9 aa, Homo sapiens
  source 1..9: note = Isolate EB9 immunoglobulin light chain variable region CDR3 amino acid sequence (IMGT)
QQSYSTPYT

SEQ ID NO: 51 - protein, 25 aa, Homo sapiens
  source 1..25: note = Isolate EB9 immunoglobulin heavy chain variable region framework region 1 amino acid sequence (IMGT)
EVQLVESGGGLIQPGGSLRLSCAAS

SEQ ID NO: 52 - protein, 17 aa, Homo sapiens
  source 1..17: note = Isolate EB9 immunoglobulin heavy chain variable region framework region 2 amino acid sequence (IMGT)
MSWVRQAPGKGLEWVSV

SEQ ID NO: 53 - protein, 38 aa, Homo sapiens; identical to SEQ ID NO: 25
  source 1..38: note = Isolate EB9 immunoglobulin heavy chain variable region framework region 3 amino acid sequence (IMGT)

SEQ ID NO: 54 - protein, 11 aa, Homo sapiens
  source 1..11: note = Isolate EB9 immunoglobulin heavy chain variable region framework region 4 amino acid sequence (IMGT)
WGQGTMVTVSS

SEQ ID NO: 55 - protein, 26 aa, Homo sapiens
  source 1..26: note = Isolate EB9 immunoglobulin light chain variable region framework region 1 amino acid sequence (IMGT)
DIQMTQSPFSLSASVGDRVTITCRAS

SEQ ID NO: 56 - protein, 17 aa, Homo sapiens
  source 1..17: note = Isolate EB9 immunoglobulin light chain variable region framework region 2 amino acid sequence (IMGT)
LNWYQQKPGKAPKFLIY

SEQ ID NO: 57 - protein, 36 aa, Homo sapiens; identical to SEQ ID NO: 29
  source 1..36: note = Isolate EB9 immunoglobulin light chain variable region framework region 3 amino acid sequence (IMGT)

SEQ ID NO: 58 - protein, 10 aa, Homo sapiens
  source 1..10: note = Isolate EB9 immunoglobulin light chain variable region framework region 4 amino acid sequence (IMGT)
FGQGTKVEIK

SEQ ID NO: 59 - protein, 9 aa, Homo sapiens
  source 1..9: note = Isolate EB9 immunoglobulin heavy chain variable region ABR1 amino acid sequence (Paratome)
FTVSSNYMS

SEQ ID NO: 60 - protein, 14 aa, Homo sapiens
  source 1..14: note = Isolate EB9 immunoglobulin heavy chain variable region ABR2 amino acid sequence (Paratome)
WVSVIYSGGSTYYA

SEQ ID NO: 61 - protein, 13 aa, Homo sapiens
  source 1..13: note = Isolate EB9 immunoglobulin heavy chain variable region ABR3 amino acid sequence (Paratome)
ARWGGKRGGAFDI

SEQ ID NO: 62 - protein, 8 aa, Homo sapiens
  source 1..8: note = Isolate EB9 immunoglobulin light chain variable region ABR1 amino acid sequence (Paratome)
QSISSHLN

SEQ ID NO: 63 - protein, 11 aa, Homo sapiens
  source 1..11: note = Isolate EB9 immunoglobulin light chain variable region ABR2 amino acid sequence (Paratome)
FLIYAASSLQS

SEQ ID NO: 64 - protein, 8 aa, Homo sapiens
  source 1..8: note = Isolate EB9 immunoglobulin light chain variable region ABR3 amino acid sequence (Paratome)
QQSYSTPY

SEQ ID NO: 65 - protein, 26 aa, Homo sapiens
  source 1..26: note = Isolate EB9 immunoglobulin heavy chain variable region framework region 1 amino acid sequence (Paratome)
EVQLVESGGGLIQPGGSLRLSCAASG

SEQ ID NO: 66 - protein, 11 aa, Homo sapiens
  source 1..11: note = Isolate EB9 immunoglobulin heavy chain variable region framework region 2 amino acid sequence (Paratome)
WVRQAPGKGLE

SEQ ID NO: 67 - protein, 35 aa, Homo sapiens; identical to SEQ ID NO: 39
  source 1..35: note = Isolate EB9 immunoglobulin heavy chain variable region framework region 3 amino acid sequence (Paratome)

SEQ ID NO: 68 - protein, 11 aa, Homo sapiens
  source 1..11: note = Isolate EB9 immunoglobulin heavy chain variable region framework region 4 amino acid sequence (Paratome)
WGQGTMVTVSS

SEQ ID NO: 69 - protein, 26 aa, Homo sapiens
  source 1..26: note = Isolate EB9 immunoglobulin light chain variable region framework region 1 amino acid sequence (Paratome)
DIQMTQSPFSLSASVGDRVTITCRAS

SEQ ID NO: 70 - protein, 11 aa, Homo sapiens
  source 1..11: note = Isolate EB9 immunoglobulin light chain variable region framework region 2 amino acid sequence (Paratome)
WYQQKPGKAPK

SEQ ID NO: 71 - protein, 32 aa, Homo sapiens; identical to SEQ ID NO: 43
  source 1..32: note = Isolate EB9 immunoglobulin light chain variable region framework region 3 amino acid sequence (Paratome)

SEQ ID NO: 72 - protein, 11 aa, Homo sapiens
  source 1..11: note = Isolate EB9 immunoglobulin light chain variable region framework region 4 amino acid sequence (Paratome)
TFGQGTKVEIK

SEQ ID NO: 73 - protein, 8 aa, Homo sapiens
  source 1..8: note = Isolate GB5 immunoglobulin heavy chain variable region CDR1 amino acid sequence (IMGT)
GFTVSSNY

SEQ ID NO: 74 - protein, 7 aa, Homo sapiens
  source 1..7: note = Isolate GB5 immunoglobulin heavy chain variable region CDR2 amino acid sequence (IMGT)
IYSGGST

SEQ ID NO: 75 - protein, 10 aa, Homo sapiens
  source 1..10: note = Isolate GB5 immunoglobulin heavy chain variable region CDR3 amino acid sequence (IMGT)
ARLIAAAGDY

SEQ ID NO: 76 - protein, 6 aa, Homo sapiens
  source 1..6: note = Isolate GB5 immunoglobulin light chain variable region CDR1 amino acid sequence (IMGT)
QSISSY

SEQ ID NO: 77 - skipped

SEQ ID NO: 78 - protein, 9 aa, Homo sapiens
  source 1..9: note = Isolate GB5 immunoglobulin light chain variable region CDR3 amino acid sequence (IMGT)
QQSYSTPWT

SEQ ID NO: 79 - protein, 25 aa, Homo sapiens
  source 1..25: note = Isolate GB5 immunoglobulin heavy chain variable region framework region 1 amino acid sequence (IMGT)
EVQLVESGGGLIQPGGSLRLSCAAS

SEQ ID NO: 80 - protein, 17 aa, Homo sapiens
  source 1..17: note = Isolate GB5 immunoglobulin heavy chain variable region framework region 2 amino acid sequence (IMGT)
MSWVRQAPGKGLEWVSV

SEQ ID NO: 81 - protein, 38 aa, Homo sapiens
  source 1..38: note = Isolate GB5 immunoglobulin heavy chain variable region framework region 3 amino acid sequence (IMGT)
YYADSVKGRFTISRDNSKNTLYLQMSSLRAEDTAVYYC

SEQ ID NO: 82 - protein, 11 aa, Homo sapiens
  source 1..11: note = Isolate GB5 immunoglobulin heavy chain variable region framework region 4 amino acid sequence (IMGT)
WGQGTMVTVSS

SEQ ID NO: 83 - protein, 26 aa, Homo sapiens
  source 1..26: note = Isolate GB5 immunoglobulin light chain variable region framework region 1 amino acid sequence (IMGT)
DIQMTQSPFTLSASVGDRVTITCRAS

SEQ ID NO: 84 - protein, 17 aa, Homo sapiens
  source 1..17: note = Isolate GB5 immunoglobulin light chain variable region framework region 2 amino acid sequence (IMGT)
LNWYQQKPGKAPKLLIY

SEQ ID NO: 85 - protein, 36 aa, Homo sapiens
  source 1..36: note = Isolate GB5 immunoglobulin light chain variable region framework region 3 amino acid sequence (IMGT)
SLQSGVPSRFSGSESGTDFTLTISSLQPEDFATYYC

SEQ ID NO: 86 - protein, 10 aa, Homo sapiens
  source 1..10: note = Isolate GB5 immunoglobulin light chain variable region framework region 4 amino acid sequence (IMGT)
FGQGTKVEIK

SEQ ID NO: 87 - protein, 9 aa, Homo sapiens
  source 1..9: note = Isolate GB5 immunoglobulin heavy chain variable region ABR1 amino acid sequence (Paratome)
FTVSSNYMS

SEQ ID NO: 88 - protein, 14 aa, Homo sapiens
  source 1..14: note = Isolate GB5 immunoglobulin heavy chain variable region ABR2 amino acid sequence (Paratome)
WVSVIYSGGSTYYA

SEQ ID NO: 89 - protein, 10 aa, Homo sapiens
  source 1..10: note = Isolate GB5 immunoglobulin heavy chain variable region ABR3 amino acid sequence (Paratome)
ARLIAAAGDY

SEQ ID NO: 90 - protein, 8 aa, Homo sapiens
  source 1..8: note = Isolate GB5 immunoglobulin light chain variable region ABR1 amino acid sequence (Paratome)
QSISSYLN

SEQ ID NO: 91 - protein, 11 aa, Homo sapiens
  source 1..11: note = Isolate GB5 immunoglobulin light chain variable region ABR2 amino acid sequence (Paratome)
LLIYAASSLQS

SEQ ID NO: 92 - protein, 8 aa, Homo sapiens
  source 1..8: note = Isolate GB5 immunoglobulin light chain variable region ABR3 amino acid sequence (Paratome)
QQSYSTPW

SEQ ID NO: 93 - protein, 26 aa, Homo sapiens
  source 1..26: note = Isolate GB5 immunoglobulin heavy chain variable region framework region 1 amino acid sequence (Paratome)
EVQLVESGGGLIQPGGSLRLSCAASG

SEQ ID NO: 94 - protein, 11 aa, Homo sapiens
  source 1..11: note = Isolate GB5 immunoglobulin heavy chain variable region framework region 2 amino acid sequence (Paratome)
WVRQAPGKGLE

SEQ ID NO: 95 - protein, 35 aa, Homo sapiens
  source 1..35: note = Isolate GB5 immunoglobulin heavy chain variable region framework region 3 amino acid sequence (Paratome)
DSVKGRFTISRDNSKNTLYLQMSSLRAEDTAVYYC

SEQ ID NO: 96 - protein, 11 aa, Homo sapiens
  source 1..11: note = Isolate GB5 immunoglobulin heavy chain variable region framework region 4 amino acid sequence (Paratome)
WGQGTMVTVSS

SEQ ID NO: 97 - protein, 26 aa, Homo sapiens
  source 1..26: note = Isolate GB5 immunoglobulin light chain variable region framework region 1 amino acid sequence (Paratome)
DIQMTQSPFTLSASVGDRVTITCRAS

SEQ ID NO: 98 - protein, 11 aa, Homo sapiens
  source 1..11: note = Isolate GB5 immunoglobulin light chain variable region framework region 2 amino acid sequence (Paratome)
WYQQKPGKAPK

SEQ ID NO: 99 - protein, 32 aa, Homo sapiens
  source 1..32: note = Isolate GB5 immunoglobulin light chain variable region framework region 3 amino acid sequence (Paratome)
GVPSRFSGSESGTDFTLTISSLQPEDFATYYC

SEQ ID NO: 100 - protein, 11 aa, Homo sapiens
  source 1..11: note = Isolate GB5 immunoglobulin light chain variable region framework region 4 amino acid sequence (Paratome)
TFGQGTKVEIK

SEQ ID NO: 101 - protein, 8 aa, Homo sapiens
  source 1..8: note = Isolate FC12 immunoglobulin heavy chain variable region CDR1 amino acid sequence (IMGT)
GFTVSSNY

SEQ ID NO: 102 - protein, 7 aa, Homo sapiens
  source 1..7: note = Isolate FC12 immunoglobulin heavy chain variable region CDR2 amino acid sequence (IMGT)
IYSGGST

SEQ ID NO: 103 - protein, 17 aa, Homo sapiens
  source 1..17: note = Isolate FC12 immunoglobulin heavy chain variable region CDR3 amino acid sequence (IMGT)
ARGPVQLERRPLGAFDI

SEQ ID NO: 104 - protein, 6 aa, Homo sapiens
  source 1..6: note = Isolate FC12 immunoglobulin light chain variable region CDR1 amino acid sequence (IMGT)
KLGDKY

SEQ ID NO: 105 - skipped

SEQ ID NO: 106 - protein, 9 aa, Homo sapiens
  source 1..9: note = Isolate FC12 immunoglobulin light chain variable region CDR3 amino acid sequence (IMGT)
QAWDSSTVV

SEQ ID NO: 107 - protein, 25 aa, Homo sapiens
  source 1..25: note = Isolate FC12 immunoglobulin heavy chain variable region framework region 1 amino acid sequence (IMGT)
EVQLVESGGGLIQPGGSLRLSCAAS

SEQ ID NO: 108 - protein, 17 aa, Homo sapiens
  source 1..17: note = Isolate FC12 immunoglobulin heavy chain variable region framework region 2 amino acid sequence (IMGT)
MSWVRQTPGKGLEWVSV

SEQ ID NO: 109 - protein, 38 aa, Homo sapiens; identical to SEQ ID NO: 25
  source 1..38: note = Isolate FC12 immunoglobulin heavy chain variable region framework region 3 amino acid sequence (IMGT)

SEQ ID NO: 110 - protein, 11 aa, Homo sapiens
  source 1..11: note = Isolate FC12 immunoglobulin heavy chain variable region framework region 4 amino acid sequence (IMGT)
WGQGTMVTVSS

SEQ ID NO: 111 - protein, 25 aa, Homo sapiens
  source 1..25: note = Isolate FC12 immunoglobulin light chain variable region framework region 1 amino acid sequence (IMGT)
SYELTQPPSVSVSPGQTASITCSGD

SEQ ID NO: 112 - protein, 17 aa, Homo sapiens
  source 1..17: note = Isolate FC12 immunoglobulin light chain variable region framework region 2 amino acid sequence (IMGT)
ACWYQQKPGQSPVLVIY

SEQ ID NO: 113 - protein, 36 aa, Homo sapiens
  source 1..36: note = Isolate FC12 immunoglobulin light chain variable region framework region 3 amino acid sequence (IMGT)
KRPSGIPERFSGSNSGNTATLTISGTQAMDEADYYC

SEQ ID NO: 114 - protein, 10 aa, Homo sapiens
  source 1..10: note = Isolate FC12 immunoglobulin light chain variable region framework region 4 amino acid sequence (IMGT)
FGGGTKLTVL

SEQ ID NO: 115 - protein, 9 aa, Homo sapiens
  source 1..9: note = Isolate FC12 immunoglobulin heavy chain variable region ABR1 amino acid sequence (Paratome)
FTVSSNYMS

SEQ ID NO: 116 - protein, 14 aa, Homo sapiens
  source 1..14: note = Isolate FC12 immunoglobulin heavy chain variable region ABR2 amino acid sequence (Paratome)
WVSVIYSGGSTYYA

SEQ ID NO: 117 - protein, 17 aa, Homo sapiens
  source 1..17: note = Isolate FC12 immunoglobulin heavy chain variable region ABR3 amino acid sequence (Paratome)
ARGPVQLERRPLGAFDI

SEQ ID NO: 118 - protein, 8 aa, Homo sapiens
  source 1..8: note = Isolate FC12 immunoglobulin light chain variable region ABR1 amino acid sequence (Paratome)
KLGDKYAC

SEQ ID NO: 119 - protein, 11 aa, Homo sapiens
  source 1..11: note = Isolate FC12 immunoglobulin light chain variable region ABR2 amino acid sequence (Paratome)
LVIYQDSKRPS

SEQ ID NO: 120 - protein, 8 aa, Homo sapiens
  source 1..8: note = Isolate FC12 immunoglobulin light chain variable region ABR3 amino acid sequence (Paratome)
QAWDSSTV

SEQ ID NO: 121 - protein, 26 aa, Homo sapiens
  source 1..26: note = Isolate FC12 immunoglobulin heavy chain variable region framework region 1 amino acid sequence (Paratome)
EVQLVESGGGLIQPGGSLRLSCAASG

SEQ ID NO: 122 - protein, 11 aa, Homo sapiens
  source 1..11: note = Isolate FC12 immunoglobulin heavy chain variable region framework region 2 amino acid sequence (Paratome)
WVRQTPGKGLE

SEQ ID NO: 123 - protein, 35 aa, Homo sapiens; identical to SEQ ID NO: 39
  source 1..35: note = Isolate FC12 immunoglobulin heavy chain variable region framework region 3 amino acid sequence (Paratome)

SEQ ID NO: 124 - protein, 11 aa, Homo sapiens
  source 1..11: note = Isolate FC12 immunoglobulin heavy chain variable region framework region 4 amino acid sequence (Paratome)
WGQGTMVTVSS

SEQ ID NO: 125 - protein, 25 aa, Homo sapiens
  source 1..25: note = Isolate FC12 immunoglobulin light chain variable region framework region 1 amino acid sequence (Paratome)
SYELTQPPSVSVSPGQTASITCSGD

SEQ ID NO: 126 - protein, 11 aa, Homo sapiens
  source 1..11: note = Isolate FC12 immunoglobulin light chain variable region framework region 2 amino acid sequence (Paratome)
WYQQKPGQSPV

SEQ ID NO: 127 - protein, 32 aa, Homo sapiens
  source 1..32: note = Isolate FC12 immunoglobulin light chain variable region framework region 3 amino acid sequence (Paratome)
GIPERFSGSNSGNTATLTISGTQAMDEADYYC

SEQ ID NO: 128 - protein, 11 aa, Homo sapiens
  source 1..11: note = Isolate FC12 immunoglobulin light chain variable region framework region 4 amino acid sequence (Paratome)
VFGGGTKLTVL

SEQ ID NO: 129 - DNA, 1056 bp, synthetic construct
  source 1..1056: mol_type = other DNA; note = The NS1 nucleotide sequence of MR766 virus (Rhesus/1947/Uganda)
gacgtggggtgctcagtggacttctcaaaaaaggaaacgagatgtggcacgggggtattcatctataatgatgttgaagcctggagggaccggtacaagtaccatcctgactccccccgcagattggcagcagcagtcaagcaggcctgggaagaggggatctgtgggatctcatccgtttcaagaatggaaaacatcatgtggaaatcagtagaaggggagctcaatgctatcctagaggagaatggagttcaactgacagttgttgtgggatctgtaaaaaaccccatgtggagaggtccacaaagattgccagtgcctgtgaatgagctgccccatggctggaaagcctgggggaaatcgtattttgttagggcggcaaagaccaacaacagttttgttgtcgacggtgacacactgaaggaatgtccgcttgagcacagagcatggaatagttttcttgtggaggatcacgggtttggagtcttccacaccagtgtctggcttaaggtcagagaagattactcattagaatgtgacccagccgtcataggaacagctgttaagggaagggaggccgcgcacagtgatctgggctattggattgaaagtgaaaagaatgacacatggaggctgaagagggcccacctgattgagatgaaaacatgtgaatggccaaagtctcacacattgtggacagatggagtagaagaaagtgatcttatcatacccaagtctttagctggtccactcagccaccacaacaccagagagggttacagaacccaagtgaaagggccatggcacagtgaagagcttgaaatccggtttgaggaatgtccaggcaccaaggtttacgtggaggagacatgcggaactagaggaccatctctgagatcaactactgcaagtggaagggtcattgaggaatggtgctgtagggaatgcacaatgcccccactatcgtttcgagcaaaagacggctgctggtatggaatggagataaggcccaggaaagaaccagagagcaacttagtgaggtcaatggtgacagcg

SEQ ID NO: 130 - DNA, 10675 bp, Zika virus
  source 1..10675: mol_type = other DNA; note = PRVABC59 virus (2015/Puerto Rico Accession: No.: KU501215)
gttgttgatctgtgtgaatcagactgcgacagttcgagtttgaagcgaaagctagcaacagtatcaacaggttttattttggatttggaaacgagagtttctggtcatgaaaaacccaaaaaagaaatccggaggattccggattgtcaatatgctaaaacgcggagtagcccgtgtgagcccctttgggggcttgaagaggctgccagccggacttctgctgggtcatgggcccatcaggatggtcttggcgattctagcctttttgagattcacggcaatcaagccatcactgggtctcatcaatagatggggttcagtggggaaaaaagaggctatggaaacaataaagaagttcaagaaagatctggctgccatgctgagaataatcaatgctaggaaggagaagaagagacgaggcgcagatactagtgtcggaattgttggcctcctgctgaccacagctatggcagcggaggtcactagacgtgggagtgcatactatatgtacttggacagaaacgatgctggggaggccatatcttttccaaccacattggggatgaataagtgttatatacagatcatggatcttggacacatgtgtgatgccaccatgagctatgaatgccctatgctggatgagggggtggaaccagatgacgtcgattgttggtgcaacacgacgtcaacttgggttgtgtacggaacctgccatcacaaaaaaggtgaagcacggagatctagaagagctgtgacgctcccctcccattccaccaggaagctgcaaacgcggtcgcaaacctggttggaatcaagagaatacacaaagcacttgattagagtcgaaaattggatattcaggaaccctggcttcgcgttagcagcagctgccatcgcttggcttttgggaagctcaacgagccaaaaagtcatatacttggtcatgatactgctgattgccccggcatacagcatcaggtgcataggagtcagcaatagggactttgtggaaggtatgtcaggtgggacttgggttgatgttgtcttggaacatggaggttgtgtcaccgtaatggcacaggacaaaccgactgtcgacatagagctggttacaacaacagtcagcaacatggcggaggtaagatcctactgctatgaggcatcaatatcagacatggcttctgacagccgctgcccaacacaaggtgaagcctaccttgacaagcaatcagacactcaatatgtctgcaaaagaacgttagtggacagaggctggggaaatggatgtggactttttggcaaagggagcctggtgacatgcgctaagtttgcatgctccaagaaaatgaccgggaagagcatccagccagagaatctggagtaccggataatgctgtcagttcatggctcccagcacagtgggatgatcgttaatgacacaggacatgaaactgatgagaatagagcgaaagttgagataacgcccaattcaccgagagccgaagccaccctggggggttttggaagcctaggacttgattgtgaaccgaggacaggccttgacttttcagatttgtattacttgactatgaataacaagcactggttggttcacaaggagtggttccacgacattccattaccttggcacgctggggcagacaccggaactccacactggaacaacaaagaagcactggtagagttcaaggacgcacatgccaaaaggcaaactgtcgtggttctagggagtcaagaaggagcagttcacacggcccttgctggagctctggaggctgagatggatggtgcaaagggaaggctgtcctctggccacttgaaatgtcgcctgaaaatggataaacttagattgaagggcgtgtcatactccttgtgtactgcagcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaagcactgagaactctaagatgatgctggaacttgatccaccatttggggactcttacattgtcataggagtcggggagaagaagatcacccaccactggcacaggagtggcagcaccattggaaaagcatttgaagccactgtgagaggtgccaagagaatggcagtcttgggagacacagcctgggactttggatcagttggaggcgctctcaactcattgggcaagggcatccatcaaatttttggagcagctttcaaatcattgtttggaggaatgtcctggttctcacaaattctcattggaacgttgctgatgtggttgggtctgaacacaaagaatggatctatttcccttatgtgcttggccttagggggagtgttgatcttcttatccacagccgtctctgctgatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgcaggatcaactgatcacatggaccacttctcccttggagtgcttgtgatcctgctcatggtgcaggaagggctgaagaagagaatgaccacaaagatcatcataagcacatcaatggcagtgctggtagctatgatcctgggaggattttcaatgagtgacctggctaagcttgcaattttgatgggtgccaccttcgcggaaatgaacactggaggagatgtagctcatctggcgctgatagcggcattcaaagtcagaccagcgttgctggtatctttcatcttcagagctaattggacaccccgtgaaagcatgctgctggccttggcctcgtgtcttttgcaaactgcgatctccgccttggaaggcgacctgatggttctcatcaatggttttgctttggcctggttggcaatacgagcgatggttgttccacgcactgataacatcaccttggcaatcctggctgctctgacaccactggcccggggcacactgcttgtggcgtggagagcaggccttgctacttgcggggggtttatgctcctctctctgaagggaaaaggcagtgtgaagaagaacttaccatttgtcatggccctgggactaaccgctgtgaggctggtcgaccccatcaacgtggtgggactgctgttgctcacaaggagtgggaagcggagctggccccctagcgaagtactcacagctgttggcctgatatgcgcattggctggagggttcgccaaggcagatatagagatggctgggcccatggccgcggtcggtctgctaattgtcagttacgtggtctcaggaaagagtgtggacatgtacattgaaagagcaggtgacatcacatgggaaaaagatgcggaagtcactggaaacagtccccggctcgatgtggcgctagatgagagtggtgatttctccctggtggaggatgacggtccccccatgagagagatcatactcaaggtggtcctgatgaccatctgtggcatgaacccaatagccataccctttgcagctggagcgtggtacgtatacgtgaagactggaaaaaggagtggtgctctatgggatgtgcctgctcccaaggaagtaaaaaagggggagaccacagatggagtgtacagagtaatgactcgtagactgctaggttcaacacaagttggagtgggagttatgcaagagggggtctttcacactatgtggcacgtcacaaaaggatccgcgctgagaagcggtgaagggagacttgatccatactggggagatgtcaagcaggatctggtgtcatactgtggtccatggaagctagatgccgcctgggatgggcacagcgaggtgcagctcttggccgtgccccccggagagagagcgaggaacatccagactctgcccggaatatttaagacaaaggatggggacattggagcggttgcgctggattacccagcaggaacttcaggatctccaatcctagacaagtgtgggagagtgataggactttatggcaatggggtcgtgatcaaaaacgggagttatgttagtgccatcacccaagggaggagggaggaagagactcctgttgagtgcttcgagccctcgatgctgaagaagaagcagctaactgtcttagacttgcatcctggagctgggaaaaccaggagagttcttcctgaaatagtccgtgaagccataaaaacaagactccgtactgtgatcttagctccaaccagggttgtcgctgctgaaatggaggaggcccttagagggcttccagtgcgttatatgacaacagcagtcaatgtcacccactctggaacagaaatcgtcgacttaatgtgccatgccaccttcacttcacgtctactacagccaatcagagtccccaactataatctgtatattatggatgaggcccacttcacagatccctcaagtatagcagcaagaggatacatttcaacaagggttgagatgggcgaggcggctgccatcttcatgaccgccacgccaccaggaacccgtgacgcatttccggactccaactcaccaattatggacaccgaagtggaagtcccagagagagcctggagctcaggctttgattgggtgacggatcattctggaaaaacagtttggtttgttccaagcgtgaggaacggcaatgagatcgcagcttgtctgacaaaggctggaaaacgggtcatacagctcagcagaaagacttttgagacagagttccagaaaacaaaacatcaagagtgggactttgtcgtgacaactgacatttcagagatgggcgccaactttaaagctgaccgtgtcatagattccaggagatgcctaaagccggtcatacttgatggcgagagagtcattctggctggacccatgcctgtcacacatgccagcgctgcccagaggagggggcgcataggcaggaatcccaacaaacctggagatgagtatctgtatggaggtgggtgcgcagagactgacgaagaccatgcacactggcttgaagcaagaatgctccttgacaatatttacctccaagatggcctcatagcctcgctctatcgacctgaggccgacaaagtagcagccattgagggagagttcaagcttaggacggagcaaaggaagacctttgtggaactcatgaaaagaggagatcttcctgtttggctggcctatcaggttgcatctgccggaataacctacacagatagaagatggtgctttgatggcacgaccaacaacaccataatggaagacagtgtgccggcagaggtgtggaccagacacggagagaaaagagtgctcaaaccgaggtggatggacgccagagtttgttcagatcatgcggccctgaagtcattcaaggagtttgccgctgggaaaagaggagcggcttttggagtgatggaagccctgggaacactgccaggacacatgacagagagattccaggaagccattgacaacctcgctgtgctcatgcgggcagagactggaagcaggccttacaaagccgcggcggcccaattgccggagaccctagagaccataatgcttttggggttgctgggaacagtctcgctgggaatcttcttcgtcttgatgaggaacaagggcatagggaagatgggctttggaatggtgactcttggggccagcgcatggctcatgtggctctcggaaattgagccagccagaattgcatgtgtcctcattgttgtgttcctattgctggtggtgctcatacctgagccagaaaagcaaagatctccccaggacaaccaaatggcaatcatcatcatggtagcagtaggtcttctgggcttgattaccgccaatgaactcggatggttggagagaacaaagagtgacctaagccatctaatgggaaggagagaggagggggcaaccataggattctcaatggacattgacctgcggccagcctcagcttgggccatctatgctgccttgacaactttcattaccccagccgtccaacatgcagtgaccacctcatacaacaactactccttaatggcgatggccacgcaagctggagtgttgtttggcatgggcaaagggatgccattctacgcatgggactttggagtcccgctgctaatgataggttgctactcacaattaacacccctgaccctaatagtggccatcattttgctcgtggcgcactacatgtacttgatcccagggctgcaggcagcagctgcgcgtgctgcccagaagagaacggcagctggcatcatgaagaaccctgttgtggatggaatagtggtgactgacattgacacaatgacaattgacccccaagtggagaaaaagatgggacaggtgctactcatagcagtagccgtctccagcgccatactgtcgcggaccgcctgggggtggggggaggctggggctctgatcacagccgcaacttccactttgtgggaaggctctccgaacaagtactggaactcctctacagccacttcactgtgtaacatttttaggggaagttacttggctggagcttctctaatctacacagtaacaagaaacgctggcttggtcaagagacgtgggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgtcgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgcaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagctgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgtataccaggaggaaggatgtatgcagatgacactgctggctgggacacccgcattagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctgtaagcaccaatcttaatgttgtcaggcctgctagtcagccacagcttggggaaagctgtgcagcctgtgacccccccaggagaagctgggaaaccaagcctatagtcaggccgagaacgccatggcacggaagaagccatgctgcctgtgagcccctcagaggacactgagtcaaaaaaccccacgcgcttggaggcgcaggatgggaaaagaaggtggcgaccttccccacccttcaatctggggcctgaactggagatcagctgtggatctccagaagagggactagtggttagagga

SEQ ID NO: 131 - protein, 24 aa, synthetic construct
  source 1..24: note = ZIKV envelope amino acid sequence
NGSISLMCLALGGVLIFLSTAVSA

SEQ ID NO: 132 - protein, 9 aa, synthetic construct
  source 1..9: note = PreScission Protease cleavage site amino acid sequence
LEVLFNGPG

SEQ ID NO: 133 - protein, 6 aa, synthetic construct
  source 1..6: note = Hexahistidine motif amino acid sequence
HHHHHH

SEQ ID NO: 134 - protein, 6 aa, Homo sapiens
  source 1..6: note = VL CDR1 (IMGT)
  VARIANT 6: note = X is Y or H
QSISSX

SEQ ID NO: 135 - skipped

SEQ ID NO: 136 - protein, 9 aa, Homo sapiens
  source 1..9: note = VL CDR3 (IMGT)
  VARIANT 3: note = X is T or S
  VARIANT 8: note = X is L, Y or W
QQXYSTPXT

SEQ ID NO: 137 - protein, 8 aa, Homo sapiens
  source 1..8: note = VL ABR1 (Paratome)
  VARIANT 6: note = X is Y or H
QSISSXLN

SEQ ID NO: 138 - protein, 11 aa, Homo sapiens
  source 1..11: note = VL ABR2 (Paratome)
  VARIANT 1: note = X is F or L
XLIYAASSLQS

SEQ ID NO: 139 - protein, 8 aa, Homo sapiens
  source 1..8: note = VL ABR3 (Paratome)
  VARIANT 1: note = X is T or S
  VARIANT 8: note = X is L, Y or W
QQXYSTPX

SEQ ID NO: 140 - protein, 6 aa, synthetic construct
  source 1..6: note = Synthetic Sequence
LALAPG

SEQ ID NO: 141 - protein, 7 aa, synthetic construct
  source 1..7: note = Example of thrombin cleavage site
LVPRGSP

SEQ ID NO: 142 - protein, 7 aa, synthetic construct
  source 1..7: note = Example of cleavage site recognized by Tobacco Etch Virus (TEV) protease
  VARIANT 7: note = X is G or S
ENLYFQX

SEQ ID NO: 143 - protein, 8 aa, Homo sapiens
  source 1..8: note = VH CDR1
GFTVSSNY

SEQ ID NO: 144 - protein, 7 aa, Homo sapiens
  source 1..7: note = VH CDR2
IYSGGST

SEQ ID NO: 145 - protein, 9 aa, Homo sapiens
  source 1..9: note = VH CDR3
ARDRRGFDY

SEQ ID NO: 146 - protein, 13 aa, Homo sapiens
  source 1..13: note = VH CDR3
ARWGGKRGGAFDI

SEQ ID NO: 147 - protein, 10 aa, Homo sapiens
  source 1..10: note = VH CDR3
ARLIAAAGDY

SEQ ID NO: 148 - protein, 17 aa, Homo sapiens
  source 1..17: note = VH CDR3
ARGPVQLERRPLGAFDI

SEQ ID NO: 149 - protein, 9 aa, Homo sapiens
  source 1..9: note = VH ABR1
FTVSSNYMS

SEQ ID NO: 150 - protein, 14 aa, Homo sapiens
  source 1..14: note = VH ABR2
WVSVIYSGGSTYYA

SEQ ID NO: 151 - protein, 9 aa, Homo sapiens
  source 1..9: note = VH ABR3
ARDRRGFDY

SEQ ID NO: 152 - protein, 13 aa, Homo sapiens
  source 1..13: note = VH ABR3
ARWGGKRGGAFDI

SEQ ID NO: 153 - protein, 10 aa, Homo sapiens
  source 1..10: note = VH ABR3
ARLIAAAGDY

SEQ ID NO: 154 - protein, 17 aa, Homo sapiens
  source 1..17: note = VH ABR3
ARGPVQLERRPLGAFDI

SEQ ID NO: 155 - protein, 11 aa, Homo sapiens [sequence omitted]

SEQ ID NO: 156 - protein, 19 aa, Homo sapiens
  source 1..19: note = CDR3
CARGPVQLERRPLGAFDIW

SEQ ID NO: 157 - protein, 15 aa, Homo sapiens
  source 1..15: note = CDR3
CARWGGKRGGAFDIW

SEQ ID NO: 158 - protein, 12 aa, Homo sapiens
  source 1..12: note = CDR3
CARLIAAAGDYW

SEQ ID NO: 159 - protein, 11 aa, Homo sapiens [sequence omitted]

SEQ ID NO: 160 - protein, 9 aa, Homo sapiens
  source 1..9: note = CDR3
CQAWDSSTV

SEQ ID NO: 161 - protein, 11 aa, Homo sapiens [sequence omitted]

SEQ ID NO: 162 - protein, 11 aa, Homo sapiens [sequence omitted]

SEQ ID NO: 163 - protein, 34 aa, synthetic construct
  source 1..34: note = Synthetic Peptide
NGSISLMCLALGGVLIFLSTAVSADVGCSVDFSK

SEQ ID NO: 164 - protein, 8 aa, synthetic construct
  source 1..8: note = Synthetic Peptide
LVRSMVTA

SEQ ID NO: 165 - protein, 23 aa, synthetic construct
  source 1..23: note = Synthetic Peptide (cleavage site and hexahistidine motif)
LVRSMVTALEVLFNGPGHHHHHH

SEQ ID NO: 166 - protein, 376 aa, synthetic construct
  source 1..376: note = Synthetic NS1 polypeptide (comprising a fragment of a Zika virus evelope protein, and a Zika virus NS1 polypeptide)
NGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTA

SEQ ID NO: 167 - protein, 392 aa, synthetic construct
  source 1..392: note = Synthetic NS1 polypeptide (comprising a fragment of a Zika virus evelope protein, a Zika virus NS1 polypeptide, a cleavage site, and a hexahistidine motif)
MNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTALEVLFQGPGHHHHHH

SEQ ID NO: 168 - DNA, 1056 bp, Zika virus
  source 1..1056: mol_type = other DNA; note = PRVABC59 virus NS1 nucleotide sequence
gatgtggggtgctcggtggacttctcaaagaaggagacgagatgcggtacaggggtgttcgtctataacgacgttgaagcctggagggacaggtacaagtaccatcctgactccccccgtagattggcagcagcagtcaagcaagcctgggaagatggtatctgcgggatctcctctgtttcaagaatggaaaacatcatgtggagatcagtagaaggggagctcaacgcaatcctggaagagaatggagttcaactgacggtcgttgtgggatctgtaaaaaaccccatgtggagaggtccacagagattgcccgtgcctgtgaacgagctgccccacggctggaaggcttgggggaaatcgtatttcgtcagagcagcaaagacaaataacagctttgtcgtggatggtgacacactgaaggaatgcccactcaaacatagagcatggaacagctttcttgtggaggatcatgggttcggggtatttcacactagtgtctggctcaaggttagagaagattattcattagagtgtgatccagccgttattggaacagctgttaagggaaaggaggctgtacacagtgatctaggctactggattgagagtgagaagaatgacacatggaggctgaagagggcccatctgatcgagatgaaaacatgtgaatggccaaagtcccacacattgtggacagatggaatagaagagagtgatctgatcatacccaagtctttagctgggccactcagccatcacaataccagagagggctacaggacccaaatgaaagggccatggcacagtgaagagcttgaaattcggtttgaggaatgcccaggcactaaggtccacgtggaggaaacatgtggaacaagaggaccatctctgagatcaaccactgcaagcggaagggtgatcgaggaatggtgctgcagggagtgcacaatgcccccactgtcgttccgggctaaagatggctgttggtatggaatggagataaggcccaggaaagaaccagaaagcaacttagtaaggtcaatggtgactgca

(19) United States
(12) Patent Application Publication
(10) Pub. No.: US 2026/0266820 A1
(43) Pub. Date: Sep. 10, 2026

(54) HUMAN ANTIBODIES TARGETING ZIKA VIRUS NS1, NS1 POLYPEPTIDES AND USES THEREOF

(71) Applicant: ICAHN SCHOOL OF MEDICINE AT MOUNT SINAI, New York, NY (US)

(72) Inventors: Mark BAILEY, New York, NY (US); Peter PALESE, New York, NY (US); Gene TAN, New York, NY (US)

(73) Assignee: ICAHN SCHOOL OF MEDICINE AT MOUNT SINAI, New York, NY (US)

(21) Appl. No.: 19/330,603

(22) Filed: Sep. 16, 2025

Related U.S. Application Data
(63) Continuation of application No. 17/289,448, filed on Oct. 30, 2019, now abandoned.
(60) Provisional application No. 62/753,727, filed on Oct. 31, 2018.

(51) Int. Cl. G01N 33/569; A61K 39/00; A61K 39/12; A61K 47/68; A61P 31/14; C07K 14/005; C07K 16/10

(52) U.S. Cl. CPC G01N 33/56983; A61K 39/12; A61K 47/6841; A61P 31/14; C07K 14/005; C07K 16/10; A61K 2039/505; A61K 2039/53; C07K 2317/54; C07K 2317/55; C07K 2317/622; C12N 2770/24122; C12N 2770/24134; G01N 2333/185; G01N 2469/10

(57) ABSTRACT

In one aspect, provided herein are antibodies that bind to Zika virus non-structural protein 1 (NS1) and compositions comprising the same. In a specific embodiment, such antibodies or compositions thereof may be used to passively immunize a subject against Zika virus. In another embodiment, such antibodies or compositions thereof may be used to diagnose a Zika virus infection. In another aspect, provided herein are recombinant NS1 polypeptides and compositions comprising the same that may be used to immunize a subject against Zika virus disease.

### CROSS-REFERENCE TO RELATED APPLICATIONS

This application is a continuation of U.S. patent application Ser. No. 17/289,448, which is a U.S. National Stage Application under 35 U.S.C. § 371 of International Patent Application No. PCT/US2019/058865, filed Oct. 30, 2019, which claims the benefit of U.S. Provisional Application No. 62/753,727, filed Oct. 31, 2018, the disclosure of each of which is incorporated by reference herein in its entirety.

### SEQUENCE LISTING

This application contains an electronic Sequence Listing which has been submitted in XML file format via Patent Center, the entire content of which is incorporated by reference herein in its entirety. The Sequence Listing XML file submitted via Patent Center is entitled “06923-452-999_SEQ_LISTING.xml”, was created on Sep. 13, 2025, and is 201,977 bytes in size.

### 1. Introduction

In one aspect, provided herein are antibodies that bind to Zika virus non-structural protein 1 (NS1) and compositions comprising the same. In a specific embodiment, such antibodies or compositions thereof may be used to passively immunize a subject against Zika virus. In another embodiment, such antibodies or compositions thereof may be used to diagnose a Zika virus infection. In another aspect, provided herein are recombinant NS1 polypeptides and compositions comprising the same that may be used to immunize a subject against Zika virus disease.

### 2. Background

Zika virus (ZIKV) is an arthropod-borne flavivirus closely related to dengue, yellow fever and West Nile viruses, which has caused an emerging epidemic in the Americas, the Caribbean, and the Pacific regions. While ZIKV is spread primarily through the bite of an infected Aedes species mosquito, cases of sexual transmission have also been reported^{1,2}. ZIKV infection is associated with severe illness in humans, including microcephaly and birth defects in newborns³⁻⁵ and Guillain-Barré syndrome in adults^{6,7}. Consequently, ZIKV infection poses significant threats to global health.

To understand the molecular determinants of immunity to ZIKV infection, several groups have isolated monoclonal antibodies (mAbs) from patients infected with ZIKV⁸⁻¹². These studies have revealed important antigenic sites on the envelope (E) protein required for virus neutralization. Quaternary epitopes such as the “envelope dimer epitope”, which are dependent on the native dimeric assembly of the E protein, are promising vaccine and therapeutic targets, as mAbs generated against these sites tend to be potently neutralizing¹⁰. However, one chief concern in the development of flavivirus vaccines targeting the E protein is the phenomenon of antibody-dependent enhancement of disease (ADE). This occurs when viral replication is enhanced by preexisting antibodies that opsonize but do not fully neutralize the virion resulting in enhanced uptake of the virion-antibody complex by FcγR-bearing target cells. The virus is then able to replicate in these cells, increasing the severity of disease¹³. Though there is no epidemiologic evidence that Zika virus can cause ADE in humans, studies have shown ZIKV-induced monoclonal antibodies targeting the E protein can enhance infection of ZIKV or Dengue virus (DENV) in vitro and induce mortality in DENV-infected mice⁸. Additionally, passive transfer of DENV or West Nile virus (WNV) immune plasma to immunocompromised mice has resulted in more severe disease progression upon ZIKV infection in vivo¹⁴. Consequently, ADE may limit the therapeutic application of E protein-specific antibodies and vaccines against Zika virus.

Other viral proteins including non-structural protein 1 (NS1) have emerged as promising targets as antibodies that do not bind the virion are unlikely to enhance disease. In a recent study of four patients infected by ZIKV, 34.4% of virus-specific mAbs target the NS1 protein⁸. This immunogenic glycoprotein plays an essential role in viral RNA replication and immune evasion. The NS1 protein is initially translated as a monomer, becomes glycosylated in the ER and subsequently forms a dimer that can potentially traffic to multiple distinct locations within the cell¹⁵. The NS1 protein of many flaviviruses is known to associate with the viral replication complex on the surface of the endoplasmic reticulum membrane, associate with the plasma membrane by a glycosylphosphatidylinositol linker, exit cells to form a lipophilic hexamer, and potentially bind to uninfected cells via glycosaminoglycan interactions¹⁶.

Protective antibodies against viral pathogens are able to protect via multiple mechanisms: neutralization, Fcγ-receptor mediated viral clearance, and complement-dependent cytotoxicity (CDC)¹⁷. Antibodies against the NS1 protein were shown to be protective against a number of different flavivirus species. In Japanese encephalitis virus, NS1-specific antibodies were found to reduce viral output from infected cells¹⁸. Yellow fever virus NS1 fragments were used as a vaccine and immunized mice had reduced neurovirulence upon viral challenge¹⁹. Later, NS1-specific antibodies were found to protect against yellow fever encephalitis in mice²⁰. Additionally, mAbs targeting the yellow fever virus NS1 protein protected monkeys against lethal challenge by invoking Fcγ-mediated effector functions²⁰⁻²². Other work has shown that mAbs against West Nile virus NS1 protein prevent lethal infection in mice through Fcγ-receptor mediated phagocytosis as well as an undetermined Fc-independent mechanism^{23,24}. The dengue virus NS1 protein has been extensively studied in the context of antiviral immunity. Successful passive protection studies were performed in mice with NS1-specific monoclonal antibodies as well as protein and DNA plasmid-based vaccines²⁵⁻³⁰. Recently, dengue virus NS1 protein was shown to induce disruption of endothelial barriers in mice, which can also be prevented by vaccination with the NS1 protein³¹. Finally, an NS1-based vaccine for Zika virus was successfully tested in a mouse challenge model, proving that NS1-mediated immunity alone is sufficient for a protective vaccine³². These studies in many related flaviviruses suggest mAbs against the ZIKV NS1 protein are likely protective.

Specific treatments and vaccines for Zika virus are not currently available. Thus, there is a need for specific treatments and vaccines for Zika virus.

### 3. Summary

In one aspect, provided herein are antibodies that specifically bind to a Zika virus NS1. In one embodiment, an antibody described herein or a composition thereof may be used to prevent a Zika virus disease. In another embodiment, an antibody described herein or a composition thereof may be used to treat a Zika virus infection or a Zika virus disease. In another embodiment, an antibody described herein or a composition thereof may be used in an immunoassay. In another embodiment, an antibody described herein or a composition thereof may be used to detect or diagnose a Zika virus infection.

In a specific embodiment, provided herein is an antibody that specifically binds to a Zika virus NS1, wherein the antibody comprises: (1) a variable heavy chain region (VH) complementarity determining region (CDR)1 comprising the amino acid sequence GFTVSSNY (SEQ ID NO:143), (2) a VH CDR2 comprising the amino acid sequence IYSGGST (SEQ ID NO: 144), (3) a VH CDR3 comprising the amino acid sequence ARDRRGFDY (SEQ ID NO: 145), ARWGGKRGGAFDI (SEQ ID NO:146), ARLIAAAGDY (SEQ ID NO:147), or ARGPVQLERRPLGAFDI (SEQ ID NO:148), (4) a variable light chain region (VL) CDR1 comprising the amino acid sequence QSISSX, X is Y or H (SEQ ID NO:134), (5) a VL CDR2 comprising the amino acid sequence X1X2S, X1 is A or Q, X2 is A or D (SEQ ID NO:135), and (6) a VL CDR3 comprising the amino acid sequence QQX1YSTPX2T, X1 is T or S, X2 is L, Y, or W (SEQ ID NO:136). In another specific embodiment, provided herein is an antibody that specifically binds to a Zika virus NS1, wherein the antibody comprises: (1) a VH antibody binding region (ABR)1 comprising the amino acid sequence FTVSSNYMS (SEQ ID NO:149), (2) a VH ABR2 comprising the amino acid sequence WVSVIYSGGSTYYA (SEQ ID NO:150), (3) a VH ABR3 comprising the amino acid sequence ARDRRGFDY (SEQ ID NO:151), ARWGGKRGGAFDI (SEQ ID NO:152), ARLIAAAGDY (SEQ ID NO:153), or ARGPVQLERRPLGAFDI (SEQ ID NO:154), (4) a VL ABR1 comprising the amino acid sequence QSISSXILN, X1 is Y or H (SEQ ID NO:137), (5) a VL ABR2 comprising the amino acid sequence XILIYAASSLQS, X1 is F or L (SEQ ID NO:138), and (6) a VL ABR3 comprising the amino acid sequence QQX1YSTPX2, X1 is T or S, X2 is L, Y or W (SEQ ID NO: 139).

In another specific embodiment, provided herein is an antibody that specifically binds to a Zika virus NS1, wherein the antibody comprises: (1) a VH CDR1 comprising the amino acid sequence GFTVSSNY (SEQ ID NO:17), (2) a VH CDR2 comprising the amino acid sequence IYSGGST (SEQ ID NO: 18), (3) a VH CDR3 comprising the amino acid sequence ARDRRGFDY (SEQ ID NO: 19), (4) a VL CDR1 comprising the amino acid sequence QSISSY (SEQ ID NO: 20), (5) a VL CDR2 comprising the amino acid sequence AAS (SEQ ID NO: 21), and (6) a VL CDR3 comprising the amino acid sequence QQTYSTPLT (SEQ ID NO:22).

In another specific embodiment, provided herein is an antibody that specifically binds to a Zika virus NS1, wherein the antibody comprises: (1) a VH CDR1 comprising the amino acid sequence GFTVSSNY (SEQ ID NO:45), (2) a VH CDR2 comprising the amino acid sequence IYSGGST (SEQ ID NO:46), (3) a VH CDR3 comprising the amino acid sequence ARWGGKRGGAFDI (SEQ ID NO:47), (4) a VL CDR1 comprising the amino acid sequence QSISSH (SEQ ID NO:48), (5) a VL CDR 2 comprising the amino acid sequence AAS (SEQ ID NO: 49), and (6) a VL CDR3 comprising the amino acid sequence QQSYSTPYT (SEQ ID NO: 50).

In another specific embodiment, provided herein is an antibody that specifically binds to a Zika virus NS1, wherein the antibody comprises: (1) a VH CDR1 comprising the amino acid sequence GFTVSSNY (SEQ ID NO:73), (2) a VH CDR2 comprising the amino acid sequence IYSGGST (SEQ ID NO:74), (3) a VH CDR3 comprising the amino acid sequence ARLIAAAGDY (SEQ ID NO:75), (4) a VL CDR1 comprising the amino acid sequence QSISSY (SEQ ID NO:76), (5) a VL CDR2 comprising the amino acid sequence AAS (SEQ ID NO:77), and (6) a VL CDR3 comprising the amino acid sequence QQSYSTPWT (SEQ ID NO:78).

In another specific embodiment, provided herein is an antibody that specifically binds to a Zika virus NS1, wherein the antibody comprises: (1) a VH CDR1 comprising the amino acid sequence GFTVSSNY (SEQ ID NO:101), (2) a VH CDR2 comprising the amino acid sequence IYSGGST (SEQ ID NO:102), (3) a VH CDR3 comprising the amino acid sequence ARGPVQLERRPLGAFDI (SEQ ID NO:103), (4) a VL CDR1 comprising the amino acid sequence KLGDKY (SEQ ID NO: 104), (5) a VL CDR2 comprising the amino acid sequence QDS (SEQ ID NO:105), and (6) a VL CDR3 comprising the amino acid sequence QAWDSSTVV (SEQ ID NO:106).

In another specific embodiment, provided herein is an antibody that specifically binds to a Zika virus NS1, wherein the antibody comprises: (1) a VH antibody binding region (ABR)1 comprising the amino acid sequence FTVSSNYMS (SEQ ID NO:31), (2) a VH ABR2 comprising the amino acid sequence WVSVIYSGGSTYYA (SEQ ID NO: 32), (3) a VH ABR3 comprising the amino acid sequence ARDRRGFDY (SEQ ID NO:33), (4) a VL ABR1 comprising the amino acid sequence QSISSYLN (SEQ ID NO:34), (5) a VL ABR2 comprising the amino acid sequence LLIYAASSLQS (SEQ ID NO: 35), and (6) a VL ABR3 comprising the amino acid sequence QQTYSTPL (SEQ ID NO: 36).

In another specific embodiment, provided herein is an antibody that specifically binds to a Zika virus NS1, wherein the antibody comprises: (1) a VH ABR1 comprising the amino acid sequence FTVSSNYMS (SEQ ID NO: 59), (2) a VH ABR2 comprising the amino acid sequence WVSVIYSGGSTYYA (SEQ ID NO: 60), (3) a VH ABR3 comprising the amino acid sequence ARWGGKRGGAFDI (SEQ ID NO:61), (4) a VL ABR1 comprising the amino acid sequence QSISSHLN (SEQ ID NO: 62), (5) a VL ABR2 comprising the amino acid sequence FLIYAASSLQS (SEQ ID NO: 63), and (6) a VL ABR3 comprising the amino acid sequence QQSYSTPY (SEQ ID NO:64).

In another specific embodiment, provided herein is an antibody that specifically binds to a Zika virus NS1, wherein the antibody comprises: (1) a VH ABR1 comprising the amino acid sequence FTVSSNYMS (SEQ ID NO:87), (2) a VH ABR2 comprising the amino acid sequence WVSVIYSGGSTYYA (SEQ ID NO:88), (3) a VH ABR3 comprising the amino acid sequence ARLIAAAGDY (SEQ ID NO:89), (4) a VL ABR1 comprising the amino acid sequence QSISSYLN (SEQ ID NO:90), (5) a VL ABR2 comprising the amino acid sequence LLIYAASSLQS (SEQ ID NO: 91), and (6) a VL ABR3 comprising the amino acid sequence QQSYSTPW (SEQ ID NO:92).

In another specific embodiment, provided herein is an antibody that specifically binds to a Zika virus NS1, wherein the antibody comprises: (1) a VH ABR1 comprising the amino acid sequence FTVSSNYMS (SEQ ID NO:115), (2) a VH ABR2 comprising the amino acid sequence WVSVIYSGGSTYYA (SEQ ID NO:116), (3) a VH ABR3 comprising the amino acid sequence ARGPVQLERRPLGAFDI (SEQ ID NO:117), (4) a VL ABR1 comprising the amino acid sequence KLGDKYAC (SEQ ID NO:118), (5) a VL ABR2 comprising the amino acid sequence LVIYQDSKRPS (SEQ ID NO:119), and (6) a VL ABR3 comprising the amino acid sequence QAWDSSTV (SEQ ID NO:120).

In another specific embodiment, provided herein is an antibody that specifically binds to a Zika virus NS1, wherein the antibody comprises a variable heavy chain region comprising the amino acid sequence of SEQ ID NO: 9 and a variable light chain region comprising the amino acid sequence of SEQ ID NO: 10. In another specific embodiment, provided herein is an antibody that specifically binds to a Zika virus NS1, wherein the antibody comprises a variable heavy chain region comprising the amino acid sequence of SEQ ID NO: 11 and a variable light chain region comprising the amino acid sequence of SEQ ID NO: 12. In another specific embodiment, provided herein is an antibody that specifically binds to a Zika virus NS1, wherein the antibody comprises a variable heavy chain region comprising the amino acid sequence of SEQ ID NO: 13 and a variable light chain region comprising the amino acid sequence of SEQ ID NO: 14. In another specific embodiment, provided herein is an antibody that specifically binds to a Zika virus NS1, wherein the antibody comprises a variable heavy chain region comprising the amino acid sequence of SEQ ID NO: 15 and a variable light chain region comprising the amino acid sequence of SEQ ID NO: 16.

In another specific embodiment, provided herein is an antibody that specifically binds to a Zika virus NS1, wherein the antibody comprises a variable heavy chain region that is at least 95% identical to the amino acid sequence of SEQ ID NO: 9 and a variable light chain region that is at least 95% identical to the amino acid sequence of SEQ ID NO: 10. In particular embodiments, the variable heavy chain region comprises a VH CDR1 comprising the amino acid sequence GFTVSSNY (SEQ ID NO:17), (2) a VH CDR2 comprising the amino acid sequence IYSGGST (SEQ ID NO:18), and (3) a VH CDR3 comprising the amino acid sequence ARDRRGFDY (SEQ ID NO:19); and the variable light chain region comprises (1) a VL CDR1 comprising the amino acid sequence QSISSY (SEQ ID NO:20), (2) a VL CDR2 comprising the amino acid sequence AAS (SEQ ID NO: 21), and (3) a VL CDR3 comprising the amino acid sequence QQTYSTPLT (SEQ ID NO:22).

In another specific embodiment, provided herein is an antibody that specifically binds to a Zika virus NS1, wherein the antibody comprises a variable heavy chain region that is at least 95% identical to the amino acid sequence of SEQ ID NO: 11 and a variable light chain region that is at least 95% identical to the amino acid sequence of SEQ ID NO: 12. In particular embodiments, the variable heavy chain region comprises (1) a VH CDR1 comprising the amino acid sequence GFTVSSNY (SEQ ID NO:45), (2) a VH CDR2 comprising the amino acid sequence IYSGGST (SEQ ID NO:46), and (3) a VH CDR3 comprising the amino acid sequence ARWGGKRGGAFDI (SEQ ID NO:47); and the variable light chain region comprises (1) a VL CDR1 comprising the amino acid sequence QSISSH (SEQ ID NO:48), (2) a VL CDR 2 comprising the amino acid sequence AAS (SEQ ID NO:49), and (3) a VL CDR3 comprising the amino acid sequence QQSYSTPYT (SEQ ID NO:50).

In another specific embodiment, provided herein is an antibody that specifically binds to a Zika virus NS1, wherein the antibody comprises a variable heavy chain region that is at least 95% identical to the amino acid sequence of SEQ ID NO: 13 and a variable light chain region that is at least 95% identical to the amino acid sequence of SEQ ID NO: 14. In particular embodiments, the variable heavy chain region comprises (1) a VH CDR1 comprising the amino acid sequence GFTVSSNY (SEQ ID NO:73), (2) a VH CDR2 comprising the amino acid sequence IYSGGST (SEQ ID NO:74), and (3) a VH CDR3 comprising the amino acid sequence ARLIAAAGDY (SEQ ID NO:75); and the variable light chain region comprises (1) a VL CDR1 comprising the amino acid sequence QSISSY (SEQ ID NO:76), (2) a VL CDR2 comprising the amino acid sequence AAS (SEQ ID NO:77), and (3) a VL CDR3 comprising the amino acid sequence QQSYSTPWT (SEQ ID NO:78).

In another specific embodiment, provided herein is an antibody that specifically binds to a Zika virus NS1, wherein the antibody comprise a variable heavy chain region comprising the amino acid sequence of SEQ ID NO: 15 and a variable light chain region comprising the amino acid sequence of SEQ ID NO: 16. In particular embodiments, the variable heavy chain region comprises (1) a VH CDR1 comprising the amino acid sequence GFTVSSNY (SEQ ID NO:101), (2) a VH CDR2 comprising the amino acid sequence IYSGGST (SEQ ID NO: 102), and (3) a VH CDR3 comprising the amino acid sequence ARGPVQLERRPLGAFDI (SEQ ID NO:103); and the variable light chain region comprises (1) a VL CDR1 comprising the amino acid sequence KLGDKY (SEQ ID NO:104), (2) a VL CDR2 comprising the amino acid sequence QDS (SEQ ID NO:105), and (3) a VL CDR3 comprising the amino acid sequence QAWDSSTVV (SEQ ID NO:106).

In certain embodiments, an antibody provided herein is a monoclonal antibody. In some embodiments, an antibody provided herein is a single chain antibody. In certain embodiments, an antibody provided herein is an Fab or F(ab′)₂ fragment. In a specific embodiment, an antibody provided herein is a recombinant antibody.

In another aspect, provided herein is an antibody conjugate comprising (a) an antibody moiety that is an antibody described herein; (b) a drug moiety; and (c) optionally a linker, wherein the drug moiety is conjugated to the antibody moiety directly or is conjugated to the antibody moiety via a linker. In certain embodiments, the drug moiety is cytotoxic. In one embodiment, an antibody conjugate described herein or a composition thereof may be used to prevent a Zika virus disease. In another embodiment, an antibody conjugate described herein or a composition thereof may be used to treat a Zika virus infection or a Zika virus disease. In another embodiment, an antibody conjugate described herein or a composition thereof may be used in an immunoassay. In another embodiment, an antibody conjugate described herein or a composition thereof may be used to detect or diagnose a Zika virus infection

In another aspect, provided herein is an antibody conjugate comprising (a) an antibody moiety that is an antibody described herein; (b) a detectable moiety (e.g., a detectable substance); and (c) optionally a linker, wherein the detectable moiety is conjugated to the antibody moiety directly or is conjugated to the antibody moiety via a linker. In some embodiments, the detectable moiety is horseradish peroxidase, alkaline phosphatase, beta-galactosidase, acetylcholinesterase, streptavidin/biotin, avidin/biotin, a fluorescent material, or a positron emitting metal.

In another aspect, provided herein is a pharmaceutical composition comprising an effective amount of an antibody described herein in an admixture with a pharmaceutically acceptable carrier. In another aspect, provided herein is a pharmaceutical composition comprising an effective amount of an antibody conjugate described herein in an admixture with a pharmaceutically acceptable carrier.

In another aspect, provided herein is a method for detecting a Zika virus infection in a biological sample, comprising contacting an antibody described herein with the biological sample and detecting the binding of the antibody or antibody conjugate to a Zika virus NS1. In another aspect, provided herein is a method for detecting a Zika virus infection in a biological sample, comprising contacting an antibody conjugate described herein with the biological sample and detecting the binding of the antibody or antibody conjugate to a Zika virus NS1. In certain embodiments, the biological sample is from a subject. The biological sample may be blood, serum, plasma, cells or a tissue sample.

In another aspect, provided herein is a method for diagnosing a Zika virus infection in a subject, comprising contacting an antibody described herein with a biological sample from the subject and detecting the binding of the antibody or antibody conjugate to a Zika virus NS1, wherein an increase in the detection of the binding of the antibody or antibody conjugate in the biological sample relative to the detection of binding of the antibody or antibody conjugate to a negative control sample indicates that the subject has a Zika virus infection. In another aspect, provided herein is a method for diagnosing a Zika virus infection in a subject, comprising contacting or an antibody conjugate described herein with a biological sample from the subject and detecting the binding of the antibody or antibody conjugate to a Zika virus NS1, wherein an increase in the detection of the binding of the antibody or antibody conjugate in the biological sample relative to the detection of binding of the antibody or antibody conjugate to a negative control sample indicates that the subject has a Zika virus infection. In certain embodiments, the biological sample is from a subject. The biological sample may be blood, serum, plasma, cells or a tissue sample.

In another aspect, provided herein is a method of distinguishing Zika virus from Dengue virus in a biological sample, comprising contacting an antibody described herein with the biological sample and detecting the binding of the antibody or antibody conjugate to a Zika virus NS1, wherein an increase in the detection of the binding of the antibody or antibody conjugate relative to the detection of binding of the antibody or antibody conjugate to a sample containing Dengue virus indicates the presence of Zika virus in the biological sample. In another aspect, provided herein is a method of distinguishing Zika virus from Dengue virus in a biological sample, comprising contacting an antibody conjugate described herein with the biological sample and detecting the binding of the antibody or antibody conjugate to a Zika virus NS1, wherein an increase in the detection of the binding of the antibody or antibody conjugate relative to the detection of binding of the antibody or antibody conjugate to a sample containing Dengue virus indicates the presence of Zika virus in the biological sample. In certain embodiments, the biological sample is from a subject. The biological sample may be blood, serum, plasma, cells or a tissue sample.

In another aspect, provided herein is a method for preventing a Zika virus infection in a subject, comprising administering to the subject a pharmaceutical composition described herein. In another aspect, provided herein is a method for treating a Zika virus infection or a Zika virus disease in a subject, comprising administering to the subject a pharmaceutical composition described herein. In specific embodiments, the subject is a human subject.

In another aspect, provided herein is an isolated nucleic acid sequence comprising a nucleotide sequence encoding an antibody described herein. In another aspect, provided herein is a vector(s) (e.g., an expression vector(s)) comprising a nucleotide sequence encoding an antibody described herein. In a specific embodiment, provided herein is a vector (e.g., an expression vector) comprising a nucleotide sequence encoding a variable heavy chain region or heavy chain of an antibody described herein. In another specific embodiment, provided herein is a vector (e.g., an expression vector) comprising a nucleotide sequence encoding a variable light chain region or light chain of an antibody described herein.

In another aspect, provided herein is a host cell(s) comprising nucleic acid sequence comprising a nucleotide sequence encoding an antibody described herein. In a specific embodiment, provided herein is a host cell(s) engineered to express a nucleotide sequence encoding an antibody described herein. In a specific embodiment, provided herein is a host cell(s) comprising a first vector (e.g., an expression vector) and a second vector (e.g., an expression vector), wherein the first vector comprises a nucleotide sequence encoding a variable heavy chain region or heavy chain of an antibody described herein and the second vector comprises a nucleotide sequence encoding a variable light chain region or light chain of the antibody described herein. In a specific embodiment, the host cell(s) is isolated.

In another aspect, provided herein is a method for producing an antibody described herein comprising culturing a host cell expressing an antibody described herein and isolating the antibody from the cell culture.

In another aspect, provided herein is an NS1 polypeptide comprising the amino acid sequence of a Zika virus NS1 and the amino acid sequence of a fragment of a Zika virus envelope protein, wherein the amino acid sequence of the fragment of the Zika virus envelope protein is at the N-terminus of the amino acid sequence of the Zika virus NS1. In a specific embodiment, the fragment of the Zika virus envelope protein comprises the last 20 to 50 carboxy-terminal amino acid residues of the Zika virus envelope protein. In certain embodiments, the fragment of the Zika virus envelope protein comprises the last 24 carboxy-terminal amino acid residues of the Zika virus envelope protein. In a specific embodiment, the fragment of the Zika virus envelope protein comprises the amino acid sequence NGSISLMCLALGGVLIFLSTAVSA (SEQ ID NO: 131). In certain embodiments, the Zika virus NS1 comprises the amino acid sequence of the NS1 of Zika virus PRVABC59. In some embodiments, the NS1 polypeptide further comprises a cleavage site and a tag. In a specific embodiment, the cleavage site is LEVLENGPG (SEQ ID NO: 132). In another specific embodiment, the tag is a hexahistidine motif. In another specific embodiment, the cleavage site and the tag are at the carboxy terminus of the NS1 polypeptide. In particular embodiment, an NS1 polypeptide provided herein is a recombinant NS1 polypeptide.

In another aspect, provided herein is an isolated nucleic acid sequence comprising a nucleotide sequence encoding an NS1 polypeptide described herein. In a specific embodiment, the nucleotide sequence is human codon-optimized.

In another aspect, provided herein is a vector (e.g., an expression vector) comprising a nucleotide sequence encoding an NS1 polypeptide described herein. In a specific embodiment, provided herein is a viral vector comprising a genome that comprises a nucleotide sequence encoding an NS1 polypeptide described herein.

In another aspect, provided herein is a host cell(s) comprising a nucleic acid sequence comprising a nucleotide sequence encoding an NS1 polypeptide described herein. In a specific embodiment, provided herein is a host cell(s) engineered to express a recombinant NS1 polypeptide. In another specific embodiment, the host cell(s) is isolated.

In another aspect, provided herein is a method for producing an NS1 polypeptide described herein comprising culturing a host cell expressing an NS1 polypeptide described herein and isolating the antibody from the cell culture.

In another aspect, provided herein is a pharmaceutical composition (e.g., an immunogenic composition) comprising a nucleic acid sequence comprising a nucleotide sequence encoding an NS1 polypeptide described herein in an admixture with a pharmaceutically acceptable carrier. In another aspect, provided herein is a pharmaceutical composition (e.g., an immunogenic composition) comprising an NS1 polypeptide described herein in an admixture with a pharmaceutically acceptable carrier.

In another aspect, provided herein is a method for immunizing against Zika virus, comprising administering to a subject a dose of an NS1 polypeptide described herein or a pharmaceutical composition thereof (e.g., an immunogenic composition). In another aspect, provide herein is method for preventing a Zika virus-mediated disease, comprising administering to a subject a dose of an NS1 polypeptide described herein or a pharmaceutical composition thereof (e.g., an immunogenic composition). In another aspect, provided herein is a method for inducing an immune response to a Zika virus NS1, comprising administering to a subject a dose of a dose of an NS1 polypeptide described herein or a pharmaceutical composition thereof (e.g., an immunogenic composition). In certain embodiments, the method further comprises the administration of one or more boost doses of an NS1 polypeptide described herein or a pharmaceutical composition thereof (e.g., an immunogenic composition). In a specific embodiment, the subject is a human. In another specific embodiment, the subject is a pregnant human subject.

In another aspect, provided herein is a method for immunizing against Zika virus, comprising: (a) administering to a subject a dose of a first pharmaceutical composition (e.g., an immunogenic composition) comprising a nucleic acid sequence comprising a nucleotide sequence encoding an NS1 polypeptide described herein or a composition thereof; and (b) after a first certain period of time administering to the subject a dose of a second pharmaceutical composition (e.g., an immunogenic composition) comprising an NS1 polypeptide described herein. In another aspect, provided herein is a method for immunizing against Zika virus, comprising: (a) administering to a subject a dose of a first pharmaceutical composition comprising a vector comprising a nucleotide sequence encoding an NS1 polypeptide described herein or a composition thereof; and (b) after a first certain period of time administering to the subject a dose of a second pharmaceutical composition comprising an NS1 polypeptide described herein. In certain embodiments, the method further comprises administering a second dose of the second pharmaceutical composition after a second certain period of time. In some embodiments, the first certain period of time, the second certain period of time, or both are 2 weeks, 3 weeks, 1 month, 3 months, or 6 months. In a specific embodiment, the subject is a human. In another specific embodiment, the subject is a pregnant human subject.

### 4. BRIEF DESCRIPTION OF THE DRAWINGS

FIGS. 1A-1C. Human ZIKV specific-antibodies bind NS1 protein of both MR766 and PRVABC59 Zika viruses. FIG. 1A. Vero cells were infected with the indicated viruses at an MOI of 1 for 24 hours. The cells were fixed with 0.5% paraformaldehyde and blocked with 5% non-fat milk. MAbs AA12, FC12, EB9 and GB5 were used at a concentration of 5 μg/mL and an anti-human antibody conjugated to Alexa Fluor 488 was used as a secondary antibody. The murine pan-flavivirus mAb 4G2 was used as a positive control and an anti-mouse antibody conjugated to Alexa Fluor 488 was used as a secondary antibody. Cells stained with mAb 4G2 were fixed and permeabilized using 80% acetone. FIGS. 1B, 1C. ELISA assays were performed using recombinant NS1 protein from either MR766 or PRVABC59 viruses to assess the binding activity of mAbs AA12, FC12, EB9 and GB5. ELISAs were performed in duplicates. Data plotted represent mean values and the standard error of the mean (SEM); a non-linear regression line was generated using GraphPad Prism 5.

FIGS. 2A-2D. NS1-specific antibodies activate Fc-FcR effector functions in vitro. To examine the ability of NS1-specific antibodies to activate Fc-FcR mediated effector functions, FIGS. 2A, 2B. Vero cells were infected with MR766 and PRVABC59 Zika viruses or FIGS. 2C, 2D. HEK 293T cells were transfected with NS1 from MR766 and PRVABC59 Zika viruses. Infected Vero cells or transfected HEK 293T cells were used as targets for measuring antibody-mediated effector functions with a genetically modified Jurkat cell line expressing the human FcRγIIIa with an inducible luciferase reporter gene. Fold induction was measured in relative light units and calculated by subtracting background signal from wells without effector cells then dividing wells with antibody by wells with no antibody added. All mAbs were tested at a starting concentration of 10 μg/mL and were serially diluted four-fold. Assays were performed twice as technical duplicates and one of two replicates is shown. A non-linear regression best-fit curve was generated for each dataset using GraphPad Prism 5. Error bars represent SEM.

FIGS. 3A-3B. NS1-specific antibodies do not cause antibody-dependent enhancement (ADE) of infection in vitro. To examine whether enhancement of ZIKV infection in vitro is observed by NS1-specific antibodies, mAbs or pooled serum from a DENV positive donor were incubated with FIG. 3A. PRVABC59 or FIG. 3B. DENV-3 viruses and added to FcγR bearing K562 cells. All mAbs were tested at a starting concentration of 100 ng/ml and were serially diluted four-fold. DENV positive control sera was diluted five-fold initially and serially diluted four-fold. The assay was run in duplicate and -fold induction was measured as number of infected cells as measured by flow cytometry divided by infected cells with no antibody or serum added. Sera were obtained through a screening of blood donations in Puerto Rico as described previously in Bardina et al. [14]. Plotted values represent mean value and standard deviation.

FIGS. 4A-4E. NS1-specific antibodies protect mice against lethal challenge in vivo. FIGS. 4A-4C Groups of 6 to 9 male and female B6.129-Stat2−/− mice were injected IP with 20 mg per kg of AA12 before a challenge with 10 mLD50 of ZIKV MR766 intradermally. A mAb (CR9114) against influenza A virus was used as an IgG control. FIGS. 4D, 4E. Mice were treated with 10 mg per kg of AA12 or 10 mg per kg of isotype control before a challenge with 500 PFU of ZIKV PAN/2015 retro-orbitally. Weight loss was monitored daily. For mice infected with ZIKV MR766, clinical scoring was conducted using the pre-defined criteria with a maximum possible score of 7: impact on walking (1), unresponsiveness (1), left hind leg paralyzed (1), right hind leg paralyzed (1), left front leg paralyzed (1), and right front leg paralyzed (1). Deceased animals were awarded a score of 7. The ratios in the figures indicate the number of animals that survived challenge over total number of animals per group. Murine challenge studies were performed as two independent replicates with at least three mice per treatment group and data shown here were pooled. The Mantel-Cox and Gehan-Breslow-Wilcoxon tests were used to analyze statistical significance of survival between two groups. A multiple t-test and the Holm-Sidak method were used to determine statistical significance at each time point for the weight curve and the clinical score. Asterisk(s) indicates statistical significance of a group (*=p<0.05 and **=p<0.005) compared to control IgG. No significant differences between groups were detected in FIG. 4D.

FIGS. 5A-5D. LALAPG mutation ablates Fc-FcR mediated effector functions without affecting affinity. The variable region of AA12 was cloned into human IgG1 or human IgG1 with L234A, L235A, and P329G mutations (LALAPG) in the backbone. FIGS. 5A, 5B. ELISA assays were performed using recombinant NS1 from either MR766 and PRVABC59 viruses to assess the binding activities of mAbs AA12 and AA12 LALAPG. FIGS. 5C, 5D. Fc-FcR mediated effector functions were tested on Vero cells infected with MR766 and PRVABC59 Zika viruses. AA12 was able to elicit Fc-FcR mediated effector functions while AA12 LALAPG was not. Assays were performed twice as technical duplicates and one of two replicates is shown. A non-linear regression best-fit curve was generated for each dataset using GraphPad Prism 5. Error bars represent SEM.

FIGS. 6A-6C. NS1-specific antibodies protect mice against lethal challenge in vivo in an Fc-dependent manner. FIGS. 6A-6C. Groups of 4 to 13 male and female B6.129-Stat2−/− mice were injected IP with 10 mg per kg of wildtype AA12, AA12 LALAPG, AA12 LALA, or AA12 PG Fc-variants before a challenge with 10 mLD50 of ZIKV MR766. The mAb CR9114 against influenza A virus was used as an IgG isotype control. Weight loss was monitored daily. Clinical scoring was conducted using the pre-defined criteria with a maximum possible score of 7: impact on walking (1), unresponsiveness (1), left hind leg paralyzed (1), right hind leg paralyzed (1), left front leg paralyzed (1), and right front leg paralyzed (1). Deceased animals were awarded a score of 7. The ratios in the figures indicate the number of animals that survived challenge over total number of animals per group. Murine challenge studies were performed as three independent replicates with at least four mice per treatment group and data shown here were pooled. Statistical analyses were performed using the Mantel-Cox and Gehan-Breslow-Wilcoxon tests for the survival curves and a multiple t-test and the Holm-Sidak method for the weight curve and the clinical score. Significance (*=p<0.05) is indicated compared to IgG control. No significant differences between the groups were detected in FIG. 6A.

FIGS. 7A-7B. Activation of human primary natural killer cells. Vero cells were infected with PRVABC59 at an MOI of 0.5. At 48 hpi, dilutions of mAb (starting at 20 μg/mL), irrelevant mAb or media containing no mAb (negative control) were added to ZIKV-infected Vero cells and incubated for 1 hour at 37° C. Primary human NK cells from donor 1 (FIG. 7A) and 2 (FIG. 7B) were then added (effector to target ratio of 2) and the culture was further incubated at 37° C. for three hours. The cells were then stained with CD56 (FITC) and CD107a (PE). Activation of NK cells (expression of CD107a) were detected using a flow cytometer and analyzed using Flowjo software. Data represent percent of NK cells expressing CD107a and are the mean value of duplicates and the SEM. A multiple t-test and the Holm-Sidak method was performed using GraphPad Prism. Significance (*=p<0.05) is indicated compared to control IgG. Dotted line on the y-axis represent the average value of the negative control group (media with no mAb).

FIGS. 8A-8C. Challenge model of NS1-specific antibodies against PAN/2015. Groups of 4 male and female B6.129-Stat2−/− mice were injected IP with 10 mg per kg of EB9, FC12, or IgG control before a challenge with 500 PFU of ZIKV PAN/2015 retro-orbitally. FIGS. 8A, 8B. Weight loss was monitored daily and mice that lost 25% of their original weight were sacrificed. FIG. 8C. Clinical scoring was conducted using the pre-defined criteria with a maximum possible score of 7: impact on walking (1), unresponsiveness (1), left hind leg paralyzed (1), right hind leg paralyzed (1), left front leg paralyzed (1), and right front leg paralyzed (1). Deceased animals were given a score of 7. The ratios in the figures indicate the number of animals that survived challenge over total number of animals per group. Statistical analyses were performed using the Mantel-Cox and Gehan-Breslow-Wilcoxon tests for the survival curves and a multiple t-test and the Holm-Sidak method for the weight curve and the clinical score. No significant differences between groups were detected in FIGS. 8A, 8B and 8C.

FIGS. 9A-9B. Viral burden in brains and spleens of infected mice. Groups of 6 male and female B6.129-Stat2−/− mice were injected IP with 10 mg per kg of AA12 or IgG control before a challenge with 500 PFU of ZIKV PAN/2015 retro-orbitally. FIG. 9A. Brains and FIG. 9B. spleens were harvested at days 3 and days 6 post infection. Viral titers were determined by plaque assay. Error bars represent SEM. A two-way ANOVA followed by a Holm-Sidak multiple comparison analysis was performed using GraphPad Prism 5 and yielded a significant difference between control and AA12-treated mice in the spleen at 3 dpi.

FIGS. 10A-10D. LALA and PG Fc-variants of AA12. FIGS. 10A, 10B. ELISA assays were performed using recombinant NS1 from either MR766 and PRVABC59 viruses to assess the binding activities of mAbs AA12 and AA12 variants. FIGS. 10C, 10D. Fc-FcR mediated effector functions were tested on Vero cells infected with MR766 and PRVABC59 Zika viruses. AA12 was able to elicit Fc-FcR mediated effector functions while AA12 variants were not.

FIG. 11. Complement levels are raised during acute ZIKV infection. Groups of 3 to 4 male and female B6.129-Stat2^{-/-}-mice were injected IP with 10 mg per kg of AA12 wild-type, AA12 LALAPG, AA12 LALA, AA12 PG or IgG control before a challenge with 10 mLD50 of ZIKV MR766 intradermally. At day 6 post infection C3 levels were determined by ELISA as per manufacturer's instructions. A one-way ANOVA and Dunnett's multiple comparison tests was performed to determine statistical significance of the mAb-treated groups to the naïve uninfected mice (*=p<0.05 **=p<0.005).

FIGS. 12A-12D. Generation of expression plasmids encoding ZIKV NS1. (FIG. 12A) The human codon-optimized NS1 of ZIKV PRVABC59 was subcloned into a mammalian expression vector, pCAGGS, which includes the last 24 amino acids of the envelope protein at the amino terminus followed by the NS1 coding region (pCAGGS NS1). Of note, the first amino acid of the NS1 coding region is indicated by a bold, red aspartic acid residue. (FIG. 12B) A second version (pCAGGS NS1-HIS) also encodes the ZIKV PRVABC59 NS1 followed by a PreScission Protease cleavage site (LEVLFNGPG (SEQ ID NO:132; blue region) and a hexahistidine motif (HHHHHH (SEQ ID NO:133); orange region) at the carboxy terminus. (FIG. 12C) HEK 293T cells were transfected with pCAGGS NS1, pCAGGS NS1-HIS or not transfected (mock). At 24 hours post transfection, the cells were fixed with 0.5% paraformaldehyde and surface expression of NS1 was detected using an anti-ZIKV NS1 monoclonal antibody AA12 or a polyclonal anti-histidine antibody. Secondary antibodies conjugated to Alexa Fluor 488 were used to visualize binding using a Celigo imaging cytometer. The scale bars are equal to 500 microns. (FIG. 12D) HEK 293F cells were transfected with pCAGGS NS1-HIS and a four days post transfection, soluble NS1 from the supernatant and cell lysates were collected and purified over a NI-NTA column. Soluble NS1 from the supernatant and lysates were resolved in an SDS-PAGE gel and detected using a polyclonal anti-histidine antibody in a Western blot assay. BSA was used as a negative control and a HIS-tagged soluble hemagglutinin of A/Perth/16/09 (H3N2) was used as a positive control. FIGS. 13A-13G. Zika Virus NS1 vaccine induces a robust and functional antibody response in mice (FIG. 13A) Schematic outlining the vaccination strategy, where mice were prime immunized with 80 μg of pCAGGS NS1 DNA plasmid via electroporation and followed by two boost immunizations of soluble NS1 proteins. All vaccinations were administered intramuscularly. (FIG. 13B) Groups of mice vaccinated with each adjuvant used for the protein components. (FIGS. 13C-13E) The antibody response to NS1 (PRVABC59 ZIKV) was measured by ELISA. Each data point denotes an individual animal, while the each color represents one group of mice. The time points are after the DNA prime at day 21, after the protein boost at day 42, or sera from the terminal bleed at day 84. ELISA data was run in duplicate and shown as area under the curve (AUC). A non-parametric multiple comparisons Kruskal-Wallis test was used to determine statistical significance at each time point. Asterisks indicates statistical significance of a group (*=p<0.05, **=p<0.01, ***=p<0.001, ****=p<0.0001) compared to naïve serum. No significance was observed between control groups and the naïve group. (FIGS. 13F-13G) To examine the ability of NS1-specific antibodies to activate Fc-mediated effector functions, Vero cells were infected with PRVABC59 ZIKV or HEK 293T cells were transfected with an NS1 expression plasmid (PCAGGS-NS1). Infected Vero cells or transfected HEK 293T cells were used as targets for measuring antibody-mediated effector functions with a genetically modified Jurkat cell line expressing the murine FcRγIV with an inducible luciferase reporter gene. Fold induction was measured in relative light units and calculated by subtracting background signal from wells without effector cells then dividing wells with sera by wells with no sera added. All sera were tested at a starting dilution of 1:75 and were serially diluted three-fold in duplicate. A non-linear regression best-fit curve was generated for each dataset using GraphPad Prism 6. Error bars represent SEM.

FIG. 14A-14F. Passive transfer of serum from vaccinated mice protects against lethal challenge (FIGS. 14A-14C) Groups of 4-5 male and female B6.129-Stat2−/− mice were injected IP with 200 μL of pooled serum before a challenge with 10LD50 (158 PFU per mouse) of MR766 ZIKV intradermally. (FIGS. 14D-14F) Groups of 4 male and female B6.129-Stat2−/− mice were injected IP with 200 μL of pooled serum before a challenge with 1000 PFU of PRVABC59 ZIKV intradermally. Weight loss was monitored daily. Clinical scoring was conducted using the pre-defined criteria with a maximum possible score of 7: impact on walking (1), unresponsiveness (1), left hind leg paralyzed (1), right hind leg paralyzed (1), left front leg paralyzed (1), and right front leg paralyzed (1). Deceased animals were awarded a score of 7. The ratios in the figures indicate the number of animals that survived challenge over total number of animals per group. The Mantel-Cox and Gehan-Breslow-Wilcoxon tests were used to analyze statistical significance of survival between two groups. A multiple t-test and the Holm-Sidak method were used to determine statistical significance at each time point for the weight curve and the clinical score. Asterisks indicates statistical significance of a group (*=p<0.05) compared to mice vaccinated with BSA.

FIG. 15A-15F. NS1-specific antibodies are long-lived and functional in ZIKV infected humans. ELISA data of individual human sera against recombinant NS1 protein from PRVABC59 ZIKV. ELISA data was run in duplicate and values represent area under the curve (AUC). (FIG. 15B) ELISA data of select human samples with repeat blood draws. Each color represents an individual patient (FIG. 15C-15F). To test the ability of human sera to activate Fc-mediated effector functions, Vero cells were infected with PRVABC59 ZIKV and an Fc-FcgR reporter assay was performed as previously shown. The legend includes the patient identifier number and days post onset of symptoms (DPO). All sera were tested at a starting dilution of 1:75 and were serially diluted three-fold in duplicate. A non-linear regression best-fit curve was generated for each dataset using GraphPad Prism 6. Error bars represent SEM.

FIG. 16A-16D. Human sera can engage FcgRs when targeting NS1 transfected cells. To examine the ability of human sera to activate NS1-specific Fc-mediated effector functions, (FIGS. 16A-16D) HEK 293T cells were transfected with an NS1 (PCAGGS-NS1) expression plasmid. Each color represents an individual sample and the legend includes the patient identifier number and days post onset (DPO) of symptoms. A surrogate in vitro reporter assay for measuring Fc-FcgR interactions was performed as previously shown. All sera were tested at a starting dilution of 1:75 and were serially diluted three-fold in duplicate. A non-linear regression best-fit curve was generated for each dataset using GraphPad Prism 6. Error bars represent SEM.

FIGS. 17A-17C. Cross-reactive envelope-specific antibodies do not elicit Fc-mediated responses. Sera from TBEV vaccinated patients were analyzed for binding to ZIKV proteins and the ability to elicit Fc-mediated effector functions. Sera were obtained through a screening of TBEV vaccinee samples as described previously in Duehr et al. (FIG. 17A) ELISAS performed on recombinant ZIKV envelope protein or (FIG. 17B) recombinant NS1 protein from PRVABC59 ZIKV. All sera were tested at a starting dilution of 1:40 and were serially diluted four-fold in duplicate. “Acute ZIKV infection” is serum from a patient with an acute infection confirmed by RT-PCR. (FIG. 17C) Vero cells were infected with PRVABC59 ZIKV and a surrogate in vitro reporter assay was performed to measure Fc-FcgR interactions. All sera were tested at a starting dilution of 1:25 and were serially diluted three-fold in duplicate.

FIG. 18. Immunofluorescence of pooled mouse serum. Vero cells were infected with PRVABC59 ZIKV at an MOI of 0.5 for 24 hours. The cells were fixed with 0.5% paraformaldehyde and blocked with 5% non-fat milk. Serum were added at a dilution of 1:100 and an anti-mouse antibody conjugated to Alexa Fluor 488 was used as a secondary antibody. Scale bars are equal to 200 microns.

FIGS. 19A-B. Amino acid sequences of the variable heavy and light chain regions of human antibody AA12 (SEQ ID Nos: 9 and 10, respectively) with the complementarity determining regions (CDRs) delineated by the IMGT numbering system in FIG. 19A and the antibody binding regions (ABR) delineated by the Paratome system in FIG. 19B.

FIGS. 20A-20B. Amino acid sequences of the variable heavy and light chain regions of human antibody EB9 (SEQ ID Nos: 11 and 12, respectively) with the complementarity determining regions (CDRs) delineated by the IMGT numbering system in FIG. 20A and the antibody binding regions (ABR) delineated by the Paratome system in FIG. 20B.

FIGS. 21A-21B. Amino acid sequences of the variable heavy and light chain regions of human antibody GB5 (SEQ ID Nos.: 13 and 14, respectively) with the complementarity determining regions (CDRs) delineated by the IMGT numbering system in FIG. 21A and the antibody binding regions (ABR) delineated by the Paratome system in FIG. 21B. FIG. 22A-22B. Amino acid sequences of the variable heavy and light chain regions of human antibody FC12 (SEQ ID Nos: 15 and 16, respectively) with the complementarity determining regions (CDRs) delineated by the IMGT numbering system in FIG. 22A and the antibody binding regions (ABR) delineated by the Paratome system in FIG. 22B.

### 5. DETAILED DESCRIPTION

### 5.1 Zika Virus NS1

Provided herein are recombinant Zika virus NS1 immunogens (e.g., NS1 polypeptides). In one aspect, provided herein is an NS1 polypeptide comprising the amino acid sequence of a Zika virus NS1 and the amino acid sequence of a fragment of the Zika virus envelope protein, wherein the fragment of the Zika virus envelope protein is N-terminal to the amino acid sequence of the Zika virus NS1. In specific embodiments, the NS1 polypeptide comprises the full-length amino acid sequence of a Zika virus NS1. In other embodiments, the NS1 polypeptide comprises an amino acid sequence of a Zika virus NS1 that is not the full-length amino acid sequence of the Zika virus NS1. In a specific embodiment, the NS1 polypeptide comprises 1 to 5, 1 to 10, or 5 to 10 amino acid residues less than the full length amino acid sequence of a Zika virus NS1. In some embodiments, the NS1 polypeptide comprises 1 to 5, 1 to 10, or 5 to 10 amino acid residues less than the full length amino acid sequence of a Zika virus NS1 at the N-terminus or C-terminus. In certain embodiments, the NS1 polypeptide comprises 1 to 5, 1 to 10, or 5 to 10 amino acid residues less than the full length amino acid sequence of a Zika virus NS1 at the N-terminus and C-terminus. In specific embodiments, the fragment of the Zika virus envelope protein is 20, 21, 22, 23, 24, 25, 26, 27, 28, 29 or 30 amino acids in length. In some embodiments, the fragment of the Zika virus envelope protein is 20 to 25, 20 to 30, 25 to 30 amino acids in length. In certain embodiments, the fragment of the Zika virus envelope protein is 35, 40, 45, 50, 55, 60, 65, 70, 75, 80, 85, 90, 95, 100 or more amino acids in length but shorter than the full length Zika virus envelope. In some embodiments, the fragment of the Zika virus envelope protein is 100, 125, 130, 135, 140, 145, 150, 155, 160, 165, 170, 175, 180, 185, 190, 195, 200, 225, 250, 275, 300, 325, 350, 375 or 400 amino acids in length but shorter than the full length Zika virus envelope. In some embodiments, the fragment of the Zika virus envelope protein is 25 to 50, 30 to 40, 40 to 50, 25 to 75, 50 to 75, 50 to 100, or 75 to 100 amino acids in length. In certain embodiments, the fragment of the Zika virus envelope protein is from the C-terminus of the full-length Zika virus envelope protein. In a specific embodiment, the fragment of the Zika virus envelope protein comprises (or consists of) the C-terminal 20 to 25, 20 to 30, 25 to 30 amino acid residues of the Zika virus envelope. In some embodiments, the fragment of the Zika virus envelope protein comprises (or consists of) the C-terminal 20 to 25, 20 to 30, 25 to 30 amino acid residues of the Zika virus envelope protein. In certain embodiments, the fragment of the Zika virus envelope protein comprises (or consists of) the C-terminal 35, 40, 45, 50, 55, 60, 65, 70, 75, 80, 85, 90, 95, or 100 amino acid residues of the Zika virus envelope protein. In a specific embodiment, the fragment of the Zika virus envelope protein comprises (or consists of) the C-terminal 24 amino acid residues of the Zika virus envelope protein. In some embodiments, the fragment of the Zika virus envelope protein is at least 24 amino acid residues in length from the C-terminal of the Zika virus envelope protein.

In a specific embodiment, an NS1 polypeptide provided herein comprises (or consists of) the amino acid sequence of any Zika virus NS1 known to those of skill in the art. Examples of Zika viruses include Zika virus/Homo sapiens/Cuba 2017, Zika virus/H. sapiens-wt/BRA/2016/FC-DQ192D1-URI, Zika virus/H. sapiens-wt/DOM/2016/MA-WGS16-011-SER, Zika virus/H. sapiens-wt/DOM/2016/BB-0085-SER, Zika virus/H. sapiens-wt/HTI/2016/MA-WGS16-022-SER, Zika virus/H. sapiens-wt/PRI/2016/MA-WGS16-005-SER, Zika virus/H. sapiens-wt/BRA/2016/FC-DQ62D1-PLA, Zika virus/H. sapiens-wt/BRA/2016/FC-DQ60D1-URI, Zika virus/H. sapiens-wt/COL/2016/SU-2293A-SER, Zika virus/H. sapiens-wt/COL/2016/SU-1856A-SER, Zika virus/H. sapiens-wt/BRA/2016/FC-DQ68D1-URI, Zika virus/A. aegypti-wt/USA/2016/FL-08-MOS, Zika virus/H. sapiens-wt/COL/2016/SU-2724A-SER, Zika virus/H. sapiens-wt/DOM/2016/MA-WGS16-014-SER, Zika virus/H. sapiens-wt/HND/2016/HU-SZ76-SER, Zika virus/A. aegypti-wt/USA/2016/FL-06-MOS, Zika virus/H. sapiens-wt/DOM/2016/BB-0180-URI, Zika virus/H. sapiens-wt/DOM/2016/BB-0091-SER, Zika virus/H. sapiens-wt/USA/2016/FL-036-SER, and Zika virus/H. sapiens-wt/DOM/2016/MA-WGS16-013-SER. Specific examples of NS1 of Zika virus include, for example, the amino acid and nucleic acid sequences of the NS1 of MR766 Zika virus, which may be found at GenBank Accession No. MK105975, the amino acid and nucleic acid sequences of NS1 of PRVABC59 Zika virus, which may be found at GenBank Accession No. KU501215, and the amino acid sequence of NS1 of Pan/2015, which may be found at GenBank Accession No. KX156774.

In certain embodiments, an NS1 polypeptide is modified by post-translational processing such as glycosylation (e.g., N-linked glycosylation).

In certain embodiments, an NS1 polypeptide provided herein further comprises one or more polypeptide domains. Useful polypeptide domains include domains that facilitate purification, folding and cleavage of a polypeptide or portions of a polypeptide. For example, a His tag (His-His-His-His-His-His; e.g., SEQ ID NO:133), a FLAG epitope or other purification tag can facilitate purification of an NS1 polypeptide provided herein. In some embodiments, the His tag has the sequence, (His) n, wherein n is 2, 3, 4, 5, 6, 7, 8, 9, 10, 11, 12, 13, 14, 15, 16, 17, 18, 19, 20 or greater. Cleavage sites can be used to facilitate cleavage of a portion of a polypeptide, for example cleavage of a purification tag. Useful cleavage sites include a thrombin cleavage site, for example one with the amino acid sequence LVPRGSP (SEQ ID NO:141). In certain embodiments, the cleavage site is a cleavage site recognized by Tobacco Etch Virus (TEV) protease (e.g., amino acid sequence Glu-Asn-Leu-Tyr-Phe-Gln-(Gly/Ser), SEQ ID NO: 142). In a specific embodiment, the cleavage site comprises (or consists of) the amino acid sequence LEVLFNGPG (SEQ ID NO:132).

In a specific embodiment, an NS1 polypeptide comprises the amino acid sequence of NS1 of a Zika virus and the last 24 amino acid residues of a Zika virus envelope protein, wherein the 24 amino acid residues of the envelope protein are N-terminal to the amino acid sequence of the amino acid sequence of the NS1 of the Zika virus. In certain embodiments, the NS1 polypeptide further comprises a PreScission protease cleavage site (SEQ ID NO:132), a hexahistidine motif (SEQ ID NO:133), or both at the carboxy terminus of the NS1 of the Zika virus.

In a specific embodiment, an NS1 polypeptide comprises the amino acid sequence of the NS1 of Zika virus PRVABC59 and the last 24 amino acid residues of the envelope protein (SEQ ID NO:131), wherein the 24 amino acid residues of the envelope protein are N-terminal to the amino acid sequence of the amino acid sequence of the NS1 of Zika virus PRVABC59. In certain embodiments, the NS1 polypeptide further comprises a PreScission protease cleavage site (SEQ ID NO:132) and a hexahistidine motif (SEQ ID NO:133) at the carboxy terminus of the NS1 of Zika virus PRVABC59.

In a specific embodiment, an NS1 polypeptide is one described in Section 6 (e.g., 6.2, infra). In another specific embodiment, an NS1 polypeptide provided herein comprises (or consists of) the amino acid sequence of SEQ ID NO: 166 or 167.

In specific embodiments, NS1 polypeptides provided herein are capable of forming a three-dimensional structure that is similar to the three-dimensional structure of a native Zika virus NS1. Structural similarity might be evaluated based on any technique deemed suitable by those of skill in the art. For instance, reaction, e.g. under non-denaturing conditions, of an NS1 polypeptide with a neutralizing antibody or antiserum that recognizes a native Zika virus NS1 might indicate structural similarity. In certain embodiments, the antibody or antiserum is an antibody or antiserum that reacts with a non-contiguous epitope (i.e., not contiguous in primary sequence) that is formed by the tertiary or quaternary structure of a Zika NS1.

In one embodiment, an NS1 polypeptide binds to the AA12 antibody described herein. In another embodiment, an NS1 polypeptide binds to the EB9 antibody described herein. In another embodiment, an NS1 polypeptide binds to the GB5 antibody described herein. In another embodiment, an NS1 polypeptide binds to the FC12 antibody described herein.

In a specific embodiment, NS1 polypeptides provided herein are soluble. In another specific embodiment, NS1 polypeptides have one, two or more of the functions of a Zika virus NS1. For example, the NS1 polypeptide functions as a Zika virus NS1 in viral RNA replication, immune evasion, or both. In another embodiment, a dimer of an NS1 polypeptide provided herein associates with the plasma membrane of Zika virus infected cells. In another embodiment, an NS1 polypeptide described herein associates with the viral replication complex on the surface of the endoplasmic reticulum.

In a specific embodiment, an NS1 polypeptide described herein is recombinant produced and isolated. In another embodiment, an NS1 polypeptide described herein is used to immunize a subject against Zika virus. In another embodiment, an NS1 polypeptide described herein is used to produce antibodies, such as described in Section 5.2, infra. The antibodies may be produced in a human or non-human subject (e.g., a mouse, rat, etc.). In one embodiment, the non-human subject is capable of producing human antibodies.

### 5.2 Anti-Zika NS1 Antibodies

In one aspect, provided herein are antibodies (e.g., monoclonal antibodies and antigen-binding fragments) that bind to a Zika virus non-structural protein 1 (NS-1). In a specific embodiment, provided herein is an antibody that binds to NS1 of one, two, three or more strains of a Zika virus (e.g., a Zika virus strain described in Section 6, infra). In a specific embodiment, an antibody described herein is isolated or purified.

Antibodies can include, for example, monoclonal antibodies, recombinantly produced antibodies, monospecific antibodies, multispecific antibodies (including bispecific antibodies), human antibodies, humanized antibodies, chimeric antibodies, synthetic antibodies, tetrameric antibodies comprising two heavy chain and two light chain molecule, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′) fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1) or subclass thereof. In certain embodiments, antibodies described herein are IgA antibodies. In a specific embodiment, an antibody includes any molecule with an antigen-binding site that binds an antigen. In some embodiments, an antibody includes an antigen-binding fragment (e.g., the region(s) of an immunoglobulin that binds to an antigen or an epitope, such as a sequence comprising complementarity determining regions (e.g., the heavy and/or light chain variable regions)). In other embodiments, an antibody does not include antigen-binding fragments.

In a specific embodiment, an antibody described herein is a monoclonal antibody. As used herein, the term “monoclonal antibody” refers to an antibody obtained from a population of homogenous or substantially homogeneous antibodies. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single cell (e.g., hybridoma or host cell producing a recombinant antibody), wherein the antibody binds to a Zika virus NS1 as determined, e.g., by ELISA or other antigen-binding or competitive binding assay known in the art or in the Examples provided herein. In particular embodiments, a monoclonal antibody can be a chimeric antibody, a human antibody, or a humanized antibody. In certain embodiments, a monoclonal antibody is a monovalent antibody or multivalent (e.g., bivalent) antibody. In particular embodiments, a monoclonal antibody is a monospecific or multispecific antibody (e.g., bispecific antibody). Monoclonal antibodies described herein can, for example, be made by the hybridoma method as described in Kohler et al.; Nature, 256:495 (1975) or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York).

In a specific embodiment, an antibody described herein is an immunoglobulin, such as an IgG, IgE, IgM, IgD, IgA or IgY. In certain embodiments, an antibody described herein is an IgG2a. In some embodiments, an antibody described herein is an IgG1. In another embodiment, antibody described herein is an antigen-binding fragment, such as, e.g., an Fab fragment or F(ab′)₂ fragment. In another embodiment, an antibody described herein is an scFv. In another embodiment, provided herein are polyclonal antibodies or monoclonal antibodies produced using an NS1 polypeptide described in Section 5.1, supra, or Section 6, infra.

In a specific embodiment, the terms “NS1” and “non-structural protein 1” refer to any Zika virus NS1 known to those of skill in the art. Examples of Zika viruses include those provided in Section 5.1, supra, and 6, infra. In certain embodiments, NS1 is modified by post-translational processing such as glycosylation (e.g., N-linked glycosylation).

In another aspect, the antibodies provided herein bind to a Zika virus NS1 with a certain affinity. “Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), equilibrium association constant (K_{A}), and IC₅₀. The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore™, Kinexa, or biolayer interferometry. See, e.g., the techniques described in Section 6, infra.

Affinity can be measured by common methods known in the art, including those described herein. For example, individual association (kₒₙ) and dissociation (k_{off}) rate constants can be calculated from the resulting binding curves using the evaluation software available through the vendor. Data can then be fit to a 1:1 binding model, which includes a term to correct for mass transport limited binding, should it be detected. From these rate constants, the apparent dissociation binding constant (K_{D}) for the interaction of the antibody (e.g., IgG) with the antigen (e.g., Zika virus NS1) can be calculated from the quotient of k_{off}/kₒₙ. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the described herein.

In a specific embodiment, provided herein are antibodies that bind to a Zika virus NS1 with a kₒₙ, k_{off}, and/or K_{D} within the range or as disclosed in Table 10. In certain embodiments, the affinity of antibodies is determined using a technique described herein (e.g., in Section 6, infra) or one by one of skill in art such as, e.g., BIAcore™ surface plasmon resonance technology, Kinexa, or biolayer interferometry.

In some embodiments, an antibody provided herein binds to a Zika virus NS1 with a K_{D} of about 10⁻⁷ molar, 5×10⁻⁷ molar, 10⁻⁸ molar, 5×10⁻⁸ molar, 10⁻⁹ molar, 5×10⁻⁹ molar, or 10⁻¹⁰ molar. In certain embodiments, an antibody provided herein binds to a Zika virus NS1 with a K_{D} of 10⁻⁷ to 10⁻⁸ molar, 10⁻⁸ to 10⁻⁹ molar, 10⁻⁸ to 10⁻¹⁰ molar, 10⁻⁷ to 10⁻¹⁰ molar, or 10⁻⁷ to 10⁻⁹ molar.

In a specific embodiment, an antibody described herein has a high avidity for a Zika virus NS1 as assessed by a technique known to one of skill in the art, such as an immunoassay, surface plasmon resonance, or kinetic exclusion assay, biolayer interferometry, or described herein. In a particular, an antibody provided herein has a higher avidity than the AA12, EB9, GB5 or FC12 antibody.

In one embodiment, provided herein are antibodies (e.g., monoclonal antibodies, such as human, chimeric or humanized antibodies, and antigen-binding fragments) that bind to the NS1 of Zika virus MR766, PRVABC59, Pan/2015 or a combination thereof. as assessed by a technique known to one of skill in the art, such as an immunoassay, surface plasmon resonance, or kinetic exclusion assay, biolayer interferometry, or described herein. In another embodiment, an antibody described herein binds to a Zika virus NS1 that has 90%, 95%, 98%, 99% or greater identity to the NS1 protein of Zika virus MR766, PRVABC59 or Pan/2015 as assessed by a technique known to one of skill in the art, such as an immunoassay, surface plasmon resonance, or kinetic exclusion assay, biolayer interferometry, or described herein.

In another embodiment, provided herein are antibodies (e.g., monoclonal antibodies, such as human, chimeric or humanized antibodies, and antigen-binding fragments) that bind to the NS1 of different strains of Zika virus (e.g., 2, 3, 4, 5, 6 or more strains) as assessed by a technique known to one of skill in the art, such as an immunoassay, surface plasmon resonance, or kinetic exclusion assay, or described herein. In another embodiment, provided herein are antibodies that bind to NS1 of African lineages of Zika virus, Asian lineages of Zika virus, or both.

In another embodiment, an antibody described herein binds to cells infected with a Zika virus. In another embodiment, an antibody described herein binds to cells infected with Zika virus MR766, PRVABC59 or Pan/2015. In another embodiment, an antibody described herein binds to cells infected with a Zika virus which expresses an NS1 that has 90%, 92%, 94%, 95%, 96%, 97%, 98%, 99% or greater identity to the NS1 of Zika virus MR766, PRVABC59, or Pan/2015.

In another embodiment, an antibody described herein binds to a recombinant NS1 protein (e.g., a recombinant form of a Zika virus NS1) such as described herein (e.g., in Section 5.1, supra, or 6, infra. In another embodiment, an antibody described herein binds to a recombinant NS1 protein that has 90%, 92%, 94%, 95%, 96%, 97%, 98%, 99% or greater identity to the NS1 protein of Zika virus MR766, PRVABC59 or Pan/2015 as assessed by a technique known to one of skill in the art, such as an immunoassay, surface plasmon resonance, or kinetic exclusion assay, biolayer interferometry, or described herein. In a particular embodiment, an antibody described herein binds to a recombinant NS1 polypeptide described in Section 5.1, supra, or 6, infra.

In some embodiments, an antibody described herein binds to Zika virus NS1 and inhibits the activity of the NS1. In certain embodiments, an antibody described herein binds to Zika virus NS1 and inhibits the role of Zika virus NS1 in genome replication, inhibits one, two or more of the immune-modulatory functions of Zika virus NS1, both inhibits the role of Zika virus NS1 in genome replication and one, two or more of the immune-modulatory functions of Zika virus NS1. The inhibition of the role of Zika virus NS1 in genome replication may be complete or partial as assessed by a technique known to one of skill in the art or described herein (e.g., Section 6, infra). The inhibition of one, two or more of the immune-modulatory functions of Zika virus NS1 may be complete or partial as assessed by a technique known to one of skill in the art.

In a specific embodiment, an antibody described herein that binds to NS1 of Zika virus is a non-neutralizing antibody. In another embodiment, an antibody described herein that binds to NS1 of Zika virus exhibits no antibody-dependent enhancement of Zika virus infection in vitro as assessed by a technique known in the art or described herein (e.g., Section 6, infra). In another embodiment, an antibody described herein that binds to NS1 of Zika virus activates Natural Killer (NK) cells.

In another aspect, an antibody provided herein demonstrates Fc-mediated antibody effector functions in an in vitro assay known to one of skill in the art or described herein (e.g., in Section 6, infra). In another embodiment, an antibody provided herein demonstrates one, two or all of the following: antibody-dependent cell-mediated cytotoxicity (ADCC), antibody-dependent cell-mediated phagocytosis (ADCP) and antibody-dependent complement-mediated lysis as assessed by techniques known to one of skill in the art or described herein. In a specific embodiment, an antibody provided herein demonstrates antibody dependent cell-mediated cytotoxicity (ADCC). In a specific embodiment, an antibody provided herein demonstrates ADCC activity in an in vitro assay known to one of skill in the art. For example, ADCC activity may be assessed using Promega's ADCC Reporter Assay Core Kit.

In another aspect, an antibody provided herein has one, two or more, or all of the characteristics/properties of one of the antibodies described in Section 6, infra. In a specific embodiment, an antibody described herein has one, two or more, or all of the characteristics/properties of the AA12 antibody described herein. In another specific embodiment, an antibody provided herein has one, two or more, or all of the characteristics/properties of the EB9 antibody described herein. In another specific embodiment, an antibody provided herein has one, two or more, or all of the characteristics/properties of the GB5 antibody described herein. In another specific embodiment, an antibody provided herein has one, two or more, or all of the characteristics/properties of the FC12 antibody described herein.

In a specific embodiment, an antibody described herein binds to NS1 of Zika virus. In another embodiment, provided herein is an antibody that specifically binds to an NS1 of one, two, three or more strains of Zika virus relative to a non-Zika virus antigen (e.g., a non-Zika virus NS1) as assessed by techniques known in the art, e.g., ELISA, Western blot, biolayer interferometry, FACS or BIACore, or described herein. In other words, the antibody binds to an NS1 from one, two, three or more strains of Zika virus with a higher affinity than the antibody binds to a non-Zika virus antigen (e.g., a non-Zika virus NS1) as assessed by techniques known in the art, e.g., ELISA, Western blot, biolayer interferometry, FACS or BIACore, or described herein. In some embodiments, an antibody described herein binds to an NS1 of one, two, three or more strains of Zika virus with a 1-fold, 1.5-fold, 2-fold, 3-fold, 4-fold, 5-fold, 6-fold, 7-fold, 8-fold, 9-fold, 10-fold, greater than 10-fold, 1- to 2-fold, 1- to 5-fold, 1- to 10-fold, 2- to 5-fold, 2- to 10-fold, 5- to 10-fold, 10- to 15-fold, or 10- to 20-fold greater affinity than that with which the antibody binds to a non-Zika virus antigen (e.g., a non-Zika virus NS1) as assessed by techniques known in the art, e.g., ELISA, Western blot, biolayer interferometry, FACS or BIACore, or described herein. In certain embodiments, an antibody described herein binds to an NS1 of one, two, three or more strains of Zika virus with a 0.5 log, 1 log, 1.5 log, 2 log, 2.5 log, 3 log, 3.5 log, or 4 log greater affinity than that with which the antibody binds to a non-Zika virus antigen (e.g., a non-Zika virus NS1) as assessed by techniques known in the art, e.g., ELISA, Western blot, biolayer interferometry, FACS or BIACore, or described herein. In some embodiments, an antibody described herein binds to an NS1 of one, two, three or more strains of Zika virus with a 5%, 10%, 15%, 20%, 25%, 30%, 35%, 40%, 45%, 50%, 55%, 60%, 65%, 70% or higher affinity than that with which the antibody binds to a non-Zika virus antigen as measured by, e.g., a radioimmunoassay, surface plasmon resonance, kinetic exclusion assay, or biolayer interferometry, or described herein.

In another embodiment, an antibody described herein does not cross-react with an NS1 from another flavivirus as assessed by techniques known in the art, e.g., ELISA, Western blot, biolayer interferometry, FACS or BIACore, or described herein. In certain embodiments, provided herein is an antibody that specifically binds to an NS1 of one, two, three or more strains of Zika virus relative to an NS1 of another flavivirus as assessed by techniques known in the art, e.g., ELISA, Western blot, biolayer interferometry, FACS or BIACore, or described herein. In other words, the antibody binds to an NS1 from one, two, three or more strains of Zika virus with a higher affinity than the antibody binds to an NS1 of another flavivirus as assessed by techniques known in the art, e.g., ELISA, Western blot, biolayer interferometry, FACS or BIACore, or described herein. In some embodiments, an antibody described herein binds to an NS1 of one, two, three or more strains of Zika virus with a 1-fold, 1.5-fold, 2-fold, 3-fold, 4-fold, 5-fold, 6-fold, 7-fold, 8-fold, 9-fold, 10-fold, greater than 10-fold, 1- to 2-fold, 1- to 5-fold, 1- to 10-fold, 2- to 5-fold, 2- to 10-fold, 5- to 10-fold, 10- to 15-fold, or 10- to 20-fold greater affinity than that which the antibody binds to an NS1 of another flavivirus as assessed by techniques known in the art, e.g., ELISA, Western blot, biolayer interferometry, FACS or BIACore, or described herein. In certain embodiments, an antibody described herein binds to an NS1 of one, two, three or more strains of Zika virus with a 0.5 log, 1 log, 1.5 log, 2 log, 2.5 log, 3 log, 3.5 log, or 4 log greater affinity than that which the antibody binds to an NS1 of another flavivirus as assessed by techniques known in the art, e.g., ELISA, Western blot, biolayer interferometry, FACS or BIACore, or described herein. In certain embodiments, an antibody described herein binds to an NS1 of one, two, three or more strains of Zika virus with a 5%, 10%, 15%, 20%, 25%, 30%, 35%, 40%, 45%, 50%, 55%, 60%, 65%, 70% or higher affinity than that which the antibody binds to an NS1 of another flavivirus as measured by, e.g., a radioimmunoassay, surface plasmon resonance, kinetic exclusion assay, or biolayer interferometry, or described herein.

In another embodiment, an antibody described herein does not cross-react with an NS1 from a Dengue virus as assessed by techniques known in the art, e.g., ELISA, Western blot, biolayer interferometry, FACS or BIACore, or described herein. In certain embodiments, provided herein is an antibody that specifically binds to an NS1 of one, two, three or more strains of Zika virus relative to an NS1 of a Dengue virus as assessed by techniques known in the art, e.g., ELISA, Western blot, biolayer interferometry, FACS or BIACore, or described herein. In other words, the antibody binds to an NS1 from one, two, three or more strains of Zika virus with a higher affinity than the antibody binds to an NS1 of a Dengue virus as assessed by techniques known in the art, e.g., ELISA, Western blot, biolayer interferometry, FACS or BIACore, or described herein. In some embodiments, an antibody described herein binds to an NS1 of one, two, three or more strains of Zika virus with a 1-fold, 1.5-fold, 2-fold, 3-fold, 4-fold, 5-fold, 6-fold, 7-fold, 8-fold, 9-fold, 10-fold, greater than 10-fold, 1- to 2-fold, 1- to 5-fold, 1- to 10-fold, 2- to 5-fold, 2- to 10-fold, 5- to 10-fold, 10- to 15-fold, or 10- to 20-fold greater affinity than that which the antibody binds to an NS1 of a Dengue virus as assessed by techniques known in the art, e.g., ELISA, Western blot, biolayer interferometry, FACS or BIACore, or described herein. In certain embodiments, an antibody described herein binds to an NS1 of one, two, three or more strains of Zika virus with a 0.5 log, 1 log, 1.5 log, 2 log, 2.5 log, 3 log, 3.5 log, or 4 log greater affinity than that which the antibody binds to an NS1 of a Dengue virus as assessed by techniques known in the art, e.g., ELISA, Western blot, biolayer interferometry, FACS or BIACore, or described herein. In certain embodiments, an antibody described herein binds to NS1 of one, two, three or more strains of Zika virus with a 5%, 10%, 15%, 20%, 25%, 30%, 35%, 40%, 45%, 50%, 55%, 60%, 65%, 70% or higher affinity than that which the antibody binds to an NS1 of a Dengue virus as measured by, e.g., a radioimmunoassay, surface plasmon resonance, kinetic exclusion assay, or biolayer interferometry, or described herein. In a specific embodiment, an antibody described herein binds to NS1 of Zika virus and does not bind to Dengue virus NS1 (e.g., DENV3 NS1) as assessed by techniques known in the art, e.g., ELISA, Western blot, biolayer interferometry, FACS or BIACore, or described herein (e.g., in Section 6, infra).

In certain embodiments, provided herein are antibodies that: (i) bind to a non-linear epitope of NS1 of a Zika virus and (ii) inhibit one, two or more functions of the NS1, as assessed by a technique known to one of skill in the art or described herein. In some embodiments, provided herein are antibodies that: (i) bind to a linear epitope of NS1 of a Zika virus and (ii) inhibit one, two or more functions of the NS1, as assessed by a technique known to one of skill in the art or described herein.

In another aspect, an antibody described herein is the AA12, EB9, GB5, or FC12 antibody or an antigen-binding fragment thereof. In another aspect, an antibody provided herein comprises the variable heavy chain region (“VH) or variable light chain region (“VL”) of the AA12, EB9, GB5, or FC12 antibody. In another aspect, an antibody provided herein comprises the variable heavy chain region (“VH”) and variable light chain region (“VL”) of the AA12, EB9, GB5, or FC12 antibody. In a specific embodiment, an antibody provided herein encoded by a nucleic acid sequence(s) comprising (1) the nucleotide sequences of SEQ ID Nos: 1 and 2; (2) nucleotide sequences of SEQ ID Nos: 3 and 4; (3) nucleotide sequences of SEQ ID Nos: 5 and 6; or (4) nucleotide sequences of SEQ ID Nos: 7 and 8. In a specific embodiment, an antibody provided herein is an antibody described in Section 6.1., infra.

As used herein, the terms “variable region” or “variable domain” are used interchangeably and are common in the art. Sometimes a variable heavy chain region is referred to herein as a VH or VH domain. Sometimes a variable light chain region is referred to as a VL or VL domain. The variable region typically refers to a portion of an antibody, generally, a portion of a light or heavy chain, typically about the amino-terminal 110 to 120 amino acids in a mature heavy chain and about the amino-terminal 90 to 100 amino acids in a mature light chain, which differs extensively in sequence among antibodies and is used in the binding and specificity of a particular antibody for its particular antigen. The variability in sequence is concentrated in those regions called complementarity determining regions (CDRs) while the more highly conserved regions in the variable domain are called framework regions (FR). CDRs are flanked by FRs. Generally, the spatial orientation of CDRs and FRs are as follows, in an N-terminal to C-terminal direction: FR1-CDR1-FR2-CDR2-FR3-CDR3-FR4. Without wishing to be bound by any particular mechanism or theory, it is believed that the CDRs of the light and heavy chains are primarily responsible for the interaction and specificity of the antibody with antigen.

In certain embodiments, the variable region is a rodent (e.g., mouse or rat) variable region. In certain embodiments, the variable region is a human variable region. In certain embodiments, the variable region comprises rodent (e.g., mouse or rat) CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

In another aspect, an antibody provided herein comprises one, two or three of the complementarity determining regions (CDRs) of the variable heavy chain region (“VH” domain) or one, two or three of the CDRs of the variable light chain region (“VL”) of the AA12, EB9, GB5, or FC12 antibody. In another aspect, an antibody provided herein comprises one, two or three of the complementarity determining regions (CDRs) of the variable heavy chain region (“VH”) and one, two or three of the CDRs of the variable light chain region (“VL”) of the AA12, EB9, GB5, or FC12 antibody. In another aspect, an antibody provided herein comprises the complementarity determining regions (CDRs) of the variable heavy chain region (“VH”) and the CDRs of the variable light chain region (“VL”) of the AA12, EB9, GB5, or FC12 antibody. In some embodiments, the antibody further comprises framework regions from a non-murine antibody (e.g., a human antibody) or framework regions derived from a non-murine antibody (e.g., a human antibody).

In certain aspects, the CDRs of an antibody can be determined according to the Kabat numbering system. The term “Kabat numbering,” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). With respect to the Kabat numbering system, (i) the VH CDR1 is typically present at amino acid positions 31 to 35 of the heavy chain, which can optionally include one or two additional amino acids following amino acid position 35 (referred to in the Kabat numbering scheme as 35A and 35B); (ii) the VH CDR2 is typically present at amino acid positions 50 to 65 of the heavy chain; and (iii) the VH CDR2 is typically present at amino acid positions 95 to 102 of the heavy chain (Kabat, Elvin A. et al., Sequences of Proteins of Immunological Interest. Bethesda: National Institutes of Health, 1983). With respect to the Kabat numbering system, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (Kabat, Elvin A. et al., Sequences of Proteins of Immunological Interest. Bethesda: National Institutes of Health, 1983). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

In certain aspects, the CDRs of an antibody can be determined according to the Chothia numbering scheme, which refers to the location of immunoglobulin structural loops (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215 (1): 175-82; and U.S. Pat. No. 7,709,226). The Chothia definition is based on the location of the structural loop regions (Chothia et al., (1987) J Mol Biol 196:901-917; and U.S. Pat. No. 7,709,226). The term “Chothia CDRs,” and like terms are recognized in the art and refer to antibody CDR sequences as determined according to the method of Chothia and Lesk, 1987, J. Mol. Biol., 196:901-917, which will be referred to herein as the “Chothia CDRs” (see also, e.g., U.S. Pat. No. 7,709,226 and Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001)). With respect to the Chothia numbering system, using the Kabat numbering system of numbering amino acid residues in the VH region, (i) the VH CDR1 is typically present at amino acid positions 26 to 32 of the heavy chain; (ii) the VH CDR2 is typically present at amino acid positions 53 to 55 of the heavy chain; and (iii) the VH CDR3 is typically present at amino acid positions 96 to 101 of the heavy chain. In a specific embodiment, with respect to the Chothia numbering system, using the Kabat numbering system of numbering amino acid residues in the VH region, (i) the VH CDR1 is typically present at amino acid positions 26 to 32 or 34 of the heavy chain; (ii) the VH CDR2 is typically present at amino acid positions 52 to 56 (in one embodiment, CDR2 is at positions 52A-56, wherein 52A follows position 52) of the heavy chain; and (iii) the VH CDR3 is typically present at amino acid positions 95 to 102 of the heavy chain (in one embodiment, there is no amino acid at positions numbered 96-100). With respect to the Chothia numbering system, using the Kabat numbering system of numbering amino acid residues in the VL region, (i) the VL CDR1 is typically present at amino acid positions 26 to 33 of the light chain; (ii) the VL CDR2 is typically present at amino acid positions 50 to 52 of the light chain; and (iii) the VL CDR3 is typically present at amino acid positions 91 to 96 of the light chain. In a specific embodiment, with respect to the Chothia numbering system, using the Kabat numbering system of numbering amino acid residues in the VL region, (i) the VL CDR1 is typically present at amino acid positions 24 to 34 of the light chain; (ii) the VL CDR2 is typically present at amino acid positions 50 to 56 of the light chain; and (iii) the VL CDR3 is typically present at amino acid positions 89 to 97 of the light chain (in one embodiment, there is no amino acid at positions numbered 96-100). These Chothia CDR positions may vary depending on the antibody, and may be determined according to methods known in the art.

In certain aspects, the CDRs of an antibody can be determined according to the IMGT numbering system as described in Lefranc, M.-P., 1999, The Immunologist, 7:132-136 and Lefranc, M.-P. et al., 1999, Nucleic Acids Res., 27:209-212. The IMGT definition is from the IMGT (“IMGT®, the international ImMunoGeneTics information System® website imgt.org, founder and director: Marie-Paule Lefranc, Montpellier, France; see, e.g., Lefranc, M.-P., 1999, The Immunologist, 7:132-136 and Lefranc, M.-P. et al., 1999, Nucleic Acids Res., 27:209-212, both of which are incorporated herein by reference in their entirety). With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

In certain aspects, the CDRs of an antibody can be determined according to MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody can be determined according to the AbM numbering scheme, which refers AbM hypervariable regions which represent a compromise between the Kabat CDRs and Chothia structural loops, and are used by Oxford Molecular's AbM antibody modeling software. In certain aspects, the CDRs or antibody binding regions (ABRs) of an antibody can be determined according to Paratome (Kunik et al., 2012, Nucleic Acids Res. Vol. 40, Web Server issue W521-W524 and see website of ranservices.biu.ac.il/site/services/paratome/index.html). In some instances herein, the term CDRs are used instead of ABRs when referring to the ABRs delineated using the Paratome system.

In a specific aspect, an antibody provided herein that binds to Zika virus NS1 (e.g., a Zika virus NS1, such as described in Section 6, infra), wherein the antibody comprises a variable heavy chain region (“VH”) or heavy chain comprising: (1) a VH CDR1 comprising the amino acid sequence GFTVSSNY (SEQ ID NO:143), (2) a VH CDR2 comprising the amino acid sequence IYSGGST (SEQ ID NO:144), and (3) a VH CDR3 comprising the amino acid sequence ARDRRGFDY (SEQ ID NO:145), ARWGGKRGGAFDI (SEQ ID NO:146), ARLIAAAGDY (SEQ ID NO:147), or ARGPVQLERRPLGAFDI (SEQ ID NO:148). In another aspect, an antibody provided herein that binds to Zika virus NS1 (e.g., a Zika virus NS1, such as described in Section 6, infra), wherein the antibody comprises a variable light chain region (“VL”) or light chain comprising: (1) a VL CDR1 comprising the amino acid sequence QSISSX, X is Y or H (SEQ ID NO:134), (2) a VL CDR2 comprising the amino acid sequence X1X2S, X1 is A or Q, X2 is A or D (SEQ ID NO:135), and (3) a VL CDR3 comprising the amino acid sequence of QQX1YSTPX2T, X1 is T or S, X2 is L, Y, or W (SEQ ID NO:136). In another aspect, provided herein is an antibody that binds to Zika virus NS1 (e.g., a Zika virus NS1, such as described in Section 6, infra), wherein the antibody comprises a variable heavy chain region (VH”) and a variable light chain region, wherein the variable heavy chain region comprises (1) a VH CDR1 comprising the amino acid sequence GFTVSSNY (SEQ ID NO:143), and (2) a VH CDR2 comprising the amino acid sequence IYSGGST (SEQ ID NO:144), and wherein the variable light chain region comprises (1) a VL CDR1 comprising the amino acid sequence QSISSX, X is Y or H (SEQ ID NO:134), (2) a VL CDR2 comprising the amino acid sequence X1X2S, X1 is A or Q, X2 is A or D (SEQ ID NO:135), and (3) a VL CDR3 comprising the amino acid sequence QQX1YSTPX2T, X1 is T or S, X2 is L, Y, or W (SEQ ID NO: 136). In another aspect, provided herein is an antibody that binds to Zika virus NS1 (e.g., a Zika virus NS1, such as described in Section 6, infra), wherein the antibody comprises a heavy chain and a light chain, wherein the heavy chain comprises (1) a VH CDR1 comprising the amino acid sequence GFTVSSNY (SEQ ID NO:143), and (2) a VH CDR2 comprising the amino acid sequence IYSGGST (SEQ ID NO:144), and wherein the light chain comprises (1) a VL CDR1 comprising the amino acid sequence QSISSX, X is Y or H (SEQ ID NO:134), (2) a VL CDR2 comprising the amino acid sequence X1X2S, X1 is A or Q, X2 is A or D (SEQ ID NO: 135), and (3) a VL CDR3 comprising the amino acid sequence QQX1YSTPX2T, X1 is T or S, X2 is L, Y, or W (SEQ ID NO:136).

In another aspect, provided herein is an antibody that binds to a Zika virus NS1 (e.g., a Zika virus NS1, such as described in Section 6, infra), wherein the antibody comprises variable heavy chain region and a variable light chain region, wherein the variable heavy chain region comprises (1) a VH CDR1 comprising the amino acid sequence GFTVSSNY (SEQ ID NO:143), (2) a VH CDR2 comprising the amino acid sequence IYSGGST (SEQ ID NO:144), and (3) a VH CDR3 comprising the amino acid sequence ARDRRGFDY (SEQ ID NO:145), ARWGGKRGGAFDI (SEQ ID NO:146), ARLIAAAGDY (SEQ ID NO:147), or ARGPVQLERRPLGAFDI (SEQ ID NO:148), and wherein the variable light chain region comprises (1) a VL CDR1 comprising the amino acid sequence QSISSX, X is Y or H (SEQ ID NO: 134), (2) a VL CDR2 comprising the amino acid sequence X1X2S, X1 is A or Q, X2 is A or D (SEQ ID NO:135), and (3) a VL CDR3 comprising the amino acid sequence QQX1YSTPX2T, X1 is T or S, X2 is L, Y, or W (SEQ ID NO:136). In another aspect, provided herein is an antibody comprising: (1) a VH CDR1 comprising the amino acid sequence GFTVSSNY (SEQ ID NO: 143), (2) a VH CDR2 comprising the amino acid sequence IYSGGST (SEQ ID NO:144), (3) a VH CDR3 comprising the amino acid sequence ARDRRGFDY (SEQ ID NO:145), ARWGGKRGGAFDI (SEQ ID NO:146), ARLIAAAGDY (SEQ ID NO:147), or ARGPVQLERRPLGAFDI (SEQ ID NO:148), (4) a VL CDR1 comprising the amino acid sequence QSISSX, X is Y or H (SEQ ID NO:134), (5) a VL CDR2 comprising the amino acid sequence X1X2S, X1 is A or Q, X2 is A or D (SEQ ID NO:135), and (6) a VL CDR3 comprising the amino acid sequence QQX1YSTPX2T, X1 is T or S, X2 is L, Y, or W (SEQ ID NO: 136).

In a specific aspect, an antibody provided herein that binds to Zika virus NS1 (e.g., a Zika virus NS1, such as described in Section 6, infra), wherein the antibody comprises a variable heavy chain region or heavy chain comprising: (1) a VH ABR1 comprising the amino acid sequence FTVSSNYMS (SEQ ID NO:149), (2) a VH ABR2 comprising the amino acid sequence WVSVIYSGGSTYYA (SEQ ID NO:150), and (3) a VH ABR3 comprising the amino acid sequence ARDRRGFDY (SEQ ID NO:151), ARWGGKRGGAFDI (SEQ ID NO:152), ARLIAAAGDY (SEQ ID NO:153), or ARGPVQLERRPLGAFDI (SEQ ID NO:154). In another aspect, an antibody provided herein that binds to Zika virus NS1 (e.g., a Zika virus NS1, such as described in Section 6, infra), wherein the antibody comprises a variable light chain region or light chain comprising: (1) a VL ABR1 comprising the amino acid sequence QSISSXILN, X1 is Y or H (SEQ ID NO:137), (2) a VL ABR2 comprising the amino acid sequence XILIYAASSLQS, X1 is F or L (SEQ ID NO:138), and (3) a VL ABR3 comprising the amino acid sequence QQX1YSTPX2, X1 is T or S, X2 is L, Y or W (SEQ ID NO:139).

In another specific aspect, an antibody provided herein that binds to a Zika virus NS1 (e.g., a Zika virus NS1, such as described in Section 6, infra), wherein the antibody comprises a variable heavy chain region and a variable light chain region, wherein the variable heavy chain region comprises: (1) a VH ABR1 comprising the amino acid sequence FTVSSNYMS (SEQ ID NO: 149), (2) a VH ABR2 comprising the amino acid sequence WVSVIYSGGSTYYA (SEQ ID NO: 150), and (3) a VH ABR3 comprising the amino acid sequence ARDRRGFDY (SEQ ID NO: 151), ARWGGKRGGAFDI (SEQ ID NO:152), ARLIAAAGDY (SEQ ID NO:153), or ARGPVQLERRPLGAFDI (SEQ ID NO:154), and wherein the variable light chain region comprises: (1) a VL ABR1 comprising the amino acid sequence QSISSXILN, X1 is Y or H (SEQ ID NO:137), (2) a VL ABR2 comprising the amino acid sequence XILIYAASSLQS, X1 is F or L (SEQ ID NO:138), and (3) a VL ABR3 comprising the amino acid sequence QQX1YSTPX2, X1 is T or S, X2 is L, Y or W (SEQ ID NO:139). In another specific aspect, an antibody provided herein that binds to a Zika virus NS1 (e.g., a Zika virus NS1, such as described in Section 6, infra), wherein the antibody comprises a heavy chain and a light chain, wherein the heavy chain comprises: (1) a VH ABR1 comprising the amino acid sequence FTVSSNYMS (SEQ ID NO:149), (2) a VH ABR2 comprising the amino acid sequence WVSVIYSGGSTYYA (SEQ ID NO:150), and (3) a VH ABR3 comprising the amino acid sequence ARDRRGFDY (SEQ ID NO:151), ARWGGKRGGAFDI (SEQ ID NO:152), ARLIAAAGDY (SEQ ID NO:153), or ARGPVQLERRPLGAFDI (SEQ ID NO:154), and wherein the light chain comprises: (1) a VL ABR1 comprising the amino acid sequence QSISSXILN, X1 is Y or H (SEQ ID NO:137), (2) a VL ABR2 comprising the amino acid sequence XILIYAASSLQS, X1 is F or L (SEQ ID NO:138), and (3) a VL ABR3 comprising the amino acid sequence QQX1YSTPX2, X1 is T or S, X2 is L, Y or W (SEQ ID NO:139). In another specific aspect, an antibody provided herein that binds to a Zika virus NS1 (e.g., a Zika virus NS1, such as described in Section 6, infra), wherein the antibody comprises: (1) a VH ABR1 comprising the amino acid sequence FTVSSNYMS (SEQ ID NO:149), (2) a VH ABR2 comprising the amino acid sequence WVSVIYSGGSTYYA (SEQ ID NO:150), (3) a VH ABR3 comprising the amino acid sequence ARDRRGFDY (SEQ ID NO:151), ARWGGKRGGAFDI (SEQ ID NO:152), ARLIAAAGDY (SEQ ID NO:153), or ARGPVQLERRPLGAFDI (SEQ ID NO: 154), (4) a VL ABR1 comprising the amino acid sequence QSISSXILN, X1 is Y or H (SEQ ID NO: 137), (5) a VL ABR2 comprising the amino acid sequence XILIYAASSLQS, X1 is F or L (SEQ ID NO:138), and (6) a VL ABR3 comprising the amino acid sequence QQX1YSTPX2, X1 is T or S, X2 is L, Y or W (SEQ ID NO:139).

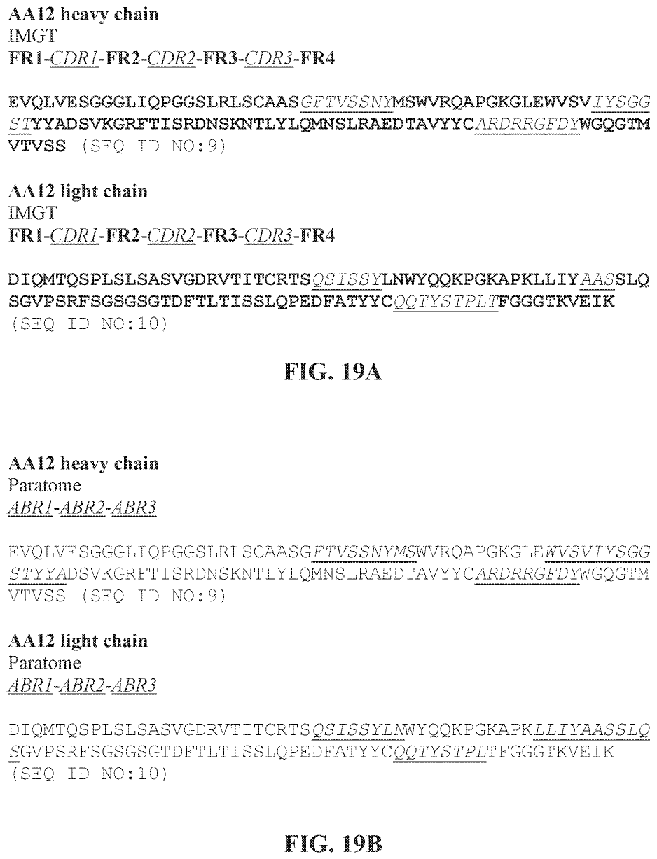

In a specific aspect, an antibody provided herein is the antibody designated AA12 or an antigen-binding fragment thereof. The AA12 antibody is a human antibody. The deduced nucleotide sequences of the variable heavy chain region and variable light chain region of the antibody AA12 are shown in Table 1. The deduced amino acid sequences of the VH and VL domains of the antibody AA12 are shown in FIGS. 19A-19B and Table 1. The CDRs and framework regions of the VH domain and VL domain are indicated in FIGS. 19A-19B. In addition, Table 1, infra, sets forth the amino acid sequences of the CDRs and framework regions of the variable regions of the antibody AA122 as determined by the IMGT numbering system. The CDRs and framework regions were determined using the International ImMunoGeneTics (“IMGT”) numbering system. See Lefranc et al., Dev. Comp. Immunol. 27:55-77 (2003), which is incorporated herein by reference in its entirety, for a description of the IMGT numbering system. As an alternative to the IMGT numbering system, the Paratome system may be used. Table 2, infra, sets forth the amino acid sequences of the ABRs and framework regions of the variable regions of the antibody AA12 as determined using the Paratome system. As an alternative to the IMGT numbering system, the Kabat numbering system can be used. Table 2 of Lefranc et al. shows the correspondence between the IMGT and the Kabat numberings. Another alternative to the IMGT numbering system is Chothia. See Chothia and Lesk, J. Mol. Biol. 196:901-917 (1987), which is incorporated herein by reference in its entirety. Further, Oxford's AbM system may be used instead of the IMGT numbering system. A person of ordinary skill in the art would be able to determine the CDRs and framework regions of the variable regions of the AA12 antibody sequence based on the Kabat numbering system, Chothia system, and/or Oxford's AbM system.

TABLE 1
AA12 Antibody

| SEQ | | |
|---|---|---|
| ID | Description | |
| NO: | of Sequence | Sequence |
| 1 | Isolate AA12 | gaggtgcagctggtggagtccggaggaggc |
| | immunoglobu- | ttgatccagcctggggggtccctgagactc |
| | lin | tcctgtgcagcctctgggttcaccgtcagt |
| | heavy chain | agcaactacatgagctgggtccgccaggct |
| | variable | ccagggaaggggctggagtgggtctcagtt |
| | region mRNA, | atttatagcggtggtagcacatactacgca |
| | partial CDS | gactccgtgaagggccgattcaccatctcc |
| | [organism = | agagacaattccaagaacacgctgtatctt |
| | Homo | caaatgaacagcctgagagccgaggacacg |
| | sapiens] | gccgtgtattactgtgcgagagatcgaagg |
| | | gggtttgactactggggccagggaacaatg |
| 2 | Isolate AA12 | gtcaccgtctcttcagacatccagatgacc |
| | immunoglobu- | cagtctccactctccctgtctgcatctgta |
| | lin | ggagacagagtcaccatcacttgccggaca |
| | light chain | agtcagagcattagcagctatttaaattgg |
| | variable | tatcagcagaaaccagggaaagcccctaag |
| | region mRNA, | ctcctgatctatgctgcatccagtttgcaa |
| | partial CDS | agtggggtcccatcaaggttcagtggcagt |
| | [organism = | ggatctgggacagatttcactcttaccatc |
| | Homo | agcagtctgcaacctgaagattttgcaact |
| | sapiens] | tactactgtcaacagacttacagtacccct |
| | | ctcactttcggcggagggaccaaggtggaa |
| | | atcaaa |
| 9 | Isolate AA12 | EVQLVESGGGLIQPGGSLRLSCAASGFT |
| | immunoglobu- | VSSNYMSWVRQAPGKGLEWVSVIYSGG |
| | lin | STYYADSVKGRFTISRDNSKNTLYLQMN |
| | heavy chain | SLRAEDTAVYYCARDRRGFDYWGQGT |
| | variable | MVTVSS |
| | region | |
| | amino acid | |
| | sequence | |
| | [organism = | |
| | Homo | |
| | sapiens] | |
| 10 | Isolate AA12 | DIQMTQSPLSLSASVGDRVTITCRTSQSIS |
| | immunoglobu- | SYLNWYQQKPGKAPKLLIYAASSLQSG |
| | lin | VPSRFSGSGSGTDFTLTISSLQPEDFATY |
| | light chain | YCQQTYSTPLTFGGGTKVEIK |
| | variable | |
| | region | |
| | amino acid | |
| | sequence | |
| | [organism = | |
| | Homo | |
| | sapiens] | |
| 17 | Isolate AA12 | GFTVSSNY |
| | immunoglobu- | |
| | lin | |
| | heavy chain | |
| | variable | |
| | region CDR1 | |
| | amino acid | |
| | sequence | |
| | (IMGT) | |
| 18 | Isolate AA12 | IYSGGST |
| | immunoglobu- | |
| | lin | |
| | heavy chain | |
| | variable | |
| | region CDR2 | |
| | amino acid | |
| | sequence | |
| | (IMGT) | |
| 19 | Isolate AA12 | ARDRRGFDY |
| | immunoglobu- | |
| | lin | |
| | heavy chain | |
| | variable | |
| | region CDR3 | |
| | amino acid | |
| | sequence | |
| | (IMGT) | |
| 20 | Isolate AA12 | QSISSY |
| | immunoglobu- | |
| | lin | |
| | light chain | |
| | variable | |
| | region CDR1 | |
| | amino acid | |
| | sequence | |
| | (IMGT) | |
| 21 | Isolate AA12 | AAS |
| | immunoglobu- | |
| | lin | |
| | light chain | |
| | variable | |
| | region CDR2 | |
| | amino acid | |
| | sequence | |
| | (IMGT) | |
| 22 | Isolate AA12 | QQTYSTPLT |
| | immunoglobu- | |
| | lin | |
| | light hain | |
| | variable | |
| | region CDR3 | |
| | amino acid | |
| | sequence | |
| | (IMGT) | |
| 23 | Isolate AA12 | EVQLVESGGGLIQPGGSLRLSCAAS |
| | immunoglobu- | |
| | lin | |
| | heavy chain | |
| | variable | |
| | region | |
| | framework | |
| | region 1 | |
| | amino acid | |
| | sequence | |
| | (IMGT) | |
| 24 | Isolate AA12 | MSWVRQAPGKGLEWVSV |
| | immunoglobu- | |
| | lin | |
| | heavy chain | |
| | variable | |
| | region | |
| | framework | |
| | region 2 | |
| | amino acid | |
| | sequence | |
| | (IMGT) | |
| 25 | Isolate AA12 | YYADSVKGRFTISRDNSKNTLYLQMNSL |
| | immunoglobu- | RAEDTAVYYC |
| | lin | |
| | heavy chain | |
| | variable | |
| | region | |
| | framework | |
| | region 3 | |
| | amino acid | |
| | sequence | |
| | (IMGT) | |
| 26 | Isolate AA12 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (IMGT) | |
| 27 | Isolate AA12 | DIQMTQSPLSLSASVGDRVTITCRTS |
| | immunoglobu- | |
| | lin | |
| | light chain | |
| | variable | |
| | region | |
| | framework | |
| | region 1 | |
| | amino acid | |
| | sequence | |
| | (IMGT) | |
| 28 | Isolate AA12 | LNWYQQKPGKAPKLLIY |
| | immunoglobu- | |
| | lin | |
| | light chain | |
| | variable | |
| | region | |
| | framework | |
| | region 2 | |
| | amino acid | |
| | sequence | |
| | (IMGT) | |
| 29 | Isolate AA12 | SLQSGVPSRFSGSGSGTDFTLTISSLQPED |
| | immunoglobu- | FATYYC |
| | lin | |
| | light chain | |
| | variable | |
| | region | |
| | framework | |
| | region 3 | |
| | amino acid | |
| | sequence | |
| | (IMGT) | |
| 30 | Isolate AA12 | FGGGTKVEIK |
| | immunoglobu- | |
| | lin | |
| | light chain | |
| | variable | |
| | region | |
| | framework | |
| | region 4 | |
| | amino acid | |
| | sequence | |
| | (IMGT) | |

TABLE 2
AA12 Antibody (Paratome)

| SEQ | | |
|---|---|---|
| ID | Description | |
| NO: | of Sequence | Sequence |
| 31 | Isolate AA12 | FTVSSNYMS |
| | immunoglobulin | |
| | heavy chain | |
| | variable | |
| | region ABR1 | |
| | amino acid | |
| | sequence | |
| | (Paratome) | |
| 32 | Isolate AA12 | WVSVIYSGGSTYYA |
| | immunoglobulin | |
| | heavy chain | |
| | variable | |
| | region ABR2 | |
| | amino acid | |
| | sequence | |
| | (Paratome) | |
| 33 | Isolate AA12 | ARDRRGFDY |
| | immunoglobulin | |
| | heavy chain | |
| | variable | |
| | region ABR3 | |
| | amino acid | |
| | sequence | |
| | (Paratome) | |
| 34 | Isolate AA12 | QSISSYLN |
| | immunoglobulin | |
| | light chain | |
| | variable | |
| | region ABR1 | |
| | amino acid | |
| | sequence | |
| | (Paratome) | |
| 35 | Isolate AA12 | LLIYAASSLQS |
| | immunoglobulin | |
| | light chain | |
| | variable | |
| | region ABR2 | |
| | amino acid | |
| | sequence | |
| | (Paratome) | |
| 36 | Isolate AA12 | QQTYSTPL |
| | immunoglobulin | |
| | light chain | |
| | variable | |
| | region ABR3 | |
| | amino acid | |
| | sequence | |
| | (Paratome) | |
| 37 | Isolate AA12 | EVQLVESGGGLIQPGGSLRLSCAASG |
| | immunoglobulin | |
| | heavy chain | |
| | variable | |
| | region | |
| | framework | |
| | region 1 | |
| | amino acid | |
| | sequence | |
| | (Paratome) | |
| 38 | Isolate AA12 | WVRQAPGKGLE |
| | immunoglobulin | |
| | heavy chain | |
| | variable | |
| | region | |
| | framework | |
| | region 2 | |
| | amino acid | |
| | sequence | |
| | (Paratome) | |
| 39 | Isolate AA12 | DSVKGRFTISRDNSKNTLYLQMNSLRAE |
| | immunoglobulin | DTAVYYC |
| | heavy chain | |
| | variable | |
| | region | |
| | framework | |
| | region 3 | |
| | amino acid | |
| | sequence | |
| | (Paratome) | |
| 40 | Isolate AA12 | WGQGTMVTVSS |
| | immunoglobulin | |
| | heavy chain | |
| | variable | |
| | region | |
| | framework | |
| | region 4 | |
| | amino acid | |
| | sequence | |
| | (Paratome) | |
| 41 | Isolate AA12 | DIQMTQSPLSLSASVGDRVTITCRTS |
| | immunoglobulin | |
| | light chain | |
| | variable | |
| | region | |
| | framework | |
| | region 1 | |
| | amino acid | |
| | sequence | |
| | (Paratome) | |
| 42 | Isolate AA12 | WYQQKPGKAPK |
| | immunoglobulin | |
| | light chain | |
| | variable | |
| | region | |
| | framework | |
| | region 2 | |
| | amino acid | |
| | sequence | |
| | (Paratome) | |
| 43 | Isolate AA12 | GVPSRFSGSGSGTDFTLTISSLQPEDFAT |
| | immunoglobulin | YYC |
| | light chain | |
| | variable | |
| | region | |
| | framework | |
| | region 3 | |
| | amino acid | |
| | sequence | |
| | (Paratome) | |
| 44 | Isolate AA12 | TFGGGTKVEIK |
| | immunoglobulin | |
| | light chain | |
| | variable | |
| | region | |
| | framework | |
| | region 4 | |
| | amino acid | |
| | sequence | |
| | (Paratome) | |

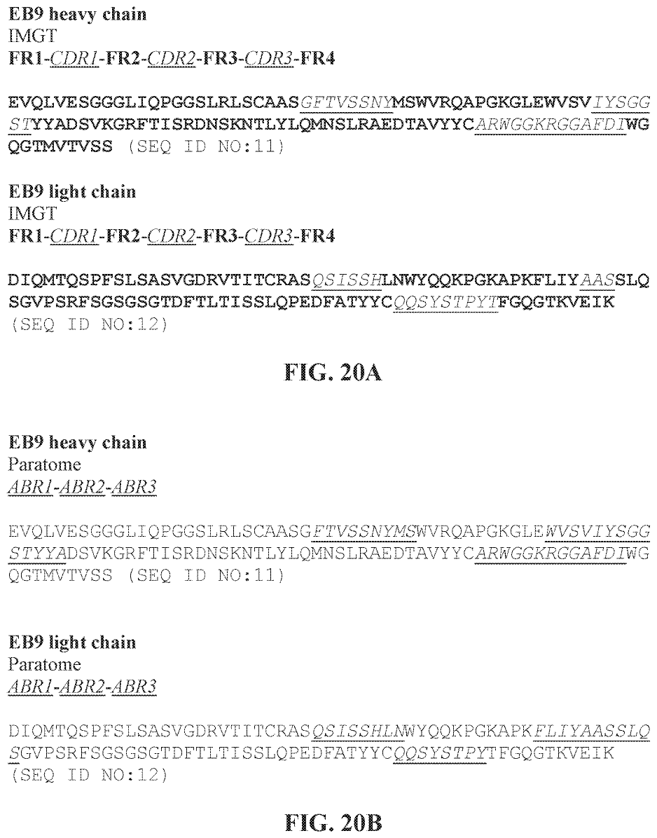

In a specific aspect, an antibody provided herein is the antibody designated EB9 or an antigen-binding fragment thereof. The EB9 antibody is a human antibody. The deduced nucleotide sequences of the variable heavy chain region (“VH” domain) and variable light chain region (“VL” domain) of the antibody EB9 are shown in Table 3. The deduced amino acid sequences of the VH and VL domains of the antibody EB9 are shown in FIGS. 20A-20B and Table 3. The CDRs and framework regions of the VH domain and VL domain are indicated in FIGS. 20A-20B. In addition, Table 3, infra, sets forth the amino acid sequences of the CDRs and framework regions of the variable regions of the antibody EB9. The CDRs and framework regions were determined using the International ImMunoGeneTics (“IMGT”) numbering system. See Lefranc et al., Dev. Comp. Immunol. 27:55-77 (2003), which is incorporated herein by reference in its entirety, for a description of the IMGT numbering system. As an alternative to the IMGT numbering system, the Paratome system may be used. Table 4, infra, sets forth the amino acid sequences of the ABRs and framework regions of the variable regions of the antibody EB9 as determined using the Paratome system. As an alternative to the IMGT numbering system, the Kabat numbering system can be used. Table 2 of Lefranc et al. shows the correspondence between the IMGT and the Kabat numberings. Another alternative to the IMGT numbering system is Chothia. See Chothia and Lesk, J. Mol. Biol. 196:901-917 (1987), which is incorporated herein by reference in its entirety. Further, Oxford's AbM system may be used instead of the IMGT numbering system. A person of ordinary skill in the art would be able to determine the CDRs and framework regions of the variable regions of the EB9 antibody sequence based on the Kabat numbering system, Chothia system, and/or Oxford's AbM system.

TABLE 3
EB9 Antibody

| SEQ | | |
|---|---|---|
| ID | Description | |
| NO: | of Sequence | Sequence |
| 3 | Isolate EB9 | gaggtgcagctggtggagtctggaggaggc |
| | immunoglobu- | ttgatccagcctggggggtccctgagactc |
| | lin | tcctgtgcagcctctgggttcaccgtcagt |
| | heavy chain | agcaactacatgagctgggtccgccaggct |
| | variable | ccagggaaggggctggagtgggtctcagtt |
| | region mRNA, | atttatagcggtggtagcacatactacgca |
| | partial CDS | gactccgtgaagggccgattcaccatctcc |
| | [organism = | agagacaattccaagaacacgctgtatctt |
| | Homo | caaatgaacagcctgagagccgaggacacg |
| | sapiens] | gccgtgtattactgtgcgagatggggaggg |
| | | aaacgggggggggcttttgatatctggggc |
| | | caagggacaatggtcaccgtctcttca |
| 4 | Isolate EB9 | gacatccagatgacccagtctccattctcc |
| | immunoglobu- | ctgtctgcatctgtaggagacagagtcacc |
| | lin | atcacttgccgggcaagtcagagcattagc |
| | light chain | agccatttaaattggtatcagcagaaacca |
| | variable | gggaaagcccctaagttcctgatctatgct |
| | region mRNA, | gcatccagtttgcaaagtggggtcccatca |
| | partial CDS | aggttcagtggcagtggatctgggacagac |
| | [organism = | ttcactctcaccatcagcagtctgcaacct |
| | Homo | gaagattttgcaacttactactgtcaacag |
| | sapiens] | agttacagtactccgtacacttttggccag |
| | | gggaccaaggtggaaatcaaac |
| 11 | Isolate EB9 | EVQLVESGGGLIQPGGSLRLSCAASGFT |
| | immunoglobu- | VSSNYMSWVRQAPGKGLEWVSVIYSGG |
| | lin | STYYADSVKGRFTISRDNSKNTLYLQMN |
| | heavy chain | SLRAEDTAVYYCARWGGKRGGAFDIW |
| | variable | GQGTMVTVSS |
| | region | |
| | amino acid | |
| | sequence | |
| | [organism = | |
| | Homo | |
| | sapiens] | |
| 12 | Isolate EB9 | DIQMTQSPFSLSASVGDRVTITCRASQSIS |
| | immunoglobu- | SHLNWYQQKPGKAPKFLIYAASSLQSGV |
| | lin | PSRFSGSGSGTDFTLTISSLQPEDFATYYC |
| | light chain | QQSYSTPYTFGQGTKVEIK |
| | variable | |
| | region | |
| | amino acid | |
| | sequence | |
| | [organism = | |
| | Homo | |
| | sapiens] | |
| 45 | Isolate EB9 | GFTVSSNY |
| | immunoglobu- | |
| | lin | |
| | heavy chain | |
| | variable | |
| | region CDR1 | |
| | amino acid | |
| | sequence | |
| | (IMGT) | |
| 46 | Isolate EB9 | IYSGGST |
| | immunoglobu- | |
| | lin | |
| | heavy chain | |
| | variable | |
| | region CDR2 | |
| | amino acid | |
| | sequence | |
| | (IMGT) | |
| 47 | Isolate EB9 | ARWGGKRGGAFDI |
| | immunoglobu- | |
| | lin | |
| | heavy chain | |
| | variable | |
| | region CDR3 | |
| | amino acid | |
| | sequence | |
| | (IMGT) | |
| 48 | Isolate EB9 | QSISSH |
| | immunoglobu- | |
| | lin | |
| | light chain | |
| | variable | |
| | region CDR1 | |
| | amino acid | |
| | sequence | |
| | (IMGT) | |
| 49 | Isolate EB9 | AAS |
| | immunoglobu- | |
| | lin | |
| | light chain | |
| | variable | |
| | region CDR2 | |
| | amino acid | |
| | sequence | |
| | (IMGT) | |
| 50 | Isolate EB9 | QQSYSTPYT |
| | immunoglobu- | |
| | lin | |
| | light chain | |
| | variable | |
| | region CDR3 | |
| | amino acid | |
| | sequence | |
| | (IMGT) | |
| 51 | Isolate EB9 | EVQLVESGGGLIQPGGSLRLSCAAS |
| | immunoglobu- | |
| | lin | |
| | heavy chain | |
| | variable | |
| | region | |
| | framework | |
| | region 1 | |
| | amino acid | |
| | sequence | |
| | (IMGT) | |
| 52 | Isolate EB9 | MSWVRQAPGKGLEWVSV |
| | immunoglobu- | |
| | lin | |
| | heavy chain | |
| | variable | |
| | region | |
| | framework | |
| | region 2 | |
| | amino acid | |
| | sequence | |
| | (IMGT) | |
| 53 | Isolate EB9 | YYADSVKGRFTISRDNSKNTLYLQMNSL |
| | immunoglobu- | RAEDTAVYYC |
| | lin | |
| | heavy chain | |
| | variable | |
| | region | |
| | framework | |
| | region 3 | |
| | amino acid | |
| | sequence | |
| | (IMGT) | |
| 54 | Isolate EB9 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (IMGT) | |
| 55 | Isolate EB9 | DIQMTQSPFSLSASVGDRVTITCRAS |
| | immunoglobu- | |
| | lin | |
| | light chain | |
| | variable | |
| | region | |
| | framework | |
| | region 1 | |
| | amino acid | |
| | sequence | |
| | (IMGT) | |
| 56 | Isolate EB9 | LNWYQQKPGKAPKFLIY |
| | immunoglobu- | |
| | lin | |
| | light chain | |
| | variable | |
| | region | |
| | framework | |
| | region 2 | |
| | amino acid | |
| | sequence | |
| | (IMGT) | |
| 57 | Isolate EB9 | SLQSGVPSRFSGSGSGTDFTLTISSLQPED |
| | immunoglobu- | FATYYC |
| | lin | |
| | light chain | |
| | variable | |
| | region | |
| | framework | |
| | region 3 | |
| | amino acid | |
| | sequence | |
| | (IMGT) | |
| 58 | Isolate EB9 | FGQGTKVEIK |
| | immunoglobu- | |
| | lin | |
| | light chain | |
| | variable | |
| | region | |
| | framework | |
| | region 4 | |
| | amino acid | |
| | sequence | |
| | (IMGT) | |

TABLE 4
EB9 Antibody (Paratome)

| SEQ | | |
|---|---|---|
| ID | Description | |
| NO: | of Sequence | Sequence |
| 59 | Isolate EB9 | FTVSSNYMS |
| | immunoglobulin | |
| | heavy chain | |
| | variable | |
| | region ABR1 | |
| | amino acid | |
| | sequence | |
| | (Paratome) | |
| 60 | Isolate EB9 | WVSVIYSGGSTYYA |
| | immunoglobulin | |
| | heavy chain | |
| | variable | |
| | region ABR2 | |
| | amino acid | |
| | sequence | |
| | (Paratome) | |
| 61 | Isolate EB9 | ARWGGKRGGAFDI |
| | immunoglobulin | |
| | heavy chain | |
| | variable | |
| | region ABR3 | |
| | amino acid | |
| | sequence | |
| | (Paratome) | |
| 62 | Isolate EB9 | QSISSHLN |
| | immunoglobulin | |
| | light chain | |
| | variable | |
| | region ABR1 | |
| | amino acid | |
| | sequence | |
| | (Paratome) | |
| 63 | Isolate EB9 | FLIYAASSLQS |
| | immunoglobulin | |
| | light chain | |
| | variable | |
| | region ABR2 | |
| | amino acid | |
| | sequence | |
| | (Paratome) | |
| 64 | Isolate EB9 | QQSYSTPY |
| | immunoglobulin | |
| | light chain | |
| | variable | |
| | region ABR3 | |
| | amino acid | |
| | sequence | |
| | (Paratome) | |
| 65 | Isolate EB9 | EVQLVESGGGLIQPGGSLRLSCAASG |
| | immunoglobulin | |
| | heavy chain | |
| | variable | |
| | region | |
| | framework | |
| | region 1 | |
| | amino acid | |
| | sequence | |
| | (Paratome) | |
| 66 | Isolate EB9 | WVRQAPGKGLE |
| | immunoglobulin | |
| | heavy chain | |
| | variable | |
| | region | |
| | framework | |
| | region 2 | |
| | amino acid | |
| | sequence | |
| | (Paratome) | |
| 67 | Isolate EB9 | DSVKGRFTISRDNSKNTLYLQMNSLRAE |
| | immunoglobulin | DTAVYYC |
| | heavy chain | |
| | variable | |
| | region | |
| | framework | |
| | region 3 | |
| | amino acid | |
| | sequence | |
| | (Paratome) | |
| 68 | Isolate EB9 | WGQGTMVTVSS |
| | immunoglobulin | |
| | heavy chain | |
| | variable | |
| | region | |
| | framework | |
| | region 4 | |
| | amino acid | |
| | sequence | |
| | (Paratome) | |
| 69 | Isolate EB9 | DIQMTQSPFSLSASVGDRVTITCRAS |
| | immunoglobulin | |
| | light chain | |
| | variable | |
| | region | |
| | framework | |
| | region 1 | |
| | amino acid | |
| | sequence | |
| | (Paratome) | |
| 70 | Isolate EB9 | WYQQKPGKAPK |
| | immunoglobulin | |
| | light chain | |
| | variable | |
| | region | |
| | framework | |
| | region 2 | |
| | amino acid | |
| | sequence | |
| | (Paratome) | |
| 71 | Isolate EB9 | GVPSRFSGSGSGTDFTLTISSLQPEDFAT |
| | immunoglobulin | YYC |
| | light chain | |
| | variable | |
| | region | |
| | framework | |
| | region 3 | |
| | amino acid | |
| | sequence | |
| | (Paratome) | |
| 72 | Isolate EB9 | TFGQGTKVEIK |
| | immunoglobulin | |
| | light chain | |
| | variable | |
| | region | |
| | framework | |
| | region 4 | |
| | amino acid | |
| | sequence | |
| | (Paratome) | |

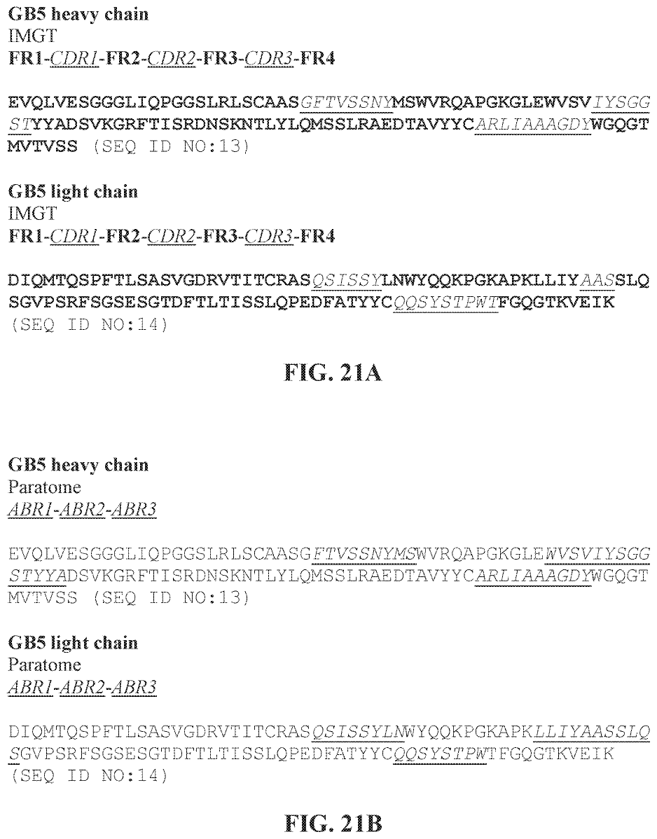

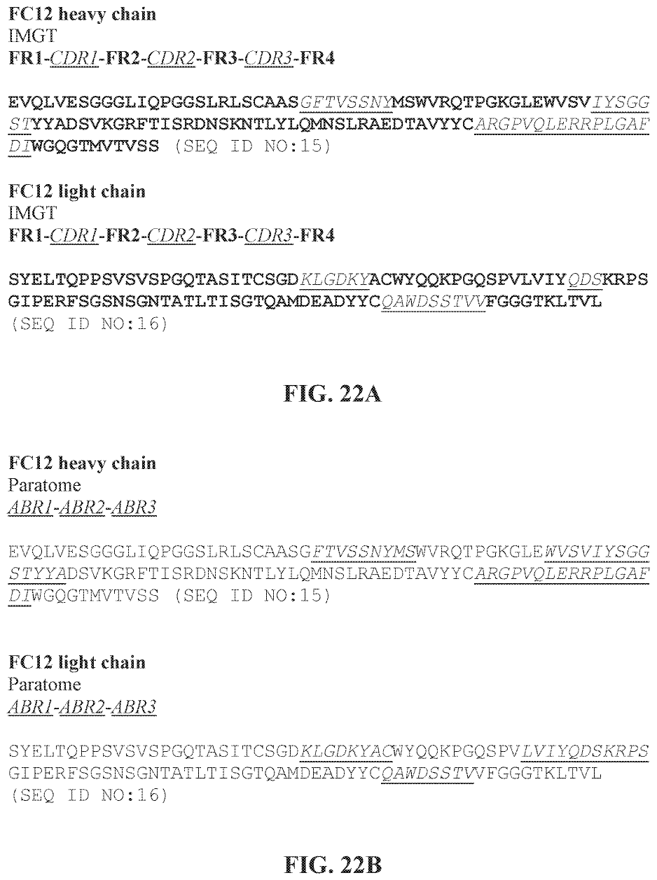

In a specific aspect, an antibody provided herein is the antibody designated GB5 or an antigen-binding fragment thereof. The GB5 antibody is a human antibody. The deduced nucleotide sequences of the variable heavy chain region (“VH” domain) and variable light chain region (“VL” domain) of the antibody GB5 are shown in Table 5. The deduced amino acid sequences of the VH and VL domains of the antibody GB5 are shown in FIGS. 21A-21B and Table 5. The CDRs and framework regions of the VH domain and VL domain are indicated in FIGS. 21A-21B. In addition, Table 5, infra, sets forth the amino acid sequences of the CDRs and framework regions of the variable regions of the antibody EB9. The CDRs and framework regions were determined using the International ImMunoGeneTics (“IMGT”) numbering system. See Lefranc et al., Dev. Comp. Immunol. 27:55-77 (2003), which is incorporated herein by reference in its entirety, for a description of the IMGT numbering system. As an alternative to the IMGT numbering system, the Paratome system may be used. Table 6, infra, sets forth the amino acid sequences of the ABRs and framework regions of the variable regions of the antibody GB5 as determined using the Paratome system. As an alternative to the IMGT numbering system, the Kabat numbering system can be used. Table 2 of Lefranc et al. shows the correspondence between the IMGT and the Kabat numberings. Another alternative to the IMGT numbering system is Chothia. See Chothia and Lesk, J. Mol. Biol. 196:901-917 (1987), which is incorporated herein by reference in its entirety. Further, Oxford's AbM system may be used instead of the IMGT numbering system. A person of ordinary skill in the art would be able to determine the CDRs and framework regions of the variable regions of the GB5 antibody sequence based on the Kabat numbering system, Chothia system, and/or Oxford's AbM system.

TABLE 5
GB5 Antibody

| SEQ | | |
|---|---|---|
| ID | Description | |
| NO: | of Sequence | Sequence |
| 5 | Isolate GB5 | gaggtgcagctggtggagtctggaggaggc |
| | immunoglobu- | ttgatccagcctggggggtccctgagactc |
| | lin | tcctgtgcagcctctgggttcaccgtcagt |
| | heavy chain | agcaactacatgagctgggtccgccaggct |
| | variable | ccagggaaggggctggagtgggtctcagtt |
| | region mRNA, | atttatagcggtggtagcacatactacgca |
| | partial CDS | gactccgtgaagggccgattcaccatctcc |
| | [organism = | agagacaattccaagaacacgctgtatctt |
| | Homo | caaatgagcagcctgagagccgaggacacg |
| | sapiens] | gccgtgtattactgtgcgagactcatagca |
| | | gcagctggtgactactggggccagggaaca |
| | | atggtcaccgtctcttcag |
| 6 | Isolate GB5 | gacatccagatgacccagtctccattcacc |
| | immunoglobu- | ctgtctgcatctgtaggagacagagtcacc |
| | lin | atcacttgccgggcaagtcagagcattagc |
| | light chain | agctatttaaattggtatcagcagaaacca |
| | variable | gggaaagcccctaagctcctgatctatgct |
| | region mRNA, | gcatccagtttgcaaagtggggtcccatca |
| | partial CDS | aggttcagtggcagtgaatctgggacagat |
| | [organism = | ttcactctcaccatcagcagtctgcaacct |
| | Homo | gaagattttgcaacttactactgtcaacag |
| | sapiens] | agttacagtaccccctggacgttcggccaa |
| | | gggaccaaggtggagatcaaac |
| 13 | Isolate GB5 | EVQLVESGGGLIQPGGSLRLSCAASGFT |
| | immunoglobu- | VSSNYMSWVRQAPGKGLEWVSVIYSGG |
| | lin | STYYADSVKGRFTISRDNSKNTLYLQMS |
| | heavy chain | SLRAEDTAVYYCARLIAAAGDYWGQGT |
| | variable | MVTVSS |
| | region | |
| | amino acid | |
| | sequence | |
| | [organism = | |
| | Homo | |
| | sapiens] | |
| 14 | Isolate GB5 | DIQMTQSPFTLSASVGDRVTITCRASQSI |
| | immunoglobu- | SSYLNWYQQKPGKAPKLLIYAASSLQSG |
| | lin | VPSRFSGSESGTDFTLTISSLQPEDFATYY |
| | light chain | CQQSYSTPWTFGQGTKVEIK |
| | variable | |
| | region | |
| | amino acid | |
| | sequence | |
| | [organism = | |
| | Homo | |
| | sapiens] | |
| 73 | Isolate GB5 | GFTVSSNY |
| | immunoglobu- | |
| | lin | |
| | heavy chain | |
| | variable | |
| | region CDR1 | |
| | amino acid | |
| | sequence | |
| | (IMGT) | |
| 74 | Isolate GB5 | IYSGGST |
| | immunoglobu- | |
| | lin | |
| | heavy chain | |
| | variable | |
| | region CDR2 | |
| | amino acid | |
| | sequence | |
| | (IMGT) | |
| 75 | Isolate GB5 | ARLIAAAGDY |
| | immunoglobu- | |
| | lin | |
| | heavy chain | |
| | variable | |
| | region CDR3 | |
| | amino acid | |
| | sequence | |
| | (IMGT) | |
| 76 | Isolate GB5 | QSISSY |
| | immunoglobu- | |
| | lin | |
| | light chain | |
| | variable | |
| | region CDR1 | |
| | amino acid | |
| | sequence | |
| | (IMGT) | |
| 77 | Isolate GB5 | AAS |
| | immunoglobu- | |
| | lin | |
| | light chain | |
| | variable | |
| | region CDR2 | |
| | amino acid | |
| | sequence | |
| | (IMGT) | |
| 78 | Isolate GB5 | QQSYSTPWT |
| | immunoglobu- | |
| | lin | |
| | light chain | |
| | variable | |
| | region CDR3 | |
| | amino acid | |
| | sequence | |
| | (IMGT) | |
| 79 | Isolate GB5 | |
| | immunoglobu- | EVQLVESGGGLIQPGGSLRLSCAAS |
| | lin | |
| | heavy chain | |
| | variable | |
| | region | |
| | framework | |
| | region 1 | |
| | amino acid | |
| | sequence | |
| | (IMGT) | |
| 80 | Isolate GB5 | MSWVRQAPGKGLEWVSV |
| | immunoglobu- | |
| | lin | |
| | heavy chain | |
| | variable | |
| | region | |
| | framework | |
| | region 2 | |
| | amino acid | |
| | sequence | |
| | (IMGT) | |
| 81 | Isolate GB5 | YYADSVKGRFTISRDNSKNTLYLQMSSL |
| | immunoglobu- | RAEDTAVYYC |
| | lin | |
| | heavy chain | |
| | variable | |
| | region | |
| | framework | |
| | region 3 | |
| | amino acid | |
| | sequence | |
| | (IMGT) | |
| 82 | Isolate GB5 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (IMGT) | |
| 83 | Isolate GB5 | DIQMTQSPFTLSASVGDRVTITCRAS |
| | immunoglobu- | |
| | lin | |
| | light chain | |
| | variable | |
| | region | |
| | framework | |
| | region 1 | |
| | amino acid | |
| | sequence | |
| | (IMGT) | |
| 84 | Isolate GB5 | LNWYQQKPGKAPKLLIY |
| | immunoglobu- | |
| | lin | |
| | light chain | |
| | variable | |
| | region | |
| | framework | |
| | region 2 | |
| | amino acid | |
| | sequence | |
| | (IMGT) | |
| 85 | Isolate GB5 | SLQSGVPSRFSGSESGTDFTLTISSLQPED |
| | immunoglobu- | FATYYC |
| | lin | |
| | light chain | |
| | variable | |
| | region | |
| | framework | |
| | region 3 | |
| | amino acid | |
| | sequence | |
| | (IMGT) | |
| 86 | Isolate GB5 | FGQGTKVEIK |
| | immunoglobu- | |
| | lin | |
| | light chain | |
| | variable | |
| | region | |
| | framework | |
| | region 4 | |
| | amino acid | |
| | sequence | |
| | (IMGT) | |

TABLE 6
GB5 Antibody (Paratome)

| SEQ | | |
|---|---|---|
| ID | Description | |
| NO: | of Sequence | Sequence |
| 87 | Isolate GB5 | |
| | immunoglobu- | FTVSSNYMS |
| | lin | |
| | heavy chain | |
| | variable | |
| | region ABR1 | |
| | amino acid | |
| | sequence | |
| | (Paratome) | |
| 88 | Isolate GB55 | WVSVIYSGGSTYYA |
| | immunoglobu- | |
| | lin | |
| | heavy chain | |
| | variable | |
| | region ABR2 | |
| | amino acid | |
| | sequence | |
| | (Paratome) | |
| 89 | Isolate GB5 | ARLIAAAGDY |
| | immunoglobu- | |
| | lin | |
| | heavy chain | |
| | variable | |
| | region ABR3 | |
| | amino acid | |
| | sequence | |
| | (Paratome) | |
| 90 | Isolate GB5 | QSISSYLN |
| | immunoglobu- | |
| | lin | |
| | light chain | |
| | variable | |
| | region ABR1 | |
| | amino acid | |
| | sequence | |
| | (Paratome) | |
| 91 | Isolate GB5 | LLIYAASSLQS |
| | immunoglobu- | |
| | lin | |
| | light chain | |
| | variable | |
| | region ABR2 | |
| | amino acid | |
| | sequence | |
| | (Paratome) | |
| 92 | Isolate GB5 | QQSYSTPW |
| | immunoglobu- | |
| | lin | |
| | light chain | |
| | variable | |
| | region ABR3 | |
| | amino acid | |
| | sequence | |
| | (Paratome) | |
| 93 | Isolate GB5 | EVQLVESGGGLIQPGGSLRLSCAASG |
| | immunoglobu- | |
| | lin | |
| | heavy chain | |
| | variable | |
| | region | |
| | framework | |
| | region 1 | |
| | amino acid | |
| | sequence | |
| | (Paratome) | |
| 94 | Isolate GB5 | WVRQAPGKGLE |
| | immunoglobu- | |
| | lin | |
| | heavy chain | |
| | variable | |
| | region | |
| | framework | |
| | region 2 | |
| | amino acid | |
| | sequence | |
| | (Paratome) | |
| 95 | Isolate GB5 | DSVKGRFTISRDNSKNTLYLQMSSLRAE |
| | immunoglobu- | DTAVYYC |
| | lin | |
| | heavy chain | |
| | variable | |
| | region | |
| | framework | |
| | region 3 | |
| | amino acid | |
| | sequence | |
| | (Paratome) | |
| 96 | Isolate GB5 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (Paratome) | |
| 97 | Isolate GB5 | DIQMTQSPFTLSASVGDRVTITCRAS |
| | immunoglobu- | |
| | lin | |
| | light chain | |
| | variable | |
| | region | |
| | framework | |
| | region 1 | |
| | amino acid | |
| | sequence | |
| | (Paratome) | |
| 98 | Isolate GB5 | WYQQKPGKAPK |
| | immunoglobu- | |
| | lin | |
| | light chain | |
| | variable | |
| | region | |
| | framework | |
| | region 2 | |
| | amino acid | |
| | sequence | |
| | (Paratome) | |
| 99 | Isolate GB5 | GVPSRFSGSESGTDFTLTISSLQPEDFATY |
| | immunoglobu- | YC |
| | lin | |
| | light chain | |
| | variable | |
| | region | |
| | framework | |
| | region 3 | |
| | amino acid | |
| | sequence | |
| | (Paratome) | |
| 100 | Isolate GB5 | TFGQGTKVEIK |
| | immunoglobu- | |
| | lin | |
| | light chain | |
| | variable | |
| | region | |
| | framework | |
| | region 4 | |
| | amino acid | |
| | sequence | |
| | (Paratome) | |

In a specific aspect, an antibody provided herein is the antibody designated FC12 or an antigen-binding fragment thereof. The FC12 antibody is a human antibody. The deduced nucleotide sequences of the variable heavy chain region (“VH” domain) and variable light chain region (“VL” domain) of the antibody FC12 are shown in Table 7. The deduced amino acid sequences of the VH and VL domains of the antibody FC12 are shown in FIGS. 22A-22B and Table 7. The CDRs and framework regions of the VH domain and VL domain are indicated in FIGS. 22A-22B. In addition, Table 7, infra, sets forth the amino acid sequences of the CDRs and framework regions of the variable regions of the antibody FC12. The CDRs and framework regions were determined using the International ImMunoGeneTics (“IMGT”) numbering system. See Lefranc et al., Dev. Comp. Immunol. 27:55-77 (2003), which is incorporated herein by reference in its entirety, for a description of the IMGT numbering system. As an alternative to the IMGT numbering system, the Paratome system may be used. Table 8, infra, sets forth the amino acid sequences of the ABRs and framework regions of the variable regions of the antibody FC12 as determined using the Paratome system. As an alternative to the IMGT numbering system, the Kabat numbering system can be used. Table 2 of Lefranc et al. shows the correspondence between the IMGT and the Kabat numberings. Another alternative to the IMGT numbering system is Chothia. See Chothia and Lesk, J. Mol. Biol. 196:901-917 (1987), which is incorporated herein by reference in its entirety. Further, Oxford's AbM system may be used instead of the IMGT numbering system. A person of ordinary skill in the art would be able to determine the CDRs and framework regions of the variable regions of the FC12 antibody sequence based on the Kabat numbering system, Chothia system, and/or Oxford's AbM system.

TABLE 7
FC12 Antibody

| SEQ | | |
|---|---|---|
| ID | Description | |
| NO: | of Sequence | Sequence |
| 7 | Isolate FC12 | gaggtgcagctggtggagtctggaggaggc |
| | immunoglobu- | ttgatccagcctggggggtccctgagactc |
| | lin | tcctgtgcagcctctgggttcaccgtcagt |
| | heavy chain | agcaactacatgagctgggtccgccagact |
| | variable | ccagggaaggggctggagtgggtctcagtt |
| | region mRNA, | atttatagcggtggtagcacatactacgca |
| | partial CDS | gactccgtgaagggccgattcaccatctcc |
| | [organism = | agagacaattccaagaacacgctgtatctt |
| | Homo | caaatgaacagcctgagagccgaggacacg |
| | sapiens] | gccgtgtattactgtgcgagagggcccgta |
| | | caactggaacgacggcctctgggtgctttt |
| | | gatatctggggccaagggacaatggtcacc |
| | | gtctcttca |
| 8 | Isolate FC12 | Tcctatgagctgactcagccaccctcagtg |
| | immunoglobu- | tccgtgtccccaggacagacagccagcatc |
| | lin | acctgctctggagataaattgggggataaa |
| | light chain | tatgcttgctggtatcagcagaagccaggc |
| | variable | cagtcccctgtgctggtcatctatcaagat |
| | region mRNA, | agcaagcggccctcagggatccctgagcga |
| | partial CDS | ttctctggctccaactctgggaacacagcc |
| | [organism = | actctgaccatcagcgggacccaggctatg |
| | Homo | gatgaggctgactattactgtcaggcgtgg |
| | sapiens] | gacagcagcaccgtggtattcggcggaggg |
| | | accaagctgaccgtcctag |
| 15 | Isolate FC12 | EVQLVESGGGLIQPGGSLRLSCAASGFT |
| | immunoglobu- | VSSNYMSWVRQTPGKGLEWVSVIYSGG |
| | lin | STYYADSVKGRFTISRDNSKNTLYLQMN |
| | heavy chain | SLRAEDTAVYYCARGPVQLERRPLGAF |
| | variable | DIWGQGTMVTVSS |
| | region | |
| | amino acid | |
| | sequence | |
| | [organism = | |
| | Homo | |
| | sapiens] | |
| 16 | Isolate FC12 | SYELTQPPSVSVSPGQTASITCSGDKLGD |
| | immunoglobu- | KYACWYQQKPGQSPVLVIYQDSKRPSGI |
| | lin | PERFSGSNSGNTATLTISGTQAMDEADY |
| | light chain | YCQAWDSSTVVFGGGTKLTVL |
| | variable | |
| | region | |
| | amino acid | |
| | sequence | |
| | [organism = | |
| | Homo | |
| | sapiens] | |
| 101 | Isolate FC12 | GFTVSSNY |
| | immunoglobu- | |
| | lin | |
| | heavy chain | |
| | variable | |
| | region CDR1 | |
| | amino acid | |
| | sequence | |
| | (IMGT) | |
| 102 | Isolate FC12 | IYSGGST |
| | immunoglobu- | |
| | lin | |
| | heavy chain | |
| | variable | |
| | region CDR2 | |
| | amino acid | |
| | sequence | |
| | (IMGT) | |
| 103 | Isolate FC12 | ARGPVQLERRPLGAFDI |
| | immunoglobu- | |
| | lin | |
| | heavy chain | |
| | variable | |
| | region CDR3 | |
| | amino acid | |
| | sequence | |
| | (IMGT) | |
| 104 | Isolate FC12 | KLGDKY |
| | immunoglobu- | |
| | lin | |
| | light chain | |
| | variable | |
| | region CDR1 | |
| | amino acid | |
| | sequence | |
| | (IMGT) | |
| 105 | Isolate FC12 | QDS |
| | immunoglobu- | |
| | lin | |
| | light chain | |
| | variable | |
| | region CDR2 | |
| | amino acid | |
| | sequence | |
| | (IMGT) | |
| 106 | Isolate FC12 | QAWDSSTVV |
| | immunoglobu- | |
| | lin | |
| | light chain | |
| | variable | |
| | region CDR3 | |
| | amino acid | |
| | sequence | |
| | (IMGT) | |
| 107 | Isolate FC12 | EVQLVESGGGLIQPGGSLRLSCAAS |
| | immunoglobu- | |
| | lin | |
| | heavy chain | |
| | variable | |
| | region | |
| | framework | |
| | region 1 | |
| | amino acid | |
| | sequence | |
| | (IMGT) | |
| 108 | Isolate FC12 | MSWVRQTPGKGLEWVSV |
| | immunoglobu- | |
| | lin | |
| | heavy chain | |
| | variable | |
| | region | |
| | framework | |
| | region 2 | |
| | amino acid | |
| | sequence | |
| | (IMGT) | |
| 109 | Isolate FC12 | YYADSVKGRFTISRDNSKNTLYLQMNSL |
| | immunoglobu- | RAEDTAVYYC |
| | lin | |
| | heavy chain | |
| | variable | |
| | region | |
| | framework | |
| | region 3 | |
| | amino acid | |
| | sequence | |
| | (IMGT) | |
| 110 | Isolate FC12 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (IMGT) | |
| 111 | Isolate FC12 | SYELTQPPSVSVSPGQTASITCSGD |
| | immunoglobu- | |
| | lin | |
| | light chain | |
| | variable | |
| | region | |
| | framework | |
| | region 1 | |
| | amino acid | |
| | sequence | |
| | (IMGT) | |
| 112 | Isolate FC12 | ACWYQQKPGQSPVLVIY |
| | immunoglobu- | |
| | lin | |
| | light chain | |
| | variable | |
| | region | |
| | framework | |
| | region 2 | |
| | amino acid | |
| | sequence | |
| | (IMGT) | |
| 113 | Isolate FC12 | KRPSGIPERFSGSNSGNTATLTISGTQAM |
| | immunoglobu- | DEADYYC |
| | lin | |
| | light chain | |
| | variable | |
| | region | |
| | framework | |
| | region 3 | |
| | amino acid | |
| | sequence | |
| | (IMGT) | |
| 114 | Isolate FC12 | FGGGTKLTVL |
| | immunoglobu- | |
| | lin | |
| | light chain | |
| | variable | |
| | region | |
| | framework | |
| | region 4 | |
| | amino acid | |
| | sequence | |
| | (IMGT) | |

TABLE 8
FC12 Antibody (Paratome)

| SEQ | | |
|---|---|---|
| ID | Description | |
| NO: | of Sequence | Sequence |
| 115 | Isolate FC12 | FTVSSNYMS |
| | immunoglobu- | |
| | lin | |
| | heavy chain | |
| | variable | |
| | region ABR1 | |
| | amino acid | |
| | sequence | |
| | (Paratome) | |
| 116 | Isolate FC12 | WVSVIYSGGSTYYA |
| | immunoglobu- | |
| | lin | |
| | heavy chain | |
| | variable | |
| | region ABR2 | |
| | amino acid | |
| | sequence | |
| | (Paratome) | |
| 117 | Isolate FC12 | ARGPVQLERRPLGAFDI |
| | immunoglobu- | |
| | lin | |
| | heavy chain | |
| | variable | |
| | region ABR3 | |
| | amino acid | |
| | sequence | |
| | (Paratome) | |
| 118 | Isolate FC12 | KLGDKYAC |
| | immunoglobu- | |
| | lin | |
| | light chain | |
| | variable | |
| | region ABR1 | |
| | amino acid | |
| | sequence | |
| | (Paratome) | |
| 119 | Isolate FC12 | LVIYQDSKRPS |
| | immunoglobu- | |
| | lin | |
| | light chain | |
| | variable | |
| | region ABR2 | |
| | amino acid | |
| | sequence | |
| | (Paratome) | |
| 120 | Isolate FC12 | QAWDSSTV |
| | immunoglobu- | |
| | lin | |
| | light chain | |
| | variable | |
| | region ABR3 | |
| | amino acid | |
| | sequence | |
| | (Paratome) | |
| 121 | Isolate FC12 | EVQLVESGGGLIQPGGSLRLSCAASG |
| | immunoglobu- | |
| | lin | |
| | heavy chain | |
| | variable | |
| | region | |
| | framework | |
| | region 1 | |
| | amino acid | |
| | sequence | |
| | (Paratome) | |
| 122 | Isolate FC12 | WVRQTPGKGLE |
| | immunoglobu- | |
| | lin | |
| | heavy chain | |
| | variable | |
| | region | |
| | framework | |
| | region 2 | |
| | amino acid | |
| | sequence | |
| | (Paratome) | |
| 123 | Isolate FC12 | DSVKGRFTISRDNSKNTLYLQMNSLRAE |
| | immunoglobu- | DTAVYYC |
| | lin | |
| | heavy chain | |
| | variable | |
| | region | |
| | framework | |
| | region 3 | |
| | amino acid | |
| | sequence | |
| | (Paratome) | |
| 124 | Isolate FC12 | WGQGTMVTVSS |
| | immunoglobu- | |
| | lin | |
| | heavy chain | |
| | variable | |
| | region | |
| | framework | |
| | region 4 | |
| | amino acid | |
| | sequence | |
| | (Paratome) | |
| 125 | Isolate FC12 | SYELTQPPSVSVSPGQTASITCSGD |
| | immunoglobu- | |
| | lin | |
| | light chain | |
| | variable | |
| | region | |
| | framework | |
| | region 1 | |
| | amino acid | |
| | sequence | |
| | (Paratome) | |
| 126 | Isolate FC12 | WYQQKPGQSPV |
| | immunoglobu- | |
| | lin | |
| | light chain | |
| | variable | |
| | region | |
| | framework | |
| | region 2 | |
| | amino acid | |
| | sequence | |
| | (Paratome) | |
| 127 | Isolate FC12 | GIPERFSGSNSGNTATLTISGTQAMDEAD |
| | immunoglobu- | YYC |
| | lin | |
| | light chain | |
| | variable | |
| | region | |
| | framework | |
| | region 3 | |
| | amino acid | |
| | sequence | |
| | (Paratome) | |
| 128 | Isolate FC12 | VFGGGTKLTVL |
| | immunoglobu- | |
| | lin | |
| | light chain | |
| | variable | |
| | region | |
| | framework | |
| | region 4 | |
| | amino acid | |
| | sequence | |
| | (Paratome) | |

In a specific embodiment, the position of a CDR along the VH and/or VL domain of an antibody described herein may vary by one, two, three or four amino acid positions so long as binding to Zika virus NS1 (e.g., NS1 of a Zika virus strain described herein, such as in Section 6, infra) is maintained or substantially maintained (for example, by at least 50%, at least 60%, at least 70%, at least 80%, at least 90%, at least 95% in an assay known in the art or described herein, such as an ELISA). For example, in one embodiment, the position defining a CDR of antibody AA12 may vary by shifting the N-terminal and/or C-terminal boundary of the CDR by one, two, three, or four amino acids, relative to the CDR position depicted in FIGS. 19A-19B (either by IMGT or Paratome), so long as binding to Zika virus NS1 (e.g., NS1 of a Zika virus strain described herein, such as in Section 6, infra) is maintained or substantially maintained (for example, by at least 50%, at least 60%, at least 70%, at least 80%, at least 90%, at least 95% in an assay known in the art or described herein, such as an ELISA). In another example, in one embodiment, the position defining a CDR of antibody EB9 may vary by shifting the N-terminal and/or C-terminal boundary of the CDR by one, two, three, or four amino acids, relative to the CDR position depicted in FIGS. 20A-20B (either by IMGT or Paratome), so long as binding to Zika virus NS1 (e.g., NS1 of a Zika virus strain described herein, such as in Section 6, infra) is maintained or substantially maintained (for example, by at least 50%, at least 60%, at least 70%, at least 80%, at least 90%, at least 95% in an assay known in the art or described herein, such as an ELISA). In another example, in one embodiment, the position defining a CDR of antibody GB5 may vary by shifting the N-terminal and/or C-terminal boundary of the CDR by one, two, three, or four amino acids, relative to the CDR position depicted in FIGS. 21A-21B (either by IMGT or Paratome), so long as binding to Zika virus NS1 (e.g., NS1 of a Zika virus strain described herein, such as in Section 6, infra) is maintained or substantially maintained (for example, by at least 50%, at least 60%, at least 70%, at least 80%, at least 90%, at least 95% in an assay known in the art or described herein, such as an ELISA). In another example, in one embodiment, the position defining a CDR of antibody FC12 may vary by shifting the N-terminal and/or C-terminal boundary of the CDR by one, two, three, or four amino acids, relative to the CDR position depicted in FIGS. 22A-22B (either by IMGT or Paratome), so long as binding to Zika virus NS1 (e.g., NS1 of a Zika virus strain described herein, such as in Section 6, infra) is maintained or substantially maintained (for example, by at least 50%, at least 60%, at least 70%, at least 80%, at least 90%, at least 95% in an assay known in the art or described herein, such as an ELISA).

In another aspect, provided herein are antibodies that bind to a Zika virus NS1 comprising one, two or three complementarity determining regions (CDRs) of the variable heavy chain region of the antibody AA12, EB9, GB5, or FC12 and one, two or three CDRs of the variable light chain region of the antibody AA12, EB9, GB5, or FC12. In certain embodiments, an antibody that binds to a Zika virus NS1 (e.g., a Zika virus NS1 described in Section 6, infra), comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of the antibody AA12, EB9, GB5, or FC12.

In a specific aspect, provided herein are antibodies that bind to a Zika virus NS1 comprising one, two or three complementarity determining regions (CDRs) of the variable heavy chain region of the antibody AA12 and one, two or three CDRs of the variable light chain region of the antibody AA12. In certain embodiments, an antibody that binds to a Zika virus NS1 (e.g., a Zika virus NS1 described in Section 6, infra), comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of the antibody AA12.

In a specific aspect, provided herein are antibodies that bind to a Zika virus NS1 comprising one, two or three complementarity determining regions (CDRs) of the variable heavy chain region of the antibody EB9 and one, two or three CDRs of the variable light chain region of the antibody EB9. In certain embodiments, an antibody that binds to a Zika virus NS1 (e.g., a Zika virus NS1 described in Section 6, infra), comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of the antibody EB9.

In a specific aspect, provided herein are antibodies that bind to a Zika virus NS1 comprising one, two or three complementarity determining regions (CDRs) of the variable heavy chain region of the antibody GB5 and one, two or three CDRs of the variable light chain region of the antibody GB5. In certain embodiments, an antibody that binds to a Zika virus NS1 (e.g., a Zika virus NS1 described in Section 6, infra), comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of the antibody GB5.

In a specific aspect, provided herein are antibodies that bind to a Zika virus NS1 comprising one, two or three complementarity determining regions (CDRs) of the variable heavy chain region of the antibody FC12 and one, two or three CDRs of the variable light chain region of the antibody FC12. In certain embodiments, an antibody that binds to a Zika virus NS1 (e.g., a Zika virus NS1 described in Section 6, infra), comprises (or alternatively, consists of) a VH CDR1 and a VL CDR1; a VH CDR1 and a VL CDR2; a VH CDR1 and a VL CDR3; a VH CDR2 and a VL CDR1; VH CDR2 and a VL CDR2; a VH CDR2 and a VL CDR3; a VH CDR3 and a VL CDR1; a VH CDR3 and a VL CDR2; a VH CDR3 and a VL CDR3; a VH1 CDR1, a VH CDR2 and a VL CDR1; a VH CDR1, a VH CDR2 and a VL CDR2; a VH CDR1, a VH CDR2 and a VL CDR3; a VH CDR2, a VH CDR3 and a VL CDR1; a VH CDR2, a VH CDR3 and a VL CDR2; a VH CDR2, a VH CDR3 and a VL CDR3; a VH CDR1, a VL CDR1 and a VL CDR2; a VH CDR1, a VL CDR1 and a VL CDR3; a VH CDR2, a VL CDR1 and a VL CDR2; a VH CDR2, a VL CDR1 and a VL CDR3; a VH CDR3, a VL CDR1 and a VL CDR2; a VH CDR3, a VL CDR1 and a VL CDR3; a VH CDR1, a VH CDR2, a VH CDR3 and a VL CDR1; a VH CDR1, a VH CDR2, a VH CDR3 and a VL CDR2; a VH CDR1, a VH CDR2, a VH CDR3 and a VL CDR3; a VH CDR1, a VH CDR2, a VL CDR1 and a VL CDR2; a VH CDR1, a VH CDR2, a VL CDR1 and a VL CDR3; a VH CDR1, a VH CDR3, a VL CDR1 and a VL CDR2; a VH CDR1, a VH CDR3, a VL CDR1 and a VL CDR3; a VH CDR2, a VH CDR3, a VL CDR1 and a VL CDR2; a VH CDR2, a VH CDR3, a VL CDR1 and a VL CDR3; a VH CDR2, a VH CDR3, a VL CDR2 and a VL CDR3; a VH CDR1, a VH CDR2, a VH CDR3, a VL CDR1 and a VL CDR2; a VH CDR1, a VH CDR2, a VH CDR3, a VL CDR1 and a VL CDR3; a VH CDR1, a VH CDR2, a VL CDR1, a VL CDR2, and a VL CDR3; a VH CDR1, a VH CDR3, a VL CDR1, a VL CDR2, and a VL CDR3; a VH CDR2, a VH CDR3, a VL CDR1, a VL CDR2, and a VL CDR3; a VH CDR1, VH CDR2, a VH CDR3, a VL CDR1, a VL CDR2, and a VL CDR3; or any combination thereof of the VH CDRs and VL CDRs of the antibody FC12.

In another embodiment, an antibody, which binds to a Zika virus NS1 (e.g., NS1 of a Zika virus strain described herein, such as in Section 6, infra), comprises one, two, three, four, five or all six complementarity determining regions (CDRs) of the antibody AA12. In certain embodiments, an antibody, which binds to a Zika virus NS1 (.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 20-22, respectively. In certain embodiments, the light chain or VL domain comprises one, two or three of framework region (FR)1, FR2, FR3, and FR4 comprising the amino acid sequences of SEQ ID NO: 27-30, respectively. In some embodiments, the light chain or VL domain comprises framework region (FR)1, FR2, FR3, and FR4 comprising the amino acid sequences of SEQ ID NO: 27-30, respectively. In other embodiments, the light chain or VL domain comprises other human framework regions or framework regions derived from another human antibody.

In some embodiments, an antibody, which binds to a Zika virus NS1 (e.g., e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 17-19, respectively. In certain embodiments, the heavy chain or VH domain comprises one, two or three of framework region (FR)1, FR2, FR3, and FR4 comprising the amino acid sequences of SEQ ID NO: 23-26, respectively. In some embodiments, the heavy chain or VH domain comprises framework region (FR)1, FR2, FR3, and FR4 comprising the amino acid sequences of SEQ ID NO: 23-26, respectively. In other embodiments, the heavy chain or VH domain comprises human framework regions or framework regions derived from a human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 20-22, respectively; and a VH domain or heavy chain comprising a VH CDR1, VH CDR2, and VH CDR3 comprising the amino acid sequences of SEQ ID NOs: 17-19, respectively. In certain embodiments, the light chain or VL domain comprises one, two or three of framework region (FR)1, FR2, FR3, and FR4 comprising the amino acid sequences of SEQ ID NO: 27-30, respectively, and the heavy chain or VH domain comprises one, two or three of framework regions (FR)1, FR2, FR3, and FR4 comprising the amino acid sequences of SEQ ID NO: 23-26, respectively. In some embodiments, the light chain or VL domain comprises framework region (FR)1, FR2, FR3, and FR4 comprising the amino acid sequences of SEQ ID NO: 27-30, respectively, and the heavy chain or VH domain comprises framework regions (FR)1, FR2, FR3, and FR4 comprising the amino acid sequences of SEQ ID NO: 23-26, respectively. In other embodiments, the light chain or VL domain and heavy chain or VH domain comprises other human framework regions or framework regions derived from another human antibody.

In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 34-36, respectively. In certain embodiments, the light chain or VL domain comprises one, two or three of framework region (FR)1, FR2, FR3, and FR4 comprising the amino acid sequences of SEQ ID NO: 41-44, respectively. In some embodiments, the light chain or VL domain comprises framework region (FR)1, FR2, FR3, and FR4 comprising the amino acid sequences of SEQ ID NO: 41-44, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 31-33, respectively. In certain embodiments, the heavy chain or VH domain comprises one, two or three of framework region (FR)1, FR2, FR3, and FR4 comprising the amino acid sequences of SEQ ID NO: 37-40, respectively. In some embodiments, the heavy chain or VH domain comprises framework region (FR)1, FR2, FR3, and FR4 comprising the amino acid sequences of SEQ ID NO: 37-40, respectively. In other embodiments, the heavy chain or VH domain comprises other human framework regions or framework regions derived from another human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 34-36, respectively; and a VH domain or heavy chain comprising a VH CDR1, VH CDR2, and VH CDR3 comprising the amino acid sequences of SEQ ID NOs: 31-33, respectively. In certain embodiments, the light chain or VL domain comprises one, two or three of framework region (FR)1, FR2, FR3, and FR4 comprising the amino acid sequences of SEQ ID NO: 41-44, respectively, and the heavy chain or VH domain comprises one, two or three of framework regions (FR)1, FR2, FR3, and FR4 comprising the amino acid sequences of SEQ ID NO: 37-40, respectively. In some embodiments, the light chain or VL domain comprises framework region (FR)1, FR2, FR3, and FR4 comprising the amino acid sequences of SEQ ID NO: 41-44, respectively, and the heavy chain or VH domain comprises framework regions (FR)1, FR2, FR3, and FR4 comprising the amino acid sequences of SEQ ID NO: 37-40, respectively. In other embodiments, the light chain or VL domain and heavy chain or VH domain comprises other human framework regions or framework regions derived from another human antibody.

In another embodiment, an antibody, which binds to a Zika virus NS1 (e.g., NS1 of a Zika virus strain described herein, such as in Section 6, infra), comprises one, two, three, four, five or all six complementarity determining regions (CDRs) of the antibody EB9. In certain embodiments, an antibody, which binds to Zika virus NS1 (.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 48-50, respectively. In certain embodiments, the light chain or VL domain comprises one, two or three of framework region (FR)1, FR2, FR3, and FR4 comprising the amino acid sequences of SEQ ID NO: 55-58, respectively. In some embodiments, the light chain or VL domain comprises framework region (FR)1, FR2, FR3, and FR4 comprising the amino acid sequences of SEQ ID NO: 55-58, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 45-47, respectively. In certain embodiments, the heavy chain or VH domain comprises one, two or three of framework region (FR)1, FR2, FR3, and FR4 comprising the amino acid sequences of SEQ ID NO: 51-54, respectively. In some embodiments, the heavy chain or VH domain comprises framework region (FR)1, FR2, FR3, and FR4 comprising the amino acid sequences of SEQ ID NO: 51-54, respectively. In other embodiments, the heavy chain or VH domain comprises other human framework regions or framework regions derived from another human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 48-50, respectively; and a VH domain or heavy chain comprising a VH CDR1, VH CDR2, and VH CDR3 comprising the amino acid sequences of SEQ ID NOs: 45-47, respectively. In certain embodiments, the light chain or VL domain comprises one, two or three of framework region (FR)1, FR2, FR3, and FR4 comprising the amino acid sequences of SEQ ID NO: 55-58, respectively, and the heavy chain or VH domain comprises one, two or three of framework regions (FR)1, FR2, FR3, and FR4 comprising the amino acid sequences of SEQ ID NO: 51-54, respectively. In some embodiments, the light chain or VL domain comprises framework region (FR)1, FR2, FR3, and FR4 comprising the amino acid sequences of SEQ ID NO: 55-58, respectively, and the heavy chain or VH domain comprises framework regions (FR)1, FR2, FR3, and FR4 comprising the amino acid sequences of SEQ ID NO: 51-54, respectively. In other embodiments, the light chain or VL domain and heavy chain or VH domain comprises other human framework regions or framework regions derived from another human antibody.

In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 62-64, respectively. In certain embodiments, the light chain or VL domain comprises one, two or three of framework region (FR)1, FR2, FR3, and FR4 comprising the amino acid sequences of SEQ ID NO:59-61, respectively. In some embodiments, the light chain or VL domain comprises framework region (FR)1, FR2, FR3, and FR4 comprising the amino acid sequences of SEQ ID NO: 69-72, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 59-61, respectively. In certain embodiments, the heavy chain or VH domain comprises one, two or three of framework region (FR)1, FR2, FR3, and FR4 comprising the amino acid sequences of SEQ ID NO: 65-68, respectively. In some embodiments, the heavy chain or VH domain comprises framework region (FR)1, FR2, FR3, and FR4 comprising the amino acid sequences of SEQ ID NO: 65-68, respectively. In other embodiments, the heavy chain or VH domain comprises other human framework regions or framework regions derived from another human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 62-64, respectively; and a VH domain or heavy chain comprising a VH CDR1, VH CDR2, and VH CDR3 comprising the amino acid sequences of SEQ ID NOs: 59-61, respectively. In certain embodiments, the light chain or VL domain comprises one, two or three of framework region (FR)1, FR2, FR3, and FR4 comprising the amino acid sequences of SEQ ID NO: 69-72, respectively, and the heavy chain or VH domain comprises one, two or three of framework regions (FR)1, FR2, FR3, and FR4 comprising the amino acid sequences of SEQ ID NO: 65-68, respectively. In some embodiments, the light chain or VL domain comprises framework region (FR)1, FR2, FR3, and FR4 comprising the amino acid sequences of SEQ ID NO: 69-72, respectively, and the heavy chain or VH domain comprises framework regions (FR)1, FR2, FR3, and FR4 comprising the amino acid sequences of SEQ ID NO: 65-68, respectively. In other embodiments, the light chain or VL domain and heavy chain or VH domain comprises other human framework regions or framework regions derived from another human antibody.

In another embodiment, an antibody, which binds to a Zika virus NS1 (e.g., NS1 of a Zika virus strain described herein, such as in Section 6, infra), comprises one, two, three, four, five or all six complementarity determining regions (CDRs) of the antibody GB5. In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 76-78, respectively. In certain embodiments, the light chain or VL domain comprises one, two or three of framework region (FR)1, FR2, FR3, and FR4 comprising the amino acid sequences of SEQ ID NO: 83-86, respectively. In some embodiments, the light chain or VL domain comprises framework region (FR)1, FR2, FR3, and FR4 comprising the amino acid sequences of SEQ ID NO: 83-86, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 73-75, respectively. In certain embodiments, the heavy chain or VH domain comprises one, two or three of framework region (FR)1, FR2, FR3, and FR4 comprising the amino acid sequences of SEQ ID NO: 79-82, respectively. In some embodiments, the heavy chain or VH domain comprises framework region (FR)1, FR2, FR3, and FR4 comprising the amino acid sequences of SEQ ID NO: 79-82, respectively. In other embodiments, the heavy chain or VH domain comprises other human framework regions or framework regions derived from another human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 76-78, respectively; and a VH domain or heavy chain comprising a VH CDR1, VH CDR2, and VH CDR3 comprising the amino acid sequences of SEQ ID NOs: 73-75, respectively. In certain embodiments, the light chain or VL domain comprises one, two or three of framework region (FR)1, FR2, FR3, and FR4 comprising the amino acid sequences of SEQ ID NO: 83-86, respectively, and the heavy chain or VH domain comprises one, two or three of framework regions (FR)1, FR2, FR3, and FR4 comprising the amino acid sequences of SEQ ID NO: 79-82, respectively. In some embodiments, the light chain or VL domain comprises framework region (FR)1, FR2, FR3, and FR4 comprising the amino acid sequences of SEQ ID NO: 83-86, respectively, and the heavy chain or VH domain comprises framework regions (FR)1, FR2, FR3, and FR4 comprising the amino acid sequences of SEQ ID NO: 79-82, respectively. In other embodiments, the light chain or VL domain and heavy chain or VH domain comprises other human framework regions or framework regions derived from another human antibody.

In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 90-92, respectively. In certain embodiments, the light chain or VL domain comprises one, two or three of framework region (FR)1, FR2, FR3, and FR4 comprising the amino acid sequences of SEQ ID NO:97-100, respectively. In some embodiments, the light chain or VL domain comprises framework region (FR)1, FR2, FR3, and FR4 comprising the amino acid sequences of SEQ ID NO: 97-100, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 87-89, respectively. In certain embodiments, the heavy chain or VH domain comprises one, two or three of framework region (FR)1, FR2, FR3, and FR4 comprising the amino acid sequences of SEQ ID NO: 93-96, respectively. In some embodiments, the heavy chain or VH domain comprises framework region (FR)1, FR2, FR3, and FR4 comprising the amino acid sequences of SEQ ID NO: 93-96, respectively. In other embodiments, the heavy chain or VH domain comprises other human framework regions or framework regions derived from another human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 90-92, respectively; and a VH domain or heavy chain comprising a VH CDR1, VH CDR2, and VH CDR3 comprising the amino acid sequences of SEQ ID NOs: 87-89, respectively. In certain embodiments, the light chain or VL domain comprises one, two or three of framework region (FR)1, FR2, FR3, and FR4 comprising the amino acid sequences of SEQ ID NO: 97-100, respectively, and the heavy chain or VH domain comprises one, two or three of framework regions (FR)1, FR2, FR3, and FR4 comprising the amino acid sequences of SEQ ID NO: 93-96, respectively. In some embodiments, the light chain or VL domain comprises framework region (FR)1, FR2, FR3, and FR4 comprising the amino acid sequences of SEQ ID NO: 97-100, respectively, and the heavy chain or VH domain comprises framework regions (FR)1, FR2, FR3, and FR4 comprising the amino acid sequences of SEQ ID NO: 93-96, respectively. In other embodiments, the light chain or VL domain and heavy chain or VH domain comprises other human framework regions or framework regions derived from another human antibody.

In another embodiment, an antibody, which binds to a Zika virus NS1 (e.g., NS1 of a Zika virus strain described herein, such as in Section 6, infra), comprises one, two, three, four, five or all six complementarity determining regions (CDRs) of the antibody FC12. In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 104-106, respectively. In certain embodiments, the light chain or VL domain comprises one, two or three of framework region (FR)1, FR2, FR3, and FR4 comprising the amino acid sequences of SEQ ID NO: 101-103, respectively. In some embodiments, the light chain or VL domain comprises framework region (FR)1, FR2, FR3, and FR4 comprising the amino acid sequences of SEQ ID NO: 111-114, respectively. In other embodiments, the light chain or VL domain comprises human framework regions or framework regions derived from a human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 101-103, respectively. In certain embodiments, the heavy chain or VH domain comprises one, two or three of framework region (FR)1, FR2, FR3, and FR4 comprising the amino acid sequences of SEQ ID NO: 107-110, respectively. In some embodiments, the heavy chain or VH domain comprises framework region (FR)1, FR2, FR3, and FR4 comprising the amino acid sequences of SEQ ID NO: 107-110, respectively. In other embodiments, the heavy chain or VH domain comprises human framework regions or framework regions derived from a human antibody.

In specific embodiments, an antibody, which binds to a Zika virus NS1 (e.g., e.g., NS1 of a Zika virus strain described herein, such as in Section 6, infra comprises: VL domain or light chain comprising a VL complementarity determining region (CDR)1, VL CDR2, and VL CDR3 comprising the amino acid sequences of SEQ ID NOs: 104-106, respectively; and a VH domain or heavy chain comprising a VH CDR1, VH CDR2, and VH CDR3 comprising the amino acid sequences of SEQ ID NOs: 101-103, respectively. In certain embodiments, the light chain or VL domain comprises one, two or three of framework region (FR)1, FR2, FR3, and FR4 comprising the amino acid sequences of SEQ ID NO: 111-114, respectively, and the heavy chain or VH domain comprises one, two or three of framework regions (FR)1, FR2, FR3, and FR4 comprising the amino acid sequences of SEQ ID NO: 107-110, respectively. In some embodiments, the light chain or VL domain comprises framework region (FR)1, FR2, FR3, and FR4 comprising the amino acid sequences of SEQ ID NO: 111-114, respectively, and the heavy chain or VH domain comprises framework regions (FR)1, FR2, FR3, and FR4 comprising the amino acid sequences of SEQ ID NO: 107-110, respectively. In other embodiments, the light chain or VL domain and heavy chain or VH domain comprises other human framework regions or framework regions derived from another human antibody.

In certain embodiments, an antibody, which binds to a Zika virus NS1 (e.g., NS1 of a Zika virus strain described herein, such as in Section 6, infra), comprises VL domain or light chain comprising a VL complementarity determining region (CDR)1, VL CDR2, and VL CDR3 comprising the amino acid sequences of SEQ ID NOs: 118-120, respectively. In certain embodiments, the light chain or VL domain comprises one, two or three of framework region (FR)1, FR2, FR3, and FR4 comprising the amino acid sequences of SEQ ID NO:125-128, respectively. In some embodiments, the light chain or VL domain comprises framework region (FR)1, FR2, FR3, and FR4 comprising the amino acid sequences of SEQ ID NO: 125-128, respectively. In other embodiments, the light chain or VL domain comprises other human framework regions or framework regions derived from another human antibody.

In some embodiments, an antibody, which binds to a Zika virus NS1 (e.g., NS1 of a Zika virus strain described herein, such as in Section 6, infra), comprises a VH domain or heavy chain comprising a VH CDR1, VH CDR2, and VH CDR3 comprising the amino acid sequences of SEQ ID NOs: 115-117, respectively. In certain embodiments, the heavy chain or VH domain comprises one, two or three of framework region (FR)1, FR2, FR3, and FR4 comprising the amino acid sequences of SEQ ID NO: 121-124, respectively. In some embodiments, the heavy chain or VH domain comprises framework region (FR)1, FR2, FR3, and FR4 comprising the amino acid sequences of SEQ ID NO: 121-124, respectively. In other embodiments, the heavy chain or VH domain comprises human framework regions or framework regions derived from a human antibody.

In specific embodiments, an antibody, which binds to a Zika virus NS1 (e.g., NS1 of a Zika virus strain described herein, such as in Section 6, infra), comprises: VL domain or light chain comprising a VL complementarity determining region (CDR)1, VL CDR2, and VL CDR3 comprising the amino acid sequences of SEQ ID NOs: 118-120, respectively; and a VH domain or heavy chain comprising a VH CDR1, VH CDR2, and VH CDR3 comprising the amino acid sequences of SEQ ID NOs: 115-117, respectively. In certain embodiments, the light chain or VL domain comprises one, two or three of framework region (FR)1, FR2, FR3, and FR4 comprising the amino acid sequences of SEQ ID NO: 125-128, respectively, and the heavy chain or VH domain comprises one, two or three of framework regions (FR)1, FR2, FR3, and FR4 comprising the amino acid sequences of SEQ ID NO: 121-124, respectively. In some embodiments, the light chain or VL domain comprises framework region (FR)1, FR2, FR3, and FR4 comprising the amino acid sequences of SEQ ID NO: 125-128, respectively, and the heavy chain or VH domain comprises framework regions (FR)1, FR2, FR3, and FR4 comprising the amino acid sequences of SEQ ID NO: 121-124, respectively. In other embodiments, the light chain or VL domain and heavy chain or VH domain comprises human framework regions or framework regions derived from a human antibody.

In some embodiments, an antibody described herein, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 10. In certain embodiments, an antibody described herein, which binds to a Zika virus NS1 (e.g., NS1 of a Zika virus strain described in Section 6, infra), comprises a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 9. In some embodiments, an antibody, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 10; and a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 9. In accordance with these embodiments, the CDRs of the antibody may, in certain embodiments, be identical to one, two, three, four, five, or all six of the CDRs of the antibody AA12.

In some embodiments, an antibody described herein, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 12. In certain embodiments, an antibody described herein, which binds to a Zika virus NS1 (e.g., NS1 of a Zika virus strain described in Section 6, infra), comprises a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 11. In some embodiments, an antibody, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 12; and a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 11. In accordance with these embodiments, the CDRs of the antibody may, in certain embodiments, be identical to one, two, three, four, five, or all six of the CDRs of the antibody EB9.

In some embodiments, an antibody described herein, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 14. In certain embodiments, an antibody described herein, which binds to a Zika virus NS1 (e.g., NS1 of a Zika virus strain described in Section 6, infra), comprises a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 13. In some embodiments, an antibody, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 14; and a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 13. In accordance with these embodiments, the CDRs of the antibody may, in certain embodiments, be identical to one, two, three, four, five, or all six of the CDRs of the antibody EB9.

In some embodiments, an antibody described herein, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 16. In certain embodiments, an antibody described herein, which binds to a Zika virus NS1 (e.g., NS1 of a Zika virus strain described in Section 6, infra), comprises a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 15. In some embodiments, an antibody, which binds to a Zika virus NS1 (e.g., NS1 of a Zika virus strain described in Section 6, infra), comprises a VL domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 16; and a VH domain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 15. In accordance with these embodiments, the CDRs of the antibody may, in certain embodiments, be identical to one, two, three, four, five, or all six of the CDRs of the antibody EB9.

In some embodiments, an antibody described herein, which binds to a Zika virus NS1, comprises a VL domain comprising the amino acid sequence of SEQ ID NO: 10; a VH domain comprising the amino acid sequence of SEQ ID NO: 9; or a VL domain comprising the amino acid sequence of SEQ ID NO:10 and VH domain comprising the amino acid sequence of SEQ ID NO:9. In some embodiments, an antibody described herein, which binds to a Zika virus NS1, comprises a VL domain comprising the amino acid sequence of SEQ ID NO:12; a VH domain comprising the amino acid sequence of SEQ ID NO: 11; or a VL domain comprising the amino acid sequence of SEQ ID NO: 12 and VH domain comprising the amino acid sequence of SEQ ID NO:11. In some embodiments, an antibody described herein, which binds to a Zika virus NS1, comprises a VL domain comprising the amino acid sequence of SEQ ID NO:14; a VH domain comprising the amino acid sequence of SEQ ID NO: 13; or a VL domain comprising the amino acid sequence of SEQ ID NO:14 and VH domain comprising the amino acid sequence of SEQ ID NO:13. In some embodiments, an antibody described herein, which binds to a Zika virus NS1, comprises a VL domain comprising the amino acid sequence of SEQ ID NO:16; a VH domain comprising the amino acid sequence of SEQ ID NO: 15; or a VL domain comprising the amino acid sequence of SEQ ID NO:16 and VH domain comprising the amino acid sequence of SEQ ID NO:15.

In some embodiments, an antibody described herein, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 10. In certain embodiments, an antibody described herein, which binds to a Zika virus NS1 (e.g., NS1 of a Zika virus strain described in Section 6, infra), comprises a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 9. In some embodiments, an antibody, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 10; and a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 9. In accordance with these embodiments, the CDRs of the antibody may, in certain embodiments, identical to one, two, three, four, five, or all six of the CDRs of the antibody AA12.

In some embodiments, an antibody described herein, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 12. In certain embodiments, an antibody described herein, which binds to a Zika virus NS1 (e.g., NS1 of a Zika virus strain described in Section 6, infra), comprises a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 11. In some embodiments, an antibody, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 12; and a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO:11. In accordance with these embodiments, the CDRs of the antibody may, in certain embodiments, identical to one, two, three, four, five, or all six of the CDRs of the antibody EB9.

In some embodiments, an antibody described herein, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 14. In certain embodiments, an antibody described herein, which binds to a Zika virus NS1 (e.g., NS1 of a Zika virus strain described in Section 6, infra), comprises a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 13. In some embodiments, an antibody, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 14; and a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 13. In accordance with these embodiments, the CDRs of the antibody may, in certain embodiments, identical to one, two, three, four, five, or all six of the CDRs of the antibody GB5.

In some embodiments, an antibody described herein, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 16. In certain embodiments, an antibody described herein, which binds to a Zika virus NS1 (e.g., NS1 of a Zika virus strain described in Section 6, infra), comprises a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 15. In some embodiments, an antibody, which binds to a Zika virus NS1 (e.g., NS1 of a Zika virus strain described in Section 6, infra), comprises a light chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 16; and a heavy chain comprising an amino acid sequence that is at least 80%, 85%, 86%, 87%, 88%, 89%, 90%, 91%, 92%, 93%, 94% 95%, 96%, 97% or 98% identical to the amino acid sequence of SEQ ID NO: 15. In accordance with these embodiments, the CDRs of the antibody may, in certain embodiments, identical to one, two, three, four, five, or all six of the CDRs of the antibody FC12.

In some embodiments, an antibody described herein, which binds to a Zika virus NS1, comprises a light chain comprising the amino acid sequence of SEQ ID NO: 10; a heavy chain comprising the amino acid sequence of SEQ ID NO: 9; or a light chain comprising the amino acid sequence of SEQ ID NO:10 and a heavy chain comprising the amino acid sequence of SEQ ID NO:9. In some embodiments, an antibody described herein, which binds to a Zika virus NS1, comprises a light chain comprising the amino acid sequence of SEQ ID NO: 12; a heavy chain comprising the amino acid sequence of SEQ ID NO: 11; or a light chain comprising the amino acid sequence of SEQ ID NO:12 and a heavy chain comprising the amino acid sequence of SEQ ID NO:11. In some embodiments, an antibody described herein, which binds to a Zika virus NS1, comprises a light chain comprising the amino acid sequence of SEQ ID NO: 14; a heavy chain comprising the amino acid sequence of SEQ ID NO:13; or a light chain comprising the amino acid sequence of SEQ ID NO:14 and a heavy chain comprising the amino acid sequence of SEQ ID NO:13. In some embodiments, an antibody described herein, which binds to a Zika virus NS1, comprises a light chain comprising the amino acid sequence of SEQ ID NO: 16; a heavy chain comprising the amino acid sequence of SEQ ID NO: 15; or a light chain comprising the amino acid sequence of SEQ ID NO:16 and a heavy chain comprising the amino acid sequence of SEQ ID NO:15.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions ×100%). In one embodiment, the two sequences are the same length. In a certain embodiment, the percent identity is determined over the entire length of an amino acid sequence or nucleotide sequence.

The determination of percent identity between two sequences (e.g., amino acid sequences or nucleic acid sequences) can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In some embodiments, an antibody described herein, which binds to a Zika virus NS1 (e.g., NS1 of a Zika virus strain in Section 6, infra), comprises the VH or VL of AA12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 9 or 10. In a specific embodiment, an antibody described herein, which binds to a Zika virus NS1 (e.g., NS1 of a Zika virus strain in Section 6, infra), comprises the VH or VL of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 9 or 10. In certain embodiments, an antibody described herein, which binds to a Zika virus NS1 (e.g., NS1 of a Zika virus strain in Section 6, infra), comprises the VH or VL of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 9 or 10, wherein the one or more amino acid substitutions is in one, two, three or more of the framework regions. In specific embodiments, none of the amino acid substitutions are located within the CDRs (e.g., SEQ ID NOs: 17-22 or SEQ ID Nos: 31-36). In specific embodiments, all of the amino acid substitutions are in the framework regions (e.g., SEQ ID NOs: 23-30 or SEQ ID NOs: 37-44).

In some embodiments, an antibody described herein, which binds to a Zika virus NS1 (e.g., NS1 of a Zika virus strain in Section 6, infra), comprises (1) the VH domain of AA12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 9 and (2) the VL domain of AA12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 10. In a specific embodiment, an antibody described herein, which binds to a Zika virus NS1 (e.g., NS1 of a Zika virus strain in Section 6, infra), comprises (1) the VH domain of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 9 and (2) the VL domain of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 10. In certain embodiments, an antibody described herein, which binds to a Zika virus NS1 (e.g., NS1 of a Zika virus strain in Section 6, infra), comprises (1) the VH domain of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 9, wherein the one or more amino acid substitutions is in one, two, three or more of the framework regions; and (2) the VL domain of AA12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 10, wherein the one or more amino acid substitutions is in one, two, three or more of the framework regions. In specific embodiments, none of the amino acid substitutions are located within the CDRs (e.g., SEQ ID NOs: 17-22 or 31-36). In specific embodiments, all of the amino acid substitutions are in the framework regions (e.g., SEQ ID NOs: 23-30 or 37-44).

In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1, the VL CDR2, the VL CDR3, the VL CDR1 and VL CDR2, the VL CDR2 and VL CDR3, the VL CDR1 and VL CDR3, or the VL CDR1, VL CDR2 and VL CDR3 of the antibody AA12. In some embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VH CDR1, the VH CDR2, the VH CDR3, the VH CDR1 and VH CDR2, the VH CDR2 and VH CDR3, the VH CDR1 and VH CDR3, or the VH CDR1, VH CDR2 and VH CDR3 of the antibody AA12. In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1; the VL CDR2; the VL CDR3; the VH CDR1; the VH CDR2; and/or the VH CDR3 of the antibody AA12.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In some embodiments, an antibody described herein, which binds to a Zika virus NS1 (e.g., NS1 of a Zika virus strain in Section 6, infra), comprises the VH or VL of EB9 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 11 or 12. In a specific embodiment, an antibody described herein, which binds to a Zika virus NS1 (e.g., NS1 of a Zika virus strain in Section 6, infra), comprises the VH or VL of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 11 or 12. In certain embodiments, an antibody described herein, which binds to a Zika virus NS1 (e.g., NS1 of a Zika virus strain in Section 6, infra), comprises the VH or VL of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 11 or 12, wherein the one or more amino acid substitutions is in one, two, three or more of the framework regions. In specific embodiments, none of the amino acid substitutions are located within the CDRs (e.g., SEQ ID NOs: 45-50 or SEQ ID Nos: 59-64). In specific embodiments, all of the amino acid substitutions are in the framework regions (e.g., SEQ ID NOs: 51-58 or SEQ ID NOs: 65-72).

In some embodiments, an antibody described herein, which binds to a Zika virus NS1 (e.g., NS1 of a Zika virus strain in Section 6, infra), comprises (1) the VH domain of EB9 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 11 and (2) the VL domain of EB9 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 12. In a specific embodiment, an antibody described herein, which binds to a Zika virus NS1 (e.g., NS1 of a Zika virus strain in Section 6, infra), comprises (1) the VH domain of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 11 and (2) the VL domain of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 12. In certain embodiments, an antibody described herein, which binds to a Zika virus NS1 (e.g., NS1 of a Zika virus strain in Section 6, infra), comprises (1) the VH domain of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 11, wherein the one or more amino acid substitutions is in one, two, three or more of the framework regions; and (2) the VL domain of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 12, wherein the one or more amino acid substitutions is in one, two, three or more of the framework regions. In specific embodiments, none of the amino acid substitutions are located within the CDRs (e.g., SEQ ID NOs: 45-50 or 59-64). In specific embodiments, all of the amino acid substitutions are in the framework regions (e.g., SEQ ID NOs: 51-58 or 65-72).

In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1, the VL CDR2, the VL CDR3, the VL CDR1 and VL CDR2, the VL CDR2 and VL CDR3, the VL CDR1 and VL CDR3, or the VL CDR1, VL CDR2 and VL CDR3 of the antibody EB9. In some embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VH CDR1, the VH CDR2, the VH CDR3, the VH CDR1 and VH CDR2, the VH CDR2 and VH CDR3, the VH CDR1 and VH CDR3, or the VH CDR1, VH CDR2 and VH CDR3 of the antibody EB9. In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1; the VL CDR2; the VL CDR3; the VH CDR1; the VH CDR2; and/or the VH CDR3 of the antibody EB9.

In some embodiments, an antibody described herein, which binds to a Zika virus NS1 (e.g., NS1 of a Zika virus strain in Section 6, infra), comprises the VH or VL of GB5 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 13 or 14. In a specific embodiment, an antibody described herein, which binds to a Zika virus NS1 (e.g., NS1 of a Zika virus strain in Section 6, infra), comprises the VH or VL of GB5 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 13 or 14. In certain embodiments, an antibody described herein, which binds to a Zika virus NS1 (e.g., NS1 of a Zika virus strain in Section 6, infra), comprises the VH or VL of GB5 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 13 or 14, wherein the one or more amino acid substitutions is in one, two, three or more of the framework regions. In specific embodiments, none of the amino acid substitutions are located within the CDRs (e.g., SEQ ID NOs: 73-78 or SEQ ID Nos: 87-92). In specific embodiments, all of the amino acid substitutions are in the framework regions (e.g., SEQ ID NOs: 79-86 or SEQ ID NOs: 93-100).

In some embodiments, an antibody described herein, which binds to a Zika virus NS1 (e.g., NS1 of a Zika virus strain in Section 6, infra), comprises (1) the VH domain of GB5 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 13 and (2) the VL domain of GB5 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 14. In a specific embodiment, an antibody described herein, which binds to a Zika virus NS1 (e.g., NS1 of a Zika virus strain in Section 6, infra), comprises (1) the VH domain of GB5 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 13 and (2) the VL domain of GB5 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 14. In certain embodiments, an antibody described herein, which binds to a Zika virus NS1 (e.g., NS1 of a Zika virus strain in Section 6, infra), comprises (1) the VH domain of GB5 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 13, wherein the one or more amino acid substitutions is in one, two, three or more of the framework regions; and (2) the VL domain of EB9 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 14, wherein the one or more amino acid substitutions is in one, two, three or more of the framework regions. In specific embodiments, none of the amino acid substitutions are located within the CDRs (e.g., SEQ ID NOs: 73-78 or 87-92). In specific embodiments, all of the amino acid substitutions are in the framework regions (e.g., SEQ ID NOs: 79-86 or 93-100).

In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1, the VL CDR2, the VL CDR3, the VL CDR1 and VL CDR2, the VL CDR2 and VL CDR3, the VL CDR1 and VL CDR3, or the VL CDR1, VL CDR2 and VL CDR3 of the antibody GB5. In some embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VH CDR1, the VH CDR2, the VH CDR3, the VH CDR1 and VH CDR2, the VH CDR2 and VH CDR3, the VH CDR1 and VH CDR3, or the VH CDR1, VH CDR2 and VH CDR3 of the antibody GB5. In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1; the VL CDR2; the VL CDR3; the VH CDR1; the VH CDR2; and/or the VH CDR3 of the antibody GB5.

In some embodiments, an antibody described herein, which binds to a Zika virus NS1 (e.g., NS1 of a Zika virus strain in Section 6, infra), comprises the VH or VL of FC12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 15 or 16. In a specific embodiment, an antibody described herein, which binds to a Zika virus NS1 (e.g., NS1 of a Zika virus strain in Section 6, infra), comprises the VH or VL of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 15 or 16. In certain embodiments, an antibody described herein, which binds to a Zika virus NS1 (e.g., NS1 of a Zika virus strain in Section 6, infra), comprises the VH or VL of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 15 or 16, wherein the one or more amino acid substitutions is in one, two, three or more of the framework regions. In specific embodiments, none of the amino acid substitutions are located within the CDRs (e.g., SEQ ID NOs: 101-106 or SEQ ID Nos: 115-120). In specific embodiments, all of the amino acid substitutions are in the framework regions (e.g., SEQ ID NOs: 107-114 or SEQ ID NOs: 121-128).

In some embodiments, an antibody described herein, which binds to a Zika virus NS1 (e.g., NS1 of a Zika virus strain in Section 6, infra), comprises (1) the VH domain of FC12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 15 and (2) the VL domain of FC12 with one or more (e.g., 1, 2, 3, 4, 5, 6, 7, 8, 9, 10, 15 or 20) amino acid substitutions (e.g., conservative amino acid substitutions), deletions, or additions relative to the amino acid sequence of the SEQ ID NO: 16. In a specific embodiment, an antibody described herein, which binds to a Zika virus NS1 (e.g., NS1 of a Zika virus strain in Section 6, infra), comprises (1) the VH domain of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 15 and (2) the VL domain of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 16. In certain embodiments, an antibody described herein, which binds to a Zika virus NS1 (e.g., NS1 of a Zika virus strain in Section 6, infra), comprises (1) the VH domain of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 15, wherein the one or more amino acid substitutions is in one, two, three or more of the framework regions; and (2) the VL domain of FC12 with one or more (e.g., 1, 2, 3, 4, 5, 6, 7, 8, 9, 10, 15 or 20) amino acid substitutions (e.g., conservative amino acid substitutions) relative to the amino acid sequence of the SEQ ID NO: 16, wherein the one or more amino acid substitutions is in one, two, three or more of the framework regions. In specific embodiments, none of the amino acid substitutions are located within the CDRs (e.g., SEQ ID NOs: 101-106 or 115-120). In specific embodiments, all of the amino acid substitutions are in the framework regions (e.g., SEQ ID NOs: 107-114 or 121-128).

In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1, the VL CDR2, the VL CDR3, the VL CDR1 and VL CDR2, the VL CDR2 and VL CDR3, the VL CDR1 and VL CDR3, or the VL CDR1, VL CDR2 and VL CDR3 of the antibody FC12. In some embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VH CDR1, the VH CDR2, the VH CDR3, the VH CDR1 and VH CDR2, the VH CDR2 and VH CDR3, the VH CDR1 and VH CDR3, or the VH CDR1, VH CDR2 and VH CDR3 of the antibody FC12. In certain embodiments, an antibody described herein, which binds to a Zika virus NS1 (e.g., NS1 of a Zika virus strain described in Section 6, infra), comprises one or more (e.g., 1, 2, 3, 4, 5 or 6) amino acid substitutions (e.g., conservative amino acid substitutions) in the amino acid sequence of one, two or more of the following: the VL CDR1; the VL CDR2; the VL CDR3; the VH CDR1; the VH CDR2; and/or the VH CDR3 of the antibody FC12.

In another aspect, provided herein are antibodies that bind to the same or an overlapping epitope of an antibody described herein (e.g., an antibody described in Section 6, infra), e.g., antibodies that compete for binding to a Zika virus NS1 with an antibody described herein, or antibodies which bind to an epitope which overlaps with an epitope to which an antibody described herein binds. As used herein, an “epitope” is a term in the art and typically refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain aspects, epitope mapping assays, well known to one of skill in the art, can be performed to ascertain the epitope (e.g., conformational epitope) to which an antibody described herein binds. In certain embodiments, the epitope can be determined by, e.g., structural mapping using negative electron microscopy (see, e.g., Section 6, infra), X-ray diffraction crystallography studies (see, e.g., Blechman et al., 1993, J. Biol. Chem. 268:4399-4406; Cho et al., 2003, Nature, 421:756-760), ELISA assays, hydrogen/deuterium exchange coupled with mass spectrometry (e.g., MALDI mass spectrometry), array-based oligo-peptide scanning assays, mutagenesis mapping (e.g., site-directed mutagenesis mapping) and/or escape binding assays.

Antibodies that recognize such epitopes can be identified using routine techniques such as an immunoassay, for example, by showing the ability of one antibody to block the binding of another antibody to a target antigen, i.e., a competitive binding assay. Competition binding assays also can be used to determine whether two antibodies have similar binding specificity for an epitope. Competitive binding can be determined in an assay in which the immunoglobulin under test inhibits specific binding of a reference antibody to a common antigen, such as a Zika virus NS1. Numerous types of competitive binding assays are known, for example: solid phase direct or indirect radioimmunoassay (RIA), solid phase direct or indirect enzyme immunoassay (EIA), sandwich competition assay (see Stahli et al., (1983) Methods in Enzymology 9:242); solid phase direct biotin-avidin EIA (see Kirkland et al., (1986) J. Immunol. 137:3614); solid phase direct labeled assay, solid phase direct labeled sandwich assay (see Harlow and Lane, (1988) Antibodies: A Laboratory Manual, Cold Spring Harbor Press); solid phase direct label RIA using I-125 label (see Morel et al., (1988) Mol. Immunol. 25 (1): 7); solid phase direct biotin-avidin EIA (Cheung et al., (1990) Virology 176:546); and direct labeled RIA. (Moldenhauer et al., (1990) Scand J. Immunol. 32:77). Typically, such an assay involves the use of purified antigen (e.g., a Zika virus NS1 described herein, such as in Section 6, infra) bound to a solid surface or cells bearing either of these, an unlabeled test immunoglobulin and a labeled reference immunoglobulin. Competitive inhibition can be measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more. A competition binding assay can be configured in a large number of different formats using either labeled antigen or labeled antibody. In a common version of this assay, the antigen is immobilized on a 96-well plate. The ability of unlabeled antibodies to block the binding of labeled antibodies to the antigen is then measured using radioactive or enzyme labels. For further details see, for example, Wagener et al., J. Immunol., 1983, 130:2308-2315; Wagener et al., J. Immunol. Methods, 1984, 68:269-274; Kuroki et al., Cancer Res., 1990, 50:4872-4879; Kuroki et al., Immunol. Invest., 1992, 21:523-538; Kuroki et al., Hybridoma, 1992, 11:391-407, and Using Antibodies: A Laboratory Manual, Ed Harlow and David Lane editors (Cold Springs Harbor Laboratory Press, Cold Springs Harbor, N.Y., 1999), pp. 386-389.

In certain aspects, competition binding assays can be used to determine whether an antibody is competitively blocked, e.g., in a dose dependent manner, by another antibody for example, an antibody binds essentially the same epitope, or overlapping epitopes, as a reference antibody, when the two antibodies recognize identical or sterically overlapping epitopes in competition binding assays such as competition ELISA assays, which can be configured in all number of different formats, using either labeled antigen or labeled antibody. In a particular embodiment, an antibody can be tested in competition binding assays with an antibody described herein, e.g., antibody AA12, EB9, GB5, or FC12, or an antibody comprising VH CDRs and VL CDRs of antibody AA12, EB9, GB5, or FC12.

In a specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 20-22, respectively; and a VH domain or heavy chain comprising a VH CDR1, VH CDR2, and VH CDR3 having the amino acid sequences of SEQ ID NOs: 17-19, respectively. In another specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 34-36, respectively; and a VH domain or heavy chain comprising a VH CDR1, VH CDR2, and VH CDR3 having the amino acid sequences of SEQ ID NOs: 31-33, respectively.

In a specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 48-50, respectively; and a VH domain or heavy chain comprising a VH CDR1, VH CDR2, and VH CDR3 having the amino acid sequences of SEQ ID NOs: 45-47, respectively. In another specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 62-64, respectively; and a VH domain or heavy chain comprising a VH CDR1, VH CDR2, and VH CDR3 having the amino acid sequences of SEQ ID NOs: 59-61, respectively.

In a specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 76-78, respectively; and a VH domain or heavy chain comprising a VH CDR1, VH CDR2, and VH CDR3 having the amino acid sequences of SEQ ID NOs: 73-75, respectively. In another specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 90-92, respectively; and a VH domain or heavy chain comprising a VH CDR1, VH CDR2, and VH CDR3 having the amino acid sequences of SEQ ID NOs: 87-89, respectively.

In a specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 104-106, respectively; and a VH domain or heavy chain comprising a VH CDR1, VH CDR2, and VH CDR3 having the amino acid sequences of SEQ ID NOs: 101-103, respectively. In another specific embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising: a VL domain or light chain comprising a VL CDR1, VL CDR2, and VL CDR3 having the amino acid sequences of SEQ ID NOs: 118-120, respectively; and a VH domain or heavy chain comprising a VH CDR1, VH CDR2, and VH CDR3 having the amino acid sequences of SEQ ID NOs: 115-117, respectively.

In another embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising a VL domain comprising the amino acid sequence of SEQ ID NO: 10; and VH domain comprising the amino acid sequence of SEQ ID NO: 9. In another embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising a VL domain comprising the amino acid sequence of SEQ ID NO: 12; and VH domain comprising the amino acid sequence of SEQ ID NO: 11. In another embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising a VL domain comprising the amino acid sequence of SEQ ID NO: 14; and VH domain comprising the amino acid sequence of SEQ ID NO: 13. In another embodiment, provided herein is an antibody, which binds to a Zika virus NS1 (e.g., NS1 of a Zika virus described in Section 6, infra), wherein said antibody competes (e.g., in a dose-dependent manner) for binding to the Zika virus NS1 with a reference antibody comprising a VL domain comprising the amino acid sequence of SEQ ID NO: 16; and VH domain comprising the amino acid sequence of SEQ ID NO: 15.

In one embodiment, an antibody described herein binds to the same epitope as the AA12 antibody described herein. In another embodiment, an antibody described herein binds to the same epitope as the EB9 antibody described herein. In another embodiment, an antibody described herein binds to the same epitope as the GB5 antibody described herein. In another embodiment, an antibody described herein binds to the same epitope as the FC12 antibody described herein.

In another specific embodiment, an antibody provided herein competes for binding to recombinant NS1 or Zika virus NS1 with an antibody comprising either the variable regions (VL and VH domains) or light and heavy chains of the antibody AA12, EB9, GB5, or FC12. In another particular embodiment, an antibody competes for binding to recombinant NS1 or Zika virus NS1 with the antibody AA12, EB9, GB5, or FC12. In a particular embodiment, the competition between an antibody for binding to recombinant NS1 or Zika virus NS1 with the antibody AA12, EB9, GB5, or FC12 is not asymmetrical.

In certain embodiments, an antibody described herein, which binds to a Zika virus NS1, comprises a VH domain or heavy chain comprising FR1, FR2, FR3 and FR4 of the VH domain or heavy chain of the antibody AA12, EB9, GB5, or FC12. In some embodiments, an antibody described herein, which binds to a Zika virus NS1, comprises a VL domain or light chain comprising FR1, FR2, FR3 and FR4 of the VL domain or light chain of the antibody AA12, EB9, GB5, or FC12. In a specific embodiment, an antibody described herein, which binds to a Zika virus NS1, comprises framework regions of the antibody AA12, EB9, GB5, or FC12.

In specific embodiments, an antibody described herein, which binds to a Zika virus NS1, comprises framework regions (e.g., framework regions of the VL domain and/or VH domain) that are human framework regions or derived from human framework regions. The framework region may be naturally occurring or consensus framework regions (see, e.g., Sui et al., 2009, Nature Structural & Molecular Biology 16:265-273). Non-limiting examples of human framework regions are described in the art, e.g., see Kabat et al. (1991) Sequences of Proteins of Immunological Interest, Fifth Edition, U.S. Department of Health and Human Services, NIH Publication No. 91-3242). In certain embodiment, an antibody described herein comprises framework regions (e.g., framework regions of the VL domain and/or VH domain) that are primate (e.g., non-human primate) framework regions or derived from primate (e.g., non-human primate) framework regions.

For example, CDRs from antigen-specific non-human antibodies, typically of rodent origin (e.g., mouse or rat), are grafted onto homologous human or non-human primate acceptor frameworks. In one embodiment, the non-human primate acceptor frameworks are from Old World apes. In a specific embodiment, the Old World ape acceptor framework is from Pan troglodytes, Pan paniscus or Gorilla gorilla. In a particular embodiment, the non-human primate acceptor frameworks are from the chimpanzee Pan troglodytes. In a particular embodiment, the non-human primate acceptor frameworks are Old World monkey acceptor frameworks. In a specific embodiment, the Old World monkey acceptor frameworks are from the genus Macaca. In a certain embodiment, the non-human primate acceptor frameworks are is derived from the cynomolgus monkey Macaca cynomolgus. Non-human primate framework sequences are described in U.S. Patent Application Publication No. US 2005/0208625.

In specific aspects, provided herein is an antibody comprising an antibody light chain and heavy chain, e.g., a separate light chain and heavy chain.

With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. In a particular embodiment, an antibody described herein, which binds to a Zika virus NS1, comprises a light chain wherein the amino acid sequence of the VL domain can comprise any amino acid sequence described herein, and wherein the constant region of the light chain comprises the amino acid sequence of a human kappa or lamda light chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In a specific embodiment, an antibody described herein comprises (i) a heavy chain comprising a VH domain described herein and a constant region; or (ii) a light chain comprising a VL domain described herein and a constant region. In a specific embodiment, an antibody described herein comprises (i) a heavy chain comprising a VH domain described herein and a constant region; and (ii) a light chain comprising a VL domain described herein and a constant region. As used herein, the term “constant region” or “constant domain” is interchangeable and has its meaning common in the art. The constant region refers to an antibody portion, e.g., a carboxyl terminal portion of a light and/or heavy chain which is not directly involved in binding of an antibody to antigen but which can exhibit various effector functions, such as interaction with the Fc receptor. The terms refer to a portion of an immunoglobulin molecule having a generally more conserved amino acid sequence relative to an immunoglobulin variable domain.

As used herein, the term “heavy chain” when used in reference to an antibody can refer to any distinct types, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to any distinct types, e.g., kappa (κ) of lambda (λ) based on the amino acid sequence of the constant domains. Light chain amino acid sequences are well known in the art. In specific embodiments, the light chain is a human light chain.

With respect to the heavy chain, in a specific embodiment, the heavy chain of an antibody described herein can be an alpha (α), delta (δ), epsilon (ε), gamma (γ) or mu (μ) heavy chain. In another specific embodiment, the heavy chain of an antibody described can comprise a human alpha (α), delta (δ), epsilon (ε), gamma (γ) or mu (μ) heavy chain. In a particular embodiment, an antibody described herein, which binds to a Zika virus NS1, comprises a heavy chain wherein the amino acid sequence of the VH domain can comprise any amino acid sequence described herein, and wherein the constant region of the heavy chain comprises the amino acid sequence of a human gamma (γ) heavy chain constant region. Non-limiting examples of human constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

In a specific embodiment, an antibody described herein, which binds to a Zika virus NS1 (e.g., NS1 of a Zika virus strain described herein in Section 6, infra) comprises a VL domain and a VH domain comprising any amino acid sequences described herein, and wherein the constant regions comprise the amino acid sequences of the constant regions of an IgG, IgE, IgM, IgD, IgA or IgY immunoglobulin molecule (e.g., a human IgG, IgE, IgM, IgD, IgA or IgY immunoglobulin molecule). In another specific embodiment, an antibody described herein, which binds to a Zika virus NS1 (e.g., NS1 of a Zika virus strain described herein in Section 6, infra) comprises a VL domain and a VH domain comprising any amino acid sequences described herein, and wherein the constant regions comprise the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. In a particular embodiment, the constant regions comprise the amino acid sequences of the constant regions of a human IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule.

The antibodies described herein can be affinity matured using techniques known to one of skill in the art. The antibodies described herein can be chimerized using techniques known to one of skill in the art. A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

The antibodies described herein can be humanized. A humanized antibody is an antibody which is capable of binding to a predetermined antigen and which comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fab, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable region of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG1, IgG2, IgG3 and IgG4. Usually the constant domain is a complement fixing constant domain where it is desired that the humanized antibody exhibits cytotoxic activity, and the class is typically IgG1. Where such cytotoxic activity is not desirable, the constant domain may be of the IgG2 class. Examples of VL and VH constant domains that can be used in certain embodiments include, but are not limited to, C-kappa and C-gamma-1 (nG1m) described in Johnson et al. (1997) J. Infect. Dis. 176, 1215-1224 and those described in U.S. Pat. No. 5,824,307. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%.

The antibodies provided herein include derivatives that are chemically modified, i.e., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

In particular embodiments, the glycosylation of antibodies described herein, in particular glycosylation of a variable region of an antibody described herein, is modified. For example, an aglycoslated antibody can be made (i.e., the antibody lacks glycosylation) or an antibody comprising a mutation or substitution at one or more glycosylation sites to eliminate glycosylation at the one or more glycosylation sites can be made. Glycosylation can be altered to, for example, increase the affinity of the antibody for a Zika virus NS1. Such carbohydrate modifications can be accomplished by, for example, altering one or more sites of glycosylation within the antibody sequence. For example, one or more amino acid substitutions can be made that result in elimination of one or more variable region (e.g., VL and/or VH CDRs or VL and/or VH FRs) glycosylation sites to thereby eliminate glycosylation at that site. Such aglycosylation can increase the affinity of the antibody for a Zika virus NS1. Such an approach is described in further detail in U.S. Pat. Nos. 5,714,350 and 6,350,861.

Glycosylation can occur via N-linked (or asparagine-linked) glycosylation or O-linked glycosylation. N-linked glycosylation involves carbohydrate modification at the side-chain NH₂ group of an asparagine amino acid in a polypeptide. O-linked glycosylation involves carbohydrate modification at the hydroxyl group on the side chain of a serine, threonine, or hydroxylysine amino acid.

In certain embodiments, aglycosylated antibodies can be produced in bacterial cells which lack the necessary glycosylation machinery. Cells with altered glycosylation machinery have been described in the art and can be used as host cells in which to express recombinant antibodies described herein to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 03/035835; WO 99/54342.

Antibodies with reduced fucose content have been reported to have an increased affinity for Fc receptors, such as, e.g., FcγRIIIa. Accordingly, in certain embodiments, the antibodies described herein have reduced fucose content or no fucose content. Such antibodies can be produced using techniques known to one skilled in the art. For example, the antibodies can be expressed in cells deficient or lacking the ability to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

In certain embodiments, one, two or more mutations (e.g., amino acid substitutions) are introduced into the Fc region of an antibody described herein or a fragment thereof (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to increase the affinity of the antibody for an Fc receptor (e.g., an activated Fc receptor) on the surface of an effector cell. Mutations in the Fc region of an antibody or fragment thereof that increase the affinity of an antibody for an Fc receptor and techniques for introducing such mutations into the Fc receptor or fragment thereof are known to one of skill in the art. Examples of mutations in the Fc receptor of an antibody that can be made to increase the affinity of the antibody for an Fc receptor are described in, e.g., Smith, P., et al. (2012) PNAS. 109:6181-6186, which is incorporated herein by reference.

### 5.2.1 Antibodies with Increased Half-Lives

Provided herein are antibodies, wherein said antibodies are modified to have an extended (or increased) half-life in vivo. In particular, provided herein are modified antibodies which have a half-life in a subject, preferably a mammal and most preferably a human, of from about 3 days to about 180 days (or more), and in some embodiments greater than 3 days, greater than 7 days, greater than 10 days, greater than 15 days, greater than 20 days, greater than 25 days, greater than 30 days, greater than 35 days, greater than 40 days, greater than 45 days, greater than 50 days, at least about 60 days, greater than 75 days, greater than 90 days, greater than 105 days, greater than 120 days, greater than 135 days, greater than 150 days, greater than 165 days, or greater than 180 days.

In a specific embodiment, modified antibodies having an increased half-life in vivo are generated by introducing one or more amino acid modifications (i.e., substitutions, insertions or deletions) into an IgG constant domain, or FcRn-binding fragment thereof (preferably a Fc or hinge-Fc domain fragment). See, e.g., International Publication Nos. WO 02/060919; WO 98/23289; and WO 97/34631; and U.S. Pat. No. 6,277,375; each of which is incorporated herein by reference in its entirety. In a specific embodiment, the modified antibodies may have one or more amino acid modifications in the second constant CH2 domain (residues 231-340 of human IgG1) and/or the third constant CH3 domain (residues 341-447 of human IgG1), with numbering according to the Kabat numbering system (e.g., the EU index in Kabat).

In some embodiments, to prolong the in vivo serum circulation of antibodies, inert polymer molecules such as high molecular weight polyethyleneglycol (PEG) are attached to the antibodies with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

In another embodiment, antibodies are conjugated to albumin in order to make the antibody more stable in vivo or have a longer half-life in vivo. The techniques are well-known in the art, see, e.g., International Publication Nos. WO 93/15199, WO 93/15200, and WO 01/77137; and European Patent No. EP 413,622, all of which are incorporated herein by reference.

### 5.3 Antibody Conjugates

In some aspects, provided herein are antibodies, conjugated or recombinantly fused to a diagnostic, detectable or therapeutic agent or any other molecule. The conjugated or recombinantly fused antibodies can be useful, e.g., for monitoring or prognosing the onset, development, progression and/or severity of a Zika virus disease as part of a clinical testing procedure, such as determining the efficacy of a particular therapy. In certain aspects, the conjugated or recombinantly fused antibodies can be useful in preventing and/or treating a Zika virus disease or Zika virus infection. In some aspects, the conjugated or recombinantly fused antibodies may be used to detect Zika virus or diagnose a Zika virus infection or Zika virus disease. Antibodies described herein can also be conjugated to a molecule (e.g., polyethylene glycol) which can affect one or more biological and/or molecular properties of the antibodies, for example, stability (e.g., in serum), half-life, solubility, and antigenicity.

In specific embodiments, a conjugate comprises an antibody described herein and a molecule (e.g., therapeutic or drug moiety), wherein the antibody is linked directly to the molecule, or by way of one or more linkers. In certain embodiments, an antibody is covalently conjugated to a molecule. In a particular embodiment, an antibody is noncovalently conjugated to a molecule.

In certain embodiments, an antibody described herein is conjugated to one or more molecules (e.g., therapeutic or drug moiety) directly or indirectly via one or more linker molecules. In particular embodiments, a linker comprises 1, 2, 3, 4, 5, 6, 7, 8, 9, 10, 11, 12, 13, 14, 15, or 20 amino acid residues. In certain embodiments, a linker consists of 1 to 10 amino acid residues, 1 to 15 amino acid residues, 5 to 20 amino acid residues, 10 to 25 amino acid residues, 10 to 30 amino acid residues, or 10 to 50 amino acid residues. In particular embodiments, a linker is an enzyme-cleavable linker or a disulfide linker. In a specific embodiment, the cleavable linker is cleavable via an enzyme such an aminopeptidase, an aminoesterase, a dipeptidyl carboxy peptidase, or a protease of the blood clotting cascade. In a specific embodiment, the linker that may be conjugated to the antibody does not interfere with the antibody binding to either a recombinant NS1 polypeptide, Zika virus NS1, or both, using techniques known in the art or described herein. In a specific embodiment, the molecule that may be conjugated to the antibody does not interfere with the antibody binding to either a recombinant NS1 polypeptide, Zika virus NS1, or both, using techniques known in the art or described herein.

In specific aspects, diagnosis and detection can be accomplished, for example, by coupling the antibody to a detectable substance(s) including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Provided are antibodies described herein conjugated or recombinantly fused to a therapeutic moiety (or one or more therapeutic moieties) and uses of such antibodies. The antibody can be conjugated or recombinantly fused to a therapeutic moiety, such as a cytotoxin, e.g., a cytostatic or cytocidal agent, a therapeutic agent or a radioactive metal ion, e.g., alpha-emitters.

Further, provided herein are uses of the antibodies conjugated or recombinantly fused to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, β-interferon, γ-interferon, α-interferon, interleukin-2 (“IL-2”), interleukin-4 (“IL-4”), interleukin-6 (“IL-6”), interleukin-7 (“IL-7”), interleukin 9 (“IL-9”), interleukin-10 (“IL-10”), interleukin-12 (“IL-12”), interleukin-15 (“IL-15”), interleukin-18 (“IL-18”), interleukin-23 (“IL-23”), granulocyte macrophage colony stimulating factor (“GM-CSF”), granulocyte colony stimulating factor (“G-CSF”)), a growth factor, or a defensin. The therapeutic moiety or drug conjugated or recombinantly fused to an antibody should be chosen to achieve the desired prophylactic or therapeutic effect(s). In certain embodiments, an antibody conjugate may be used for the prophylactic or therapeutic uses described herein. In certain embodiments, the antibody is a modified antibody. A clinician or other medical personnel should consider the following when deciding on which therapeutic moiety or drug to conjugate or recombinantly fuse to an antibody: the nature of the disease, the severity of the disease, and the condition of the subject.

In addition, an antibody described herein can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4 (10): 2483-90; Peterson et al., 1999, Bioconjug. Chem. 10 (4): 553-7; and Zimmerman et al., 1999, Nucl. Med. Biol. 26 (8): 943-50, each incorporated by reference in their entireties.

Provided herein are antibodies recombinantly fused or chemically conjugated (including both covalent and non-covalent conjugations) to a heterologous protein or polypeptide (or fragment thereof, preferably to a polypeptide of about 10, about 20, about 30, about 40, about 50, about 60, about 70, about 80, about 90 or about 100 amino acids) to generate fusion proteins. In particular, provided herein are fusion proteins comprising an antigen-binding fragment of a monoclonal antibody (e.g., a Fab fragment, Fd fragment, Fv fragment, F(ab)₂ fragment, a VH domain, a VH CDR, a VL domain or a VL CDR) and a heterologous protein, polypeptide, or peptide. In a specific embodiment, the heterologous protein, polypeptide, or peptide that the antibody is fused to is useful for targeting the antibody to a particular cell type.

In one embodiment, a fusion protein provided herein comprises the antibody AA12, EB9, GB5, or FC12 and a heterologous polypeptide. In another embodiment, a fusion protein provided herein comprises an antigen-binding fragment of the antibody AA12, EB9, GB5, or FC12 and a heterologous polypeptide. In another embodiment, a fusion protein provided herein comprises (i) a VH domain having the amino acid sequence of the VH domain of the antibody AA12, EB9, GB5, or FC12 or a VL domain having the amino acid sequence of the VL domain of the antibody AA12, EB9, GB5, or FC12; and (ii) a heterologous polypeptide. In another embodiment, a fusion protein provided herein comprises one, two, or more VH CDRs having the amino acid sequence of the VH CDRs of the antibody AA12, EB9, GB5, or FC12 and a heterologous polypeptide. In another embodiment, a fusion protein comprises one, two, or more VL CDRs having the amino acid sequence of the VL CDRs of the antibody AA12, EB9, GB5, or FC12 and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein.

In another embodiment, a fusion protein provided herein comprises at least one VH domain and at least one VL domain of the antibody AA12 and a heterologous polypeptide. In yet another embodiment, a fusion protein provided herein comprises at least one VH CDR and at least one VL CDR of the antibody AA12 and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein. In certain embodiments, the above-referenced antibodies comprise a modified IgG (e.g., IgG1) constant domain, or FcRn binding fragment thereof (e.g., the Fc domain or hinge-Fc domain), described herein.

In another embodiment, a fusion protein provided herein comprises at least one VH domain and at least one VL domain of the antibody EB9 and a heterologous polypeptide. In yet another embodiment, a fusion protein provided herein comprises at least one VH CDR and at least one VL CDR of the antibody EB9 and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein.

In another embodiment, a fusion protein provided herein comprises at least one VH domain and at least one VL domain of the antibody GB5 and a heterologous polypeptide. In yet another embodiment, a fusion protein provided herein comprises at least one VH CDR and at least one VL CDR of the antibody GB5 and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein.

In another embodiment, a fusion protein provided herein comprises at least one VH domain and at least one VL domain of the antibody FC12 and a heterologous polypeptide. In yet another embodiment, a fusion protein provided herein comprises at least one VH CDR and at least one VL CDR of the antibody FC12 and a heterologous polypeptide. In certain embodiments, the above-referenced antibodies comprise a modified IgG (e.g., IgG1) constant domain, or FcRn binding fragment thereof (e.g., the Fc domain or hinge-Fc domain), described herein.

Moreover, antibodies can be fused to marker sequences, such as a peptide to facilitate purification. In preferred embodiments, the marker amino acid sequence is a hexa-histidine peptide (i.e., His-tag), such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783, 181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88:10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992; which are incorporated herein by reference in their entireties.

In particular, fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the monoclonal antibodies described herein (or an antigen-binding fragment thereof) (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16 (2): 76-82; Hansson, et al., 1999, J. Mol. Biol. 287:265-76; and Lorenzo and Blasco, 1998, Biotechniques 24 (2): 308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding a monoclonal antibody described herein (or an antigen-binding fragment thereof) may be recombined with one or more components, motifs, sections, parts, domains, fragments, etc. of one or more heterologous molecules.

An antibody can also be conjugated to a second antibody to form an antibody heteroconjugate as described by Segal in U.S. Pat. No. 4,676,980, which is incorporated herein by reference in its entirety.

An antibody can also be linked directly or indirectly to one or more antibodies to produce bispecific/multispecific antibodies.

An antibody can also be attached to solid supports, which are particularly useful for immunoassays or purification of an antigen. Such solid supports include, but are not limited to, glass, cellulose, polyacrylamide, nylon, polystyrene, polyvinyl chloride or polypropylene.

### 5.4 Nucleic Acid Sequences

### 5.4.1 Nucleic Acid Sequences Encoding Antibodies

In certain aspects, provided herein are polynucleotides comprising a nucleotide sequence encoding an antibody described herein or a fragment thereof (e.g., a VL domain, a VH domain, or both) that binds to a Zika virus NS1 (e.g., NS1 of a Zika virus strain described in Section 6, infra), and vectors, e.g., vectors comprising such polynucleotides for recombinant expression in host cells (e.g., E. coli and mammalian cells). Provided herein are polynucleotides comprising nucleotide sequences encoding any of the antibodies provided herein (see, e.g., Section 5.2), as well as vectors comprising such polynucleotide sequences, e.g., expression vectors for their efficient expression in host cells, e.g., mammalian cells.

As used herein, an “isolated” polynucleotide, nucleic acid sequence or nucleic acid molecule is one that is separated from other nucleic acid molecules that are present in the natural source (e.g., in a mouse or a human) of the nucleic acid molecule. Moreover, an “isolated” nucleic acid molecule, such as a cDNA molecule or RNA molecule, can be substantially free of other cellular material, or culture medium when produced by recombinant techniques, or substantially free of chemical precursors or other chemicals when chemically synthesized. For example, the language “substantially free” includes preparations of polynucleotide, nucleic acid sequence or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an antibody described herein is isolated or purified.

As used herein, the terms “polynucleotide(s)” “nucleic acid” and “nucleotide” include deoxyribonucleotides, deoxyribonucleic acids, ribonucleotides, and ribonucleic acids, and polymeric forms thereof, and includes either single- or double-stranded forms. In certain embodiments, the terms “polynucleotide(s)” “nucleic acid” and “nucleotide” include known analogues of natural nucleotides, for example, peptide nucleic acids (“PNA”s), that have similar binding properties as the reference nucleic acid. In some embodiments, the terms “polynucleotide(s)” “nucleic acid” and “nucleotide” refer to deoxyribonucleic acids (e.g., cDNA or DNA). In other embodiments, the terms “polynucleotide(s)” “nucleic acid” and “nucleotide” refer to ribonucleic acids (e.g., mRNA or RNA).

In particular aspects, provided herein are polynucleotides comprising a nucleic acid sequence encoding an antibody (e.g., a murine, chimeric, or humanized antibody, or antigen-binding fragments thereof), which binds to a Zika virus NS1 (e.g., NS1 of a Zika virus strain described in Section 6, infra) and comprises an amino acid sequence as described herein, as well as antibodies which compete with such antibodies for binding to a Zika virus NS1 (e.g., NS1 of a Zika virus strain described in Section 6, infra) (e.g., in a dose-dependent manner), or which binds to the same epitope as that of such antibodies. In particular embodiments, a nucleic acid sequence described herein encodes an antibody which comprises a VL domain and a VH domain of antibody AA12, EB9, GB5, or FC12.

In particular embodiments, a polynucleotide described herein comprises a nucleic acid sequence encoding an antibody which comprises a VL domain comprising the amino acid sequence of SEQ ID NO: 10 and/or a VH domain comprising the amino acid of SEQ ID NO: 9. In certain embodiments, a polynucleotide described herein comprises a nucleic acid sequence encoding such a VL domain (e.g., a VL domain comprising the amino acid sequence of SEQ ID NO: 10). In certain embodiments, a polynucleotide described herein comprises a nucleic acid sequence encoding such a VH domain (e.g., a VH domain comprising the amino acid sequence of SEQ ID NO: 9). In some embodiments, a polynucleotide described herein comprising a nucleic acid sequence encoding for an antibody that binds to Zika virus NS1 (e.g., NS1 of a Zika virus strain described in Section 6, infra), wherein the antibody comprises 1, 2, or 3 VH CDRs and/or 1, 2, or 3 VL CDRs of the antibody AA12.

In particular embodiments, a polynucleotide described herein comprises a nucleic acid sequence encoding an antibody which comprises a VL domain comprising the amino acid sequence of SEQ ID NO: 12 and/or a VH domain comprising the amino acid of SEQ ID NO: 11. In certain embodiments, a polynucleotide described herein comprises a nucleic acid sequence encoding such a VL domain (e.g., a VL domain comprising the amino acid sequence of SEQ ID NO: 12). In certain embodiments, a polynucleotide described herein comprises a nucleic acid sequence encoding such a VH domain (e.g., a VH domain comprising the amino acid sequence of SEQ ID NO: 11). In some embodiments, a polynucleotide described herein comprising a nucleic acid sequence encoding for an antibody that binds to Zika virus NS1 (e.g., NS1 of a Zika virus strain described in Section 6, infra), wherein the antibody comprises 1, 2, or 3 VH CDRs and/or 1, 2, or 3 VL CDRs of the antibody EB9.

In particular embodiments, a polynucleotide described herein comprises a nucleic acid sequence encoding an antibody which comprises a VL domain comprising the amino acid sequence of SEQ ID NO: 14 and/or a VH domain comprising the amino acid of SEQ ID NO:13. In certain embodiments, a polynucleotide described herein comprises a nucleic acid sequence encoding such a VL domain (e.g., a VL domain comprising the amino acid sequence of SEQ ID NO: 14). In certain embodiments, a polynucleotide described herein comprises a nucleic acid sequence encoding such a VH domain (e.g., a VH domain comprising the amino acid sequence of SEQ ID NO: 13). In some embodiments, a polynucleotide described herein comprising a nucleic acid sequence encoding for an antibody that binds to Zika virus NS1 (e.g., NS1 of a Zika virus strain described in Section 6, infra), wherein the antibody comprises 1, 2, or 3 VH CDRs and/or 1, 2, or 3 VL CDRs of the antibody GB5.

In particular embodiments, a polynucleotide described herein comprises a nucleic acid sequence encoding an antibody which comprises a VL domain comprising the amino acid sequence of SEQ ID NO: 16 and/or a VH domain comprising the amino acid of SEQ ID NO: 15. In certain embodiments, a polynucleotide described herein comprises a nucleic acid sequence encoding such a VL domain (e.g., a VL domain comprising the amino acid sequence of SEQ ID NO: 16). In certain embodiments, a polynucleotide described herein comprises a nucleic acid sequence encoding such a VH domain (e.g., a VH domain comprising the amino acid sequence of SEQ ID NO: 15). In some embodiments, a polynucleotide described herein comprising a nucleic acid sequence encoding for an antibody that binds to Zika virus NS1 (e.g., NS1 of a Zika virus strain described in Section 6, infra), wherein the antibody comprises 1, 2, or 3 VH CDRs and/or 1, 2, or 3 VL CDRs of the antibody FC12.

In particular embodiments, a polynucleotide described herein comprises a nucleic acid sequence encoding an antibody, which binds to a Zika virus NS1 (e.g., NS1 of a Zika virus strain described in Section 6, infra), comprising VL CDRs and/or VH CDRs of antibody AA12. For example, in a specific embodiment, a polynucleotide described herein comprises a nucleic acid sequence encoding an antibody which comprises a VL domain comprising CDR1, CDR2, and CDR3 comprising the amino acid sequences of SEQ ID NOs: 20-22, respectively, and/or a VH domain comprising CDR1, CDR2, and CDR3 comprising the amino acid sequences of SEQ ID NOs: 17-19, respectively. In another specific embodiment, a polynucleotide described herein comprises a nucleic acid sequence encoding an antibody which comprises a VL domain comprising CDR1, CDR2, and CDR3 comprising the amino acid sequences of SEQ ID NOs: 34-36, respectively, and/or a VH domain comprising CDR1, CDR2, and CDR3 comprising the amino acid sequences of SEQ ID NOs: 31-33, respectively. In certain aspects, provided herein are polynucleotides comprising a nucleic acid sequence encoding the light chain or heavy chain of an antibody described herein. The polynucleotides can comprise a nucleic acid sequence encoding a light chain or a VL domain, comprising the VL FRs and CDRs of an antibody described herein. The polynucleotides can comprise a nucleic acid sequence encoding a heavy chain, or a VH domain, comprising the VH FRs and CDRs of antibodies described herein. In specific embodiments, a polynucleotide described herein comprise a nucleic acid sequence encoding a VL domain comprising the amino acid sequence of SEQ ID NO: 10. In specific embodiments, a polynucleotide described herein encodes a VH domain comprising the amino acid sequence of SEQ ID NO: 9.

In particular embodiments, a polynucleotide described herein comprises a nucleic acid sequence encoding an antibody, which binds to a Zika virus NS1 (e.g., NS1 of a Zika virus strain described in Section 6, infra), comprising VL CDRs and/or VH CDRs of antibody EB9. For example, in a specific embodiment, a polynucleotide described herein comprises a nucleic acid sequence encoding an antibody which comprises a VL domain comprising CDR1, CDR2, and CDR3 comprising the amino acid sequences of SEQ ID NOs: 48-50, respectively, and/or a VH domain comprising CDR1, CDR2, and CDR3 comprising the amino acid sequences of SEQ ID NOs: 45-47, respectively. In another specific embodiment, a polynucleotide described herein comprises a nucleic acid sequence encoding an antibody which comprises a VL domain comprising CDR1, CDR2, and CDR3 comprising the amino acid sequences of SEQ ID NOs: 62-64, respectively, and/or a VH domain comprising CDR1, CDR2, and CDR3 comprising the amino acid sequences of SEQ ID NOs: 59-61, respectively. In certain aspects, provided herein are polynucleotides comprising a nucleic acid sequence encoding the light chain or heavy chain of an antibody described herein. The polynucleotides can comprise a nucleic acid sequence encoding a light chain or a VL domain, comprising the VL FRs and CDRs of an antibody described herein. The polynucleotides can comprise a nucleic acid sequence encoding a heavy chain, or a VH domain, comprising the VH FRs and CDRs of antibodies described herein. In specific embodiments, a polynucleotide described herein comprise a nucleic acid sequence encoding a VL domain comprising the amino acid sequence of SEQ ID NO: 12. In specific embodiments, a polynucleotide described herein encodes a VH domain comprising the amino acid sequence of SEQ ID NO: 11.

In particular embodiments, a polynucleotide described herein comprises a nucleic acid sequence encoding an antibody, which binds to a Zika virus NS1 (e.g., NS1 of a Zika virus strain described in Section 6, infra), comprising VL CDRs and/or VH CDRs of antibody GB5. For example, in a specific embodiment, a polynucleotide described herein comprises a nucleic acid sequence encoding an antibody which comprises a VL domain comprising CDR1, CDR2, and CDR3 comprising the amino acid sequences of SEQ ID NOs: 76-78, respectively, and/or a VH domain comprising CDR1, CDR2, and CDR3 having the amino acid sequences of SEQ ID NOs: 73-75, respectively. In another specific embodiment, a polynucleotide described herein comprises a nucleic acid sequence encoding an antibody which comprises a VL domain comprising CDR1, CDR2, and CDR3 comprising the amino acid sequences of SEQ ID NOs: 90-92, respectively, and/or a VH domain comprising CDR1, CDR2, and CDR3 having the amino acid sequences of SEQ ID NOs: 87-89, respectively. In certain aspects, provided herein are polynucleotides comprising a nucleic acid sequence encoding the light chain or heavy chain of an antibody described herein. The polynucleotides can comprise a nucleic acid sequence encoding a light chain or a VL domain, comprising the VL FRs and CDRs of an antibody described herein. The polynucleotides can comprise a nucleic acid sequence encoding a heavy chain, or a VH domain, comprising the VH FRs and CDRs of antibodies described herein. In specific embodiments, a polynucleotide described herein comprise a nucleic acid sequence encoding a VL domain comprising the amino acid sequence of SEQ ID NO: 14. In specific embodiments, a polynucleotide described herein encodes a VH domain comprising the amino acid sequence of SEQ ID NO: 13.

In particular embodiments, a polynucleotide described herein comprises a nucleic acid sequence encoding an antibody, which binds to a Zika virus NS1 (e.g., NS1 of a Zika virus strain described in Section 6, infra), comprising VL CDRs and/or VH CDRs of antibody FC12. For example, in a specific embodiment, a polynucleotide described herein comprises a nucleic acid sequence encoding an antibody which comprises a VL domain comprising CDR1, CDR2, and CDR3 comprising the amino acid sequences of SEQ ID NOs: 104-106, respectively, and/or a VH domain comprising CDR1, CDR2, and CDR3 having the amino acid sequences of SEQ ID NOs: 101-103, respectively. In another specific embodiment, a polynucleotide described herein comprises a nucleic acid sequence encoding an antibody which comprises a VL domain comprising CDR1, CDR2, and CDR3 comprising the amino acid sequences of SEQ ID NOs: 118-120, respectively, and/or a VH domain comprising CDR1, CDR2, and CDR3 having the amino acid sequences of SEQ ID NOs: 115-117, respectively. In certain aspects, provided herein are polynucleotides comprising a nucleic acid sequence encoding the light chain or heavy chain of an antibody described herein. The polynucleotides can comprise a nucleic acid sequence encoding a light chain or a VL domain, comprising the VL FRs and CDRs of an antibody described herein. The polynucleotides can comprise a nucleic acid sequence encoding a heavy chain, or a VH domain, comprising the VH FRs and CDRs of antibodies described herein. In specific embodiments, a polynucleotide described herein comprise a nucleic acid sequence encoding a VL domain comprising the amino acid sequence of SEQ ID NO: 16. In specific embodiments, a polynucleotide described herein encodes a VH domain comprising the amino acid sequence of SEQ ID NO: 15.

In particular embodiments, provided herein is a polynucleotide encoding a VL domain, wherein the polynucleotide comprises the nucleic acid sequence of SEQ ID NO: 2. In particular embodiments, provided herein is a polynucleotide encoding a VH domain, wherein the polynucleotide comprises the nucleic acid sequence of SEQ ID NO: 1. In certain embodiments, provided herein is a polynucleotide encoding an antibody described herein, wherein the polynucleotide comprises the nucleic acid sequence of SEQ ID NO: 2 encoding a VL domain and the nucleic acid sequence of SEQ ID NO: 1 encoding a VH domain.

In particular embodiments, provided herein is a polynucleotide encoding a VL domain, wherein the polynucleotide comprises the nucleic acid sequence of SEQ ID NO: 4. In particular embodiments, provided herein is a polynucleotide encoding a VH domain, wherein the polynucleotide comprises the nucleic acid sequence of SEQ ID NO: 3. In certain embodiments, provided herein is a polynucleotide encoding an antibody described herein, wherein the polynucleotide comprises the nucleic acid sequence of SEQ ID NO: 4 encoding a VL domain and the nucleic acid sequence of SEQ ID NO: 3 encoding a VH domain.

In particular embodiments, provided herein is a polynucleotide encoding a VL domain, wherein the polynucleotide comprises the nucleic acid sequence of SEQ ID NO: 6. In particular embodiments, provided herein is a polynucleotide encoding a VH domain, wherein the polynucleotide comprises the nucleic acid sequence of SEQ ID NO: 5. In certain embodiments, provided herein is a polynucleotide encoding an antibody described herein, wherein the polynucleotide comprises the nucleic acid sequence of SEQ ID NO: 6 encoding a VL domain and the nucleic acid sequence of SEQ ID NO: 5 encoding a VH domain.

In particular embodiments, provided herein is a polynucleotide encoding a VL domain, wherein the polynucleotide comprises the nucleic acid sequence of SEQ ID NO: 8. In particular embodiments, provided herein is a polynucleotide encoding a VH domain, wherein the polynucleotide comprises the nucleic acid sequence of SEQ ID NO: 7. In certain embodiments, provided herein is a polynucleotide encoding an antibody described herein, wherein the polynucleotide comprises the nucleic acid sequence of SEQ ID NO: 8 encoding a VL domain and the nucleic acid sequence of SEQ ID NO: 7 encoding a VH domain.

In particular embodiments, a polynucleotide described herein encodes a VL domain, wherein the polynucleotide comprises a nucleic acid sequence that is at least 80%, 85%, 90%, 91%, 92%, 93%, 94%, 95%, 96%, 97%, or 98% identical to the nucleic acid sequence of SEQ ID NO: 2, 4, 6, or 8. In particular embodiments, a polynucleotide described herein encodes a VH domain, wherein the polynucleotide comprises a nucleic acid sequence that is at least 80%, 85%, 90%, 91%, 92%, 93%, 94%, 95%, 96%, 97%, or 98% identical to the nucleic acid sequence of SEQ ID NO: 1, 3, 5 or 7.

In particular embodiments, a polynucleotide described herein comprises nucleic acid sequences that encode a VL domain and a VH domain, wherein the nucleic acid sequence encoding the VL domain is at least 80%, 85%, 90%, 91%, 92%, 93%, 94%, 95%, 96%, 97%, or 98% identical to the nucleic acid sequence of SEQ ID NO: 2 and the nucleic acid sequence encoding the VH domain is at least 80%, 85%, 90%, 91%, 92%, 93%, 94%, 95%, 96%, 97%, or 98% identical to the nucleic acid sequence of SEQ ID NO: 1.

In particular embodiments, a polynucleotide described herein comprises nucleic acid sequences that encode a VL domain and a VH domain, wherein the nucleic acid sequence encoding the VL domain is at least 80%, 85%, 90%, 91%, 92%, 93%, 94%, 95%, 96%, 97%, or 98% identical to the nucleic acid sequence of SEQ ID NO: 4 and the nucleic acid sequence encoding the VH domain is at least 80%, 85%, 90%, 91%, 92%, 93%, 94%, 95%, 96%, 97%, or 98% identical to the nucleic acid sequence of SEQ ID NO: 3.

In particular embodiments, a polynucleotide described herein comprises nucleic acid sequences that encode a VL domain and a VH domain, wherein the nucleic acid sequence encoding the VL domain is at least 80%, 85%, 90%, 91%, 92%, 93%, 94%, 95%, 96%, 97%, or 98% identical to the nucleic acid sequence of SEQ ID NO: 6 and the nucleic acid sequence encoding the VH domain is at least 80%, 85%, 90%, 91%, 92%, 93%, 94%, 95%, 96%, 97%, or 98% identical to the nucleic acid sequence of SEQ ID NO: 5.

In particular embodiments, a polynucleotide described herein comprises nucleic acid sequences that encode a VL domain and a VH domain, wherein the nucleic acid sequence encoding the VL domain is at least 80%, 85%, 90%, 91%, 92%, 93%, 94%, 95%, 96%, 97%, or 98% identical to the nucleic acid sequence of SEQ ID NO: 8 and the nucleic acid sequence encoding the VH domain is at least 80%, 85%, 90%, 91%, 92%, 93%, 94%, 95%, 96%, 97%, or 98% identical to the nucleic acid sequence of SEQ ID NO: 7.

In particular embodiments, a polynucleotide described herein encodes a light chain, wherein the polynucleotide comprises the nucleic acid sequence of SEQ ID NO: 2, 4, 6, or 8. In particular embodiments, a polynucleotide described herein encodes a heavy chain, wherein the polynucleotide comprises the nucleic acid sequence of SEQ ID NO: 1, 3, 5, or 7.

In particular embodiments, a polynucleotide(s) described herein encodes a light chain and a heavy chain, wherein the polynucleotide(s) comprises the nucleic acid sequence of SEQ ID NO: 2 and the nucleic acid sequence of SEQ ID NO: 1. In particular embodiments, a polynucleotide(s) described herein encodes a light chain and a heavy chain, wherein the polynucleotide(s) comprises the nucleic acid sequence of SEQ ID NO: 4 and the nucleic acid sequence of SEQ ID NO: 3. In particular embodiments, a polynucleotide(s) described herein encodes a light chain and a heavy chain, wherein the polynucleotide(s) comprises the nucleic acid sequence of SEQ ID NO: 6 and the nucleic acid sequence of SEQ ID NO: 5. In particular embodiments, a polynucleotide(s) described herein encodes a light chain and a heavy chain, wherein the polynucleotide(s) comprises the nucleic acid sequence of SEQ ID NO: 8 and the nucleic acid sequence of SEQ ID NO: 7.

In particular embodiments, a polynucleotide described herein comprises nucleic acid sequences that encode a light chain and a heavy chain, wherein the nucleic acid sequence encoding the light chain comprises a nucleotide sequence that is at least 80%, 85%, 90%, 91%, 92%, 93%, 94%, 95%, 96%, 97%, or 98% identical to the nucleic acid sequence of SEQ ID NO: 2 and/or the nucleic acid sequence encoding the heavy chain comprises a nucleotide sequence that is at least 80%, 85%, 90%, 91%, 92%, 93%, 94%, 95%, 96%, 97%, or 98% identical to the nucleic acid sequence of SEQ ID NO: 1. In particular embodiments, a polynucleotide described herein comprises nucleic acid sequences that encode a light chain and a heavy chain, wherein the nucleic acid sequence encoding the light chain comprises a nucleotide sequence that is at least 80%, 85%, 90%, 91%, 92%, 93%, 94%, 95%, 96%, 97%, or 98% identical to the nucleic acid sequence of SEQ ID NO: 4 and/or the nucleic acid sequence encoding the heavy chain comprises a nucleotide sequence that is at least 80%, 85%, 90%, 91%, 92%, 93%, 94%, 95%, 96%, 97%, or 98% identical to the nucleic acid sequence of SEQ ID NO: 3. In particular embodiments, a polynucleotide described herein comprises nucleic acid sequences that encode a light chain and a heavy chain, wherein the nucleic acid sequence encoding the light chain comprises a nucleotide sequence that is at least 80%, 85%, 90%, 91%, 92%, 93%, 94%, 95%, 96%, 97%, or 98% identical to the nucleic acid sequence of SEQ ID NO: 6 and/or the nucleic acid sequence encoding the heavy chain comprises a nucleotide sequence that is at least 80%, 85%, 90%, 91%, 92%, 93%, 94%, 95%, 96%, 97%, or 98% identical to the nucleic acid sequence of SEQ ID NO: 5. In particular embodiments, a polynucleotide described herein comprises nucleic acid sequences that encode a light chain and a heavy chain, wherein the nucleic acid sequence encoding the light chain comprises a nucleotide sequence that is at least 80%, 85%, 90%, 91%, 92%, 93%, 94%, 95%, 96%, 97%, or 98% identical to the nucleic acid sequence of SEQ ID NO: 8 and/or the nucleic acid sequence encoding the heavy chain comprises a nucleotide sequence that is at least 80%, 85%, 90%, 91%, 92%, 93%, 94%, 95%, 96%, 97%, or 98% identical to the nucleic acid sequence of SEQ ID NO: 7.

In specific aspects, provided herein is a polynucleotide comprising a nucleotide sequence encoding an antibody provided herein (e.g., murine, chimeric, human or humanized antibody) which competitively blocks (e.g., in a dose dependent manner), antibody AA12, EB9, GB5, or FC12 from binding to a Zika virus NS1, as determined using assays known to one of skill in the art or described herein (e.g., ELISA competitive assays).

In a specific embodiment, a polynucleotide provided herein comprises a nucleotide sequence encoding a kappa light chain (e.g., human kappa light chain). In another specific embodiment, a polynucleotide provided herein comprises a nucleotide sequence encoding a lambda light chain (e.g., human lambda light chain).

In a specific embodiment, a polynucleotide provided herein comprises a nucleotide sequence encoding an IgG1 heavy chain (e.g., human IgG1 heavy chain) of an antibody described herein. In another specific embodiment, a polynucleotide provided herein comprises a nucleotide sequence encoding IgG4 heavy chain (e.g., human IgG4 heavy chain). In another specific embodiment, a polynucleotide provided herein comprises a nucleotide sequence encoding IgG2 heavy chain (e.g., human IgG2 heavy chain).

In a specific embodiment, a polynucleotide provided herein encodes an antigen-binding domain, e.g., an Fab or F(ab′)₂.

In another particular embodiment, a polynucleotide provided herein comprises a nucleotide sequence encoding an antibody described herein, which binds to a Zika virus NS1, wherein the antibody comprises a light chain and a heavy chain, and wherein (i) the light chain comprises a VL domain comprising a VL CDR1, VL CDR2, and VL CDR3 having the amino acid sequences of the VL CDRs of antibody AA12; (ii) the heavy chain comprises a VH domain comprising a VH CDR1, VH CDR2, and VH CDR3 having the amino acid sequences of the VH CDRs of antibody AA12; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 heavy chain or human IgG2a heavy chain.

In another particular embodiment, a polynucleotide provided herein comprises a nucleotide sequence encoding an antibody described herein, which binds to a Zika virus NS1, wherein the antibody comprises a light chain and a heavy chain, and wherein (i) the light chain comprises a VL domain comprising a VL CDR1, VL CDR2, and VL CDR3 having the amino acid sequences of the VL CDRs of antibody EB9; (ii) the heavy chain comprises a VH domain comprising a VH CDR1, VH CDR2, and VH CDR3 having the amino acid sequences of the VH CDRs of antibody EB9; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 heavy chain or human IgG2a heavy chain.

In another particular embodiment, a polynucleotide provided herein comprises a nucleotide sequence encoding an antibody described herein, which binds to a Zika virus NS1, wherein the antibody comprises a light chain and a heavy chain, and wherein (i) the light chain comprises a VL domain comprising a VL CDR1, VL CDR2, and VL CDR3 having the amino acid sequences of the VL CDRs of antibody GB5; (ii) the heavy chain comprises a VH domain comprising a VH CDR1, VH CDR2, and VH CDR3 having the amino acid sequences of the VH CDRs of antibody GB5; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 heavy chain or human IgG2a heavy chain.

In another particular embodiment, a polynucleotide provided herein comprises a nucleotide sequence encoding an antibody described herein, which binds to a Zika virus NS1, wherein the antibody comprises a light chain and a heavy chain, and wherein (i) the light chain comprises a VL domain comprising a VL CDR1, VL CDR2, and VL CDR3 having the amino acid sequences of the VL CDRs of antibody FC12; (ii) the heavy chain comprises a VH domain comprising a VH CDR1, VH CDR2, and VH CDR3 having the amino acid sequences of the VH CDRs of antibody FC12; (iii) the light chain further comprises a constant light chain domain comprising the amino acid sequence of the constant domain of a human kappa light chain; and (iv) the heavy chain further comprises a constant heavy chain domain comprising the amino acid sequence of the constant domain of a human IgG1 heavy chain or human IgG2a heavy chain.

In certain embodiments, with respect to a polynucleotide provided herein comprising a nucleotide sequence encoding a VL domain and VH domain of any of the antibodies described herein, the polynucleotide of the VL domain further comprises primate (e.g., human) framework regions; and the VH domain further comprises primate (e.g., human) framework regions.

In a specific embodiment, provided herein are polynucleotides comprising a nucleotide sequence encoding an antibody, or a fragment or domain thereof (e.g., VL domain or VH domain), designated herein as antibody AA12, EB9, GB5, or FC12.

Also provided are polynucleotides that hybridize under high stringency, intermediate or lower stringency hybridization conditions to antisense polynucleotides of polynucleotides that encode an antibody described herein or a fragment thereof (e.g., VL domain or VH domain). In specific embodiments, a polynucleotide described herein hybridizes under high stringency, or intermediate stringency hybridization conditions to an antisense polynucleotide of a polynucleotide encoding a VL domain, e.g., SEQ ID NO: 2, and/or VH domain, e.g., SEQ ID NO: 1, provided herein. In specific embodiments, a polynucleotide described herein hybridizes under high stringency, or intermediate stringency hybridization conditions to an antisense polynucleotide of a polynucleotide comprising SEQ ID NO: 1 or 2.

In specific embodiments, a polynucleotide described herein hybridizes under high stringency, or intermediate stringency hybridization conditions to an antisense polynucleotide of a polynucleotide encoding a VL domain, e.g., SEQ ID NO: 4, and/or VH domain, e.g., SEQ ID NO: 3, provided herein. In specific embodiments, a polynucleotide described herein hybridizes under high stringency, or intermediate stringency hybridization conditions to an antisense polynucleotide of a polynucleotide comprising SEQ ID NO: 3 or 4.

In specific embodiments, a polynucleotide described herein hybridizes under high stringency, or intermediate stringency hybridization conditions to an antisense polynucleotide of a polynucleotide encoding a VL domain, e.g., SEQ ID NO: 6, and/or VH domain, e.g., SEQ ID NO: 5, provided herein. In specific embodiments, a polynucleotide described herein hybridizes under high stringency, or intermediate stringency hybridization conditions to an antisense polynucleotide of a polynucleotide comprising SEQ ID NO: 5 or 6.

In specific embodiments, a polynucleotide described herein hybridizes under high stringency, or intermediate stringency hybridization conditions to an antisense polynucleotide of a polynucleotide encoding a VL domain, e.g., SEQ ID NO: 8, and/or VH domain, e.g., SEQ ID NO: 7, provided herein. In specific embodiments, a polynucleotide described herein hybridizes under high stringency, or intermediate stringency hybridization conditions to an antisense polynucleotide of a polynucleotide comprising SEQ ID NO: 7 or 8.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3.

Also provided herein are polynucleotides encoding an antibody or a fragment thereof (e.g., an antigen-binding fragment thereof) that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an antibody encoded by polynucleotides that have not been optimized.

In certain embodiments, an optimized polynucleotide sequence encoding an antibody described herein or a fragment thereof (e.g., VL region and/or VH region) can hybridize to an antisense polynucleotide of an unoptimized polynucleotide encoding an antibody described herein or a fragment thereof (e.g., VL region and/or VH region). In specific embodiments, an optimized nucleotide sequence encoding an antibody described herein or a fragment thereof (e.g., VL region and/or VH region) hybridizes under high stringency conditions to an antisense polynucleotide of an unoptimized polynucleotide encoding an antibody described herein or a fragment thereof (e.g., VL region and/or VH region). In a specific embodiment, an optimized nucleotide sequence encoding an antibody described herein or a fragment thereof (e.g., VL region and/or VH region) hybridizes under intermediate or lower stringency hybridization conditions to an antisense polynucleotide of an unoptimized polynucleotide encoding an antibody described herein or a fragment thereof (e.g., VL region and/or VH region). Information regarding hybridization conditions have been described, see, e.g., U.S. Patent Application Publication No. US 2005/0048549 (e.g., paragraphs 72-73), which is incorporated herein by reference in its entirety.

The polynucleotides can be obtained, and the nucleotide sequence of the polynucleotides determined, by any method known in the art. Nucleotide sequences encoding antibodies described herein, and modified forms of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light domain and/or the variable heavy domain of an antibody. The amplified nucleic acids can be cloned into vectors for expression in host cells and for further cloning, for example, to generate chimeric and humanized antibodies.

If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding an antibody can be readily isolated and sequenced using conventional procedures (e.g., by using oligonucleotide probes that are capable of binding specifically to genes encoding the heavy and light chains of the antibody). Hybridoma cells can serve as a source of such DNA. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of antibodies in the recombinant host cells.

In phage display methods, functional antibody domains are displayed on the surface of phage particles which carry the polynucleotide sequences encoding them. In particular, a library of DNA sequences encoding VH and VL domains are generated (e.g., amplified from animal cDNA libraries such as human cDNA libraries or random libraries are generated by chemical synthesis). The DNA encoding the VH and VL domains are recombined together with an scFv linker by PCR and cloned into a phagemid vector. The vector is electroporated in E. coli and the E. coli is infected with helper phage. Phage expressing an antigen-binding domain that binds to a particular antigen can be selected or identified with antigen, e.g., using labeled antigen or antigen bound or captured to a solid surface or bead. After phage selection, the antibody coding regions from the phage can be isolated and used to generate whole antibodies, including human antibodies, or any other desired antigen-binding fragment, and expressed in any desired host, including mammalian cells, insect cells, plant cells, yeast, and bacteria, e.g., as described below. Techniques to recombinantly produced Fab, Fab′ and F(ab′)₂ fragments can also be employed using methods known in the art such as those disclosed in PCT Publication No. WO 92/22324; Mullinax et al., 1992, BioTechniques, 12 (6): 864-869; Sawai et al., 1995, AJRI, 34:26-34; and Better et al., 1988, Science, 240:1041-1043.

Antibodies can be isolated from antibody phage libraries generated using the techniques described in McCafferty et al., Nature, 348:552-554 (1990). Clackson et al., Nature, 352:624-628 (1991). Marks et al., J. Mol. Biol., 222:581-597 (1991) describe the isolation of murine and human antibodies, respectively, using phage libraries. Chain shuffling can be used in the production of high affinity (nM range) human antibodies (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise a promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

In a specific embodiment, provided herein are two vectors (e.g., plasmids or viruses), wherein one vector comprises the VH domain of an antibody described herein, and the second vector comprises the VL domain of an antibody described herein.

In a non-limiting example, the Dyax (Cambridge, MA) technology platform can be used to convert Fab-phage or Fabs to complete IgG antibodies, such as the Dyax pR rapid reformatting vectors (RR). Briefly, by PCR, a Fab-encoding DNA fragment is inserted into a Dyax pR-RRV between a eukaryotic leader sequence and an IgG heavy chain constant region cDNA. Antibody expression is driven by the human cytomegalovirus (hCMV). In a second cloning step, bacterial regulatory elements are replaced by the appropriate eukaryotic sequences (i.e., the IRES (internal ribosome entry site) motif). The expression vector can also include the SV40 origin of replication. The Dyax pRh1(a, z), pRh1(f), pRh4 and pRm2a are expression vectors allowing expression of reformatted FAbs as human IgG1 (isotype a, z), human IgG1 (isotype F), human IgG4, and mouse IgG2a, respectively. Expressing vectors can be introduced into a suitable host cell (e.g., HEK293T cells, CHO cells)) for expression and purification.

The DNA also can be modified, for example, by substituting the coding sequence for human heavy and light chain constant domains in place of e.g., murine sequences, or by covalently joining to the immunoglobulin coding sequence all or part of the coding sequence for a non-immunoglobulin polypeptide.

In some embodiments, a polynucleotide(s) encoding an antibody provided herein is isolated. In other embodiments, a polynucleotide(s) encoding an antibody provided herein is not isolated. In yet other embodiments, a polynucleotide(s) encoding an antibody provided herein is integrated, e.g., into chromosomal DNA or an expression vector. In specific embodiments, a polynucleotide(s) encoding an antibody provided herein is not integrated into chromosomal DNA.

### 5.4.2 Nucleic Acid Sequences Encoding NS1 Polypeptides

Provided herein are polynucleotides that comprising a nucleotide sequence encoding an NS1 polypeptide described herein. Due to the degeneracy of the genetic code, any nucleotide sequence that encodes an NS1 polypeptide described herein is encompassed herein. In a specific embodiment, the polynucleotides that encode an NS1 polypeptide described herein is an RNA sequence (e.g., mRNA) or a cDNA sequence.

Also provided herein are polynucleotides capable of hybridizing to a polynucleotide encoding an NS1 polypeptide. In certain embodiments, provided herein are polynucleotides capable of hybridizing to a fragment of a nucleic acid encoding an NS1 polypeptide described herein. In other embodiments, provided herein are polynucleotides capable of hybridizing to the full length of a polynucleotide encoding an NS1 polypeptide described herein. General parameters for hybridization conditions for nucleic acids are described in Sambrook et al., Molecular Cloning—A Laboratory Manual (2nd Ed.), Vols. 1-3, Cold Spring Harbor Laboratory, Cold Spring Harbor, New York (1989), and in Ausubel et al., Current Protocols in Molecular Biology, vol. 2, Current Protocols Publishing, New York (1994). Hybridization may be performed under high stringency conditions, medium stringency conditions, or low stringency conditions. Those of skill in the art will understand that low, medium and high stringency conditions are contingent upon multiple factors all of which interact and are also dependent upon the nucleic acids in question. For example, high stringency conditions may include temperatures within 5° C. melting temperature of the nucleic acid(s), a low salt concentration (e.g., less than 250 mM), and a high co-solvent concentration (e.g., 1-20% of co-solvent, e.g., DMSO). Low stringency conditions, on the other hand, may include temperatures greater than 10° C. below the melting temperature of the nucleic acid(s), a high salt concentration (e.g., greater than 1000 mM) and the absence of co-solvents.

Any codon optimization technique known to one of skill in the art may be used to codon optimize a nucleic acid sequence encoding a protein of interest (e.g., an NS1 polypeptide described herein). In a specific embodiment, a polynucleotide sequence encoding an NS1 polypeptide is codon optimized or the nucleic acid sequence encoding a fragment of the NS1 polypeptide comprising (or consisting of) the Zika virus NS1 coding region is codon optimized. As an exemplary method for codon optimization, each codon in the open frame of the nucleic acid sequence encoding an NS1 polypeptide or an NS1 coding region thereof is replaced by the codon most frequently used in mammalian proteins (e.g., humans). Methods of codon optimization are known in the art, e.g, the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety. In a specific embodiment, the nucleic acid sequence is human codon optimized. Codon optimization may be done using a web-based program (www.encorbio.com/protocols/Codon.htm) that uses the Codon Usage Database, maintained by the Department of Plant Gene Research in Kazusa, Japan.

In some embodiments, a polynucleotide encoding an NS1 polypeptide described herein is isolated.

### 5.5 Antibody Production

In one aspect, provided herein are methods for making an antibody described herein, which binds to a Zika virus NS1. In a specific embodiment, an antibody described herein (e.g., an antigen-binding fragment), which binds to a Zika virus NS1, may be prepared, expressed, created or isolated by any means that involves creation, e.g., via synthesis or genetic engineering of sequences. In a specific embodiments, such an antibody comprises sequences that are encoded by DNA sequences that do not naturally exist withing the antibody germline repertoire of an animal or mammal (e.g., a human).

In certain aspects, a method for making an antibody described herein, which binds to a Zika virus NS1, comprises the step of culturing a cell (e.g., host cell or hybridoma cell) that expresses the antibody. In certain embodiments, the method for making an antibody described herein further comprises the step of purifying the antibody expressed by the cell. In certain aspects, a method for making an antibody described herein (e.g., an antigen-binding fragment thereof), which binds to a Zika virus NS1, comprises the step of culturing a cell (e.g., host cell or hybridoma cell) that comprises polynucleotides or vectors encoding the antibody. In a particular aspect, provided herein are methods for producing an antibody described herein (e.g., an antigen-binding fragment thereof), comprising expressing such antibody from a host cell. In a specific embodiment, provided herein is a method for producing an antibody comprising culturing a host cell(s) expressing an antibody described herein and isolating the antibody from the cell(s).

In certain aspects, provided herein are cells (e.g., host cells) expressing (e.g., recombinantly expressing) the antibodies described herein (e.g., an antigen-binding fragment thereof) and related expression vectors. In another aspect, provided herein are vectors (e.g., expression vectors) comprising polynucleotides comprising nucleotide sequences encoding antibodies (e.g., an antigen-binding fragment) for recombinant expression in host cells, preferably in mammalian cells. Also provided herein are host cells comprising a polynucleotide encoding an antibody, or vectors comprising a polynucleotide encoding an antibody for recombinantly expressing an antibody described herein (e.g., antibody AA12, EB9, GB5 or FC12, or another antibody described in Section 5.2, supra). In a specific embodiment, provided herein is a host cell comprising two vectors, wherein the first vector comprises a polynucleotide encoding a VH domain or heavy chain of an antibody described herein (e.g., antibody AA12, EB9, GB5 or FC12, or another antibody described in Section 5.2, supra) and the second vector comprises a polynucleotide encoding a VL domain or light chain of the antibody. Examples of cells that may be used include those described in this section and in Section 6, infra. The cells may be primary cells or cell lines. In a particular aspect, provided herein are hybridoma cells expressing an antibody described herein, e.g., antibody AA12, EB9, GB5 or FC12. In a particular embodiment, the host cell is isolated from other cells. In another embodiment, the host cell is not found within the body of a subject.

Antibodies described herein (e.g., monoclonal antibodies, such as chimeric, human or humanized antibodies, or an antigen-binding fragment thereof) that bind to a Zika virus NS1 can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will bind to the protein (e.g., Zika virus NS1) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilpatrick et al., 1997 Hybridoma 16:381-9, incorporated by reference in its entirety).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific embodiments employ myeloma cells that fuse efficiently, support stable high-level production of antibody by the selected antibody-producing cells, and are sensitive to a medium such as HAT medium. Among these myeloma cell lines are murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against a Zika virus NS1. The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI 1640 medium. Alternatively, clonal cells can be isolated using a semi-solid agar supplemented with HAT (Stemcell Technologies). In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

In some embodiments, mice (or other animals, such as rats, monkeys, donkeys, pigs, sheep, goats, hamsters, or dogs) can be immunized with an antigen (e.g., a Zika virus NS1 or recombinant NS1 polypeptide, such as described herein) and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP2/0 available from the American Type Culture Collection (ATCC®) (Manassas, VA), to form hybridomas. Hybridomas are selected and cloned by limited dilution. In certain embodiments, lymph nodes of the immunized mice are harvested and fused with NS0 myeloma cells.

The hybridoma clones are then assayed by methods known in the art for cells that secrete antibodies capable of binding a polypeptide of the antigen (e.g., a Zika virus NS1 or a recombinant NS1 polypeptide, such as described herein). Ascites fluid, which generally contains high levels of antibodies, can be generated by immunizing mice with positive hybridoma clones.

Accordingly, described herein are methods of making antibodies described herein by culturing a hybridoma cell secreting an antibody. In certain embodiments, the method of making an antibody described herein further comprises the step of purifying the antibody.

In specific embodiments, the hybridoma is generated by fusing splenocytes isolated from a mouse (or other animal, such as rat, monkey, donkey, pig, sheep, or dog) immunized with a Zika virus NS1 with myeloma cells and then screening the hybridomas resulting from the fusion for hybridoma clones that secrete an antibody able to bind to the Zika virus NS1. In certain embodiments, the hybridoma is generated by fusing lymph nodes isolated from a mouse (or other animal, such as rat, monkey, donkey, pig, sheep, or dog) immunized with a Zika virus NS1 with myeloma cells, and then screening the hybridomas resulting from the fusion for hybridoma clones that secrete an antibody able to bind to the Zika virus NS1.

Antibodies described herein include antibody fragments that recognize a Zika virus NS1 and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CH1 domains of the heavy chain. A F(ab′)₂ fragment contains the two antigen-binding arms of an antibody molecule linked by disulfide bonds in the hinge region.

Further, the antibodies described herein can also be generated using various phage display methods known in the art. In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)₂ fragments can also be employed using methods known in the art such as those disclosed in PCT publication No. WO 92/22324; Mullinax et al., 1992, BioTechniques 12 (6): 864-869; Sawai et al., 1995, AJRI 34:26-34; and Better et al., 1988, Science 240:1041-1043.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it can be preferable to use human, humanized or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO 98/16654, WO 96/34096, WO 96/33735, and WO 91/10741.

A chimeric antibody is a molecule in which different portions of the antibody are derived from different immunoglobulin molecules. For example, a chimeric antibody can contain a variable region of a mouse monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

In some embodiments, humanized antibodies are produced. A humanized antibody is capable of binding to a predetermined antigen and comprises a framework region having substantially the amino acid sequence of a human immunoglobulin and CDRs having substantially the amino acid sequence of a non-human immunoglobulin (e.g., a murine immunoglobulin). Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28 (4/5): 489-498; Studnicka et al., 1994, Protein Engineering 7 (6): 805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13 (5): 353-60 (2000), Morea et al., Methods 20 (3): 267 79 (2000), Baca et al., J. Biol. Chem. 272 (16): 10678-84 (1997), Roguska et al., Protein Eng. 9 (10): 895 904 (1996), Couto et al., Cancer Res. 55 (23 Supp): 5973s-5977s (1995), Couto et al., Cancer Res. 55 (8): 1717-22 (1995), Sandhu J S, Gene 150 (2): 409-10 (1994), and Pedersen et al., J. Mol. Biol. 235 (3): 959-73 (1994). See also U.S. Patent Pub. No. US 2005/0042664 A1 (Feb. 24, 2005), which is incorporated by reference herein in its entirety.

In some embodiments, humanized antibodies are produced. In particular embodiments, an antibody described herein, which binds to the same epitope of a Zika virus NS1 as antibody AA12, EB9, GB5, or FC12, is a humanized antibody. In particular embodiments, an antibody described herein, which competitively blocks (e.g., in a dose-dependent manner) antibody AA12, EB9, GB5, or FC12 from binding to a Zika virus NS1, is a humanized antibody.

Human antibodies can be produced using any method known in the art. In certain embodiments, provided herein are human antibodies which can compete with antibody AA12, EB9, GB5, or FC12 for specific binding to a Zika virus NS1 or a recombinant NS1 polypeptide, such as described herein. In certain embodiments, provided herein are human antibodies which bind to the same epitope of a Zika virus NS1 or a recombinant NS1 polypeptide as the epitope to which antibody AA12, EB9, GB5, or FC12 binds. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., a Zika virus NS1). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, 1995, Int. Rev. Immunol. 13:65-93. For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598.

In some embodiments, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that bind to a target antigen (e.g., an Zika virus NS1). Such methods are known and are described in the art, see, e.g., Shinmoto et al., Cytotechnology, 2004, 46:19-23; Naganawa et al., Human Antibodies, 2005, 14:27-31.

In some embodiments, human antibodies can be generated by inserting polynucleotides encoding human CDRs (e.g., VL CDRs and/or VH CDRs) of an antibody into an expression vector containing nucleotide sequences encoding human framework region sequences. In certain embodiments, such expression vectors further comprise nucleotide sequences encoding a constant region of a human light and/or heavy chain. In some embodiments, human antibodies can be generated by inserting human CDRs (e.g., VL CDRs and/or VH CDRs) of an antibody obtained from a phage library into such human expression vectors.

In certain embodiments, a human antibody can be generated by selecting human CDR sequences that are homologous (or substantially homologous) to non-human CDR sequences of a non-human antibody and selecting human framework sequences that are homologous (or substantially homologous) to non-human framework sequences of a non-human antibody.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1 (3): 253-263; Muylderman, 2001, J. Biotechnol. 74 (4): 277302; U.S. Pat. No. 6,005,079; and International Publication Nos. WO 94/04678, WO 94/25591, and WO 01/44301.

Bispecific antibodies are antibodies that have binding specificities for at least two different epitopes. Exemplary bispecific antibodies may bind to two different epitopes of an antigen or to two different epitopes of two different antigens. In specific embodiments, a bispecific antibody has two distinct antigen-binding domains, wherein each domain specifically binds to a different antigen. Other such antibodies may bind a first antigen (e.g., a Zika virus NS1) and further bind a second antigen. Bispecific antibodies can be prepared as full-length antibodies or antibody fragments (e.g., F(ab′): bispecific antibodies).

Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148 (5): 1547-1553 (1992); Hollinger et al., Proc. Natl. Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731, 168; 4,676,980; and 4,676,980, WO 94/04690; WO 91/00360; WO 92/200373; WO 93/17715; WO 92/08802; and EP 03089.)

Further, antibodies that bind to a Zika virus NS1 can, in turn, be utilized to generate anti-idiotype antibodies that “mimic” an antigen using techniques well known to those skilled in the art. (See, e.g., Greenspan & Bona, 1989, FASEB J. 7 (5): 437-444; and Nissinoff, 1991, J. Immunol. 147 (8): 2429-2438).

Recombinant expression of an antibody described herein (e.g., a full-length antibody, heavy and/or light chain of an antibody, or a single chain antibody described herein) that binds to a Zika virus NS1 or a recombinant NS1 polypeptide, such as described herein, can for example, involve construction of vectors (e.g., expression vectors) containing a polynucleotide that encodes the antibody or fragments thereof (e.g., VL domain and/or VH domain). Once a polynucleotide encoding an antibody molecule, heavy and/or light chain of an antibody, or antigen-binding fragment thereof described herein has been obtained, a vector for the production of the antibody molecule can be produced by recombinant DNA technology using techniques well-known in the art. Methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and the variable domain of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein or a fragment thereof. Thus, provided herein are host cells containing a polynucleotide encoding an antibody described herein or fragments thereof, or a heavy or light chain thereof, or antigen-binding fragment thereof, or a single chain antibody described herein, operably linked to a promoter for expression of such sequences in the host cell. In certain embodiments, e.g., for the expression of double-chained antibodies, vectors encoding both the heavy and light chains individually can be co-expressed in the host cell for expression of the entire immunoglobulin molecule, as detailed below. In certain embodiments, a host cell contains a vector comprising a polynucleotide encoding both the heavy chain and light chain of an antibody described herein, or a fragment thereof. In specific embodiments, a host cell contains two different vectors, a first vector comprising a polynucleotide encoding a heavy chain of an antibody described herein, or a fragment thereof (e.g., a VH domain), and a second vector comprising a polynucleotide encoding a light chain of an antibody described herein, or a fragment thereof (e.g., a VL domain). In other embodiments, a first host cell comprises a first vector comprising a polynucleotide encoding a heavy chain of an antibody described herein, or a fragment thereof (e.g., a VH domain), and a second host cell comprises a second vector comprising a polynucleotide encoding a light chain of an antibody described herein, or a fragment thereof (e.g., a VL domain).

A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NS0, PER.C6, VERO, CRL7O3O, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, a mammalian expression vector is pOptiVEC™ or pcDNA3.3. Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In one embodiment, a mammalian cell (e.g., a human cell) is used to express an antibody described herein. In certain embodiments, antibodies described herein are produced by CHO cells or NS0 cells. In a specific embodiment, the expression of nucleotide sequences encoding antibodies described herein (or fragments thereof) which bind to a Zika B virus NS1 is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

As used herein, the term “host cell” refers to any type of cell, e.g., a primary cell or a cell from a cell line. In specific embodiments, the term “host cell” refers a cell transfected with a polynucleotide and the progeny or potential progeny of such a cell. Progeny of such a cell may not be identical to the parent cell transfected with the polynucleotide due to mutations or environmental influences that may occur in succeeding generations or integration of the polynucleotide into the host cell genome. In a specific embodiment, a host cell described herein is isolated.

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT2O and T47D, NS0 (a murine myeloma cell line that does not endogenously produce any immunoglobulin chains), CRL7O3O and HsS78Bst cells. In certain embodiments, humanized monoclonal antibodies described herein are produced in mammalian cells, such as CHO cells.

For long-term, high-yield production of recombinant proteins, stable expression is preferred. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compositions that interact directly or indirectly with the antibody molecule.

A number of selection systems can be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11 (5): 155-2 15); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. In a specific embodiment, a host cell comprises two expression vectors: one vector comprising a polynucleotide sequence comprising a nucleotide sequence encoding a heavy chain variable region of an antibody described herein (e.g., AA12, EB9, GB5, or FC12) and a second vector comprising a polynucleotide sequence comprising a nucleotide sequence encoding a light chain variable region of an antibody described herein (e.g., AA12, EB9, GB5, or FC12). The two vectors can contain identical selectable markers which enable equal expression of heavy and light chain polypeptides. The host cells can be co-transfected with different amounts of the two or more expression vectors.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody (e.g., a monoclonal antibody, such as a humanized, human or chimeric antibody or an antigen-binding fragment thereof) described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals that are involved in the synthesis of the protein. Accordingly, such preparations of the antibody have less than about 30%, 20%, 10%, 5% (by dry weight) of chemical precursors or compounds other than the antibody of interest. In a specific embodiment, antibodies described herein are isolated or purified.

In certain aspects, an antibody that binds to a Zika virus NS1, such as an antibody described herein, may be generated by immunization of a subject (e.g., a non-human subject) with an immunogen. In specific embodiments, a method for generating an antibody that binds to a Zika virus NS1, such as an antibody described herein, comprises administering to a subject (e.g., a non-human subject) one, two or more doses of one or more immunogens (e.g., a Zika virus NS1 known in the art or described herein, or a recombinant NS1 polypeptide known in the art or described herein). The spleen from the subject may be harvested, hybridomas produced and screened for antibodies that bind to one or more different Zika virus strains and/or NS1 of one or more different Zika virus strains. Techniques known to one of skill in the art or described herein may be used to harvest the spleen, produce hybridomas and screen for binding. The antibodies of interest may then be isolated.

In a specific embodiment, an antibody binds to a Zika virus NS1, such as an antibody described herein, may be generated by following the methodology described in Section 6, infra.

### 5.6 Expression of Zika NS1

Provided herein are vectors, including expression vectors, containing a polynucleotide encoding an NS1 polypeptide described herein. In a specific embodiment, the vector is an expression vector that is capable of directing the expression of a polynucleotide encoding an NS1 polypeptide described herein. Non-limiting examples of expression vectors include, but are not limited to, plasmids and viral vectors, such as a paramyxovirus (e.g., NDV), an adenovirus, an adeno-associated viruses, a baculovirus, a vaccinina virus, a retrovirus, a hepatitis virus, a poxvirus, a herpes virus, a rhabdovirus (e.g., vesticular stomatitis virus) or other virus known to one of skill in the art. Expression vectors also may include, without limitation, transgenic animals and non-mammalian cells/organisms, e.g., non-mammalian cells/organisms that have been engineered to perform mammalian N-linked glycosylation.

An expression vector comprises a polynucleotide encoding an NS1 polypeptide in a form suitable for expression of the nucleic acid in a host cell. In a specific embodiment, an expression vector includes one or more regulatory sequences, selected on the basis of the host cells to be used for expression, which is operably linked to the nucleic acid to be expressed. Within an expression vector, “operably linked” is intended to mean that a polynucleotide of interest is linked to the regulatory sequence(s) in a manner which allows for expression of the nucleic acid (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as the choice of the host cell to be transformed, the level of expression of protein desired, etc. In specific embodiments, the host cell is a cell line.

See Section 5.5, supra, for examples of expression vectors and host cells. In addition, a viral vector, virus-like particles, virosomes, bacterial vectors, and plant vectors may be used to express an NS1 polypeptide described herein, and/or may comprise such a polypeptide. See, e.g., Sections 5.8-5.12 of International Patent Application Publication No. WO 2016/118937, which is incorporated herein by reference in its entirety, for a discussion of such vectors, how to produce such vectors, and how to use such vectors. In a specific embodiment, a mammalian host cell (e.g., a human host cell), such as described in Section 5.5, is used to express an NS1 polypeptide described herein. In other embodiments, insech, bacterial or plant cells, such as described in Section 5.5, used to express an NS1 polypeptide described herein.

As an alternative to recombinant expression of an NS1 polypeptide described herein using a host cell, an expression vector containing a polynucleotide encoding an NS1 polypeptide can be transcribed and translated in vitro using, e.g., T7 promoter regulatory sequences and T7 polymerase. In a specific embodiment, a coupled transcription/translation system, such as Promega TNT®, or a cell lysate or cell extract comprising the components necessary for transcription and translation may be used to produce an NS1 polypeptide.

Once an NS1 polypeptide has been produced, it may be isolated or purified by any method known in the art for isolation or purification of a protein, for example, by chromatography (e.g., ion exchange, affinity, particularly by affinity for the specific antigen, by Protein A, and sizing column chromatography), centrifugation, differential solubility, or by any other standard technique for the isolation or purification of proteins.

Accordingly, provided herein are methods for producing an NS1 polypeptide described herein. In one embodiment, the method comprises culturing a host cell containing a nucleic acid sequence comprising a nucleotide sequence encoding the NS1 polypeptide in a suitable medium such that the polypeptide is produced. In some embodiments, the method further comprises isolating the polypeptide from the medium or the host cell.

In a specific embodiment, an NS1 polypeptide is produced using methods described in Section 6, infra.

### 5.7 Compositions

### 5.7.1 Antibody Compositions

Provided herein are compositions (e.g., pharmaceutical compositions) comprising an antibody having the desired degree of purity in a physiologically acceptable carrier, excipient or stabilizer (Remington's Pharmaceutical Sciences (1990) Mack Publishing Co., Easton, PA). In a specific embodiment, a composition comprises an antibody described herein and an acceptable carrier or excipient. In a specific embodiment, the compositions comprise an antibody conjugated to a moiety such as described in Section 5.2.2, supra. In certain embodiments, the compositions comprise an antibody that has been modified to increase its half-life, such as described in Section 5.2.1, supra. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In a specific embodiment, pharmaceutical compositions comprise an antibody, and optionally one or more additional prophylactic or therapeutic agents, in a pharmaceutically acceptable carrier. In a specific embodiment, pharmaceutical compositions comprise an effective amount of an antibody, and optionally one or more additional prophylactic of therapeutic agents, in a pharmaceutically acceptable carrier. See Section 5.8.3, infra, for examples of prophylactic or therapeutic agents. In some embodiments, the antibody is the only active ingredient included in the pharmaceutical composition. Pharmaceutical compositions described herein can be useful in the prevention of Zika virus disease, or treatment of a Zika virus infection or Zika virus disease. Further, pharmaceutical compositions described herein can be useful in the prevention, treatment and/or management of Zika virus disease.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition may be formulated for any route of administration to a subject. In certain embodiments, a pharmaceutical composition is formulated for systemic administration to a subject. Specific examples of routes of administration include intranasal, oral, transdermal, intradermal, parenteral, and mucosal. In a specific embodiment, the composition is formulated for intranasal or intramuscular administration. In a specific embodiment, the composition is formulation for intramuscular administration. In a specific embodiment, the composition is formulated for intranasal or mucosal administration. In a particular embodiment, the composition is formulated for subcutaneous or intravenous administration.

Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of an antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

In certain embodiments, a pharmaceutical composition comprising an antibody is a lyophilized powder, which can be reconstituted for administration as solutions, emulsions and other mixtures. It may also be reconstituted and formulated as solids or gels. The lyophilized powder is prepared by dissolving an antibody provided herein, or a pharmaceutically acceptable derivative thereof, in a suitable solvent. In some embodiments, the lyophilized powder is sterile. The solvent may contain an excipient that improves the stability or other pharmacological component of the powder or reconstituted solution, prepared from the powder. Excipients that may be used include, but are not limited to, dextrose, sorbitol, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

An antibody can also, for example, be formulated in liposomes. Liposomes containing the molecule of interest are prepared by methods known in the art, such as described in Epstein et al. (1985) Proc. Natl. Acad. Sci. USA 82:3688; Hwang et al. (1980) Proc. Natl. Acad. Sci. USA 77:4030; and U.S. Pat. Nos. 4,485,045 and 4,544,545. Liposomes with enhanced circulation time are disclosed in U.S. Pat. No. 5,013,556. In one embodiment, liposomal suspensions may also be suitable as pharmaceutically acceptable carriers. These can be prepared according to methods known to those skilled in the art. For example, liposome formulations can be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound comprising an antibody described herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

An antibody can also be entrapped in a microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1990) Mack Publishing Co., Easton, PA.

Sustained-release preparations can also be prepared. Suitable examples of sustained-release preparations include semipermeable matrices of solid hydrophobic polymers containing the antagonist, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The antibodies and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The compositions to be used for in vivo administration can be sterile. This is readily accomplished by filtration through, e.g., sterile filtration membranes.

In a specific embodiment, nucleic acids comprising sequences encoding an antibody described herein are administered to a subject by way of gene therapy. Gene therapy refers to therapy performed by the administration to a subject of an expressed or expressible nucleic acid. Encompassed herein are any of the methods for gene therapy available in the art. For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIBTECH 11 (5): 155-215. For a review of methods of delivery of transgenes encoding antibodies, see, e.g., Deal, 2015, Curr. Opin. Immunol. 2015 August, 35:113-22; Deal, 2015, Curr Opin HIV AIDS. 2015 May 10 (3): 190-7; Marschall, 2015, MAbs. 7 (6): 1010-35. In a specific embodiment, an mRNA encoding an antibody described herein is administered to a subject. Techniques known to one of skill in the art may be used to administer an mRNA encoding an antibody to a subject. For methods of delivery of mRNA encoding antibodies, see, e.g., U.S. Patent Application Publication No. US20130244282A1; U.S. Patent Application Publication No. US 2016/0158354A1; and International Patent Application No. WO2016014846A1, each of which is incorporated herein by reference in its entirety. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

### 5.7.2 NS1 Polypeptide Compositions

In another aspect, provided herein are compositions (e.g., pharmaceutical compositions, such as immunogenic compositions, e.g., vaccines) comprising an NS1 polypeptide described herein. In a specific embodiment, provided herein is a composition (e.g., pharmaceutical compositions, such as immunogenic compositions, e.g., vaccines) comprising an NS1 polypeptide described herein, and a pharmaceutically acceptable carrier. In certain embodiments, the pharmaceutical compositions (e.g., immunogenic compositions, such as e.g., vaccines) comprise one or more adjuvants.

In a specific embodiment, pharmaceutical compositions (e.g., immunogenic compositions, such as e.g., vaccines) are formulated to be suitable for the intended route of administration to a subject, including any route of administration described herein. For example, the pharmaceutical composition (e.g., immunogenic compositions, such as e.g., vaccines) may be formulated to be suitable for parenteral, intradermal, transdermal, and, intraperitoneal administration. In a specific embodiment, the pharmaceutical compositions (e.g., immunogenic compositions, such as e.g., vaccines) may be formulated for intravenous, intraperitoneal, intranasal, intratracheal, subcutaneous, intramuscular, topical, intradermal, transdermal or pulmonary administration.

In certain embodiments, immunogenic compositions described herein comprise a nucleic acid sequence comprising a nucleotide sequence (e.g., an RNA, an mRNA or cDNA) encoding an NS1 polypeptide. Such compositions may be formulated as a nanoparticle (e.g., a lipid nanoparticle) encapsulating or containing such a polynucleotide. See, e.g., Richner et al., 2017, Cell 168:1114 and Richner et al., 2017, Cell 170 (2): 273 for examples of such formulations for mRNA delivery.

In specific embodiments, immunogenic compositions described herein are monovalent formulations. In other embodiments, immunogenic compositions described herein are multivalent formulations.

In some embodiments, provided herein are immunogenic compositions comprising a live virus containing an NS1 polypeptide described herein. In certain embodiments, provided herein are immunogenic compositions comprising a live virus encoding an NS1 described herein. In some embodiments, provided herein are immunogenic compositions comprising a live virus encoding and containing an NS1 polypeptide described herein. The live virus may be a paramyxovirus (e.g., NDV), an adenovirus, an AAV, vaccinina virus, retrovirus, hepatitis virus, poxvirus, herpes virus, rhabdovirus (e.g., VSV) or other virus known to one of skill in the art or described in, e.g., Section 5.9 of International Patent Application Publication No. WO 2016/118937, which is incorporated herein by reference in its entirety.

In some embodiments, provided herein are immunogenic compositions comprising an inactivated virus containing an NS1 polypeptide described herein. Such an immunogenic composition may comprise an adjuvant. See, e.g., Section 5.15.3 of International Patent Application Publication No. WO 2016/118937, which is incorporated herein by reference in its entirety, for a discussion of types of inactivated viruses and compositions comprising them.

In certain embodiments, an immunogenic composition comprising an NS1 polypeptide is split vaccine. The split vaccine may comprise an adjuvant. See, e.g., Section 5.15.4 of International Patent Application Publication No. WO 2016/118937, which is incorporated herein by reference in its entirety, for a discussion of examples of subunit vaccines.

In certain embodiments, provided herein are subunit vaccines comprising an NS1 polypeptide described herein. The subunit vaccines may comprise one or more adjuvants. See, e.g., Section 5.15.1 of International Patent Application Publication No. WO 2016/118937, which is incorporated herein by reference in its entirety, for a discussion of examples of subunit vaccines.

In certain embodiments, the compositions described herein comprise, or are administered in combination with, an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to an NS1 polypeptide, but when the compound is administered alone does not generate an immune response to the polypeptide. In some embodiments, the adjuvant generates an immune response to the polypeptide and does not produce an allergy or other adverse reaction. Adjuvants can enhance an immune response by several mechanisms including, e.g., lymphocyte recruitment, stimulation of B and/or T cells, and stimulation of macrophages.

In certain embodiments, an adjuvant augments the intrinsic response to the NS1 polypeptide without causing conformational changes in the polypeptide that affect the qualitative form of the response. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS01 (GlaxoSmithKline), AS03 (GlaxoSmithKline), AS04 (GlaxoSmithKline), AddaVax, imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as polyglutamic acid or polylysine, or other immunopotentiating agents. In a specific embodiment, the adjuvant is one described in Section 6, infra.

In a specific embodiment, an NS1 polypeptide described herein is encapsulated in a liposome or nanoparticle described herein or known in the art. For examples of liposomes, see Section 5.7.1, supra. In certain embodiments, a pharmaceutical composition (e.g., an immunogenic composition) comprises an NS1 polypeptide described herein encapsulated by a liposome or nanoparticle. In another specific embodiment, an NS1 polypeptide described herein may be included in a sustained-release preparation. See Section 5.7.1, supra, for examples of sustained release preparations. In certain embodiments, a pharmaceutical composition (e.g., an immunogenic composition) comprises a sustained-release preparation of an NS1 polypeptide described herein.

### 5.8 Prophylactic and Therapeutic Uses

### 5.8.1 Active Immunization

In another aspect, provided herein are methods for inducing an immune response in a subject utilizing an NS1 polypeptide described herein, a polynucleotide sequence comprising a nucleic acid sequence encoding such a polypeptide(s), a vector (e.g., a viral vector, or a bacteria) containing, expressing or both containing and expressing such a polypeptide(s), or a composition described herein. In a specific embodiment, a method for inducing an immune response to a Zika virus NS1 in a subject comprises administering to a subject in need thereof an effective amount of an NS1 polypeptide described herein, or an immunogenic composition thereof. In another embodiment, a method for inducing an immune response to a Zika virus NS1 in a subject comprises administering to a subject in need thereof an effective amount of a polynucleotide (e.g., mRNA or DNA) comprising a nucleic acid sequence encoding an NS1 polypeptide described herein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168:1114 for examples of such formulations for mRNA delivery. In another embodiment, a method for inducing an immune response to a Zika virus NS1 in a subject comprises administering to a subject in need thereof an effective amount of a viral vector containing, encoding, or containing and encoding a Zika virus NS1 polypeptide described herein, or an immunogenic composition thereof. The methods described in this paragraph may further comprise administering another therapy (e.g., a therapy described in Section 5.8.3, infra). The other therapy may be administered prior to, concurrently, or after the administration of a NS1 polypeptide described herein, a polynucleotide comprising a nucleic acid sequence (e.g., an RNA sequence, such as a self-replicating RNA) encoding such a polypeptide(s), a vector (e.g., a viral vector, or a bacteria) containing, expressing or both containing and expressing such a polypeptide(s), or a composition described herein.

In another aspect, provided herein are methods for immunizing against Zika virus in a subject utilizing an NS1 polypeptide described herein, a polynucleotide sequence comprising a nucleic acid sequence encoding such a polypeptide(s), a vector (e.g., a viral vector, or a bacteria) containing or expressing such a polypeptide(s), or a composition described herein. In a specific embodiment, a method for immunizing against Zika virus comprises administering to a subject in need thereof an effective amount of an NS1 polypeptide described herein, or an immunogenic composition thereof. In another embodiment, a method for immunizing against Zika virus comprises administering to a subject in need thereof an effective amount of a polynucleotide (e.g., mRNA or DNA) comprising a nucleic acid sequence encoding an NS1 polypeptide described herein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168:1114 for examples of such formulations for mRNA delivery. In another embodiment, a method for immunizing against Zika virus comprises administering to a subject in need thereof an effective amount of a viral vector containing, encoding, or containing and encoding a Zika virus NS1 polypeptide described herein, or an immunogenic composition thereof. The methods described in this paragraph may further comprise administering another therapy (e.g., a therapy described in Section 5.8.3, infra). The other therapy may be administered prior to, concurrently with, or after the administration of a NS1 polypeptide described herein, a polynucleotide comprising a nucleic acid sequence encoding such a polypeptide(s), a vector (e.g., a viral vector, or a bacteria) containing, expressing or both containing and expressing such a polypeptide(s), or a composition described herein.

In another embodiment, provided herein are immunization regimens involving a first immunization (e.g., priming) with an immunogenic composition (e.g., a vaccine) described herein followed by one, two, or more additional immunizations (e.g., boostings) with an immunogenic composition (e.g., a vaccine). In a specific embodiment, the immunogenic composition (e.g., a vaccine) used in the first immunization is the same type of an immunogenic composition (e.g., a vaccine) used in one, two or more additional immunizations. For example, if the immunogenic composition (e.g., vaccine) used in the first immunization is NS1 polypeptide vaccine formulation, the immunogenic composition (e.g., vaccine) used for the one, two or more additional immunizations may be the same type of vaccine formulation, i.e., NS1 polypeptide vaccine formulation. In other specific embodiments, the immunogenic composition (e.g., vaccine) used in the first immunization is different from the type of immunogenic composition (e.g., vaccine) used in one, two or more additional immunizations. For example, if the immunogenic composition (e.g., vaccine) used in the first immunization is nucleic acid-based vaccine formulation, the immunogenic composition (e.g., vaccine) used for the one, two or more additional immunizations may be an NS1 polypeptide vaccine formulation. In a specific embodiment, an immunization regimen such as described in Section 6, infra, is used to immunize a subject against Zika virus.

In another aspect, provided herein are methods for preventing Zika virus disease in a subject utilizing an NS1 polypeptide described herein, a polynucleotide sequence comprising a nucleic acid sequence encoding such a polypeptide(s), a vector (e.g., a viral vector, or a bacteria) containing, expressing or both containing and expressing such a polypeptide(s), or a composition described herein. In a specific embodiment, a method for preventing Zika virus disease comprises administering to a subject in need thereof an effective amount of an NS1 polypeptide described herein, or an immunogenic composition thereof. In another embodiment, a method for preventing Zika virus disease comprises administering to a subject in need thereof an effective amount of a polynucleotide (e.g., mRNA or DNA) comprising a nucleic acid sequence encoding an NS1 polypeptide described herein, or an immunogenic composition thereof. The polynucleotide may be administered using a gene therapy technique known to one of skill in the art or described herein. In a specific embodiment, the polynucleotide may be administered, e.g., as an mRNA using techniques known to one of skill in the art, including, as described in, e.g., U.S. Patent Application Publication No. 2016/0158354 and Richner et al., 2017, Cell 168:1114 for examples of such formulations for mRNA delivery. In another embodiment, a method for preventing Zika virus disease comprises administering to a subject in need thereof an effective amount of a viral vector containing, encoding, or containing and encoding a Zika virus NS1 polypeptide described herein, or an immunogenic composition thereof. The methods described in this paragraph may further comprise administering another therapy (e.g., a therapy described in Section 5.8.3, infra). The other therapy may be administered prior to, concurrently with, or after the administration of an NS1 polypeptide described herein, a polynucleotide comprising a nucleic acid sequence (RNA sequence, such as an RNA replicon) encoding such a polypeptide(s), a vector (e.g., a viral vector, or a bacteria) containing, expressing or both containing and expressing such a polypeptide(s), or a composition described herein.

The amount of an NS1 polypeptide described herein, a polynucleotide comprising a nucleic acid sequence encoding such a polypeptide(s), a vector (e.g., a viral vector, or a bacteria) containing, expressing or both containing and expressing such a polypeptide(s), or a composition described herein which will be effective in the prevention of a Zika virus disease will depend on the nature of the disease, and can be determined by standard clinical techniques.

The precise dose to be employed in the formulation will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but non-human mammals including transgenic mammals can also be treated. Treatment dosages are optimally titrated to optimize safety and efficacy.

As used herein, the term “effective amount” in the context of administering a therapy to a subject refers to the amount of a therapy which may have a prophylactic effect(s), therapeutic effect(s), or both a prophylactic and therapeutic effect(s). In certain embodiments, an “effective amount” in the context of administration of a therapy to a subject refers to the amount of a therapy which is sufficient to achieve one, two, three, four, or more of the following effects: (i) reduce or ameliorate the severity of a Zika virus infection, disease or symptom associated therewith; (ii) reduce the duration of a Zika virus infection, disease or symptom associated therewith; (iii) prevent the progression of a Zika virus infection, disease or symptom associated therewith; (iv) cause regression of a Zika virus infection, disease or symptom associated therewith; (v) prevent the development or onset of a Zika virus infection, disease or symptom associated therewith; (vi) prevent the recurrence of a Zika virus infection, disease or symptom associated therewith; (vii) reduce or prevent the spread of a Zika virus from one cell to another cell, one tissue to another tissue, or one organ to another organ; (viii) prevent or reduce the spread of a Zika virus from one subject to another subject; (ix) reduce organ failure associated with a Zika virus infection; (x) reduce hospitalization of a subject; (xi) reduce hospitalization length; (xii) increase the survival of a subject with a Zika virus infection or disease associated therewith; (xiii) eliminate a Zika virus infection or disease associated therewith; (xiv) inhibit or reduce Zika virus replication; (xv) inhibit or reduce the entry of a Zika virus into a host cell(s); (xvi) inhibit or reduce replication of the Zika virus genome; (xvii) inhibit or reduce synthesis of Zika virus proteins; (xviii) inhibit or reduce assembly of Zika virus particles; (xix) inhibit or reduce release of Zika virus particles from a host cell(s); (xx) reduce Zika virus titer; and/or (xxi) enhance or improve the prophylactic or therapeutic effect(s) of another therapy. In a specific embodiment, the effective amount reduces the neurological symptoms associated with a Zika virus infection. In another specific embodiment, the effective amount reduces lethality associated with a Zika virus infection. In another specific embodiment, the effective amount limits disease associated or caused by a Zika virus infection. In another specific embodiment, the effective amount prevents Gullian-Barré syndrome. In another specific embodiment, the effective amount prevents microcephaly in an infant born from an pregnant woman infected with a Zika virus.

In certain embodiments, the effective amount does not result in complete protection from a Zika virus disease, but results in a lower titer or reduced number of Zika viruses compared to an untreated subject with a Zika virus infection. In certain embodiments, the effective amount results in a 0.5 fold, 1 fold, 1.5 fold, 2 fold, 3 fold, 4 fold, 5 fold, 6 fold, 7 fold, 8 fold, 9 fold, 10 fold, 15 fold, 20 fold, 25 fold, 50 fold, 75 fold, 100 fold, 125 fold, 150 fold, 175 fold, 200 fold, 300 fold, 400 fold, 500 fold, 750 fold, or 1,000 fold or greater reduction in titer of a Zika virus relative to an untreated subject with a Zika virus infection. In some embodiments, the effective amount results in a reduction in titer of Zika virus relative to an untreated subject with a Zika virus infection of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 3 logs, 1 to 5 logs, 1 to 8 logs, 1 to 9 logs, 2 to 10 logs, 2 to 5 logs, 2 to 7 logs, 2 logs to 8 logs, 2 to 9 logs, 2 to 10 logs 3 to 5 logs, 3 to 7 logs, 3 to 8 logs, 3 to 9 logs, 4 to 6 logs, 4 to 8 logs, 4 to 9 logs, 5 to 6 logs, 5 to 7 logs, 5 to 8 logs, 5 to 9 logs, 6 to 7 logs, 6 to 8 logs, 6 to 9 logs, 7 to 8 logs, 7 to 9 logs, or 8 to 9 logs. Benefits of a reduction in the titer, number or total burden of Zika virus include, but are not limited to, less severe symptoms of the infection, fewer symptoms of the infection and a reduction in the length of the disease associated with the infection.

In certain embodiments, an effective amount of a therapy (e.g., a composition thereof) results in an anti-Zika virus NS1 titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-Zika virus NS1 titer in a blood sample from the subject prior to immunization. In certain embodiments, an effective amount of a therapy (e.g., a composition thereof) results in an anti-Zika virus NS1 stalk titer in a blood sample from a subject administered the effective amount 0.5 fold to 10 fold, 0.5 fold to 4 fold, 0.5 fold to 3 fold, 0.5 fold to 2 fold, 0.5 fold, 1 fold, 1.5 fold, 2 fold, 3 fold, 4 fold, 5 fold, 6 fold, 7 fold, 8 fold, 9 fold, 10 fold higher post-immunization relative to the anti-Zika virus NS1 stalk titer in a blood sample from the subject prior to immunization.

In certain embodiments, a subject is administered a dose of 0.1-100 mg/kg (e.g., 1-15 mg/kg or 10-15 mg/kg) of an NS1 polypeptide described herein. In some embodiments, a subject is administered dose of 1-100 μg (e.g., 25 μg, 40 μg, 50 μg or 75 μg) of a polynucleotide encoding an NS1 polypeptide described herein or an expression vector comprising such a polynucleotide. In certain embodiments, a subject is administered a viral vector at a dose of 10⁴, 5×10⁴, 105, 5×10⁵, 10⁶, 5×10⁶, or 10⁷ pfu.

An NS1 polypeptide described herein, a polynucleotide encoding such a polypeptide(s), a vector (e.g., a viral vector, or a bacteria) containing, expressing, or both containing and expressing such a polypeptide(s), or a composition described herein may be administered by any route known to one of skill in the art or described herein. For example, it may be administered parenterally (e.g., intravenously, intramuscularly, subcutaneous, etc.), intranasally, transdermally, etc.

The subject that may be administered an NS1 polypeptide described herein, a polynucleotide comprising a nucleic acid sequence encoding such a polypeptide(s), a vector (e.g., a viral vector, or a bacteria) containing, expressing, or both containing and expressing such a polypeptide(s), or a composition described herein includes those subjects described in herein (e.g., in Section 5.8.3, infra). In certain embodiments, the subject is a non-human animal, such as, e.g., a pet or farm animal (e.g., a cow, pig, bird, horse or dog). In a specific embodiment, the subject is a human. In another embodiment, the subject is a human infant, human toddler, human adult, or elderly human. In certain embodiments, an NS1 polypeptide described herein, a polynucleotide comprising a nucleic acid sequence encoding such a polypeptide(s), a vector (e.g., a viral vector, or a bacteria) containing, expressing, or both containing and expressing such a polypeptide(s), or a composition described herein may not be administered to a subject that is immunocompetent or immunocomprised. In some embodiments, an NS1 polypeptide described herein, a polynucleotide comprising a nucleic acid sequence encoding such a polypeptide(s), a vector (e.g., a viral vector, or a bacteria) containing, expressing, or both containing and expressing such a polypeptide(s), or a composition described herein may not be administered to a subject with an infection (e.g., a bacterial, fungal or viral infection) or a disease caused by an infection (e.g., a bacterial, fungal or viral infection), including, e.g., acute and chronic disease caused by an infection (e.g., a bacterial, fungal or viral infection). In a specific embodiment, an NS1 polypeptide described herein, a polynucleotide comprising a nucleic acid sequence encoding such a polypeptide(s), a vector (e.g., a viral vector, or a bacteria) containing, expressing, or both containing and expressing such a polypeptide(s), or a composition described herein may not be administered to a pregnant woman.

In a specific embodiment, an NS1 polypeptide described herein is used to generate antibodies using techniques known to one of skill in the art or described herein.

### 5.8.2 Passive Immunization

In one aspect, provided herein are methods for preventing Zika virus disease comprising administering an antibody described herein. In a specific embodiment, provided herein is a method for preventing Zika virus disease in a subject comprising administering to the subject an effective amount of an antibody described herein. In a specific embodiment, provided herein is a method for preventing Zika virus disease in a subject comprising administering to the subject a pharmaceutical composition comprising an effective amount of an antibody described herein. In a specific embodiment, the antibody is a protein or a protein conjugate. In a specific embodiment, the antibody is a polynucleotide sequence encoding an antibody. In a specific embodiment, provided herein is a method for preventing Zika virus disease in a subject comprising administering to the subject an effective amount of an antibody described herein and another therapy, such as known to one of skill in the art or described herein (e.g., in Section 5.8.3, infra). In a specific embodiment, provided herein is a method for preventing Zika virus disease in a subject comprising administering to the subject a pharmaceutical composition comprising an effective amount of an antibody described herein, and another therapy, such as known to one of skill in the art or described herein (e.g., in Section 5.8.3, infra). In a particular embodiment, the administration of an effective amount of the antibody to the subject inhibits or reduces in the development or onset of a Zika virus disease. In another embodiment, the administration of an effective amount of the antibody to the subject inhibits or reduces onset, development and/or severity of a symptom thereof (e.g., fever, muscle pain, joint pain, inflammation, rash, headache, etc.) of Zika virus disease. In another embodiment, the administration of an effective amount of the antibody inhibits or reduces in the recurrence of a Zika virus disease or a symptom associated therewith. In a specific embodiment, the administration of an effective amount of the antibody reduces the neurological symptoms associated with a Zika virus infection. In another specific embodiment, the administration of an effective amount of the antibody reduces lethality associated with a Zika virus infection. In another specific embodiment, the administration of an effective amount of the antibody limits disease associated or caused by a Zika virus infection. In another specific embodiment, the administration of an effective amount of the antibody prevents Gullian-Barre syndrome. In another specific embodiment, the administration of an effective amount of the antibody prevents microcephaly in an infant born from an pregnant woman infected with a Zika virus. The antibody administered to the pregnant woman may be transferred to the fetus.

In a specific embodiment, provided herein is a method for preventing Zika virus disease in a subject comprising: (a) administering to the subject a dose of an NS1 polypeptide described herein and (b) after a certain period of time administering a dose of an antibody described herein. In some embodiments, the certain period of time is 2 to 3 weeks, 1 month, 2 months, 3 months, 4 months, 6 months, 7 months, 8 months, 9 months or 12 months after step (a). In another embodiment, provided herein is a method for preventing Zika virus disease in a subject comprising administering an antibody described herein to the subject, wherein the subject has been administered an NS1 polypeptide described herein a certain period of time before the administration of the antibody. In some embodiments, the certain period of time is 2 to 3 weeks, 1 month, 2 months, 3 months, 4 months, 6 months, 7 months, 8 months, 9 months or 12 months before the administration of the antibody.

In specific embodiments, the administration of an effective amount of an antibody to a subject results in one, two, three, four, or more of the following: (i) the reduction or inhibition of the spread of Zika virus from one cell to another cell; (ii) the reduction or inhibition of the spread of Zika virus from one organ or tissue to another organ or tissue; (iii) the reduction or inhibition of the spread of Zika virus from one region of an organ or tissue to another region of the organ or tissue (e.g., the reduction in the spread of Zika virus from the upper to lower respiratory tract); (iv) the prevention of Zika virus disease after exposure to a Zika virus; (v) the reduction or inhibition in Zika virus infection and/or replication; and/or (vi) prevention of the onset or development of one or more symptoms associated with Zika virus disease or infection.

In another aspect, provided herein are methods for treating a Zika virus infection or a Zika virus disease comprising administering an antibody described herein. In a specific embodiment, provided herein is a method for treating a Zika virus infection or a Zika virus disease in a subject comprising administering to the subject an effective amount of an antibody described herein. In another specific embodiment, provided herein is a method for treating a Zika virus infection or a Zika virus disease in a subject comprising administering to the subject a pharmaceutical composition comprising an effective amount of an antibody described herein. In another specific embodiment, provided herein is a method for treating a Zika virus infection or a Zika virus disease comprising administering to the subject an effective amount of an antibody described herein and another therapy, such as known to one of skill in the art or described herein (e.g., in Section 5.8.3, infra). In another specific embodiment, provided herein is a method for treating a Zika virus infection or a Zika virus disease in a subject comprising administering to the subject a pharmaceutical composition comprising an effective amount of an antibody described herein, and another therapy, such as known to one of skill in the art or described herein (e.g., in Section 5.8.3, infra). In a particular embodiment, the administration of an effective amount of the antibody to the subject inhibits or reduces in the development of a Zika virus disease. In another embodiment, the administration of an effective amount of the antibody to the subject inhibits or reduces onset, development and/or severity of a symptom thereof (e.g., fever, muscle pain, joint pain, inflammation, rash, headache, etc.) of Zika virus disease. In another embodiment, the administration of an effective amount of the antibody inhibits or reduces duration of a Zika virus disease or a symptom associated therewith. In another embodiment, the administration of an effective amount of the antibody reduces organ failure associated with a Zika virus infection or Zika virus disease. In another embodiment, the administration of an effective amount of the antibody reduces the hospitalization of the subject. In another embodiment, the administration of an effective amount of the antibody reduces the length of hospitalization of the subject. In another embodiment, the administration of an effective amount of the antibody increases the overall survival of subjects with a Zika virus infection or a disease associated therewith. In another embodiment, the administration of an effective amount of the antibody prevents the onset or progression of a secondary infection associated with a Zika virus infection.

In a specific embodiment, administration of an antibody(ies) to a subject reduces the incidence of hospitalization by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the incidence of hospitalization in the absence of administration of said antibody(ies).

In a specific embodiment, administration of an antibody(ies) to a subject reduces mortality by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the mortality in the absence of administration of said antibody(ies).

In certain embodiments, the administration of an effective amount of an antibody described herein to a subject results in one, two, three, four, five, or more of the following effects: (i) reduction or amelioration in the severity of a Zika virus infection, a Zika virus disease or a symptom associated therewith; (ii) reduction in the duration of a Zika virus infection, a Zika virus disease or a symptom associated therewith; (iii) prevention of the progression of a Zika virus infection, a Zika virus disease or a symptom associated therewith; (iv) regression of a Zika virus infection, a Zika virus disease or a symptom associated therewith; (v) prevention of the development or onset of a Zika virus infection, a Zika virus disease or a symptom associated therewith; (vi) prevention of the recurrence of a Zika virus infection, a Zika virus disease or a symptom associated therewith; (vii) reduction or prevention of the spread of a Zika virus from one cell to another cell, one tissue to another tissue, or one organ to another organ; (viii) prevention or reduction of the spread/transmission of a Zika virus from one subject to another subject; (ix) reduction in organ failure associated with a Zika infection or a Zika virus disease; (x) reduction in the hospitalization of a subject; (xi) reduction in the hospitalization length; (xii) an increase in the survival of a subject with a Zika infection or a disease associated therewith; (xiii) elimination of a Zika virus infection or a disease associated therewith; (xiv) inhibition or reduction a Zika virus replication; (xv) inhibition or reduction in the binding or fusion of a Zika virus to a host cell(s); (xvi) inhibition or reduction in the entry of a Zika virus into a host cell(s); (xvii) inhibition or reduction of replication of the Zika virus genome; (xviii) inhibition or reduction in the synthesis of Zika virus proteins; (xix) inhibition or reduction in the assembly of Zika virus particles; (xx) inhibition or reduction in the release of Zika virus particles from a host cell(s); (xxi) reduction in Zika virus titer; (xxii) the reduction in the number of symptoms associated with a Zika virus infection or a Zika virus disease; (xxiii) enhancement, improvement, supplementation, complementation, or augmentation of the prophylactic or therapeutic effect(s) of another therapy; and/or (xxiv) prevention of the onset or progression of a secondary infection associated with a Zika virus infection.

In a specific embodiment, administration of an antibody(ies) results in reduction of about 1-fold, about 1.5-fold, about 2-fold, about 3-fold, about 4-fold, about 5-fold, about 8-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105 fold, about 110-fold, about 115-fold, about 120 fold, about 125-fold or higher in Zika virus titer in the subject. The fold-reduction in Zika virus titer may be as compared to a negative control, as compared to another treatment, or as compared to the titer in the patient prior to antibody administration.

In a specific embodiment, administration of an antibody(ies) results in a reduction of approximately 1 log or more, approximately 2 logs or more, approximately 3 logs or more, approximately 4 logs or more, approximately 5 logs or more, approximately 6 logs or more, approximately 7 logs or more, approximately 8 logs or more, approximately 9 logs or more, approximately 10 logs or more, 1 to 5 logs, 2 to 10 logs, 2 to 5 logs, or 2 to 10 logs in Zika virus titer in the subject. The log-reduction in Zika virus titer may be as compared to a negative control (e.g., PBS or a negative control antibody), as compared to another treatment, or as compared to the titer in the patient prior to antibody administration.

In a specific embodiment, administration of an antibody(ies) inhibits or reduces Zika virus infection of a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to Zika virus infection of a subject in the absence of said antibody(ies) or in the presence of a negative control (e.g., PBS or a negative control antibody) in an assay known to one of skill in the art or described herein.

In a specific embodiment, administration of an antibody(ies) inhibits or reduces the spread of Zika virus in a subject by at least 99%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10% relative to the spread of Zika virus in a subject in the absence of said an antibody(ies) or in the presence of a negative control (e.g., PBS or a negative control antibody) in an assay known to one of skill in the art or described herein.

In a specific embodiment, administration of an antibody(ies) to a subject reduces the number of and/or the frequency of symptoms of Zika virus disease or infection in the subject (exemplary symptoms of Zika virus disease include, but are not limited to, body aches (especially joints and throat), fever, nausea, headaches, irritated eyes, fatigue, sore throat, reddened eyes or skin, and abdominal pain).

An antibody(ies) may be administered alone or in combination with another/other type of therapy known in the art to reduce Zika virus infection, to reduce titers of Zika virus in a subject, and/or to reduce the spread of Zika virus between subjects.

One or more of the antibodies described herein may be used locally or systemically in the body as a prophylactic or therapeutic agent.

### 5.8.2.1 Routes of Administration and Dosages

An antibody (e.g., a monoclonal antibody or an antigen-binding fragment thereof) or composition described herein may be delivered to a subject by a variety of routes, including any route described herein. These include, but are not limited to, intratracheal, oral, intradermal, intramuscular, intraperitoneal, transdermal, intravenous, and subcutaneous routes. In a specific embodiment, an antibody described herein is administered to a subject subcutaneously, intravenously, intranasally, or intramuscularly.

The amount of an antibody (e.g., a monoclonal antibody or an antigen-binding fragment thereof) or composition which will be effective in the treatment and/or prevention of a Zika virus infection or a Zika virus disease will depend on the nature of the disease and can be determined by standard clinical techniques.

The precise dose to be employed in a composition will also depend on the route of administration, and the seriousness of the infection or disease caused by it, and should be decided according to the judgment of the practitioner and each subject's circumstances. For example, effective doses may also vary depending upon means of administration, target site, physiological state of the patient (including age, body weight, health), whether the patient is human or an animal, other medications administered, and whether treatment is prophylactic or therapeutic. Usually, the patient is a human but non-human mammals including transgenic mammals can also be treated. Treatment dosages are optimally titrated to optimize safety and efficacy.

In certain embodiments, an in vitro assay is employed to help identify optimal dosage ranges. Effective doses may be extrapolated from dose response curves derived from in vitro or animal model test systems.

For passive immunization with an (e.g., a monoclonal antibody or an antigen-binding fragment thereof), the dosage ranges from about 0.0001 to 100 mg/kg, and more usually 0.01 to 5 mg/kg, of the patient body weight. For example, dosages can be 1 mg/kg body weight, 10 mg/kg body weight, or within the range of 1-10 mg/kg or in other words, 70 mg or 700 mg or within the range of 70-700 mg, respectively, for a 70 kg patient. In some embodiments, the dosage administered to the patient is about 3 mg/kg to about 60 mg/kg of the patient's body weight. Preferably, the dosage administered to a patient is between 0.025 mg/kg and 20 mg/kg of the patient's body weight, more preferably 1 mg/kg to 15 mg/kg of the patient's body weight. In a specific embodiment, a dose of an antibody administered to a subject is a dose described in Section 6, infra. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies described herein (e.g., a monoclonal antibody or an antigen-binding fragment thereof) may be reduced by enhancing uptake and tissue penetration of the antibodies by modifications such as, for example, lipidation.

An exemplary treatment regime entails administration once per every two weeks or once a month or once every 3 to 6 months for a period of one year or over several years, or over several year-intervals. In some methods, two or more antibodies with different binding specificities are administered simultaneously to a subject. An antibody is usually administered on multiple occasions. Intervals between single dosages can be weekly, monthly, every 3 months, every 6 months or yearly. Intervals can also be irregular as indicated by measuring blood levels of antibody to the Zika virus NS1 in the patient.

In some embodiments, the plasma or serum level of an antibody described herein in a patient is measured prior to administration of a subsequent dose of an antibody described herein, or a composition thereof. The plasma or serum level of the antibody may be considered in determining the eligibility of a patient to receive a subsequent dose of an antibody described herein. For example, a patient's plasma or serum level of an antibody described herein may suggest not administering an antibody described herein; alternatively, a patient's plasma level of an antibody described herein may suggest administering an antibody described herein at a particular dosage, at a particular frequency, and/or for a certain period of time.

In certain embodiments, the route of administration for a dose of an antibody described herein, or a composition thereof to a patient is intramuscular, subcutaneous intravenous, or a combination thereof, but other routes described herein are also acceptable. Each dose may or may not be administered by an identical route of administration. In some embodiments, an antibody described herein, or composition thereof, may be administered via multiple routes of administration simultaneously or subsequently to other doses of the same or a different antibody described herein.

### 5.8.3 Combination Therapies

In various embodiments, an antibody described herein or a nucleic acid encoding such an antibody may be administered to a subject in combination with one or more other therapies (e.g., antiviral or immunomodulatory therapies). In certain embodiments, a NS1 polypeptide described herein or a nucleic acid encoding such a polypeptide may be administered to a subject in combination with one or more other therapies (e.g., antiviral or immunomodulatory therapies). In some embodiments, a pharmaceutical composition (e.g., an immunogenic composition) described herein may be administered to a subject in combination with one or more therapies. The one or more other therapies may be beneficial in the treatment or prevention of an Zika virus disease or may ameliorate a condition associated with a Zika virus disease. The one or more other therapies may be beneficial in the treatment or prevention of a Zika virus infection or a disease associated therewith.

In some embodiments, the one or more other therapies that are supportive measures, such as pain relievers, anti-fever medications, or therapies that alleviate or assist with breathing. Specific examples of supportive measures include humidification of the air by an ultrasonic nebulizer, aerolized racemic epinephrine, an anti-inflammatory, oral dexamethasone, intravenous fluids, intubation, fever reducers (e.g., ibuprofen, acetometaphin), and antibiotic and/or antifungal therapy (i.e., to prevent or treat secondary bacterial and/or fungal infections).

In some embodiments, an antibody described herein or a nucleic acid sequence encoding an antibody described herein, or a composition thereof is administered to a subject with an antibody that binds to a Zika virus envelope protein. In other embodiments, an antibody described herein or a nucleic acid sequence encoding an antibody described herein, or a composition thereof is not administered to a subject with an antibody that binds to a Zika virus envelope protein.

In certain embodiments, the therapies are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In specific embodiments, two or more therapies are administered within the same patent visit. In some embodiments, two or more therapies are administered concurrently. The two or more therapies can be administered in the same composition or a different composition. Further, the two or more therapies can be administered by the same route of administration of a different route of administration.

In a specific embodiment, an antibody described herein is administered to a subject in combination with an antibody that binds to a Zika virus envelope protein.

In some embodiments, a combination therapy comprises the administration of one or more antibodies described herein. In some embodiments, a combination therapy comprises administration of two or more antibodies described herein. In a specific embodiment, a combination therapy comprises the administration of the AA12, EB9, GB5, or FC12 antibody and one or more other therapies.

### 5.8.4 Patient Populations

As used herein, the terms “subject” and “patient” are used interchangeably to refer to an animal (e.g., birds, reptiles, and mammals).

In one embodiment, a patient treated in accordance with the methods provided herein is a naïve subject, i.e., a subject that does not have a disease caused by Zika virus infection or has not been and is not currently infected with a Zika virus infection. In another embodiment, a patient treated in accordance with the methods provided herein is a subject that is at risk of acquiring a Zika virus infection. In another embodiment, a patient treated in accordance with the methods provided herein is a naïve subject that is at risk of acquiring a Zika virus infection. In another embodiment, a patient treated in accordance with the methods provided herein is a patient suffering from or expected to suffer from a Zika virus disease. In another embodiment, a patient treated in accordance with the methods provided herein is a patient diagnosed with a Zika virus infection or a disease associated therewith. In some embodiments, a patient treated in accordance with the methods provided herein is a patient infected with a Zika virus that does not manifest any symptoms of Zika virus disease.

In another embodiment, a patient treated in accordance with the methods provided herein is a patient experiencing one or more symptoms of Zika virus disease. Symptoms of Zika virus disease include, but are not limited to, body aches (especially joints, muscles and back), fever, headaches, itching, rash, and reddened eyes. In another embodiment, a patient treated in accordance with the methods provided herein is a patient with Zika virus disease who does not manifest symptoms of the disease that are severe enough to require hospitalization.

In some embodiments, a patient treated in accordance with the methods provided herein is an animal. In certain embodiments, the animal is a bird. In certain embodiments, the animal is a mammal, e.g., a horse, swine, mouse, or primate, preferably a human. In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human).

In a specific embodiment, a patient treated in accordance with the methods provided herein is a human. In certain embodiments, a patient treated in accordance with the methods provided herein is a human infant. In some embodiments, a patient treated in accordance with the methods provided herein is a human toddler. In certain embodiments, a patient treated in accordance with the methods provided herein is a human child. In other embodiments, a patient treated in accordance with the methods provided herein is a human adult. In some embodiments, a patient treated in accordance with the methods provided herein is an elderly human.

As used herein, the term “human adult” refers to a human that is 18 years or older.

As used herein, the term “human child” refers to a human that is 1 year to 18 years old. As used herein, the term “human infant” refers to a newborn to 1 year old human. As used herein, the term “human toddler” refers to a human that is 1 years to 3 years old.

In certain embodiments, a patient treated in accordance with the methods provided herein is a patient that is pregnant. In some embodiments, a patient treated in accordance with the methods provided herein is a patient is a fetus.

In some embodiments, a patient treated or prevented in accordance with the methods provided herein is any subject at increased risk of Zika virus infection or disease resulting from Zika virus infection (e.g., an immunocompromised or immunodeficient individual).

### 5.9 Diagnostic Uses

The antibodies described herein (e.g., a monoclonal antibody or an antigen-binding fragment thereof) or an antibody conjugate described herein can be used for diagnostic purposes to detect a Zika virus as well as detect, diagnose, or monitor a Zika virus infection. In specific embodiments, the antibodies (e.g., a monoclonal antibody or an antigen-binding fragment thereof) or antibody conjugates described herein can be used to discriminate between a Zika virus infection and a Dengue virus infection.

Provided herein are methods for the detection of a Zika virus infection comprising: (a) assaying the expression of a Zika virus NS1 in a biological specimen (e.g., blood, serum, plasma, cells or tissue samples) from a subject using an antibody described herein (e.g., a monoclonal antibody or an antigen-binding fragment thereof) or an antibody conjugate described herein; and (b) comparing the level of the Zika virus NS1 with a control level, e.g., levels in a biological specimen from a subject not infected with a Zika virus, wherein an increase in the assayed level of Zika virus NS1 compared to the control level of the Zika virus NS1 is indicative of a Zika virus infection.

Provided herein is a diagnostic assay for diagnosing a Zika virus infection comprising: (a) assaying for the level of a Zika virus NS1 in a biological specimen (e.g., blood, serum, plasma, cells, or tissue samples) from a subject using an antibody described herein (e.g., a monoclonal antibody or an antigen-binding fragment thereof) or an antibody conjugate described herein; and (b) comparing the level of the Zika virus NS1 with a control level, e.g., levels in a biological specimen from a subject not infected with Zika virus, wherein an increase in the assayed Zika virus NS1 level compared to the control level of the Zika virus NS1 is indicative of a Zika virus infection. A more definitive diagnosis of a Zika virus infection may allow health professionals to employ preventative measures or aggressive treatment earlier thereby preventing the development or further progression of the Zika virus infection.

In a specific embodiment, provided herein is a method for detecting a Zika virus, comprising: (a) contacting a biological sample (e.g., blood, serum, plasma, cells, tissue samples, etc.) with the antibody described herein or an antibody conjugate described herein; (b) detecting the binding of the antibody or antibody conjugate to an NS1 of a Zika virus, wherein Zika virus is detected if the level of binding of the antibody or antibody conjugate to an NS1 of a Zika virus is greater than the level of binding of the antibody or antibody conjugate to non-Zika virus infected cells (e.g., cells not infected with a virus or cells infected with another virus, such as Dengue virus) or a biological sample not infected with a Zika virus (e.g., a biological sample not infected with a virus or cells infected with another virus, such as Dengue virus or another flavivirus). In a particular embodiment, the detection is done in vitro. In other embodiments, the detection is done in vivo. Techniques known to one of skill in the art may be used to detect the binding of the antibody or the antibody conjugate to the NS1 of a Zika virus. In a specific embodiment, provided herein is a method for detecting a Zika virus infection in a biological sample, comprising contacting an antibody described herein or an antibody conjugate described herein with the biological sample and detecting the binding of the antibody or antibody conjugate to a Zika virus NS1. In another specific embodiment, provided herein is a method for diagnosing a Zika virus infection in a subject, comprising contacting an antibody described herein or an antibody conjugate described herein with a biological sample from the subject and detecting the binding of the antibody or antibody conjugate to a Zika virus NS1, wherein an increase in the detection of the binding of the antibody or antibody conjugate in the biological sample relative to the detection of binding of the antibody or antibody conjugate to a negative control sample indicates that the subject has a Zika virus infection. In another specific embodiment, provided herein is a method of distinguishing Zika virus from Dengue virus in a biological sample, comprising contacting an antibody described herein or an antibody conjugate described herein with the biological sample and detecting the binding of the antibody or antibody conjugate to a Zika virus NS1, wherein an increase in the detection of the binding of the antibody or antibody conjugate relative to the detection of binding of the antibody or antibody conjugate to a sample containing Dengue virus indicates the presence of Zika virus in the biological sample. In a specific embodiment, the biological sample is a blood, serum, plasma, cell, or tissue sample.

Antibodies described herein (e.g., a monoclonal antibody or an antigen-binding fragment thereof) can be used to assay Zika virus NS1 levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Antibody-based methods useful for detecting protein expression include immunoassays, such as the enzyme linked immunosorbent assay (ELISA) and the radioimmunoassay (RIA). An antibody described herein or generated in accordance with the methods described herein may be labeled with a detectable label or a secondary antibody that binds to such an antibody may be labeled with a detectable label. Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. See, Section 5.2.2, supra, for examples of antibody conjugates that might be useful in the detection and diagnosis of Zika virus infection.

Also provided herein is the detection and diagnosis of a Zika virus infection in a human. In one embodiment, diagnosis comprises: a) administering (for example, parenterally, intranasally, subcutaneously, or intraperitoneally) to a subject an effective amount of a labeled monoclonal antibody described herein (e.g., a monoclonal antibody or an antigen-binding fragment thereof); b) waiting for a time interval following the administering for permitting the labeled antibody to preferentially concentrate at sites in the subject (e.g., the nasal passages, lungs, mouth and ears) where the Zika virus antigen is expressed (and for unbound labeled molecule to be cleared to background level); c) determining background level; and d) detecting the labeled antibody in the subject, such that detection of labeled antibody above the background level indicates that the subject has a Zika virus infection or a symptom relating thereto. Background level can be determined by various methods, including comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to preferentially concentrate at sites in the subject and for unbound labeled antibody to be cleared to background level is 6 to 48 hours, or 6 to 24 hours or 6 to 12 hours. In another embodiment the time interval following administration is 5 to 20 days or 5 to 10 days.

In one embodiment, monitoring of a Zika virus infection is carried out by repeating the method for diagnosing the Zika virus infection, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

In a specific embodiment, an antibody or antibody conjugate described herein is used in an immunoassay to detect Zika virus NS1. Immunoassays may include Western blots, ELISAs, etc. In another aspect, an NS1 polypeptide described herein is used to measure the concentration of antibodies that bind to Zika virus NS1 in a biological sample (e.g., a biological sample from a subject). For example, a biological sample may be contacted with an NS1 polypeptide in an immunoassay and the binding of antibody in the biological sample may be determined by techniques known to one of skill in the art. In another aspect, an NS1 polypeptide described herein is used to detect a subject that has or is infected with a Zika virus. In a specific, an NS1 polypeptide described herein is used to determine whether a subject has been exposed to Zika virus. In another aspect, an NS1 polypeptide described herein may be used to diagnose a Zika virus infection. For example, a biological sample from a subject may be contacted with an NS1 polypeptide in an immunoassay and the binding of antibody in the biological sample may be determined by techniques known to one of skill in the art. In certain embodiments, a positive control (e.g. a biological sample infected with a Zika virus), a negative control (e.g., a biological sample that is not infected with a Zika virus), or both are included in the immunoassays. In another aspect, an NS1 polypeptide described herein may be used to prognose a Zika virus infection or a Zika virus disease. For example, a higher level of antibody that specifically binds to the NS1 polypeptide detected in a biological sample from a subject may indicate that the subject has a better chance of successfully recovery from the infection or disease, or that the infection or disease symptoms will not be severe.

### 5.10 Biological Assays

An antibody described herein (e.g., a monoclonal antibody or an antigen-binding fragment thereof) may be characterized using any assay known to one of skill in the art or described herein (e.g., as described in Section 5.2 or 6 herein). In addition, an NS1 polypeptide may be characterized using any assay known to one of skill in the art or described herein (e.g., as described in Section 5.1 or 6 herein).

### 5.10.1 Assays for Testing Antibody Activity

An antibody may be characterized in a variety of ways known to one of skill in the art (e.g., ELISA, biolayer interferometry, surface plasmon resonance display (BIAcore kinetic), Western blot, immunofluorescence, immunostaining and/or microneutralization assays). In some embodiments, an antibody is assayed for its ability to bind to a Zika virus NS1, or a recombinant NS1 protein.

The specificity or selectivity of an antibody for a Zika virus NS1 protein and cross-reactivity with other antigens (e.g., Dengue virus NS1) can be assessed by any method known in the art. Immunoassays which can be used to analyze specific binding and cross-reactivity include, but are not limited to, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of an antibody to a Zika virus NS1 protein and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody for a Zika virus NS1 protein or a recombinant NS1 protein and the binding off-rates can be determined from the data by Scatchard plot analysis. Competition with a second antibody can also be determined using radioimmunoassays. In this case, a Zika virus NS1 protein is incubated with the test antibody conjugated to a detectable labeled (e.g., ³H or ¹²⁵I) in the presence of increasing amounts of an unlabeled second antibody.

In some embodiments, surface plasmon resonance (e.g., BIAcore kinetic) analysis is used to determine the binding on and off rates of an antibody to a Zika virus NS1 protein or a recombinant NS1 protein. BIAcore kinetic analysis typically comprises analyzing the binding and dissociation of a Zika virus NS1 protein or a recombinant NS1 protein from chips with immobilized antibodies to a Zika virus NS1 protein or a recombinant NS1 protein on their surface. Briefly, a typical BIAcore kinetic study involves the injection of 250 μL of an antibody reagent (mAb, Fab) at varying concentration in HBS buffer containing 0.005% Tween-20 over a sensor chip surface, onto which has been immobilized the a Zika virus NS1 protein or a recombinant NS1 protein. The flow rate is maintained constant at 75 μL/min. Dissociation data is collected for 15 min or longer as necessary. Following each injection/dissociation cycle, the bound antibody is removed from the a Zika virus NS1 protein or a recombinant NS1 protein surface using brief, 1 min pulses of dilute acid, typically 10⁻¹⁰⁰ mM HCl, though other regenerants are employed as the circumstances warrant. More specifically, for measurement of the rates of association, kₒₙ, and dissociation, k_{off}, the polypeptide is directly immobilized onto the sensor chip surface through the use of standard amine coupling chemistries, namely the EDC/NHS method (EDC=N-diethylaminopropyl)-carbodiimide). Briefly, a 5-100 nM solution of the polypeptide in 10 mM NaOAc, pH 4 or pH 5 is prepared and passed over the EDC/NHS-activated surface until approximately 30-50 RU's worth of polypeptide are immobilized. Following this, the unreacted active esters are “capped” off with an injection of 1M Et-NH₂. A blank surface, containing no polypeptide, is prepared under identical immobilization conditions for reference purposes. Once an appropriate surface has been prepared, a suitable dilution series of each one of the antibody reagents is prepared in HBS/Tween-20, and passed over both the polypeptide and reference cell surfaces, which are connected in series. The range of antibody concentrations that are prepared varies, depending on what the equilibrium binding constant, K_{D}, is estimated to be. As described above, the bound antibody is removed after each injection/dissociation cycle using an appropriate regenerant.

In a specific embodiment, the affinity of an antibody described herein for a Zika virus NS1 or a recombinant NS1 polypeptide described herein is determined by a technique described in Section 6, infra. In another embodiment, the neutralizing activity of an antibody described herein is measured by a technique described in Section 6, infra. In another embodiment, one, two or all of the following of an antibody described herein is assessed by a technique known in the art or described herein (e.g., in Section 6, infra): antibody-dependent cell-mediated cytotoxity, antibody-dependent cell-mediated phagocytosis, antibody-dependent complement-mediated lysis. In another embodiment, antibody-dependent enhancement of a Zika virus infection is determined in vitro using a technique known in the art or described herein (e.g., in Section 6, infra).

### 5.10.2 Assays for Testing NS1 Protein

Assays for testing the expression of an NS1 polypeptide disclosed herein may be conducted using any assay known in the art. For example, an immunoassay, such as a Western blot, may be used to assess the expression of an NS1 polypeptide. An assay for incorporation of an NS1 polypeptide into a viral vector may comprise growing a virus as described herein, purifying the viral particles by centrifugation through a sucrose cushion, and subsequent analysis for the NS1 polypeptide expression by an immunoassay, such as Western blotting, using methods well known in the art. In a specific embodiment, expression of an NS1 polypeptide is determined using a technique described in Section 6, infra.

In another embodiment, an NS1 polypeptide disclosed herein is assayed for proper folding by determination of the structure or conformation of the NS1 polypeptide using any method known in the art such as, e.g., NMR, X-ray crystallographic methods, or secondary structure prediction methods, e.g., circular dichroism.

In another embodiment, an NS1 polypeptide disclosed herein is tested for the ability to form dimers using a technique known in the art or described herein (e.g., in Section 6, infra). In another embodiment, an NS1 polypeptide disclosed herein or a virus containing or expressing an NS1 polypeptide disclosed herein is assessed for Zika virus NS1 activity using a technique known to one of skill in the art or described herein (e.g., in Section 6, infra).

### 5.11 Kits

In another aspect, provided herein is a pharmaceutical pack or kit comprising one or more containers filled with one or more of the ingredients of a composition (e.g., a pharmaceutical compositions) described herein, such as one or more antibodies provided herein (e.g., a monoclonal antibody or an antigen-binding fragment thereof) or one or more antibody conjugates described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in the above methods. In one embodiment, a kit comprises an antibody described herein, preferably an isolated antibody, in one or more containers. In a specific embodiment, the kits encompassed herein contain an isolated Zika virus antigen that the antibodies encompassed herein react with (e.g., the antibody binds to the antigen) as a control. In a specific embodiment, the kits provided herein further comprise a control antibody which does not react with a Zika virus NS1 (e.g., the antibody does not bind to the Zika virus NS1, such as a control IgG). In another specific embodiment, the kits provided herein contain a means for detecting the binding of an antibody to a Zika virus NS1 (e.g., the antibody may be conjugated to a detectable substrate such as a fluorescent compound, an enzymatic substrate, a radioactive compound, a luminescent compound, or another antibody that is conjugated to a detectable substrate (e.g., the antibody may be conjugated to a second antibody which recognizes/binds to the first antibody)). In certain embodiments, the kits comprise a second antibody which is labeled with a detectable substance and which binds to an antibody described herein. In specific embodiments, the kit may include a recombinantly produced or chemically synthesized Zika virus NS1 (such as, e.g., described in Section 6, infra). The Zika NS1 provided in the kit may also be attached to a solid support. In a more specific embodiment the detecting means of the above described kit includes a solid support to which a Zika virus NS1 is attached. Such a kit may also include a non-attached reporter-labeled antibody. In this embodiment, binding of the antibody to the Zika virus NS1 can be detected by binding of the said reporter-labeled antibody.

In another aspect, provided herein is a pharmaceutical pack or kit comprising one or more containers filled with one or more of the ingredients of a composition (e.g., an immunogenic compositions) described herein, such an NS1 polypeptide described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, provided herein are kits comprising an immunogen described herein in a container. In a specific embodiment, provided herein are kits comprising an immunogen described in Section 5.1 or 6, supra, in a container. In a specific embodiment, provided herein are kits comprising an NS1 polypeptide described in Section 5.1 or 6, supra, in a container.

### 5.12 Sequences

| SEQ ID | | |
|---|---|---|
| NO: | Description of Sequence | Sequence |
| 1 | Isolate AA12 immunoglobulin | gaggtgcagctggtggagtccggaggaggcttgatccagcct |
| | heavy chain variable region | ggggggtccctgagactctcctgtgcagcctctgggttcaccgt |
| | mRNA, partial CDS | cagtagcaactacatgagctgggtccgccaggctccagggaa |
| | [organism = Homo sapiens] | ggggctggagtgggtctcagttatttatagcggtggtagcacat |
| | | actacgcagactccgtgaagggccgattcaccatctccagaga |
| | | caattccaagaacacgctgtatcttcaaatgaacagcctgagag |
| | | ccgaggacacggccgtgtattactgtgcgagagatcgaaggg |
| | | ggtttgactactggggccagggaacaatggtcaccgtctcttca |
| 2 | Isolate AA12 immunoglobulin | gacatccagatgacccagtctccactctccctgtctgcatctgta |
| | light chain variable region | ggagacagagtcaccatcacttgccggacaagtcagagcatta |
| | mRNA, partial CDS | gcagctatttaaattggtatcagcagaaaccagggaaagcccct |
| | [organism = Homo sapiens] | aagctcctgatctatgctgcatccagtttgcaaagtggggtccca |
| | | tcaaggttcagtggcagtggatctgggacagatttcactcttacc |
| | | atcagcagtctgcaacctgaagattttgcaacttactactgtcaa |
| | | cagacttacagtacccctctcactttcggcggagggaccaagg |
| | | tggaaatcaaa |
| 3 | Isolate EB9 immunoglobulin | gaggtgcagctggtggagtctggaggaggcttgatccagcct |
| | heavy chain variable region | ggggggtccctgagactctcctgtgcagcctctgggttcaccgt |
| | mRNA, partial CDS | cagtagcaactacatgagctgggtccgccaggctccagggaa |
| | [organism = Homo sapiens] | ggggctggagtgggtctcagttatttatagcggtggtagcacat |
| | | actacgcagactccgtgaagggccgattcaccatctccagaga |
| | | caattccaagaacacgctgtatcttcaaatgaacagcctgagag |
| | | ccgaggacacggccgtgtattactgtgcgagatggggaggga |
| | | aacgggggggggcttttgatatctggggccaagggacaatgg |
| | | tcaccgtctcttca |
| 4 | Isolate EB9 immunoglobulin | gacatccagatgacccagtctccattctccctgtctgcatctgta |
| | light chain variable region | ggagacagagtcaccatcacttgccgggcaagtcagagcatta |
| | mRNA, partial CDS | gcagccatttaaattggtatcagcagaaaccagggaaagcccc |
| | [organism = Homo sapiens] | taagttcctgatctatgctgcatccagtttgcaaagtggggtccc |
| | | atcaaggttcagtggcagtggatctgggacagacttcactctca |
| | | ccatcagcagtctgcaacctgaagattttgcaacttactactgtc |
| | | aacagagttacagtactccgtacacttttggccaggggaccaa |
| | | ggtggaaatcaaac |
| 5 | Isolate GB5 immunoglobulin | gaggtgcagctggtggagtctggaggaggcttgatccagcct |
| | heavy chain variable region | ggggggtccctgagactctcctgtgcagcctctgggttcaccgt |
| | mRNA, partial CDS | cagtagcaactacatgagctgggtccgccaggctccagggaa |
| | [organism = Homo sapiens] | ggggctggagtgggtctcagttatttatagcggtggtagcacat |
| | | actacgcagactccgtgaagggccgattcaccatctccagaga |
| | | caattccaagaacacgctgtatcttcaaatgagcagcctgagag |
| | | ccgaggacacggccgtgtattactgtgcgagactcatagcagc |
| | | agctggtgactactggggccagggaacaatggtcaccgtctct |
| | | tcag |
| 6 | Isolate GB5 immunoglobulin | gacatccagatgacccagtctccattcaccctgtctgcatctgta |
| | light chain variable region | ggagacagagtcaccatcacttgccgggcaagtcagagcatta |
| | mRNA, partial CDS | gcagctatttaaattggtatcagcagaaaccagggaaagcccct |
| | [organism = Homo sapiens] | aagctcctgatctatgctgcatccagtttgcaaagtggggtccca |
| | | tcaaggttcagtggcagtgaatctgggacagatttcactctcacc |
| | | atcagcagtctgcaacctgaagattttgcaacttactactgtcaa |
| | | cagagttacagtaccccctggacgttcggccaagggaccaag |
| | | gtggagatcaaac |
| 7 | Isolate FC12 immunoglobulin | gaggtgcagctggtggagtctggaggaggcttgatccagcct |
| | heavy chain variable region | ggggggtccctgagactctcctgtgcagcctctgggttcaccgt |
| | mRNA, partial CDS | cagtagcaactacatgagctgggtccgccagactccagggaa |
| | [organism = Homo sapiens] | ggggctggagtgggtctcagttatttatagcggtggtagcacat |
| | | actacgcagactccgtgaagggccgattcaccatctccagaga |
| | | caattccaagaacacgctgtatcttcaaatgaacagcctgagag |
| | | ccgaggacacggccgtgtattactgtgcgagagggcccgtac |
| | | aactggaacgacggcctctgggtgcttttgatatctggggccaa |
| | | gggacaatggtcaccgtctcttca |
| 8 | Isolate FC12 immunoglobulin | Tcctatgagctgactcagccaccctcagtgtccgtgtccccag |
| | light chain variable region | gacagacagccagcatcacctgctctggagataaattggggg |
| | mRNA, partial CDS | ataaatatgcttgctggtatcagcagaagccaggccagtcccct |
| | [organism = Homo sapiens] | gtgctggtcatctatcaagatagcaagcggccctcagggatcc |
| | | ctgagcgattctctggctccaactctgggaacacagccactctg |
| | | accatcagcgggacccaggctatggatgaggctgactattact |
| | | gtcaggcgtgggacagcagcaccgtggtattcggcggaggg |
| | | accaagctgaccgtcctag |
| 9 | Isolate AA12 immunoglobulin | EVQLVESGGGLIQPGGSLRLSCAASGFT |
| | heavy chain variable region | VSSNYMSWVRQAPGKGLEWVSVIYSGG |
| | amino acid sequence | STYYADSVKGRFTISRDNSKNTLYLQMN |
| | [organism = Homo sapiens] | SLRAEDTAVYYCARDRRGFDYWGQGT |
| | | MVTVSS |
| 10 | Isolate AA12 immunoglobulin | DIQMTQSPLSLSASVGDRVTITCRTSQSIS |
| | light chain variable region | SYLNWYQQKPGKAPKLLIYAASSLQSG |
| | amino acid sequence | VPSRFSGSGSGTDFTLTISSLQPEDFATY |
| | [organism = Homo sapiens] | YCQQTYSTPLTFGGGTKVEIK |
| 11 | Isolate EB9 immunoglobulin | EVQLVESGGGLIQPGGSLRLSCAASGFT |
| | heavy chain variable region | VSSNYMSWVRQAPGKGLEWVSVIYSGG |
| | amino acid sequence | STYYADSVKGRFTISRDNSKNTLYLQMN |
| | [organism = Homo sapiens] | SLRAEDTAVYYCARWGGKRGGAFDIW |
| | | GQGTMVTVSS |
| 12 | Isolate EB9 immunoglobulin | DIQMTQSPFSLSASVGDRVTITCRASQSIS |
| | light chain variable region | SHLNWYQQKPGKAPKFLIYAASSLQSGV |
| | amino acid sequence | PSRFSGSGSGTDFTLTISSLQPEDFATYYC |
| | [organism = Homo sapiens] | QQSYSTPYTFGQGTKVEIK |
| 13 | Isolate GB5 immunoglobulin | EVQLVESGGGLIQPGGSLRLSCAASGFT |
| | heavy chain variable region | VSSNYMSWVRQAPGKGLEWVSVIYSGG |
| | amino acid sequence | STYYADSVKGRFTISRDNSKNTLYLQMS |
| | [organism = Homo sapiens] | SLRAEDTAVYYCARLIAAAGDYWGQGT |
| | | MVTVSS |
| 14 | Isolate GB5 immunoglobulin | DIQMTQSPFTLSASVGDRVTITCRASQSI |
| | light chain variable region | SSYLNWYQQKPGKAPKLLIYAASSLQSG |
| | amino acid sequence | VPSRFSGSESGTDFTLTISSLQPEDFATYY |
| | [organism = Homo sapiens] | CQQSYSTPWTFGQGTKVEIK |
| 15 | Isolate FC12 immunoglobulin | EVQLVESGGGLIQPGGSLRLSCAASGFT |
| | heavy chain variable region | VSSNYMSWVRQTPGKGLEWVSVIYSGG |
| | amino acid sequence | STYYADSVKGRFTISRDNSKNTLYLQMN |
| | [organism = Homo sapiens] | SLRAEDTAVYYCARGPVQLERRPLGAF |
| | | DIWGQGTMVTVSS |
| 16 | Isolate FC12 immunoglobulin | SYELTQPPSVSVSPGQTASITCSGDKLGD |
| | light chain variable region | KYACWYQQKPGQSPVLVIYQDSKRPSGI |
| | amino acid sequence | PERFSGSNSGNTATLTISGTQAMDEADY |
| | [organism = Homo sapiens] | YCQAWDSSTVVFGGGTKLTVL |
| 17 | Isolate AA12 immunoglobulin | GFTVSSNY |
| | heavy chain variable region | |
| | CDR1 amino acid sequence | |
| | (IMGT) | |
| 18 | Isolate AA12 immunoglobulin | IYSGGST |
| | heavy chain variable region | |
| | CDR2 amino acid sequence | |
| | (IMGT) | |
| 19 | Isolate AA12 immunoglobulin | ARDRRGFDY |
| | heavy chain variable region | |
| | CDR3 amino acid sequence | |
| | (IMGT) | |
| 20 | Isolate AA12 immunoglobulin | QSISSY |
| | light chain variable region | |
| | CDR1 amino acid sequence | |
| | (IMGT) | |
| 21 | Isolate AA12 immunoglobulin | AAS |
| | light chain variable region | |
| | CDR2 amino acid sequence | |
| | (IMGT) | |
| 22 | Isolate AA12 immunoglobulin | QQTYSTPLT |
| | light chain variable region | |
| | CDR3 amino acid sequence | |
| | (IMGT) | |
| 23 | Isolate AA12 immunoglobulin | EVQLVESGGGLIQPGGSLRLSCAAS |
| | heavy chain variable region | |
| | framework region 1 amino | |
| | acid sequence (IMGT) | |
| 24 | Isolate AA12 immunoglobulin | MSWVRQAPGKGLEWVSV |
| | heavy chain variable region | |
| | framework region 2 amino | |
| | acid sequence (IMGT) | |
| 25 | Isolate AA12 immunoglobulin | YYADSVKGRFTISRDNSKNTLYLQMNSL |
| | heavy chain variable region | RAEDTAVYYC |
| | framework region 3 amino | |
| | acid sequence (IMGT) | |
| 26 | Isolate AA12 immunoglobulin | WGQGTMVTVSS |
| | heavy chain variable region | |
| | framework region 4 amino | |
| | acid sequence (IMGT) | |
| 27 | Isolate AA12 immunoglobulin | DIQMTQSPLSLSASVGDRVTITCRTS |
| | light chain variable region | |
| | framework region 1 amino | |
| | acid sequence (IMGT) | |
| 28 | Isolate AA12 immunoglobulin | LNWYQQKPGKAPKLLIY |
| | light chain variable region | |
| | framework region 2 amino | |
| | acid sequence (IMGT) | |
| 29 | Isolate AA12 immunoglobulin | SLQSGVPSRFSGSGSGTDFTLTISSLQPED |
| | light chain variable region | FATYYC |
| | framework region 3 amino | |
| | acid sequence (IMGT) | |
| 30 | Isolate AA12 immunoglobulin | FGGGTKVEIK |
| | light chain variable region | |
| | framework region 4 amino | |
| | acid sequence (IMGT) | |
| 31 | Isolate AA12 immunoglobulin | FTVSSNYMS |
| | heavy chain variable region | |
| | ABR1 amino acid sequence | |
| | (Paratome) | |
| 32 | Isolate AA12 immunoglobulin | WVSVIYSGGSTYYA |
| | heavy chain variable region | |
| | ABR2 amino acid sequence | |
| | (Paratome) | |
| 33 | Isolate AA12 immunoglobulin | ARDRRGFDY |
| | heavy chain variable region | |
| | ABR3 amino acid sequence | |
| | (Paratome) | |
| 34 | Isolate AA12 immunoglobulin | QSISSYLN |
| | light chain variable region | |
| | ABR1 amino acid sequence | |
| | (Paratome) | |
| 35 | Isolate AA12 immunoglobulin | LLIYAASSLQS |
| | light chain variable region | |
| | ABR2 amino acid sequence | |
| | (Paratome) | |
| 36 | Isolate AA12 immunoglobulin | QQTYSTPL |
| | light chain variable region | |
| | ABR3 amino acid sequence | |
| | (Paratome) | |
| 37 | Isolate AA12 immunoglobulin | EVQLVESGGGLIQPGGSLRLSCAASG |
| | heavy chain variable region | |
| | framework region 1 amino | |
| | acid sequence (Paratome) | |
| 38 | Isolate AA12 immunoglobulin | WVRQAPGKGLE |
| | heavy chain variable region | |
| | framework region 2 amino | |
| | acid sequence (Paratome) | |
| 39 | Isolate AA12 immunoglobulin | DSVKGRFTISRDNSKNTLYLQMNSLRAE |
| | heavy chain variable region | DTAVYYC |
| | framework region 3 amino | |
| | acid sequence (Paratome) | |
| 40 | Isolate AA12 immunoglobulin | WGQGTMVTVSS |
| | heavy chain variable region | |
| | framework region 4 amino | |
| | acid sequence (Paratome) | |
| 41 | Isolate AA12 immunoglobulin | DIQMTQSPLSLSASVGDRVTITCRTS |
| | light chain variable region | |
| | framework region 1 amino | |
| | acid sequence (Paratome) | |
| 42 | Isolate AA12 immunoglobulin | WYQQKPGKAPK |
| | light chain variable region | |
| | framework region 2 amino | |
| | acid sequence (Paratome) | |
| 43 | Isolate AA12 immunoglobulin | GVPSRFSGSGSGTDFTLTISSLQPEDFAT |
| | light chain variable region | YYC |
| | framework region 3 amino | |
| | acid sequence (Paratome) | |
| 44 | Isolate AA12 immunoglobulin | TFGGGTKVEIK |
| | light chain variable region | |
| | framework region 4 amino | |
| | acid sequence (Paratome) | |
| 45 | Isolate EB9 immunoglobulin | GFTVSSNY |
| | heavy chain variable region | |
| | CDR1 amino acid sequence | |
| | (IMGT) | |
| 46 | Isolate EB9 immunoglobulin | IYSGGST |
| | heavy chain variable region | |
| | CDR2 amino acid sequence | |
| | (IMGT) | |
| 47 | Isolate EB9 immunoglobulin | ARWGGKRGGAFDI |
| | heavy chain variable region | |
| | CDR3 amino acid sequence | |
| | (IMGT) | |
| 48 | Isolate EB9 immunoglobulin | QSISSH |
| | light chain variable region | |
| | CDR1 amino acid sequence | |
| | (IMGT) | |
| 49 | Isolate EB9 immunoglobulin | AAS |
| | light chain variable region | |
| | CDR2 amino acid sequence | |
| | (IMGT) | |
| 50 | Isolate EB9 immunoglobulin | QQSYSTPYT |
| | light chain variable region | |
| | CDR3 amino acid sequence | |
| | (IMGT) | |
| 51 | Isolate EB9 immunoglobulin | EVQLVESGGGLIQPGGSLRLSCAAS |
| | heavy chain variable region | |
| | framework region 1 amino | |
| | acid sequence (IMGT) | |
| 52 | Isolate EB9 immunoglobulin | MSWVRQAPGKGLEWVSV |
| | heavy chain variable region | |
| | framework region 2 amino | |
| | acid sequence (IMGT) | |
| 53 | Isolate EB9 immunoglobulin | YYADSVKGRFTISRDNSKNTLYLQMNSL |
| | heavy chain variable region | RAEDTAVYYC |
| | framework region 3 amino | |
| | acid sequence (IMGT) | |
| 54 | Isolate EB9 immunoglobulin | WGQGTMVTVSS |
| | heavy chain variable region | |
| | framework region 4 amino | |
| | acid sequence (IMGT) | |
| 55 | Isolate EB9 immunoglobulin | DIQMTQSPFSLSASVGDRVTITCRAS |
| | light chain variable region | |
| | framework region 1 amino | |
| | acid sequence (IMGT) | |
| 56 | Isolate EB9 immunoglobulin | LNWYQQKPGKAPKFLIY |
| | light chain variable region | |
| | framework region 2 amino | |
| | acid sequence (IMGT) | |
| 57 | Isolate EB9 immunoglobulin | SLQSGVPSRFSGSGSGTDFTLTISSLQPED |
| | light chain variable region | FATYYC |
| | framework region 3 amino | |
| | acid sequence (IMGT) | |
| 58 | Isolate EB9 immunoglobulin | FGQGTKVEIK |
| | light chain variable region | |
| | framework region 4 amino | |
| | acid sequence (IMGT) | |
| 59 | Isolate EB9 immunoglobulin | FTVSSNYMS |
| | heavy chain variable region | |
| | ABR1 amino acid sequence | |
| | (Paratome) | |
| 60 | Isolate EB9 immunoglobulin | WVSVIYSGGSTYYA |
| | heavy chain variable region | |
| | ABR2 amino acid sequence | |
| | (Paratome) | |
| 61 | Isolate EB9 immunoglobulin | ARWGGKRGGAFDI |
| | heavy chain variable region | |
| | ABR3 amino acid sequence | |
| | (Paratome) | |
| 62 | Isolate EB9 immunoglobulin | QSISSHLN |
| | light chain variable region | |
| | ABR1 amino acid sequence | |
| | (Paratome) | |
| 63 | Isolate EB9 immunoglobulin | FLIYAASSLQS |
| | light chain variable region | |
| | ABR2 amino acid sequence | |
| | (Paratome) | |
| 64 | Isolate EB9 immunoglobulin | QQSYSTPY |
| | light chain variable region | |
| | ABR3 amino acid sequence | |
| | (Paratome) | |
| 65 | Isolate EB9 immunoglobulin | EVQLVESGGGLIQPGGSLRLSCAASG |
| | heavy chain variable region | |
| | framework region 1 amino | |
| | acid sequence (Paratome) | |
| 66 | Isolate EB9 immunoglobulin | WVRQAPGKGLE |
| | heavy chain variable region | |
| | framework region 2 amino | |
| | acid sequence (Paratome) | |
| 67 | Isolate EB9 immunoglobulin | DSVKGRFTISRDNSKNTLYLQMNSLRAE |
| | heavy chain variable region | DTAVYYC |
| | framework region 3 amino | |
| | acid sequence (Paratome) | |
| 68 | Isolate EB9 immunoglobulin | WGQGTMVTVSS |
| | heavy chain variable region | |
| | framework region 4 amino | |
| | acid sequence (Paratome) | |
| 69 | Isolate EB9 immunoglobulin | DIQMTQSPFSLSASVGDRVTITCRAS |
| | light chain variable region | |
| | framework region 1 amino | |
| | acid sequence (Paratome) | |
| 70 | Isolate EB9 immunoglobulin | WYQQKPGKAPK |
| | light chain variable region | |
| | framework region 2 amino | |
| | acid sequence (Paratome) | |
| 71 | Isolate EB9 immunoglobulin | GVPSRFSGSGSGTDFTLTISSLQPEDFAT |
| | light chain variable region | YYC |
| | framework region 3 amino | |
| | acid sequence (Paratome) | |
| 72 | Isolate EB9 immunoglobulin | TFGQGTKVEIK |
| | light chain variable region | |
| | framework region 4 amino | |
| | acid sequence (Paratome) | |
| 73 | Isolate GB5 immunoglobulin | GFTVSSNY |
| | heavy chain variable region | |
| | CDR1 amino acid sequence | |
| | (IMGT) | |
| 74 | Isolate GB5 immunoglobulin | IYSGGST |
| | heavy chain variable region | |
| | CDR2 amino acid sequence | |
| | (IMGT) | |
| 75 | Isolate GB5 immunoglobulin | ARLIAAAGDY |
| | heavy chain variable region | |
| | CDR3 amino acid sequence | |
| | (IMGT) | |
| 76 | Isolate GB5 immunoglobulin | QSISSY |
| | light chain variable region | |
| | CDR1 amino acid sequence | |
| | (IMGT) | |
| 77 | Isolate GB5 immunoglobulin | AAS |
| | light chain variable region | |
| | CDR2 amino acid sequence | |
| | (IMGT) | |
| 78 | Isolate GB5 immunoglobulin | QQSYSTPWT |
| | light chain variable region | |
| | CDR3 amino acid sequence | |
| | (IMGT) | |
| 79 | Isolate GB5 immunoglobulin | EVQLVESGGGLIQPGGSLRLSCAAS |
| | heavy chain variable region | |
| | framework region 1 amino | |
| | acid sequence (IMGT) | |
| 80 | Isolate GB5 immunoglobulin | MSWVRQAPGKGLEWVSV |
| | heavy chain variable region | |
| | framework region 2 amino | |
| | acid sequence (IMGT) | |
| 81 | Isolate GB5 immunoglobulin | YYADSVKGRFTISRDNSKNTLYLQMSSL |
| | heavy chain variable region | RAEDTAVYYC |
| | framework region 3 amino | |
| | acid sequence (IMGT) | |
| 82 | Isolate GB5 immunoglobulin | WGQGTMVTVSS |
| | heavy chain variable region | |
| | framework region 4 amino | |
| | acid sequence (IMGT) | |
| 83 | Isolate GB5 immunoglobulin | DIQMTQSPFTLSASVGDRVTITCRAS |
| | light chain variable region | |
| | framework region 1 amino | |
| | acid sequence (IMGT) | |
| 84 | Isolate GB5 immunoglobulin | LNWYQQKPGKAPKLLIY |
| | light chain variable region | |
| | framework region 2 amino | |
| | acid sequence (IMGT) | |
| 85 | Isolate GB5 immunoglobulin | SLQSGVPSRFSGSESGTDFTLTISSLQPED |
| | light chain variable region | FATYYC |
| | framework region 3 amino | |
| | acid sequence (IMGT) | |
| 86 | Isolate GB5 immunoglobulin | FGQGTKVEIK |
| | light chain variable region | |
| | framework region 4 amino | |
| | acid sequence (IMGT) | |
| 87 | Isolate GB5 immunoglobulin | FTVSSNYMS |
| | heavy chain variable region | |
| | ABR1 amino acid sequence | |
| | (Paratome) | |
| 88 | Isolate GB5 immunoglobulin | WVSVIYSGGSTYYA |
| | heavy chain variable region | |
| | ABR2 amino acid sequence | |
| | (Paratome) | |
| 89 | Isolate GB5 immunoglobulin | ARLIAAAGDY |
| | heavy chain variable region | |
| | ABR3 amino acid sequence | |
| | (Paratome) | |
| 90 | Isolate GB5 immunoglobulin | QSISSYLN |
| | light chain variable region | |
| | ABR1 amino acid sequence | |
| | (Paratome) | |
| 91 | Isolate GB5 immunoglobulin | LLIYAASSLQS |
| | light chain variable region | |
| | ABR2 amino acid sequence | |
| | (Paratome) | |
| 92 | Isolate GB5 immunoglobulin | QQSYSTPW |
| | light chain variable region | |
| | ABR3 amino acid sequence | |
| | (Paratome) | |
| 93 | Isolate GB5 immunoglobulin | EVQLVESGGGLIQPGGSLRLSCAASG |
| | heavy chain variable region | |
| | framework region 1 amino | |
| | acid sequence (Paratome) | |
| 94 | Isolate GB5 immunoglobulin | WVRQAPGKGLE |
| | heavy chain variable region | |
| | framework region 2 amino | |
| | acid sequence (Paratome) | |
| 95 | Isolate GB5 immunoglobulin | DSVKGRFTISRDNSKNTLYLQMSSLRAE |
| | heavy chain variable region | DTAVYYC |
| | framework region 3 amino | |
| | acid sequence (Paratome) | |
| 96 | Isolate GB5 immunoglobulin | WGQGTMVTVSS |
| | heavy chain variable region | |
| | framework region 4 amino | |
| | acid sequence (Paratome) | |
| 97 | Isolate GB5 immunoglobulin | DIQMTQSPFTLSASVGDRVTITCRAS |
| | light chain variable region | |
| | framework region 1 amino | |
| | acid sequence (Paratome) | |
| 98 | Isolate GB5 immunoglobulin | WYQQKPGKAPK |
| | light chain variable region | |
| | framework region 2 amino | |
| | acid sequence (Paratome) | |
| 99 | Isolate GB5 immunoglobulin | GVPSRFSGSESGTDFTLTISSLQPEDFATY |
| | light chain variable region | YC |
| | framework region 3 amino | |
| | acid sequence (Paratome) | |
| 100 | Isolate GB5 immunoglobulin | TFGQGTKVEIK |
| | light chain variable region | |
| | framework region 4 amino | |
| | acid sequence (Paratome) | |
| 101 | Isolate FC12 immunoglobulin | GFTVSSNY |
| | heavy chain variable region | |
| | CDR1 amino acid sequence | |
| | (IMGT) | |
| 102 | Isolate FC12 immunoglobulin | IYSGGST |
| | heavy chain variable region | |
| | CDR2 amino acid sequence | |
| | (IMGT) | |
| 103 | Isolate FC12 immunoglobulin | ARGPVQLERRPLGAFDI |
| | heavy chain variable region | |
| | CDR3 amino acid sequence | |
| | (IMGT) | |
| 104 | Isolate FC12 immunoglobulin | KLGDKY |
| | light chain variable region | |
| | CDR1 amino acid sequence | |
| | (IMGT) | |
| 105 | Isolate FC12 immunoglobulin | QDS |
| | light chain variable region | |
| | CDR2 amino acid sequence | |
| | (IMGT) | |
| 106 | Isolate FC12 immunoglobulin | QAWDSSTVV |
| | light chain variable region | |
| | CDR3 amino acid sequence | |
| | (IMGT) | |
| 107 | Isolate FC12 immunoglobulin | EVQLVESGGGLIQPGGSLRLSCAAS |
| | heavy chain variable region | |
| | framework region 1 amino | |
| | acid sequence (IMGT) | |
| 108 | Isolate FC12 immunoglobulin | MSWVRQTPGKGLEWVSV |
| | heavy chain variable region | |
| | framework region 2 amino | |
| | acid sequence (IMGT) | |
| 109 | Isolate FC12 immunoglobulin | YYADSVKGRFTISRDNSKNTLYLQMNSL |
| | heavy chain variable region | RAEDTAVYYC |
| | framework region 3 amino | |
| | acid sequence (IMGT) | |
| 110 | Isolate FC12 immunoglobulin | WGQGTMVTVSS |
| | heavy chain variable region | |
| | framework region 4 amino | |
| | acid sequence (IMGT) | |
| 111 | Isolate FC12 immunoglobulin | SYELTQPPSVSVSPGQTASITCSGD |
| | light chain variable region | |
| | framework region 1 amino | |
| | acid sequence (IMGT) | |
| 112 | Isolate FC12 immunoglobulin | ACWYQQKPGQSPVLVIY |
| | light chain variable region | |
| | framework region 2 amino | |
| | acid sequence (IMGT) | |
| 113 | Isolate FC12 immunoglobulin | KRPSGIPERFSGSNSGNTATLTISGTQAM |
| | light chain variable region | DEADYYC |
| | framework region 3 amino | |
| | acid sequence (IMGT) | |
| 114 | Isolate FC12 immunoglobulin | FGGGTKLTVL |
| | light chain variable region | |
| | framework region 4 amino | |
| | acid sequence (IMGT) | |
| 115 | Isolate FC12 immunoglobulin | FTVSSNYMS |
| | heavy chain variable region | |
| | ABR1 amino acid sequence | |
| | (Paratome) | |
| 116 | Isolate FC12 immunoglobulin | WVSVIYSGGSTYYA |
| | heavy chain variable region | |
| | ABR2 amino acid sequence | |
| | (Paratome) | |
| 117 | Isolate FC12 immunoglobulin | ARGPVQLERRPLGAFDI |
| | heavy chain variable region | |
| | ABR3 amino acid sequence | |
| | (Paratome) | |
| 118 | Isolate FC12 immunoglobulin | KLGDKYAC |
| | light chain variable region | |
| | ABR1 amino acid sequence | |
| | (Paratome) | |
| 119 | Isolate FC12 immunoglobulin | LVIYQDSKRPS |
| | light chain variable region | |
| | ABR2 amino acid sequence | |
| | (Paratome) | |
| 120 | Isolate FC12 immunoglobulin | QAWDSSTV |
| | light chain variable region | |
| | ABR3 amino acid sequence | |
| | (Paratome) | |
| 121 | Isolate FC12 immunoglobulin | EVQLVESGGGLIQPGGSLRLSCAASG |
| | heavy chain variable region | |
| | framework region 1 amino | |
| | acid sequence (Paratome) | |
| 122 | Isolate FC12 immunoglobulin | WVRQTPGKGLE |
| | heavy chain variable region | |
| | framework region 2 amino | |
| | acid sequence (Paratome) | |
| 123 | Isolate FC12 immunoglobulin | DSVKGRFTISRDNSKNTLYLQMNSLRAE |
| | heavy chain variable region | DTAVYYC |
| | framework region 3 amino | |
| | acid sequence (Paratome) | |
| 124 | Isolate FC12 immunoglobulin | WGQGTMVTVSS |
| | heavy chain variable region | |
| | framework region 4 amino | |
| | acid sequence (Paratome) | |
| 125 | Isolate FC12 immunoglobulin | SYELTQPPSVSVSPGQTASITCSGD |
| | light chain variable region | |
| | framework region 1 amino | |
| | acid sequence (Paratome) | |
| 126 | Isolate FC12 immunoglobulin | WYQQKPGQSPV |
| | light chain variable region | |
| | framework region 2 amino | |
| | acid sequence (Paratome) | |
| 127 | Isolate FC12 immunoglobulin | GIPERFSGSNSGNTATLTISGTQAMDEAD |
| | light chain variable region | YYC |
| | framework region 3 amino | |
| | acid sequence (Paratome) | |
| 128 | Isolate FC12 immunoglobulin | VFGGGTKLTVL |
| | light chain variable region | |
| | framework region 4 amino | |
| | acid sequence (Paratome) | |
| 129 | The NS1 nucleotide sequence | gacgtggggtgctcagtggacttctcaaaaaaggaaacgagat |
| | of MR766 virus | gtggcacgggggtattcatctataatgatgttgaagcctggagg |
| | (Rhesus/1947/Uganda) | gaccggtacaagtaccatcctgactccccccgcagattggcag |
| | | cagcagtcaagcaggcctgggaagaggggatctgtgggatct |
| | | catccgtttcaagaatggaaaacatcatgtggaaatcagtagaa |
| | | ggggagctcaatgctatcctagaggagaatggagttcaactga |
| | | cagttgttgtgggatctgtaaaaaaccccatgtggagaggtcca |
| | | caaagattgccagtgcctgtgaatgagctgccccatggctgga |
| | | aagcctgggggaaatcgtattttgttagggcggcaaagaccaa |
| | | caacagttttgttgtcgacggtgacacactgaaggaatgtccgc |
| | | ttgagcacagagcatggaatagttttcttgtggaggatcacggg |
| | | tttggagtcttccacaccagtgtctggcttaaggtcagagaagat |
| | | tactcattagaatgtgacccagccgtcataggaacagctgttaa |
| | | gggaagggaggccgcgcacagtgatctgggctattggattga |
| | | aagtgaaaagaatgacacatggaggctgaagagggcccacct |
| | | gattgagatgaaaacatgtgaatggccaaagtctcacacattgt |
| | | ggacagatggagtagaagaaagtgatcttatcatacccaagtct |
| | | ttagctggtccactcagccaccacaacaccagagagggttaca |
| | | gaacccaagtgaaagggccatggcacagtgaagagcttgaaa |
| | | tccggtttgaggaatgtccaggcaccaaggtttacgtggagga |
| | | gacatgcggaactagaggaccatctctgagatcaactactgca |
| | | agtggaagggtcattgaggaatggtgctgtagggaatgcacaa |
| | | tgcccccactatcgtttcgagcaaaagacggctgctggtatgga |
| | | atggagataaggcccaggaaagaaccagagagcaacttagtg |
| | | aggtcaatggtgacagcg |
| 130 | PRVABC59 virus (2015/ | GTTGTTGATCTGTGTGAATCAGACTGC |
| | Puerto Rico Accession | GACAGTTCGAGTTTGAAGCGAAAGCT |
| | No.: KU501215) | AGCAACAGTATCAACAGGTTTTATTTT |
| | | GGATTTGGAAACGAGAGTTTCTGGTCA |
| | | TGAAAAACCCAAAAAAGAAATCCGGA |
| | | GGATTCCGGATTGTCAATATGCTAAAA |
| | | CGCGGAGTAGCCCGTGTGAGCCCCTTT |
| | | GGGGGCTTGAAGAGGCTGCCAGCCGG |
| | | ACTTCTGCTGGGTCATGGGCCCATCAG |
| | | GATGGTCTTGGCGATTCTAGCCTTTTT |
| | | GAGATTCACGGCAATCAAGCCATCACT |
| | | GGGTCTCATCAATAGATGGGGTTCAGT |
| | | GGGGAAAAAAGAGGCTATGGAAACAA |
| | | TAAAGAAGTTCAAGAAAGATCTGGCT |
| | | GCCATGCTGAGAATAATCAATGCTAGG |
| | | AAGGAGAAGAAGAGACGAGGCGCAG |
| | | ATACTAGTGTCGGAATTGTTGGCCTCC |
| | | TGCTGACCACAGCTATGGCAGCGGAG |
| | | GTCACTAGACGTGGGAGTGCATACTAT |
| | | ATGTACTTGGACAGAAACGATGCTGG |
| | | GGAGGCCATATCTTTTCCAACCACATT |
| | | GGGGATGAATAAGTGTTATATACAGAT |
| | | CATGGATCTTGGACACATGTGTGATGC |
| | | CACCATGAGCTATGAATGCCCTATGCT |
| | | GGATGAGGGGGTGGAACCAGATGACG |
| | | TCGATTGTTGGTGCAACACGACGTCAA |
| | | CTTGGGTTGTGTACGGAACCTGCCATC |
| | | ACAAAAAAGGTGAAGCACGGAGATCT |
| | | AGAAGAGCTGTGACGCTCCCCTCCCAT |
| | | TCCACCAGGAAGCTGCAAACGCGGTC |
| | | GCAAACCTGGTTGGAATCAAGAGAAT |
| | | ACACAAAGCACTTGATTAGAGTCGAA |
| | | AATTGGATATTCAGGAACCCTGGCTTC |
| | | GCGTTAGCAGCAGCTGCCATCGCTTGG |
| | | CTTTTGGGAAGCTCAACGAGCCAAAA |
| | | AGTCATATACTTGGTCATGATACTGCT |
| | | GATTGCCCCGGCATACAGCATCAGGTG |
| | | CATAGGAGTCAGCAATAGGGACTTTGT |
| | | GGAAGGTATGTCAGGTGGGACTTGGG |
| | | TTGATGTTGTCTTGGAACATGGAGGTT |
| | | GTGTCACCGTAATGGCACAGGACAAA |
| | | CCGACTGTCGACATAGAGCTGGTTACA |
| | | ACAACAGTCAGCAACATGGCGGAGGT |
| | | AAGATCCTACTGCTATGAGGCATCAAT |
| | | ATCAGACATGGCTTCTGACAGCCGCTG |
| | | CCCAACACAAGGTGAAGCCTACCTTGA |
| | | CAAGCAATCAGACACTCAATATGTCTG |
| | | CAAAAGAACGTTAGTGGACAGAGGCT |
| | | GGGGAAATGGATGTGGACTTTTTGGCA |
| | | AAGGGAGCCTGGTGACATGCGCTAAG |
| | | TTTGCATGCTCCAAGAAAATGACCGGG |
| | | AAGAGCATCCAGCCAGAGAATCTGGA |
| | | GTACCGGATAATGCTGTCAGTTCATGG |
| | | CTCCCAGCACAGTGGGATGATCGTTAA |
| | | TGACACAGGACATGAAACTGATGAGA |
| | | ATAGAGCGAAAGTTGAGATAACGCCC |
| | | AATTCACCGAGAGCCGAAGCCACCCT |
| | | GGGGGGTTTTGGAAGCCTAGGACTTGA |
| | | TTGTGAACCGAGGACAGGCCTTGACTT |
| | | TTCAGATTTGTATTACTTGACTATGAA |
| | | TAACAAGCACTGGTTGGTTCACAAGGA |
| | | GTGGTTCCACGACATTCCATTACCTTG |
| | | GCACGCTGGGGCAGACACCGGAACTC |
| | | CACACTGGAACAACAAAGAAGCACTG |
| | | GTAGAGTTCAAGGACGCACATGCCAA |
| | | AAGGCAAACTGTCGTGGTTCTAGGGA |
| | | GTCAAGAAGGAGCAGTTCACACGGCC |
| | | CTTGCTGGAGCTCTGGAGGCTGAGATG |
| | | GATGGTGCAAAGGGAAGGCTGTCCTCT |
| | | GGCCACTTGAAATGTCGCCTGAAAATG |
| | | GATAAACTTAGATTGAAGGGCGTGTCA |
| | | TACTCCTTGTGTACTGCAGCGTTCACA |
| | | TTCACCAAGATCCCGGCTGAAACACTG |
| | | CACGGGACAGTCACAGTGGAGGTACA |
| | | GTACGCAGGGACAGATGGACCTTGCA |
| | | AGGTTCCAGCTCAGATGGCGGTGGAC |
| | | ATGCAAACTCTGACCCCAGTTGGGAGG |
| | | TTGATAACCGCTAACCCCGTAATCACT |
| | | GAAAGCACTGAGAACTCTAAGATGAT |
| | | GCTGGAACTTGATCCACCATTTGGGGA |
| | | CTCTTACATTGTCATAGGAGTCGGGGA |
| | | GAAGAAGATCACCCACCACTGGCACA |
| | | GGAGTGGCAGCACCATTGGAAAAGCA |
| | | TTTGAAGCCACTGTGAGAGGTGCCAAG |
| | | AGAATGGCAGTCTTGGGAGACACAGC |
| | | CTGGGACTTTGGATCAGTTGGAGGCGC |
| | | TCTCAACTCATTGGGCAAGGGCATCCA |
| | | TCAAATTTTTGGAGCAGCTTTCAAATC |
| | | ATTGTTTGGAGGAATGTCCTGGTTCTC |
| | | ACAAATTCTCATTGGAACGTTGCTGAT |
| | | GTGGTTGGGTCTGAACACAAAGAATG |
| | | GATCTATTTCCCTTATGTGCTTGGCCTT |
| | | AGGGGGAGTGTTGATCTTCTTATCCAC |
| | | AGCCGTCTCTGCTGATGTGGGGTGCTC |
| | | GGTGGACTTCTCAAAGAAGGAGACGA |
| | | GATGCGGTACAGGGGTGTTCGTCTATA |
| | | ACGACGTTGAAGCCTGGAGGGACAGG |
| | | TACAAGTACCATCCTGACTCCCCCCGT |
| | | AGATTGGCAGCAGCAGTCAAGCAAGC |
| | | CTGGGAAGATGGTATCTGCGGGATCTC |
| | | CTCTGTTTCAAGAATGGAAAACATCAT |
| | | GTGGAGATCAGTAGAAGGGGAGCTCA |
| | | ACGCAATCCTGGAAGAGAATGGAGTT |
| | | CAACTGACGGTCGTTGTGGGATCTGTA |
| | | AAAAACCCCATGTGGAGAGGTCCACA |
| | | GAGATTGCCCGTGCCTGTGAACGAGCT |
| | | GCCCCACGGCTGGAAGGCTTGGGGGA |
| | | AATCGTATTTCGTCAGAGCAGCAAAGA |
| | | CAAATAACAGCTTTGTCGTGGATGGTG |
| | | ACACACTGAAGGAATGCCCACTCAAA |
| | | CATAGAGCATGGAACAGCTTTCTTGTG |
| | | GAGGATCATGGGTTCGGGGTATTTCAC |
| | | ACTAGTGTCTGGCTCAAGGTTAGAGAA |
| | | GATTATTCATTAGAGTGTGATCCAGCC |
| | | GTTATTGGAACAGCTGTTAAGGGAAA |
| | | GGAGGCTGTACACAGTGATCTAGGCTA |
| | | CTGGATTGAGAGTGAGAAGAATGACA |
| | | CATGGAGGCTGAAGAGGGCCCATCTG |
| | | ATCGAGATGAAAACATGTGAATGGCC |
| | | AAAGTCCCACACATTGTGGACAGATG |
| | | GAATAGAAGAGAGTGATCTGATCATA |
| | | CCCAAGTCTTTAGCTGGGCCACTCAGC |
| | | CATCACAATACCAGAGAGGGCTACAG |
| | | GACCCAAATGAAAGGGCCATGGCACA |
| | | GTGAAGAGCTTGAAATTCGGTTTGAGG |
| | | AATGCCCAGGCACTAAGGTCCACGTG |
| | | GAGGAAACATGTGGAACAAGAGGACC |
| | | ATCTCTGAGATCAACCACTGCAAGCGG |
| | | AAGGGTGATCGAGGAATGGTGCTGCA |
| | | GGGAGTGCACAATGCCCCCACTGTCGT |
| | | TCCGGGCTAAAGATGGCTGTTGGTATG |
| | | GAATGGAGATAAGGCCCAGGAAAGAA |
| | | CCAGAAAGCAACTTAGTAAGGTCAAT |
| | | GGTGACTGCAGGATCAACTGATCACAT |
| | | GGACCACTTCTCCCTTGGAGTGCTTGT |
| | | GATCCTGCTCATGGTGCAGGAAGGGCT |
| | | GAAGAAGAGAATGACCACAAAGATCA |
| | | TCATAAGCACATCAATGGCAGTGCTGG |
| | | TAGCTATGATCCTGGGAGGATTTTCAA |
| | | TGAGTGACCTGGCTAAGCTTGCAATTT |
| | | TGATGGGTGCCACCTTCGCGGAAATGA |
| | | ACACTGGAGGAGATGTAGCTCATCTGG |
| | | CGCTGATAGCGGCATTCAAAGTCAGAC |
| | | CAGCGTTGCTGGTATCTTTCATCTTCA |
| | | GAGCTAATTGGACACCCCGTGAAAGC |
| | | ATGCTGCTGGCCTTGGCCTCGTGTCTTT |
| | | TGCAAACTGCGATCTCCGCCTTGGAAG |
| | | GCGACCTGATGGTTCTCATCAATGGTT |
| | | TTGCTTTGGCCTGGTTGGCAATACGAG |
| | | CGATGGTTGTTCCACGCACTGATAACA |
| | | TCACCTTGGCAATCCTGGCTGCTCTGA |
| | | CACCACTGGCCCGGGGCACACTGCTTG |
| | | TGGCGTGGAGAGCAGGCCTTGCTACTT |
| | | GCGGGGGGTTTATGCTCCTCTCTCTGA |
| | | AGGGAAAAGGCAGTGTGAAGAAGAAC |
| | | TTACCATTTGTCATGGCCCTGGGACTA |
| | | ACCGCTGTGAGGCTGGTCGACCCCATC |
| | | AACGTGGTGGGACTGCTGTTGCTCACA |
| | | AGGAGTGGGAAGCGGAGCTGGCCCCC |
| | | TAGCGAAGTACTCACAGCTGTTGGCCT |
| | | GATATGCGCATTGGCTGGAGGGTTCGC |
| | | CAAGGCAGATATAGAGATGGCTGGGC |
| | | CCATGGCCGCGGTCGGTCTGCTAATTG |
| | | TCAGTTACGTGGTCTCAGGAAAGAGTG |
| | | TGGACATGTACATTGAAAGAGCAGGT |
| | | GACATCACATGGGAAAAAGATGCGGA |
| | | AGTCACTGGAAACAGTCCCCGGCTCGA |
| | | TGTGGCGCTAGATGAGAGTGGTGATTT |
| | | CTCCCTGGTGGAGGATGACGGTCCCCC |
| | | CATGAGAGAGATCATACTCAAGGTGG |
| | | TCCTGATGACCATCTGTGGCATGAACC |
| | | CAATAGCCATACCCTTTGCAGCTGGAG |
| | | CGTGGTACGTATACGTGAAGACTGGA |
| | | AAAAGGAGTGGTGCTCTATGGGATGT |
| | | GCCTGCTCCCAAGGAAGTAAAAAAGG |
| | | GGGAGACCACAGATGGAGTGTACAGA |
| | | GTAATGACTCGTAGACTGCTAGGTTCA |
| | | ACACAAGTTGGAGTGGGAGTTATGCA |
| | | AGAGGGGGTCTTTCACACTATGTGGCA |
| | | CGTCACAAAAGGATCCGCGCTGAGAA |
| | | GCGGTGAAGGGAGACTTGATCCATACT |
| | | GGGGAGATGTCAAGCAGGATCTGGTG |
| | | TCATACTGTGGTCCATGGAAGCTAGAT |
| | | GCCGCCTGGGATGGGCACAGCGAGGT |
| | | GCAGCTCTTGGCCGTGCCCCCCGGAGA |
| | | GAGAGCGAGGAACATCCAGACTCTGC |
| | | CCGGAATATTTAAGACAAAGGATGGG |
| | | GACATTGGAGCGGTTGCGCTGGATTAC |
| | | CCAGCAGGAACTTCAGGATCTCCAATC |
| | | CTAGACAAGTGTGGGAGAGTGATAGG |
| | | ACTTTATGGCAATGGGGTCGTGATCAA |
| | | AAACGGGAGTTATGTTAGTGCCATCAC |
| | | CCAAGGGAGGAGGGAGGAAGAGACTC |
| | | CTGTTGAGTGCTTCGAGCCCTCGATGC |
| | | TGAAGAAGAAGCAGCTAACTGTCTTA |
| | | GACTTGCATCCTGGAGCTGGGAAAACC |
| | | AGGAGAGTTCTTCCTGAAATAGTCCGT |
| | | GAAGCCATAAAAACAAGACTCCGTAC |
| | | TGTGATCTTAGCTCCAACCAGGGTTGT |
| | | CGCTGCTGAAATGGAGGAGGCCCTTA |
| | | GAGGGCTTCCAGTGCGTTATATGACAA |
| | | CAGCAGTCAATGTCACCCACTCTGGAA |
| | | CAGAAATCGTCGACTTAATGTGCCATG |
| | | CCACCTTCACTTCACGTCTACTACAGC |
| | | CAATCAGAGTCCCCAACTATAATCTGT |
| | | ATATTATGGATGAGGCCCACTTCACAG |
| | | ATCCCTCAAGTATAGCAGCAAGAGGA |
| | | TACATTTCAACAAGGGTTGAGATGGGC |
| | | GAGGCGGCTGCCATCTTCATGACCGCC |
| | | ACGCCACCAGGAACCCGTGACGCATTT |
| | | CCGGACTCCAACTCACCAATTATGGAC |
| | | ACCGAAGTGGAAGTCCCAGAGAGAGC |
| | | CTGGAGCTCAGGCTTTGATTGGGTGAC |
| | | GGATCATTCTGGAAAAACAGTTTGGTT |
| | | TGTTCCAAGCGTGAGGAACGGCAATG |
| | | AGATCGCAGCTTGTCTGACAAAGGCTG |
| | | GAAAACGGGTCATACAGCTCAGCAGA |
| | | AAGACTTTTGAGACAGAGTTCCAGAA |
| | | AACAAAACATCAAGAGTGGGACTTTG |
| | | TCGTGACAACTGACATTTCAGAGATGG |
| | | GCGCCAACTTTAAAGCTGACCGTGTCA |
| | | TAGATTCCAGGAGATGCCTAAAGCCG |
| | | GTCATACTTGATGGCGAGAGAGTCATT |
| | | CTGGCTGGACCCATGCCTGTCACACAT |
| | | GCCAGCGCTGCCCAGAGGAGGGGGCG |
| | | CATAGGCAGGAATCCCAACAAACCTG |
| | | GAGATGAGTATCTGTATGGAGGTGGGT |
| | | GCGCAGAGACTGACGAAGACCATGCA |
| | | CACTGGCTTGAAGCAAGAATGCTCCTT |
| | | GACAATATTTACCTCCAAGATGGCCTC |
| | | ATAGCCTCGCTCTATCGACCTGAGGCC |
| | | GACAAAGTAGCAGCCATTGAGGGAGA |
| | | GTTCAAGCTTAGGACGGAGCAAAGGA |
| | | AGACCTTTGTGGAACTCATGAAAAGA |
| | | GGAGATCTTCCTGTTTGGCTGGCCTAT |
| | | CAGGTTGCATCTGCCGGAATAACCTAC |
| | | ACAGATAGAAGATGGTGCTTTGATGGC |
| | | ACGACCAACAACACCATAATGGAAGA |
| | | CAGTGTGCCGGCAGAGGTGTGGACCA |
| | | GACACGGAGAGAAAAGAGTGCTCAAA |
| | | CCGAGGTGGATGGACGCCAGAGTTTGT |
| | | TCAGATCATGCGGCCCTGAAGTCATTC |
| | | AAGGAGTTTGCCGCTGGGAAAAGAGG |
| | | AGCGGCTTTTGGAGTGATGGAAGCCCT |
| | | GGGAACACTGCCAGGACACATGACAG |
| | | AGAGATTCCAGGAAGCCATTGACAAC |
| | | CTCGCTGTGCTCATGCGGGCAGAGACT |
| | | GGAAGCAGGCCTTACAAAGCCGCGGC |
| | | GGCCCAATTGCCGGAGACCCTAGAGA |
| | | CCATAATGCTTTTGGGGTTGCTGGGAA |
| | | CAGTCTCGCTGGGAATCTTCTTCGTCTT |
| | | GATGAGGAACAAGGGCATAGGGAAGA |
| | | TGGGCTTTGGAATGGTGACTCTTGGGG |
| | | CCAGCGCATGGCTCATGTGGCTCTCGG |
| | | AAATTGAGCCAGCCAGAATTGCATGTG |
| | | TCCTCATTGTTGTGTTCCTATTGCTGGT |
| | | GGTGCTCATACCTGAGCCAGAAAAGC |
| | | AAAGATCTCCCCAGGACAACCAAATG |
| | | GCAATCATCATCATGGTAGCAGTAGGT |
| | | CTTCTGGGCTTGATTACCGCCAATGAA |
| | | CTCGGATGGTTGGAGAGAACAAAGAG |
| | | TGACCTAAGCCATCTAATGGGAAGGA |
| | | GAGAGGAGGGGGCAACCATAGGATTC |
| | | TCAATGGACATTGACCTGCGGCCAGCC |
| | | TCAGCTTGGGCCATCTATGCTGCCTTG |
| | | ACAACTTTCATTACCCCAGCCGTCCAA |
| | | CATGCAGTGACCACCTCATACAACAAC |
| | | TACTCCTTAATGGCGATGGCCACGCAA |
| | | GCTGGAGTGTTGTTTGGCATGGGCAAA |
| | | GGGATGCCATTCTACGCATGGGACTTT |
| | | GGAGTCCCGCTGCTAATGATAGGTTGC |
| | | TACTCACAATTAACACCCCTGACCCTA |
| | | ATAGTGGCCATCATTTTGCTCGTGGCG |
| | | CACTACATGTACTTGATCCCAGGGCTG |
| | | CAGGCAGCAGCTGCGCGTGCTGCCCA |
| | | GAAGAGAACGGCAGCTGGCATCATGA |
| | | AGAACCCTGTTGTGGATGGAATAGTGG |
| | | TGACTGACATTGACACAATGACAATTG |
| | | ACCCCCAAGTGGAGAAAAAGATGGGA |
| | | CAGGTGCTACTCATAGCAGTAGCCGTC |
| | | TCCAGCGCCATACTGTCGCGGACCGCC |
| | | TGGGGGTGGGGGGAGGCTGGGGCTCT |
| | | GATCACAGCCGCAACTTCCACTTTGTG |
| | | GGAAGGCTCTCCGAACAAGTACTGGA |
| | | ACTCCTCTACAGCCACTTCACTGTGTA |
| | | ACATTTTTAGGGGAAGTTACTTGGCTG |
| | | GAGCTTCTCTAATCTACACAGTAACAA |
| | | GAAACGCTGGCTTGGTCAAGAGACGT |
| | | GGGGGTGGAACAGGAGAGACCCTGGG |
| | | AGAGAAATGGAAGGCCCGCTTGAACC |
| | | AGATGTCGGCCCTGGAGTTCTACTCCT |
| | | ACAAAAAGTCAGGCATCACCGAGGTG |
| | | TGCAGAGAAGAGGCCCGCCGCGCCCT |
| | | CAAGGACGGTGTGGCAACGGGAGGCC |
| | | ATGCTGTGTCCCGAGGAAGTGCAAAG |
| | | CTGAGATGGTTGGTGGAGCGGGGATA |
| | | CCTGCAGCCCTATGGAAAGGTCATTGA |
| | | TCTTGGATGTGGCAGAGGGGGCTGGA |
| | | GTTACTACGTCGCCACCATCCGCAAAG |
| | | TTCAAGAAGTGAAAGGATACACAAAA |
| | | GGAGGCCCTGGTCATGAAGAACCCGT |
| | | GTTGGTGCAAAGCTATGGGTGGAACAT |
| | | AGTCCGTCTTAAGAGTGGGGTGGACGT |
| | | CTTTCATATGGCGGCTGAGCCGTGTGA |
| | | CACGTTGCTGTGTGACATAGGTGAGTC |
| | | ATCATCTAGTCCTGAAGTGGAAGAAGC |
| | | ACGGACGCTCAGAGTCCTCTCCATGGT |
| | | GGGGGATTGGCTTGAAAAAAGACCAG |
| | | GAGCCTTTTGTATAAAAGTGTTGTGCC |
| | | CATACACCAGCACTATGATGGAAACCC |
| | | TGGAGCGACTGCAGCGTAGGTATGGG |
| | | GGAGGACTGGTCAGAGTGCCACTCTCC |
| | | CGCAACTCTACACATGAGATGTACTGG |
| | | GTCTCTGGAGCGAAAAGCAACACCAT |
| | | AAAAAGTGTGTCCACCACGAGCCAGC |
| | | TCCTCTTGGGGCGCATGGACGGGCCTA |
| | | GGAGGCCAGTGAAATATGAGGAGGAT |
| | | GTGAATCTCGGCTCTGGCACGCGGGCT |
| | | GTGGTAAGCTGCGCTGAAGCTCCCAAC |
| | | ATGAAGATCATTGGTAACCGCATTGAA |
| | | AGGATCCGCAGTGAGCACGCGGAAAC |
| | | GTGGTTCTTTGACGAGAACCACCCATA |
| | | TAGGACATGGGCTTACCATGGAAGCTA |
| | | TGAGGCCCCCACACAAGGGTCAGCGT |
| | | CCTCTCTAATAAACGGGGTTGTCAGGC |
| | | TCCTGTCAAAACCCTGGGATGTGGTGA |
| | | CTGGAGTCACAGGAATAGCCATGACC |
| | | GACACCACACCGTATGGTCAGCAAAG |
| | | AGTTTTCAAGGAAAAAGTGGACACTA |
| | | GGGTGCCAGACCCCCAAGAAGGCACT |
| | | CGTCAGGTTATGAGCATGGTCTCTTCC |
| | | TGGTTGTGGAAAGAGCTAGGCAAACA |
| | | CAAACGGCCACGAGTCTGCACCAAAG |
| | | AAGAGTTCATCAACAAGGTTCGTAGCA |
| | | ATGCAGCATTAGGGGCAATATTTGAAG |
| | | AGGAAAAAGAGTGGAAGACTGCAGTG |
| | | GAAGCTGTGAACGATCCAAGGTTCTGG |
| | | GCTCTAGTGGACAAGGAAAGAGAGCA |
| | | CCACCTGAGAGGAGAGTGCCAGAGCT |
| | | GTGTGTACAACATGATGGGAAAAAGA |
| | | GAAAAGAAACAAGGGGAATTTGGAAA |
| | | GGCCAAGGGCAGCCGCGCCATCTGGT |
| | | ATATGTGGCTAGGGGCTAGATTTCTAG |
| | | AGTTCGAAGCCCTTGGATTCTTGAACG |
| | | AGGATCACTGGATGGGGAGAGAGAAC |
| | | TCAGGAGGTGGTGTTGAAGGGCTGGG |
| | | ATTACAAAGACTCGGATATGTCCTAGA |
| | | AGAGATGAGTCGTATACCAGGAGGAA |
| | | GGATGTATGCAGATGACACTGCTGGCT |
| | | GGGACACCCGCATTAGCAGGTTTGATC |
| | | TGGAGAATGAAGCTCTAATCACCAACC |
| | | AAATGGAGAAAGGGCACAGGGCCTTG |
| | | GCATTGGCCATAATCAAGTACACATAC |
| | | CAAAACAAAGTGGTAAAGGTCCTTAG |
| | | ACCAGCTGAAAAAGGGAAAACAGTTA |
| | | TGGACATTATTTCGAGACAAGACCAAA |
| | | GGGGGAGCGGACAAGTTGTCACTTAC |
| | | GCTCTTAACACATTTACCAACCTAGTG |
| | | GTGCAACTCATTCGGAATATGGAGGCT |
| | | GAGGAAGTTCTAGAGATGCAAGACTT |
| | | GTGGCTGCTGCGGAGGTCAGAGAAAG |
| | | TGACCAACTGGTTGCAGAGCAACGGA |
| | | TGGGATAGGCTCAAACGAATGGCAGT |
| | | CAGTGGAGATGATTGCGTTGTGAAGCC |
| | | AATTGATGATAGGTTTGCACATGCCCT |
| | | CAGGTTCTTGAATGATATGGGAAAAGT |
| | | TAGGAAGGACACACAAGAGTGGAAAC |
| | | CCTCAACTGGATGGGACAACTGGGAA |
| | | GAAGTTCCGTTTTGCTCCCACCACTTC |
| | | AACAAGCTCCATCTCAAGGACGGGAG |
| | | GTCCATTGTGGTTCCCTGCCGCCACCA |
| | | AGATGAACTGATTGGCCGGGCCCGCGT |
| | | CTCTCCAGGGGCGGGATGGAGCATCC |
| | | GGGAGACTGCTTGCCTAGCAAAATCAT |
| | | ATGCGCAAATGTGGCAGCTCCTTTATT |
| | | TCCACAGAAGGGACCTCCGACTGATG |
| | | GCCAATGCCATTTGTTCATCTGTGCCA |
| | | GTTGACTGGGTTCCAACTGGGAGAACT |
| | | ACCTGGTCAATCCATGGAAAGGGAGA |
| | | ATGGATGACCACTGAAGACATGCTTGT |
| | | GGTGTGGAACAGAGTGTGGATTGAGG |
| | | AGAACGACCACATGGAAGACAAGACC |
| | | CCAGTTACGAAATGGACAGACATTCCC |
| | | TATTTGGGAAAAAGGGAAGACTTGTG |
| | | GTGTGGATCTCTCATAGGGCACAGACC |
| | | GCGCACCACCTGGGCTGAGAACATTA |
| | | AAAACACAGTCAACATGGTGCGCAGG |
| | | ATCATAGGTGATGAAGAAAAGTACAT |
| | | GGACTACCTATCCACCCAAGTTCGCTA |
| | | CTTGGGTGAAGAAGGGTCTACACCTGG |
| | | AGTGCTGTAAGCACCAATCTTAATGTT |
| | | GTCAGGCCTGCTAGTCAGCCACAGCTT |
| | | GGGGAAAGCTGTGCAGCCTGTGACCC |
| | | CCCCAGGAGAAGCTGGGAAACCAAGC |
| | | CTATAGTCAGGCCGAGAACGCCATGG |
| | | CACGGAAGAAGCCATGCTGCCTGTGA |
| | | GCCCCTCAGAGGACACTGAGTCAAAA |
| | | AACCCCACGCGCTTGGAGGCGCAGGA |
| | | TGGGAAAAGAAGGTGGCGACCTTCCC |
| | | CACCCTTCAATCTGGGGCCTGAACTGG |
| | | AGATCAGCTGTGGATCTCCAGAAGAG |
| | | GGACTAGTGGTTAGAGGA |
| 131 | ZIKV envelope amino acid | NGSISLMCLALGGVLIFLSTAVSA |
| | sequence | |
| 132 | PreScission Protease | LEVLFNGPG |
| | cleavage site amino acid | |
| | sequence | |
| 133 | Hexahistidine motif amino | HHHHHH |
| | acid sequence | |
| 134 | VL CDR1 (IMGT) | QSISSX |
| | | X is Y or H |
| 135 | VL CDR2 (IMGT) | X1X2S |
| | | X1 is A or Q |
| | | X2 is A or D |
| 136 | VL CDR3 (IMGT) | QQX1YSTPX2T |
| | | X1 is T or S |
| | | X2 is L, Y, or W |
| 137 | VL ABR1 (Paratome) | QSISSX1LN |
| | | X1 is Y or H |
| 138 | VL ABR2 (Paratome) | X1LIYAASSLQS |
| | | X1 is F or L |
| 139 | VL ABR3 (Paratome) | QQX1YSTPX2 |
| | | X1 is T or S |
| | | X2 is L, Y or W |
| 140 | Synthetic Sequence | LALAPG |
| 141 | Example of thrombin | LVPRGSP |
| | cleavage site | |
| 142 | Example of cleavage site | ENLYFQX |
| | recognized by Tobacco | X is G or S |
| | Etch Virus (TEV) protease | |
| 143 | VH CDR1 | GFTVSSNY |
| 144 | VH CDR2 | IYSGGST |
| 145 | VH CDR3 | ARDRRGFDY |
| 146 | VH CDR3 | ARWGGKRGGAFDI |
| 147 | VH CDR3 | ARLIAAAGDY |
| 148 | VH CDR3 | ARGPVQLERRPLGAFDI |
| 149 | VH ABR1 | FTVSSNYMS |
| 150 | VH ABR2 | WVSVIYSGGSTYYA |
| 151 | VH ABR3 | ARDRRGFDY |
| 152 | VH ABR3 | ARWGGKRGGAFDI |
| 153 | VH ABR3 | ARLIAAAGDY |
| 154 | VH ABR3 | ARGPVQLERRPLGAFDI |
| 155 | CDR3 | CARDRRGFDYW |
| 156 | CDR3 | CARGPVQLERRPLGAFDIW |
| 157 | CDR3 | CARWGGKRGGAFDIW |
| 158 | CDR3 | CARLIAAAGDYW |
| 159 | CDR3 | CQQTYSTPLTF |
| 160 | CDR3 | CQAWDSSTV |
| 161 | CDR3 | CQQSYSTPYTF |
| 162 | CDR3 | CQQSYSTPWTF |
| 163 | Synthetic Peptide | NGSISLMCLALGGVLIFLSTAVSADVGCS |
| | | VDFSK |
| 164 | Synthetic Peptide | LVRSMVTA |
| 165 | Synthetic Peptide | LVRSMVTALEVLFNGPGHHHHHH |
| | (cleavage site and | |
| | hexahistidine motif) | |
| 166 | Synthetic NS1 polypeptide | NGSISLMCLALGGVLIFLS |
| | (comprising a fragment of | TAVSADVGCSVDFSKKETRC |
| | a Zika virus evelope | GTGVFVYNDVEAWRDRYKYH |
| | protein, and a Zika virus | PDSPRRLAAAVKQAWEDGIC |
| | NS1 polypeptide) | GISSVSRMENIMWRSVEGEL |
| | | NAILEENGVQLTVVVGSVKN |
| | | PMWRGPQRLPVPVNELPHGW |
| | | KAWGKSYFVRAAKTNNSFVV |
| | | DGDTLKECPLKHRAWNSFLV |
| | | EDHGFGVFHTSVWLKVREDY |
| | | SLECDPAVIGTAVKGKEAVH |
| | | SDLGYWIESEKNDTWRLKRA |
| | | HLIEMKTCEWPKSHTLWTDG |
| | | IEESDLIIPKSLAGPLSHHN |
| | | TREGYRTQMKGPWHSEELEI |
| | | RFEECPGTKVHVEETCGTRG |
| | | PSLRSTTASGRVIEEWCCRE |
| | | CTMPPLSFRAKDGCWYGMEI |
| | | RPRKEPESNLVRSMVTA |
| 167 | Synthetic NS1 polypeptide | MNGSISLMCLALGGVLIFLS |
| | (comprising a fragment of | TAVSADVGCSVDFSKKETRC |
| | a Zika virus evelope | GTGVFVYNDVEAWRDRYKYH |
| | protein, a Zika virus NS1 | PDSPRRLAAAVKQAWEDGIC |
| | polypeptide, a cleavage | GISSVSRMENIMWRSVEGEL |
| | site, and a hexahistidine | NAILEENGVQLTVVVGSVKN |
| | motif) | PMWRGPQRLPVPVNELPHGW |
| | | KAWGKSYFVRAAKTNNSFVV |
| | | DGDTLKECPLKHRAWNSFLV |
| | | EDHGFGVFHTSVWLKVREDY |
| | | SLECDPAVIGTAVKGKEAVH |
| | | SDLGYWIESEKNDTWRLKRA |
| | | HLIEMKTCEWPKSHTLWTDG |
| | | IEESDLIIPKSLAGPLSHHN |
| | | TREGYRTQMKGPWHSEELEI |
| | | RFEECPGTKVHVEETCGTRG |
| | | PSLRSTTASGRVIEEWCCRE |
| | | CTMPPLSFRAKDGCWYGMEI |
| | | RPRKEPESNLVRSMVTALEV |
| | | LFQGPGHHHHHH |
| 168 | PRVABC59 virus NS1 | gatgtggggtgctcggtggacttctcaaagaaggagacgagat |
| | nucleotide sequence | gcggtacaggggtgttcgtctataacgacgttgaagcctggag |
| | | ggacaggtacaagtaccatcctgactccccccgtagattggca |
| | | gcagcagtcaagcaagcctgggaagatggtatctgcgggatct |
| | | cctctgtttcaagaatggaaaacatcatgtggagatcagtagaa |
| | | ggggagctcaacgcaatcctggaagagaatggagttcaactg |
| | | acggtcgttgtgggatctgtaaaaaaccccatgtggagaggtc |
| | | cacagagattgcccgtgcctgtgaacgagctgccccacggct |
| | | ggaaggcttgggggaaatcgtatttcgtcagagcagcaaagac |
| | | aaataacagctttgtcgtggatggtgacacactgaaggaatgcc |
| | | cactcaaacatagagcatggaacagctttcttgtggaggatcat |
| | | gggttcggggtatttcacactagtgtctggctcaaggttagaga |
| | | agattattcattagagtgtgatccagccgttattggaacagctgtt |
| | | aagggaaaggaggctgtacacagtgatctaggctactggattg |
| | | agagtgagaagaatgacacatggaggctgaagagggcccat |
| | | ctgatcgagatgaaaacatgtgaatggccaaagtcccacacatt |
| | | gtggacagatggaatagaagagagtgatctgatcatacccaag |
| | | tctttagctgggccactcagccatcacaataccagagagggcta |
| | | caggacccaaatgaaagggccatggcacagtgaagagcttga |
| | | aattcggtttgaggaatgcccaggcactaaggtccacgtggag |
| | | gaaacatgtggaacaagaggaccatctctgagatcaaccactg |
| | | caagcggaagggtgatcgaggaatggtgctgcagggagtgc |
| | | acaatgcccccactgtcgttccgggctaaagatggctgttggta |
| | | tggaatggagataaggcccaggaaagaaccagaaagcaactt |
| | | agtaaggtcaatggtgactgca |

### 6. EXAMPLES

### 6.1 Example 1: Human Antibodies Targeting Zika Virus NS1 Provide Protection Against Disease in a Mouse Model

Zika virus is a mosquito-borne flavivirus closely related to dengue virus that can cause severe disease in humans, including microcephaly in newborns and Guillain-Barré syndrome in adults. Specific treatments and vaccines for Zika virus are not currently available. Here, four monoclonal antibodies (mAbs) from an infected patient that target the non-structural protein NS1 were isolated and characterized. While these antibodies are non-neutralizing, NS1-specific mAbs can engage FcγR without inducing antibody dependent enhancement (ADE) of infection in vitro. Moreover, the results demonstrate that mAb AA12 has protective efficacy against lethal challenges of African and Asian lineage strains of Zika virus in Stat2−/− mice. Protection is Fc-dependent, as a mutated antibody unable to activate known Fc effector functions or complement is not protective in vivo. This study highlights the importance of the ZIKV NS1 protein as a potential vaccine.

### 6.1.1 Introduction

This study assesses if NS1-specific monoclonal antibodies can provide protection in a murine model and whether this protection relies upon Fcγ-receptor effector functions. Using a well-established protocol for the generation of fully human monoclonal antibodies³³, ZIKV specific mAbs were isolated from the plasmablast compartment of a patient recently infected by ZIKV. The variable regions of the heavy and light chains of isolated plasmablasts were then sequenced, cloned, and recombinantly expressed. The characteristics of four NS1-specific antibodies found in an individual with symptomatic ZIKV infection are reported herein. The data demonstrate that while NS1-specific mAbs do not neutralize virus in vitro, they can confer FcγR-mediated protection in vivo in a murine challenge model, which highlights the importance of NS1 epitopes in vaccine development.

### 6.1.2 Materials and Methods

Cells and Viruses: Human embryonic kidney (HEK) 293 T cells (American Type Culture Collection; ATCC Cat. No. CRL-1573) and African green monkey kidney cells (Vero) were grown in Dulbecco modified Eagle medium (DMEM, Gibco) supplemented with 10% fetal bovine serum (FBS) (Hyclone) and antibiotics (100 units/ml penicillin-100 μg/ml streptomycin [Pen-Strep]; Gibco). Human embryonic kidney Expi293F cells (Gibco) were grown in Expi293 expression media. MR766 virus (Rhesus/1947/Uganda BEI NR-50065), PRVABC59 virus (2015/Puerto Rico BEI NR-50684) and PAN/2015 virus (H/PAN/2015/CDC-259359) were obtained from BEI resources. Fcγ-receptor expressing K562 cells were obtained through ATCC (Cat #CCL-243). Pan-flavivirus antibody 4G2 was obtained through ATCC D1-4G2-4-15 (ATCC® HB-112™). ZIKV were propagated in Vero cells in 1× Minimum Essential Medium (MEM); after 72 hours post infection (hpi), cell culture supernatants were harvested, aliquoted and stored at −80° C. until use.

Human plasmablast isolation: Plasmablasts were isolated at approximately two weeks after onset of symptoms. Plasmablasts (CD19⁺/CD3⁻/CD20⁻/CD38^{high}/CD27^{high}) were isolated and monoclonal antibodies were generated as previously described³³ in accordance with the Icahn School of Medicine at Mount Sinai Institutional Review Board. Briefly, Ficoll density (GE Healthcare) centrifugation was performed to isolate the buffy coat, and peripheral blood mononuclear cells (PBMCs) were single-cell sorted onto freshly made catch buffer (5 mL of RNAse water/50 μL of 1 M Tris pH 8/125 μL Rnasin) on 96-well plates using a BD FACSARIA III. Reverse transcription reactions were performed to generate cDNA as previously described^{33,34}. Two nested PCRs incorporating IgG-, IgA-, IgM-, kappa- and lambda-specific primers were performed on the cDNA to amplify heavy and light chains. IMGT/V-QUEST software (The International Immunogenetics Information System) was used to view productive immunoglobulin sequence rearrangements. Sixteen Zika virus antibodies were isolated from one patient, four of which are NS1-specific and further characterized in this study. All four NS1-specific antibodies, AA12, EB9, FC12 and GB5 have the VH3-53/JH3 heavy chain and are of the IgG1 isotype (Table 10).

Recombinant human antibodies: The human heavy (VH) and kappa (VK) variable regions of the antibodies AA12, FC12, EB9 and GB5 were amplified by PCR and cloned into human IgG1 and kappa mammalian expression vectors, respectively (pFUESss-CHIg-hIgG1 and pFUESss-CLIg-hK Invivogen). The L234A, L235A, and P329G (LALAPG) mutations in the IgG1 heavy chain were introduced by site-directed mutagenesis. The variable region of the heavy chain of AA12 was then cloned into the modified expression vector to make AA12-LALAPG. Wild-type or LALAPG antibodies (mutated IgG1 heavy chain with wild-type kappa chain) were expressed and purified as previously described³³.

Recombinant ZIKV NS1: The NS1 gene segments from MR766 virus (Rhesus/1947/Uganda Accession: NC_012432, SEQ ID NO: 129) and PRVABC59 virus (2015/Puerto Rico Accession: KU501215, SEQ ID NO: 130) were human codon optimized using Integrated DNA Technologies Codon Optimization Tool (http://www.idtdna.com/CodonOpt) and modified to contain a C-terminal hexahistidine-tag. NS1 gene segments were subcloned into the expression plasmid pCAGGS using restriction endonucleases NotI and XhoI (New England Biosciences) and inserted into the digested plasmid by homologous recombination (In-Fusion, Takara) to construct pCAGGS-MR766-NS1 and pCAGGS-PRVABC59-NS1. To generate recombinant NS1 proteins, 30 mL of Expi293 cells were transfected with 30 ug of pCAGGS-MR766-NS1 or pCAGGS-PRVABC59-NS1 plasmids and 81 uL of expifectamine reagent as per manufacturer's instructions. After 120 hours, supernatants were cleared by low-speed centrifugation and incubated with Ni-NTA resin overnight at 4° C. The resin-supernatant mixture was then passed over 10 mL polypropylene columns (Qiagen). The retained resin was washed four times with 15 ml of washing buffer (50 mM Na₂HCO₃, 300 mM NaCl, 20 mM imidazole, pH 8) and protein was eluted with elution buffer (50 mM Na₂HCO₃, 300 mM NaCl, 300 mM imidazole, pH 8). The eluate was concentrated using Amicon Ultracell (Millipore) centrifugation units with a cut-off of 10 kDa and buffer was changed to phosphate buffered saline (PBS) of pH 7.4. Protein concentration was quantified using Pierce Bicinchoninic Acid Protein Assay Kit (Thermo Scientific) with a bovine serum albumin standard curve. Purified soluble NS1 proteins were resolved in a reducing and denatured SDS-PAGE gel (in monomeric forms of around 45 kDa and homodimeric forms of around 90 kDa) and visualized using SimplyBlue SafeStain (Thermofisher, Inc.).

Enzyme-linked immunosorbent assay (ELISA): Plates were coated with recombinant ZIKV NS1 at 2 μg/mL in pH 9.41 carbonate buffer overnight at 4° C. After blocking in 5% non-fat (NF) milk for 1 hour, mAbs were incubated at a starting concentration of 10 μg/mL and serially diluted 3-fold and incubated 2 hours at room temperature. Horseradish peroxidase (HRP)-conjugated goat anti-human IgG antibody (AP504P; Millipore Sigma) was used to detect binding of the mAbs, followed by development with HRP substrate (Sigmafast OPD; Sigma-Aldrich). Reactions were stopped by addition of 3M HCl and absorbance was measured at 490 nm on a microplate spectrophotometer (BioRad). Experiments were performed in duplicates and repeated twice. Graphpad Prism 5 was used to visualized the mean values and the standard error of the mean (SEM) and generate a non-linear regression curve.

Immunofluorescence: Vero cells were infected with ZIKV MR766, ZIKV PRVABC59 or dengue virus type 3, Philippines/H87/1956 with a multiplicity of infection (MOI) of 1. After 24 hours post infection, the monolayer of Vero cells was fixed with 0.5% of paraformaldehyde (PFA)/1×PBS. Cells were blocked with 5% nonfat milk for 30 minutes at room temperature. Blocking buffer was then discarded and NS1-specific mAbs were added at a concentration of 5 μg/mL in nonfat milk. Primary antibodies were incubated for 2 hours at room temperature after which the monolayer was washed three times with 1×PBS. An anti-human or anti-mouse IgG secondary antibody conjugated to Alexa Fluor 488 (ThermoFisher) diluted (1:000) in nonfat milk was added to the monolayer and incubated in the dark at RT for 1 hour. The monolayer was then washed three times with 1×PBS. Cells were then visualized using an inverted fluorescent microscope (Olympus IX70).

Microneutralization (MN) Assay: To assess the in vitro neutralizing activity of the mAbs we performed a MN assay. Three-fold serially diluted antibody (starting at 100 μg/mL) in serum-free minimum essential medium (MEM) was mixed with an equal volume of virus (100 TCID₅₀) and incubated for 1 hour at room temperature. Monolayers of Vero cells were washed once with PBS and the virus/antibody mixture was added to the cells and incubated for 1 hour at 37° C. After the infection, the virus/antibody mixture was removed and replaced with serum-free MEM with antibody added at the appropriate dilution. The cells were then incubated at 37° C. for 72 hours. Cytopathic effect (CPE) was scored at three days post infection and IC₅₀ was quantified by the Reed and Muench method.

Antibody-dependent effector functions: For experiments involving infected cells, Vero cells were seeded on 96-well flat white-bottom plates (Corning) and infected after 24 hours with ZIKV (MR766 or PRVABC59) at an MOI of 0.01. For experiments involving transfected cells, HEK 293T cells were seeded onto poly-D-lysine coated 96-well flat white-bottom plates (Corning). After 24 hours, the cells were transfected with 100 ng per well of expression plasmid encoding NS1 from MR766 or PRVABC59. At 16 hours post transfection or 40 hours post infection, the medium was removed and 25 μL of assay buffer (RPMI 1640 with 4% low-IgG FBS) was added to each well. Then mAbs were added in a volume of 25 μL at 30 μg/mL and serially diluted fourfold in assay buffer (in duplicate). The mAbs were then incubated with the transfected or infected cells for 30 minutes at 37° C. Genetically modified Jurkat cells expressing the human FcγRIIIa with a luciferase reporter gene under the transcriptional control of nuclear factor-activated T cells (NFAT) promoter were added at 7.5×10⁴ cells at 25 μL per well, which is approximately a 1:2 ratio of target cells to effector cells, followed by incubation for another 6 h at 37° C. (Promega). Bio-Glo Luciferase assay reagent was added after 6 h and luminescence was quantified using a plate reader. Fold induction was measured in relative light units and calculated by subtracting background signal from wells without effector cells then dividing wells with antibody by with no antibody added. Specifically, fold induction was calculated as follows: (RLU_{induced}−RLU_{background})/(RLU_{uninduced}−RLU_{background}). The mean values and SEM were reported and a nonlinear regression curve was generated using GraphPad Prism 5.

Alternatively, we measured antibody-dependent effector functions by detecting activation of primary human natural killer (NK) cells (expression of CD107a) as previously described⁴⁷. Briefly, Vero cells were seeded at 2×10⁴ cells/well in 96-well cell culture-treated plates and infected with PRVABC59 ZIKV with an MOI of 0.5 (adjusted for cell growth overnight). At 48 hpi, growth media from infected Vero cells were aspirated and incubated with dilutions (50 μL total volume) of NS1-specific mAbs (diluted in 1× Iscove's media supplemented with 10% FBS) starting at 20 μg/mL, serially diluted 3-fold and incubated at 37° C., CO₂ for 1.5 hours. A human mAb specific for the influenza B virus hemagglutinin, II2C7, was used as an irrelevant mAb and a group containing no mAb was used as background control. Human NK cells were isolated (Lymphoprep; Stemcell Technologies, Inc.) from buffy coat donors (San Diego Blood Center) through negative selection (EasySep Human NK cell isolation kit; Stemcell Technologies, Inc.) and 8×10⁵ CD56⁺ cells/well (in 50 μL 1× Iscoves's media supplemented with 10% FBS; effector cells to target ratio of 2) were subsequently added to the ZIKV-infected Vero cells and mAb mixture (total volume of 100 μL). Cells were incubated for 3 hours at 37° C., CO₂. Cells were then washed with wash buffer (1×PBS/1% BSA) and stained with CD56-FITC (Clone B159 BD Biosciences; 5 μL per 1e6 cells) and CD107a-PE (Clone H4A3 BD Biosciences; 20 μL per 1e6 cells) for 15 minutes at 4° C. (in the dark). Samples were then resolved in a BD FACS ARIA II flow cytometer sorter (BD Biosciences) and analyzed using FlowJo 10.5.0. Experiments were performed in duplicates and the means/standard error were graphed using GraphPad Prism 5.

K_{D} determination. Biolayer interferometry assays were performed with an Octet RED instrument (ForteBio, Inc.) to determine K_{D} values. Purified recombinant NS1 was loaded onto a Ni-NTA biosensor (ForteBio, Inc.) in kinetics buffer (1×PBS pH 7.4, 0.01% BSA, 0.002% Tween-20) for 3 min. To determine kₒₙ, association was measured for 3 min by exposing the sensors to seven concentrations of antibody diluted in kinetics buffer. To determine k_{off}, dissociation was measured for 3 min in kinetics buffer. K_{D} values were calculated as the ratios of k_{off} to kₒₙ. We used a 2:1 binding model to reflect two identical binding sites of homodimeric NS1 proteins. K1 and K2 reflect the kinetics constants of the first and seconding binding interaction between the mAb and a homodimeric NS1.

Antibody-dependent enhancement of infection: Enhancement of ZIKV infection was measured using a flow-cytometry-based assay¹⁴. Serial dilutions of purified monoclonal antibody were mixed with ZIKV (PRVABC59 MOI of 1) for 1 hour at 37° C. in RPMI 1640 media supplemented with 10% FBS, 2 mM L-glutamine, and antibiotics (100 units/ml penicillin-100 μg/ml streptomycin [Pen-Strep]; Gibco). The mixture was then added to K562 cells in 96-well U bottom plates. After two days, cells were fixed with 4% PFA/1×PBS, permeabilized with PBS containing 0.2% BSA and 0.05% saponin and stained with 4G2 pan-flavivirus anti-envelope antibody (1 μg/mL) for 1 h at RT. Cells were then incubated with goat anti-mouse IgG conjugated to phycoerythrin (1 μg/mL; Invitrogen) for 1 h at RT. The number of infected cells was determined by flow cytometry using a FACS Caliber and analyzed using FlowJo2 software version 10.1.r7. Area under the curve was calculated using GraphPad Prism.

Passive transfer studies: All animal experiments were performed in an animal biosafety level 2 plus facility in accordance with the Icahn School of Medicine at Mount Sinai and the University of California, Riverside Institutional Animal Care and Use Committees (IACUC). Groups of 5 to 9 male and female B6.129-Stat2^{-/-} mice (kindly provided by Dr. Christian Schindler) were passively transferred with 20 mg/kg or 10 mg/kg AA12, or 10 mg/kg AA12-LALAPG antibody intraperitoneally. Control mice received the human anti-influenza antibody CR911454 at a dose of 10 mg/kg. Mice were challenged intradermally with 10 LD50 ZIKV MR766 or retro-orbitally with 500 PFU of ZIKV PAN/2015 and evaluated for 14 days. Mice were monitored daily for weight and clinical signs. Clinical scoring was conducted using the pre-defined criteria with a maximum possible score of 7: impact on walking, unresponsiveness, left hind leg paralyzed, right hind leg paralyzed, left front leg paralyzed, and right front leg paralyzed. Deceased animals were given a score of 7^{14,55}. Animals that showed more than 25% weight loss or full paralysis were humanely euthanized. Experiments were conducted with a balanced amount of male and female mice and with an even distribution of mice from different litters whenever possible. To determine statistical significance, the Mantel-Cox and Gehan-Breslow-Wilcoxon tests were used for survival curves and a multiple t-test and the Holm-Sidak method utilized to analyze the weight curve and clinical scores. Asterisk(s) on graphs indicates statistical significance (*=p value<0.05 and **=p value<0.005) of a group compared to the IgG control group.

Viral titers: Tissue samples were harvested from infected mice, placed in PBS, and homogenized using ceramic beads. ZIKV quantification was conducted via plaque assay. Briefly, Vero cells were plated in 24 well plates and infected after 24 hours with dilutions of virus made in serum-free 1×MEM medium. Infectious medium was aspirated and 600 μL of methylcellulose agar equivalent medium was added to each well. At day 4 post-infection plates were fixed with 4% PFA for 1 hour at RT, washed, and stained with 4G2 in milk at 5 μg/mL. Secondary anti-mouse HRP was then added at 1:5000 in milk and the assay was resolved with TrueBlue Peroxidase Substrate (VWR). Plaques were manually counted and plaque forming units (PFU) per mL of homogenized tissue was calculated.

Complement ELISA: A mouse complement C3 ELISA kit (Abcam: ab157711) was used as per the manufacturer's instructions to measure C3 levels in the serum of infected or naïve mice. Briefly, serum was diluted 1:50,000 and pipetted into designated wells. In parallel, a standard curve was generated from known concentrations of C3. The plate was incubated for twenty minutes, washed, and a 1× enzyme-antibody conjugate was added. After incubation for twenty minutes and additional washing, TMB substrate was added and absorbance was measured at 450 nm on a microplate spectrophotometer (BioRad).

Study approval: An IRB approved written informed consent was obtained from the patient prior to study participation. No further demographic data are included here in order to protect the participant's privacy.

Statistical analysis: Results from multiple experiments are presented as mean #SEM. Student's t-tests were used to test for statistical differences between mean values. Data were analyzed with GraphPad Prism 7 software and p values of <0.05 were considered statistically significant.

### 6.1.3 Results

### Antibodies Targeting the Zika Virus NS1 Protein are Induced in Humans

To investigate the antibody response to ZIKV infection, plasma and isolated peripheral blood mononuclear cells (PBMCs) was obtained from a patient who was infected with ZIKV while traveling in Central America. The patient was likely not pre-exposed to dengue based on history and past travel. Blood was collected ten days after the patient tested positive for ZIKV RNA by RT-PCR. A published protocol was adapted to isolate Zika virus-specific mAbs from the plasmablast compartment of the infected patient^{33,34}.

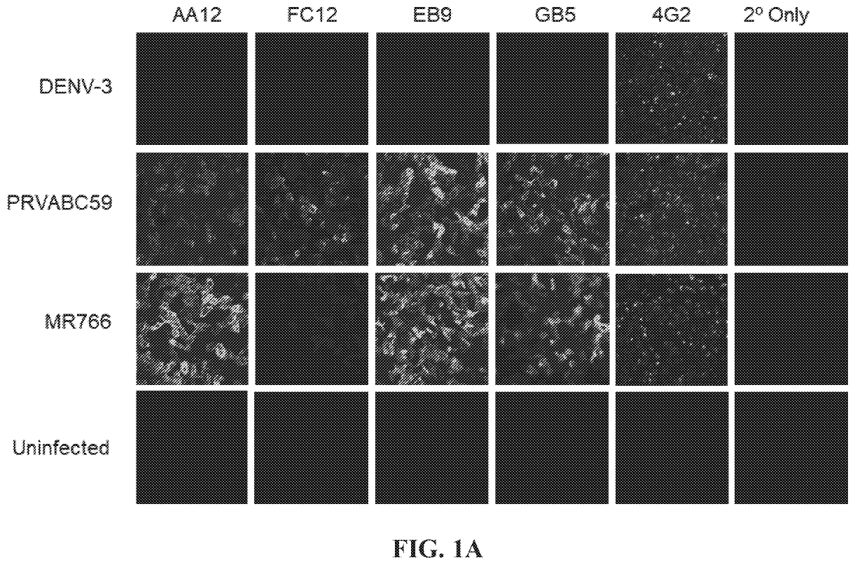

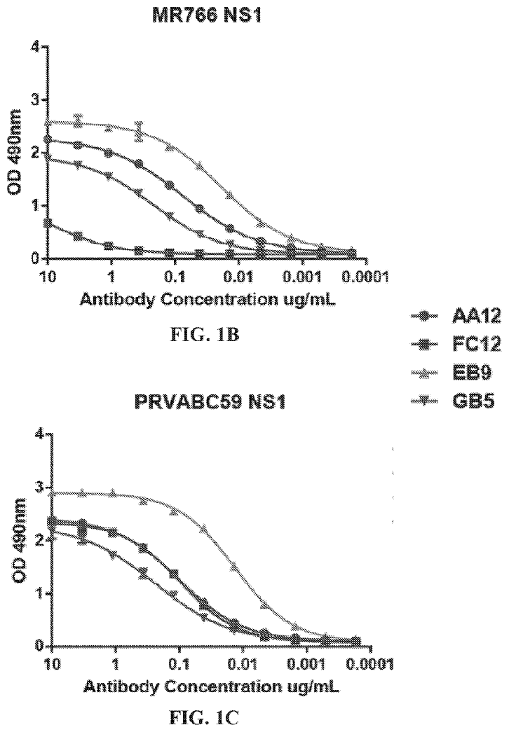

Following single cell sorting of B cells, the variable regions of the immunoglobulins were sequenced, cloned into a human IgG1 expression vector, and subsequently expressed in HEK 293F cells as previously described^{33,34}. The mAbs were then initially screened for reactivity to ZIKV-infected Vero cells by immunofluorescence. Vero cells were infected with one of two strains of Zika virus: either the African lineage MR766 which was isolated in Uganda from a rhesus macaque in 1947 and subsequently passaged in mice, or the Asian lineage PRVABC59, which was isolated in Puerto Rico from a human patient in 2015 and is representative of the current circulating strain. Additionally, a DENV-3 isolate from the Philippines was tested to determine if the antibodies cross-reacted with other flaviviruses. Three of the mAbs (AA12, EB9, and GB5) bound cells infected by both MR766 and PRVABC59 and one antibody (FC12) that bound cells infected by only PRVABC59 (FIG. 1A). None of the antibodies cross-reacted with DENV-3 infected cells. Next, it was tested whether the antibodies bound by ELISA to recombinant NS1 protein (FIGS. 1B, 1C). NS1 from both MR766 and PRVABC59 strains of ZIKV were expressed and purified. As expected, three antibodies bound NS1 (AA12, EB9, and GB5) from both strains of ZIKV while FC12 only bound NS1 from the recent PRVABC59 ZIKV isolate. All mAbs were originally found to be of the IgG1 isotype and carried a low number of somatic mutations (Table 10). Neutralization activity was examined by microneutralization assays and none of the NS1-specific mAbs exhibited neutralization activity against either PRVABC59 or MR766 (Table 10). The binding affinities of each antibody to PRVABC59 and MR766 were then determined using biolayer interferometry (Table 9). The binding constants using biolayer interferometry (Table 9) were consistent with observed ELISA data (FIGS. 1B, 1C). As expected, FC12 only bound NS1 from the recent PRVABC59 ZIKV isolate while AA12, GB5 and EB9 both bound potently to PRVABC59 or MR766.

### NS1-Specific Antibodies Activate Fc-FcγR Mediated Effector Functions In Vitro

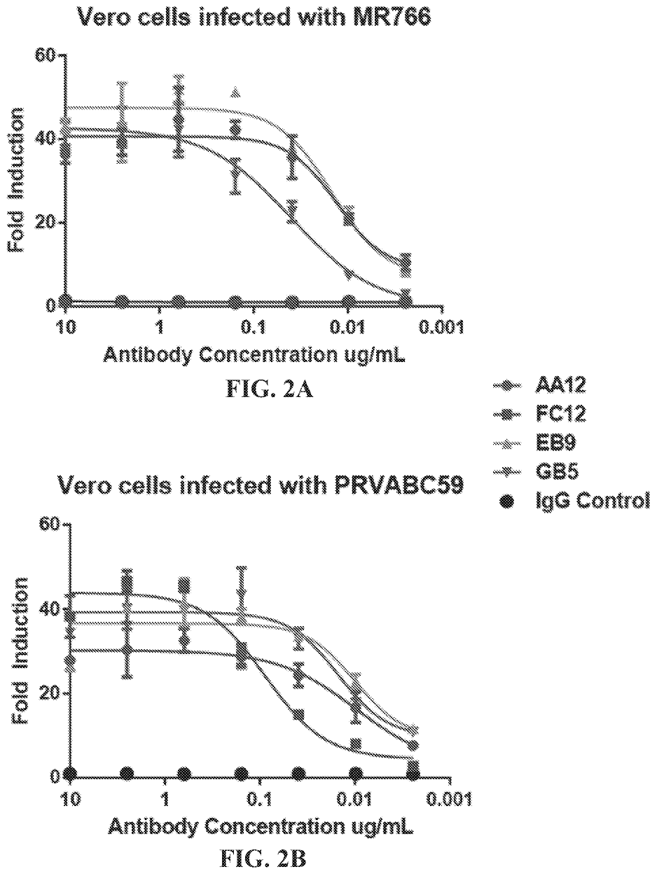

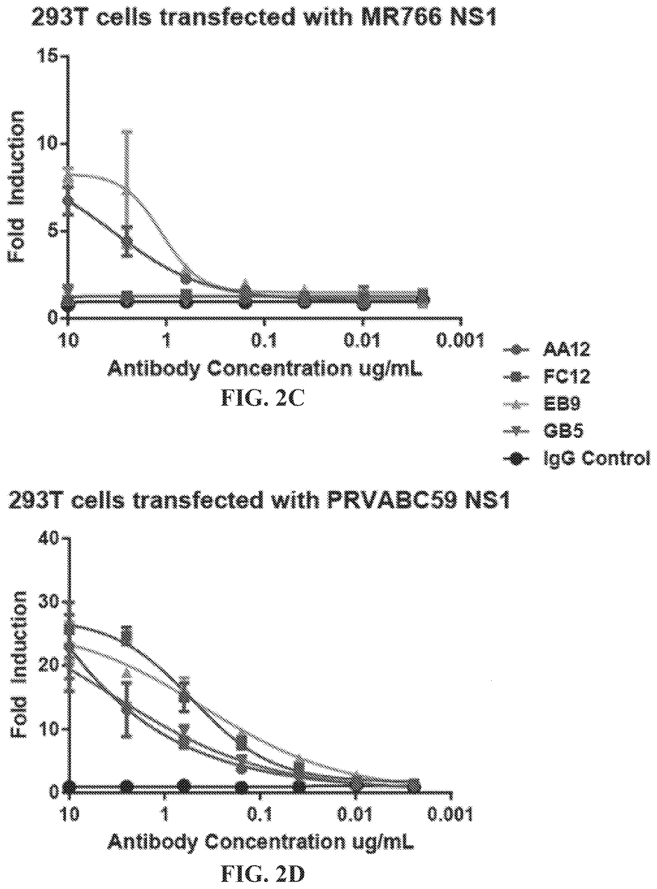

Next, the ability of NS1-specific mAbs to engage in Fcγ-mediated effector functions was evaluated. To model the activation of ADCC, the following was used a genetically modified Jurkat cell line expressing human FcγRIIIa and a luciferase reporter under a nuclear factor of activated T-cells (NFAT) promoter as a surrogate to examine the ability of these mAbs to engage and then activate Fcγ-mediated effector functions. First, Vero cells were infected with either MR766 or PRVABC59 ZIKV. Next, NS1-specific mAbs at concentrations ranging from 10 to 0.002 μg/mL were added. Consistent with the earlier ELISA results, three mAbs (AA12, EB9, and GB5) induced effector functions on both MR766 and PRVABC59 ZIKV infected cells and one antibody (FC12) induced effector functions only on PRVABC59 ZIKV (FIG. 2A, 2B).

Next, it was determined whether transfection of NS1 is sufficient to activate Fc-FcγR effector functions by transfecting HEK 293T cells with an expression (pCAGGS) plasmid expressing NS1 from either MR766 or PRVABC59 ZIKV. After incubation with the same mAbs at the same concentrations, it was found that only two of the mAbs (AA12 and EB9) induced effector functions on both MR766 and PRVABC59 ZIKV transfected cells while the remaining two antibodies induced effector functions on cells transfected by NS1 from PRVABC59 ZIKV (FIG. 2C, 2D). The discrepancy observed in antibody GB5 may be due to a limited transfection efficiency of NS1 compared to NS1 being expressed from infected cells. Alternatively, the conformation of transfected NS1 from the PRVABC59 ZIKV isolate may limit the GB5 epitope as compared to the NS1 protein expressed by infected cells. Nevertheless, it can be concluded that the surface NS1 protein in the absence of viral infection is sufficient to activate Fcγ-mediated effector functions induced by NS1-specific mAbs.

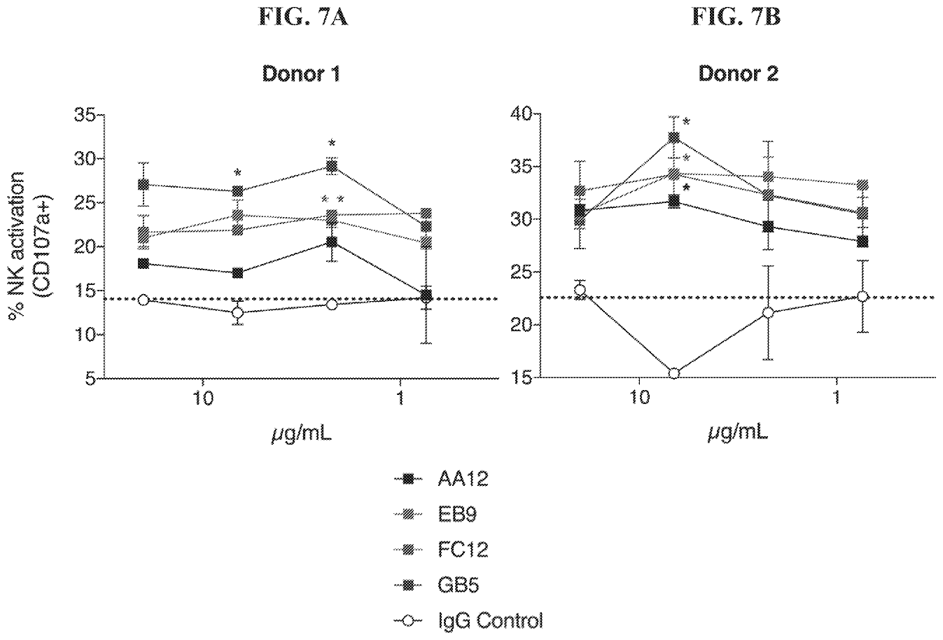

Lastly, it was demonstrated that NS1-specific mAbs can direct the activation of human primary NK cells. Here, Vero cells were infected with PRVABC59 ZIKV at an MOI of 0.5. At 48 hours post infection, dilutions of NS1-specific IgG1 mAbs, an irrelevant mAb, or no mAb (starting at 20 μg/mL) in combination with isolated primary human NK cells were incubated with the ZIKV-infected Vero cells. After three hours, activation of NK cells was measured as percent of CD56+ cells expressing CD107a. As shown in FIGS. 7A-7B, all NS1-specific mAbs can measurably activate CD107a expression of NK cells from two donors, ranging from 32% to 18% at 20 μg/mL. Of note, CD107a expression between the irrelevant IgG and baseline control (with no mAb added) groups correlated with each other at around 14.06% for donor 1 and 22.6% for donor 2.

### NS1-Specific Antibodies do not Enhance Zika or Dengue Virus Infection In Vitro

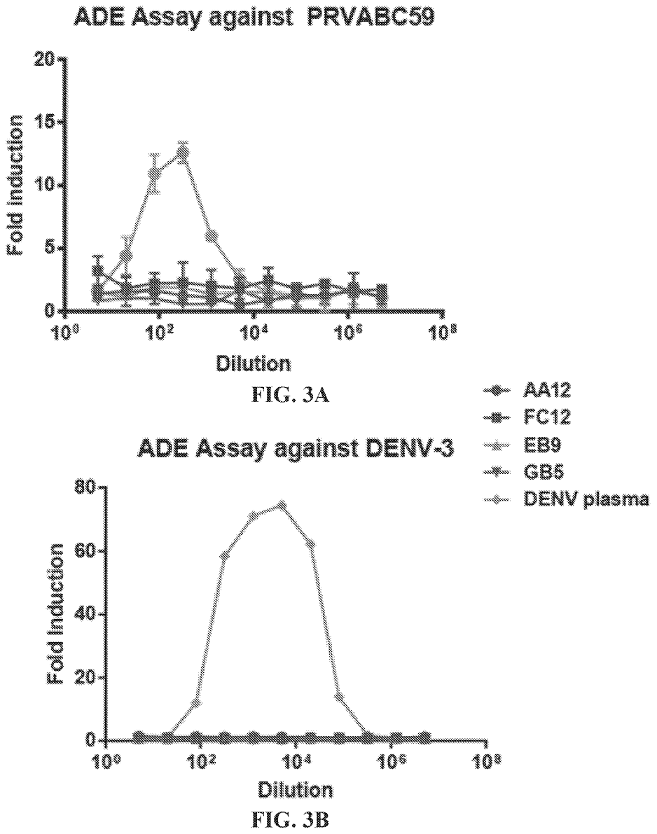

Next, it was assessed whether NS1-specific mAbs were able to enhance infection of target cells in vitro. ADE is commonly observed when an antibody that opsonizes but does not fully neutralize a virion facilitates infection of Fcγ-receptor bearing target cells. ADE was measured using a flow cytometry-based assay in which serial dilutions of monoclonal antibody or serum were mixed with PRVABC59 ZIKV and added to FcγR bearing K562 cells, which are typically non-permissible to ZIKV infection. After 48 hours, cells were fixed and stained for the envelope protein using murine 4G2 antibody and the number of infected cells was determined by flow cytometry. It was found that none of the NS1-specific mAbs enhance Zika infection in vitro (FIGS. 3A-3B). In contrast, a high level of ADE activity was observed when K562 cells were infected in the presence of DENV-immune plasma, indicating the presence of cross-reactive antibodies between ZIKV and DENV consistent with published literature^{14,35}.

### Monoclonal Antibody AA12 Provides Protection Against Lethal Heterologous Challenges

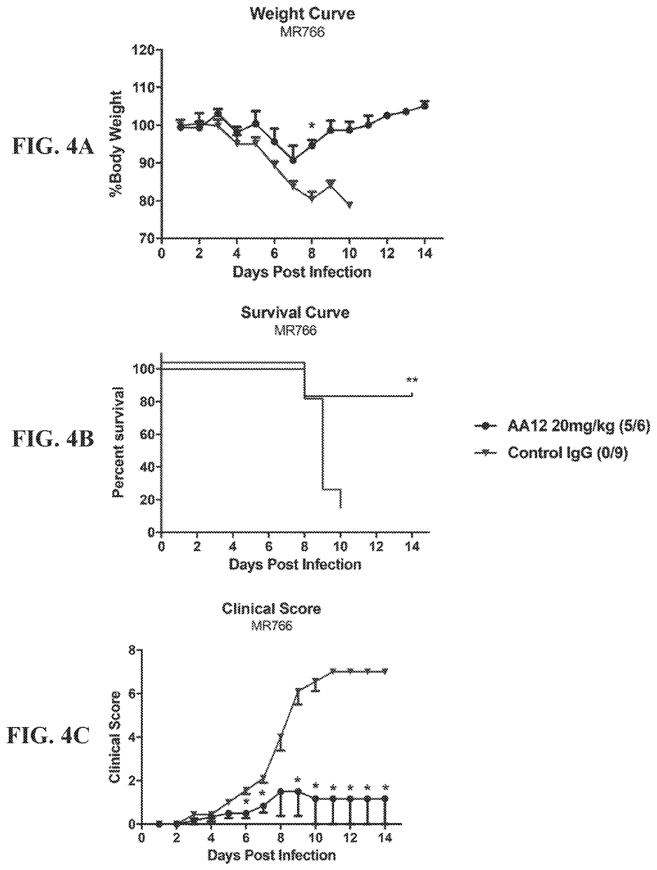

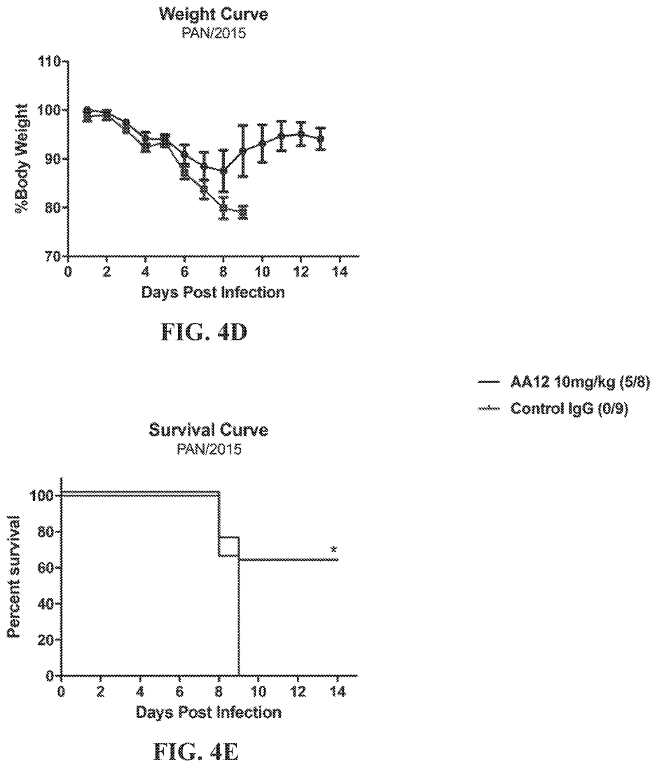

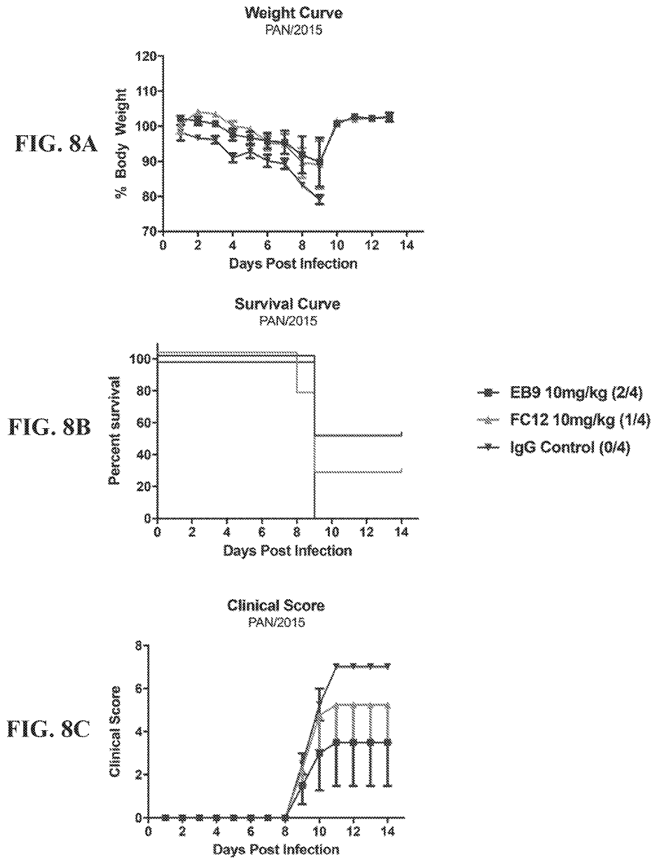

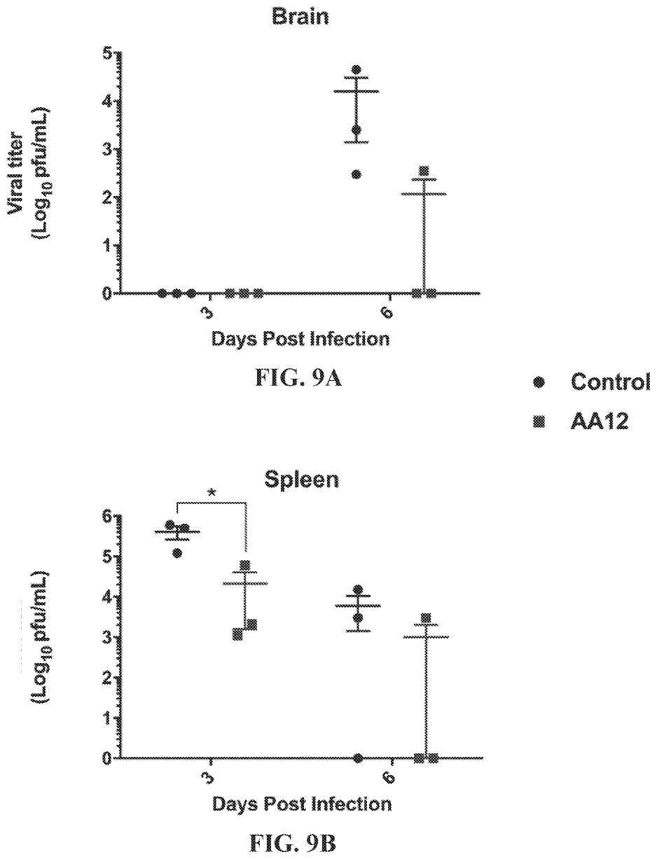

To assess the ability of NS1-specific mAbs to protect against ZIKV disease in vivo, lethal challenge experiments in a mouse model were performed. As Zika virus does not replicate in wild-type mice, Stat2^{-/-} mice, which are permissive to Zika virus infection and can display clinical signs of disease³⁶, were used. Antibody AA12 was administered intraperitoneally at either 20 mg/kg two hours before challenge. Irrelevant mAb at 20 mg/kg was used as an isotype negative control. Mice were then infected with ten 50% mouse lethal doses (10LD₅₀) of the Zika virus strain MR766 and weight loss, clinical scores, and survival were monitored daily. Stat2^{-/-} mice were bred in-house and colony sizes were a limiting factor in the number of mice in each group to be tested. Therefore, murine challenge studies were performed as two or three independent replicates with at least three mice per treatment group and data were then pooled. Mice that received 20 mg/kg of AA12 showed minimal weight loss (FIG. 4A), significantly improved survival rate (FIG. 4B) and significantly lower clinical scores on 6 to 14 days post infection (dpi) (except 8 dpi) as compared to the IgG control (FIG. 4C). Specifically, mice that received 20 mg/kg of AA12 had an 83% survival rate while the IgG control all succumbed to disease (0% survival). To determine whether protection is relevant to the recent Zika virus outbreak or limited to the mouse-adapted MR766 strain, the mAbs were tested against a contemporary Panama 2015 strain (H/PAN/2015/CDC-259359) from the Asian Zika virus lineage (FIGS. 4D, 4E). Mice were challenged with the PAN/2015 isolate as this particular virus demonstrated consistent mortality at the dose of 500 PFU in Stat2^{-/-} mice. In line with previous data above, AA12 was able to significantly protect animals from mortality at 10 mg/kg (63% survival), while the IgG control was not (0% survival). AA12 trended towards higher protection in the Panama 2015 strain challenge than the MR766 challenge likely due to the increased neurovirulence of the mouse-adapted MR766 strain. The two other monoclonal antibodies EB9 and FC12 were also tested in the same passive transfer challenge using PAN/2015 (FIGS. 8A-8C). The results demonstrate that EB9 protected 50% of the mice and FC12 only protected 25% of the mice compared to the IgG control group. Since the AA12 provided high levels of protection against lethal challenge from both the historic African and more modern Asian lineage of Zika viruses as shown in the survival rates and disease clinical scores, this antibody was used for all future experiments. Additionally, viral burden was measured in mice infected with PAN/2015 and treated with either 10 mg/kg of AA12 or IgG control (FIGS. 9A-9B). On days 3 and 6 post infection, mice were euthanized and spleens and brains were harvested and homogenized. Viral titers were then determined by plaque assay. Interestingly, no virus was detected in the brains of infected mice on day 3 post challenge. However, on day 6, virus was detected in the brains of all three mice administered IgG control and only one of the three mice in the AA12 treatment group. In the mouse spleens on day 3, there was a significant reduction of viral load between the control and AA12 treatment groups, while only one mouse given AA12 had a detectable load of virus on day 6. Collectively, the data demonstrate that administration of AA12 can significantly improve survival rates against two ZIKV strains, prevent disease severity and decrease viral titers in the spleen of mice.

### NS1-Mediated Protection is Dependent on FcγR Engagement on Host Cells

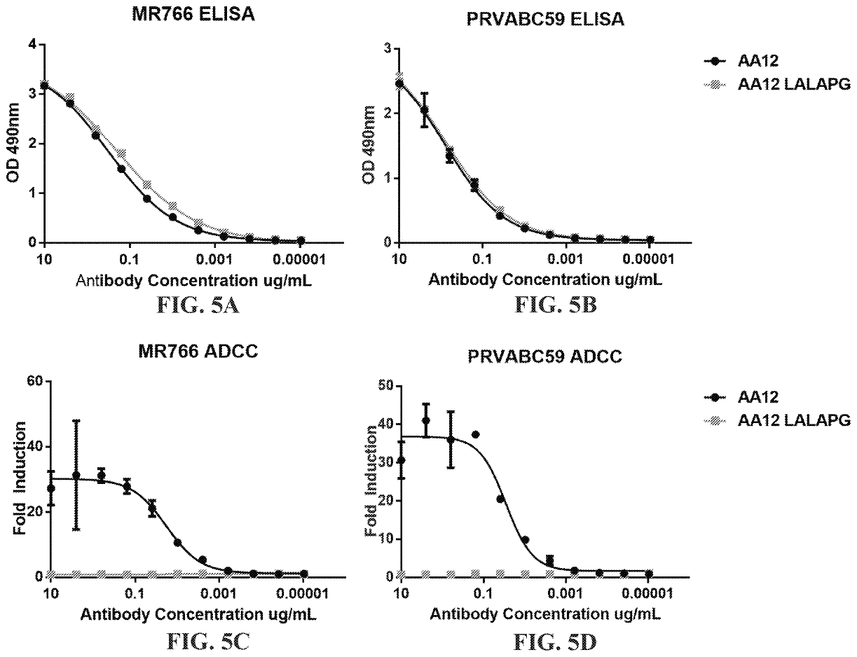

Next, it was examined whether Fc-FcγR or Fc-complement interactions are required for providing protection in vivo. First, the heavy chain variable regions of AA12 was cloned into expression vectors containing the human IgG1 framework with the amino acid mutations L234A, L235A, and P329G³⁷⁻³⁹. These mutations abolish the interaction of the Fc region with Fcγ receptors and complement proteins. To confirm that the mutations do not interfere with antigen binding, both variants were tested by ELISA (FIGS. 5A and 5B). Both the WT and mutated form of AA12 bound to the MR766 and PRVABC59 NS1 proteins identically. These variants were tested again in an Fc-FcγR engagement assay, and, as expected, only the wild-type AA12 variant showed activity (FIGS. 5C and 5D).

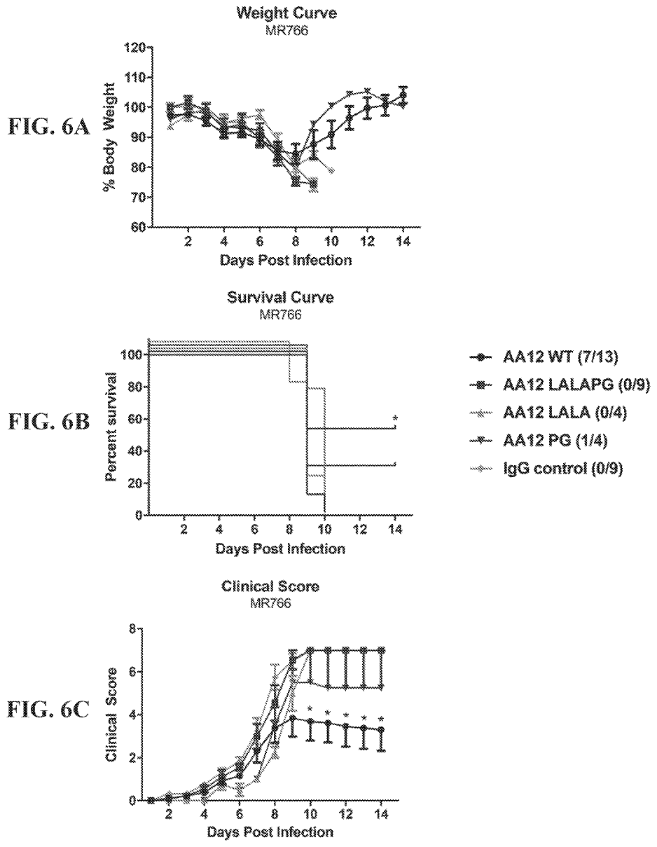

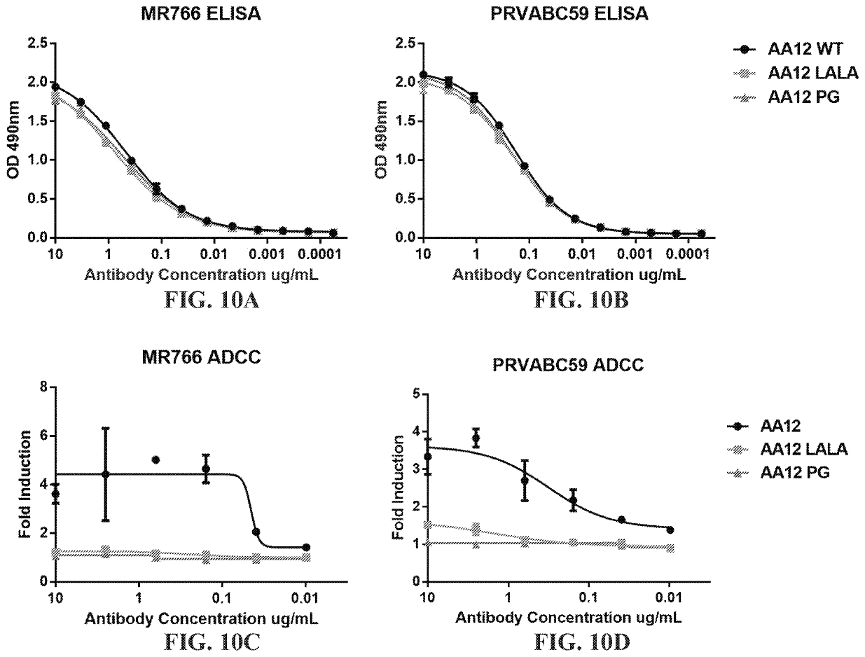

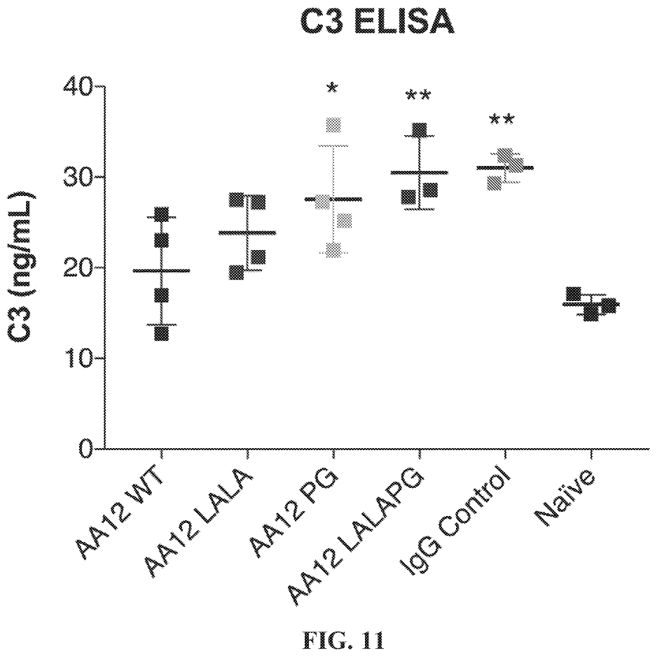

Then, it was examined if the AA12 variant has any protective activity in vivo, by performing the same prophylactic passive transfer challenge as done previously with ZIKV MR766. While weight changes were not substantially different from the different groups (FIG. 6A), administration of wildtype AA12 (10 mg/kg) significantly improved the survival rate (~53%) and clinical score over mice receiving the AA12 with ablated Fc-FcγR and complement interactions (LALAPG) or the IgG control (FIG. 6B). Lastly, the data demonstrate that Fc-FcγR or Fc-complement interactions are required to prevent onset of severe disease as wildtype AA12 can significantly decrease the clinical score (days 10 to 14) (FIG. 6C). Next, the L234A, L235A mutations or the P329G mutation alone were tested by introducing the LALA or PG point mutations into the AA12 antibody. Antibodies with either of these mutations alone had comparable binding affinity with the wildtype, were inactive in the Fc-FcγR engagement assay (FIGS. 10A-10D) and were tested in a prophylactic passive transfer challenge with ZIKV MR766. The AA12 LALA was not protective and AA12 PG only protected 25% of mice. It is possible that the single point mutation in PG did not completely disrupt Fc-FcγR engagement and some Fc-FcγR effector functions remained resulting in modest protection in the challenge model. However, as shown in FIG. 6C, both AA12 LALA or AA12 PG failed to decrease onset of severe disease. Overall, the data strengthens the previous findings that Fc-FcγR effector functions remain critical in protection afforded by mAb AA12. Next, it was determined whether complement was activated during infection by Zika virus. It was tested whether treatment with the AA12 variants ablated complement activation as compared to the wild-type AA12. Complement in serum at day 6 post infection was measured by ELISA in four of the mice used in the challenge from FIGS. 6A-6C. That day 6 was chosen as mice typically begin to show clinical signs of infection and high viral burden at this point. All mice undergoing the challenge had higher levels of complement activation as compared to naïve uninfected STAT2^{-/-} mice (FIG. 11). However, complement levels were elevated most in infected mice receiving the IgG control. It is therefore likely that these levels correlate most with morbidity and are activated by increased viral replication and the resulting heightened proinflammatory state. As wild-type AA12 protected mice against challenge more effectively than AA12 LALA or AA12 PG, it is unsurprising that complement levels were more elevated in the latter two groups.

### 6.1.4 Discussion

Several other groups have isolated and characterized human monoclonal antibodies to ZIKV⁸⁻¹², however, the main focus of these studies was on potently neutralizing antibodies targeting E, the surface envelope glycoprotein present on the virion. While a strong neutralizing antibody response to structural viral proteins contributes to protection against infection and disease, less is known about non-neutralizing antibodies that target the nonstructural proteins, such as NS1. Currently, there is a paucity of data on the protective efficacy of monoclonal antibodies that target the Zika virus NS1 protein. As many candidate flavivirus vaccines omit the NS1 component⁴⁰, it is possible that antibodies targeting the NS1 are overlooked in the protective immune response to ZIKV infection in humans. Of note, a recent study highlights the importance of incorporating NS1 in a multivalent vaccine against ZIKV⁴¹. Though Zika virus has one serotype with regards to a neutralizing response⁴², less is known about whether non-neutralizing antibodies against NS1 are able to target multiple strains of the virus. NS1 is highly conserved amongst Zika virus strains, approaching 99.3% sequence identity⁴³. The high level of conservation in NS1 proteins implies an immune response targeting NS1 may protect against all circulating strains and is a good candidate target for a vaccine. In fact, a recent study demonstrated that a ZIKV NS1-based vaccine using a Modified Vaccinia Ankara (MVA) vector is protective against a heterologous ZIKV challenge in mice³². Notably, the vaccine was given to wild-type mice who were subsequently challenged intracerebrally. As passive transfer studies have not been conducted, it is unclear which arm of the adaptive immune response, cell-mediated or humoral antibody immunity, contributed most to protection against disease. The studies described herein build on previous work and indicate that NS1-specific mAbs contribute to protection and should play in important role in the formulation of novel flavivirus vaccines.

In the present study, plasmablasts isolated from the PBMCs of a ZIKV-infected individual around 15 to 20 days after infection. Cloning of the variable regions of the antibody sequences isolated from plasmablasts revealed four NS1-specific mAbs that can bind to the recent Puerto Rico (PRVABC59) isolate of ZIKV. Interestingly, only the antibody FC12 is unable to bind the historic Uganda (MR766) strain of ZIKV, which has been isolated from rhesus macaques in 1947 and subsequently passaged in mice. While the ZIKV NS1 protein is highly conserved across many strains, the finding that one (FC12) of four mAbs isolated recognized only a recent ZIKV strain suggest that there may be different immunodominant regions of NS1 that vary between isolates. By isolating and characterizing more NS1-specific mAbs, the antigenic regions of the NS1 protein can be mapped and used for incorporating the NS1 protein in candidate ZIKV vaccines. With biolayer interferometry, we show the affinity of the antibodies to be between 10⁻⁷ to 10⁻⁸ molar, which suggests a moderate level of affinity. However, the bivalent manner by which NS1-specific mAbs bind to homodimeric NS1 suggest that avidity may play a role in increasing their biological function in vivo. Though the calculated affinity is lower than many potently neutralizing antibodies, it must be noted that these antibodies have lower levels of somatic hypermutation. It is speculated that more NS1-specific antibodies may be found with higher affinities and levels of somatic hypermutation in the memory B cell compartment of the same individual or in individuals who have had repeated exposures to the virus.

Vaccines that elicit NS1-specific mAbs do not risk inducing antibody-dependent enhancement of disease (ADE). In contrast, the recent Dengvaxia® vaccine, which induced antibodies to the structural components of dengue virus, caused an increased risk of severe disease in flavivirus naïve children, resulting in the suspension of the sale and distribution of the vaccine in the Philippines^{44,45}. To date, there are no clear cases involving ADE induced by ZIKV infection in humans. However, passive transfer of DENV or WNV immune plasma to immunocompromised mice has resulted in more severe disease progression upon ZIKV infection¹⁴. Additionally, ZIKV-induced monoclonal antibodies can enhance infection of DENV in vitro⁸. This is a major concern as ZIKV and dengue viruses are closely related, share the same mosquito vector, and impact the same geographic regions. The studies described herein suggest that as the NS1-specific mAbs are unable to enhance viral uptake in vitro, an NS1-based vaccine will be a safer alternative to current flavivirus vaccine preparations.

Using a murine challenge model, it is demonstrated herein that NS1-specific mAbs can prevent death and disease in vivo. The studies herein use B6.129-Stat2^{-/-} mice that are challenged intradermally with MR766 or retro-orbitally with PAN/2015. Though the two ZIKV sequences are highly conserved, the viruses were isolated 68 years apart and display different phenotypes in mice. Therefore, both strains were tested in the studies described herein. Infection with the MR766 strain represents a stringent challenge, inducing a higher level of inflammatory cytokines and severe neurological symptoms when mice are infected intradermally³⁶. Mice infected with the Asian lineage strain PAN/2015 were infected retro-orbitally to display consistent lethality in the challenge models. We found that mAb AA12 is able to significantly improve the survival rates of mice at doses of 20 mg/kg (83%) or 10 mg/kg (53%) during MR766 challenge and at a dose of 10 mg/kg (63%) during PAN/2015 challenge. Moreover, both doses of wildtype AA12 significantly decreased disease as measured by clinical score. Treatment of AA12 was also found to greatly reduce viral burden in the spleens of mice infected with PAN/2015 at day 3 post infection. Moreover, the data show that EB9 and FC12 trend towards partial protection against the recent PAN/2015 isolate of ZIKV at 10 mg/kg. Future experiments will determine the optimal range by which full protection is achieved.

Fcγ-mediated protection induced by non-neutralizing or poorly neutralizing antibodies has been found to play an important role in the context of many other viral infections including influenza A virus⁴⁶⁻⁴⁹, and it is unsurprising that these functions may protect against Zika virus disease. To explore whether Fcγ-mediated immunity is required for protection against ZIKV challenge, the heavy chain variable region of AA12 was cloned into an expression plasmid with the mutations L234A, L235A, and P329G in the Fc region³⁷⁻³⁹. These mutations resulted in ablated Fcγ-effector functions as measured by a surrogate reporter assay. The finding that the mutant AA12 mAb (AA12-LALAPG (SEQ ID NO:140) is unable to protect mice against lethal challenge suggest that activation of Fcγ-mediated effector functions is the mechanism by which protection is achieved. Additionally, AA12 antibodies with the L234A, L235A or the P329G mutations alone are also unable to protect mice against lethal challenge. To determine whether complement plays a role in reduction of viral burden, C3 levels were measured in mice undergoing lethal challenge. It was found that C3 levels are elevated in mice treated with AA12 LALAPG, AA12 PG, and IgG Control compared to mice treated with wild-type AA12. As wild-type AA12 suppresses viral burden and decreases disease severity, similar suppression of complement activation is seen. However, as mutant antibodies are unable to protect against lethal challenge, complement levels are increased—likely correlating with increased levels of disease and viral burden.

Notably, a high concentration of mAbs is required to confer protection against lethal challenge. As these mAbs are non-neutralizing and target a nonstructural protein, sterilizing immunity is not achieved. Though NS1-specific antibodies may not protect against initial infection, these antibodies limit disease severity as measured by decreasing weight loss and clinical score in antibody-treated animals. The induction of Fcγ-mediated protection by NS1-specific antibodies may therefore be an overlooked correlate of protection in the hunt for promising Zika virus vaccines. A complete vaccine that elicits not only neutralizing but also NS1-specific antibodies may increase protection against Zika virus disease in humans. A lack of established diagnostics also hampers ZIKV virus vaccine development.

Often, neutralizing antibody titers are used as a readout if viral RNA levels are not detected. Testing serum by plaque reduction neutralization tests may be additionally complicated by high levels of dengue virus cross-reactive antibodies. As the NS1 protein is highly conserved amongst ZIKV strains but only exhibits 55% identity with dengue virus⁴³, testing for NS1-specific antibodies may lead to better ZIKV diagnostics. Recent studies demonstrate a rapid NS1-based antigen test using monoclonal antibodies⁵⁰⁻⁵². The data herein adds to this work by reporting a highly specific antibody (FC12) only able to recognize the more recent ZIKV isolate. This antibody may provide a high-level of sensitivity and specificity in detecting serum levels of ZIKV NS1 in patients infected by recent outbreaks.

It is also notable that all four NS1-specific antibodies isolated from this patient had the same VH3-53/JH3 rearrangement but with different light chains (Table 1). This same rearrangement was found in NS1-specific antibodies isolated from two different patients from a separate recent study⁵³. Therefore, this rearrangement is found in many expanded B cell clones across the human population that target Zika virus NS1. Further investigation into how this germline rearrangement affects antibody binding to NS1 is warranted. This is also the first report of recurring antibodies that share the same IGV genes in the context of Zika virus NS1.

Teratogenic effects of ZIKV on the developing fetus in pregnant mothers is a major concern in the ongoing epidemic. In fact, it was the causal relationship between ZIKV infection during pregnancy and microcephaly that led the WHO to declare Zika virus a ‘public health emergency of international concern.’ In light of the work described herein, future studies should examine the prevention of viremia and maternal-fetal transfer of virus by NS1-specific mAbs or NS1-based vaccines.

In summary, the work described herein helps to further dissect the components of the antibody response against Zika virus. The importance of mAbs targeting the NS1 protein, which can dramatically protect against disease and death in a murine challenge model, has been highlighted. Furthermore, it is demonstrated that NS1 antibody-based protection against ZIKV disease is Fc-mediated. Lastly, the lack of ADE induction as measured by an in vitro assay suggests an NS1-based vaccine can reduce the risk of severe disease in flavivirus naïve patients as compared to a structural protein-based vaccine.

### 6.1.5 References Cited in Background and Example 1

1. Petersen, L. R., Jamieson, D. J. & Honein, M. A. Zika Virus. N. Engl. J. Med. 375, 294-295 (2016).
2. Hills, S. L. Transmission of Zika Virus Through Sexual Contact with Travelers to Areas of Ongoing Transmission—Continental United States, 2016. MMWR Morb. Mortal. Wkly. Rep. 65, (2016).
3. Schuler-Faccini, L. Possible Association Between Zika Virus Infection and Microcephaly—Brazil, 2015. MMWR Morb. Mortal. Wkly. Rep. 65, (2016).
4. Cauchemez, S. et al. Association between Zika virus and microcephaly in French Polynesia, 2013-15: a retrospective study. Lancet 387, 2125-2132 (2016).
5. Mlakar, J. et al. Zika virus associated with microcephaly. N. Engl. J. Med. 374, 951-958 (2016).
6. Cao-Lormeau, V.-M. et al. Guillain-Barré Syndrome outbreak associated with Zika virus infection in French Polynesia: A case-control study. The Lancet 387, 1531-1539 (2016).
7. Krauer, F. et al. Zika Virus Infection as a Cause of Congenital Brain Abnormalities and Guillain-Barré Syndrome: Systematic Review. PLoS Med. 14, e1002203 (2017).
8. Stettler, K. et al. Specificity, cross-reactivity, and function of antibodies elicited by Zika virus infection. Science 353, 823-826 (2016).
9. Zhao, H. Structural basis of Zika virus-specific antibody protection. Cell 166, 1016-1027 (2016).
10. Sapparapu, G. et al. Neutralizing human antibodies prevent Zika virus replication and fetal disease in mice. Nature 540, (2016).
11. Wang, Q. et al. Molecular determinants of human neutralizing antibodies isolated from a patient infected with Zika virus. Sci. Transl. Med. 8, 369ra179-369ra179 (2016).
12. Robbiani, D. F. et al. Recurrent Potent Human Neutralizing Antibodies to Zika Virus in Brazil and Mexico. Cell 169, 597-609.e11 (2017).
13. Whitehead, S. S., Blaney, J. E., Durbin, A. P. & Murphy, B. R. Prospects for a dengue virus vaccine. Nat. Rev. Microbiol. 5, 518-528 (2007).
14. Bardina, S. V. et al. Enhancement of Zika virus pathogenesis by preexisting antiflavivirus immunity. Science 356, 175-180 (2017).
15. Rastogi, M., Sharma, N. & Singh, S. K. Flavivirus NS1: a multifaceted enigmatic viral protein. Virol. J. 13, 131 (2016).
16. Avirutnan, P. et al. Secreted NS1 of Dengue Virus Attaches to the Surface of Cells via Interactions with Heparan Sulfate and Chondroitin Sulfate E. PLOS Pathog. 3, e183 (2007).
17. Petitdemange, C. et al. Control of Acute Dengue Virus Infection by Natural Killer Cells. Front. Immunol. 5, (2014).
18. Krishna, V. D., Rangappa, M. & Satchidanandam, V. Virus-specific cytolytic antibodies to nonstructural protein 1 of Japanese encephalitis virus effect reduction of virus output from infected cells. J. Virol. 83, 4766-4777 (2009).
19. Cane, P. A. & Gould, E. A. Reduction of yellow fever virus mouse neurovirulence by immunization with a bacterially synthesized non-structural protein (NS1) fragment. J. Gen. Virol. 69 (Pt 6), 1241-1246 (1988).
20. Schlesinger, J. J., Foltzer, M. & Chapman, S. The Fc Portion of Antibody to Yellow Fever Virus NS1 Is a Determinant of Protection against YF Encephalitis in Mice. Virology 192, 132-141 (1993).
21. Schlesinger, J. J., Brandriss, M. W. & Walsh, E. E. Protection against 17D yellow fever encephalitis in mice by passive transfer of monoclonal antibodies to the nonstructural glycoprotein gp48 and by active immunization with gp48. J. Immunol. Baltim. Md 1950 135, 2805-2809 (1985).
22. Schlesinger, J. J., Brandriss, M. W., Cropp, C. B. & Monath, T. P. Protection against yellow fever in monkeys by immunization with yellow fever virus nonstructural protein NS1. J. Virol. 60, 1153-1155 (1986).
23. Chung, K. M. et al. Antibodies against West Nile Virus Nonstructural Protein NS1 Prevent Lethal Infection through Fc γ Receptor-Dependent and -Independent Mechanisms. J. Virol. 80, 1340-1351 (2006).
24. Chung, K. M., Thompson, B. S., Fremont, D. H. & Diamond, M. S. Antibody recognition of cell surface-associated NS1 triggers Fc-gamma receptor-mediated phagocytosis and clearance of West Nile Virus-infected cells. J. Virol. 81, 9551-9555 (2007).
25. Chu, Y.-T. et al. Antibodies against nonstructural protein 1 protect mice from dengue virus-induced mast cell activation. Lab. Investig. J. Tech. Methods Pathol. (2017). doi: 10.1038/labinvest.2017.10
26. Wan, S.-W. et al. Protection against dengue virus infection in mice by administration of antibodies against modified nonstructural protein 1. PloS One 9, e92495 (2014).
27. Costa, S. M. et al. Protection against dengue type 2 virus induced in mice immunized with a DNA plasmid encoding the non-structural 1 (NS1) gene fused to the tissue plasminogen activator signal sequence. Vaccine 24, 195-205 (2006).
28. Tan, C. H., Yap, E. H., Singh, M., Deubel, V. & Chan, Y. C. Passive protection studies in mice with monoclonal antibodies directed against the non-structural protein NS3 of dengue 1 virus. J. Gen. Virol. 71 (Pt 3), 745-749 (1990).
29. Henchal, E. A., Henchal, L. S. & Schlesinger, J. J. Synergistic interactions of anti-NS1 monoclonal antibodies protect passively immunized mice from lethal challenge with dengue 2 virus. J. Gen. Virol. 69 (Pt 8), 2101-2107 (1988).
30. Schlesinger, J. J., Brandriss, M. W. & Walsh, E. E. Protection of mice against dengue 2 virus encephalitis by immunization with the dengue 2 virus non-structural glycoprotein NS1. J. Gen. Virol. 68 (Pt 3), 853-857 (1987).
31. Beatty, P. R. et al. Dengue virus NS1 triggers endothelial permeability and vascular leak that is prevented by NS1 vaccination. Sci. Transl. Med. 7, 304ra141-304ra141 (2015).
32. Brault, A. C. et al. A Zika Vaccine Targeting NS1 Protein Protects Immunocompetent Adult Mice in a Lethal Challenge Model. Sci. Rep. 7, 14769 (2017).
33. Smith, K. et al. Rapid generation of fully human monoclonal antibodies specific to a vaccinating antigen. Nat. Protoc. 4, 372-384 (2009).
34. Ho, I. Y. et al. Refined protocol for generating monoclonal antibodies from single human and murine B cells. J. Immunol. Methods 438, 67-70 (2016).
35. Priyamvada, L., Suthar, M. S., Ahmed, R. & Wrammert, J. Humoral Immune Responses Against Zika Virus Infection and the Importance of Preexisting Flavivirus Immunity. J. Infect. Dis. 216, S906-S911 (2017).
36. Tripathi, S. et al. A novel Zika virus mouse model reveals strain specific differences in virus pathogenesis and host inflammatory immune responses. PLoS Pathog. (2017).
37. Hezareh, M., Hessell, A. J., Jensen, R. C., van de Winkel, J. G. & Parren, P. W. Effector function activities of a panel of mutants of a broadly neutralizing antibody against human immunodeficiency virus type 1. J. Virol. 75, 12161-12168 (2001).
38. Vafa, O. et al. An engineered Fc variant of an IgG eliminates all immune effector functions via structural perturbations. Methods San Diego Calif 65, 114-126 (2014).
39. Schlothauer, T. et al. Novel human IgG1 and IgG4 Fc-engineered antibodies with completely abolished immune effector functions. Protein Eng. Des. Sel. PEDS 29, 457-466 (2016).
40. Barouch, D. H., Thomas, S. J. & Michael, N. L. Prospects for a Zika Virus Vaccine. Immunity 46, 176-182 (2017).
41. Liu, X. et al. Incorporation of NS1 and prM/M are important to confer effective protection of adenovirus-vectored Zika virus vaccine carrying E protein. Npj Vaccines 3, 29 (2018).
42. Dowd, K. A. et al. Broadly Neutralizing Activity of Zika Virus-Immune Sera Identifies a Single Viral Serotype. Cell Rep. 16, 1485-1491 (2016).
43. Xu, X. et al. Identifying Candidate Targets of Immune Responses in Zika Virus Based on Homology to Epitopes in Other Flavivirus Species. PLoS Curr. 8, (2016).
44. Halstead, S. B. & Russell, P. K. Protective and immunological behavior of chimeric yellow fever dengue vaccine. Vaccine 34, 1643-1647 (2016).
45. Ferguson, N. M. et al. Benefits and risks of the Sanofi-Pasteur dengue vaccine: Modeling optimal deployment. Science 353, 1033-1036 (2016).
46. Henry Dunand, C. J. et al. Both Neutralizing and Non-Neutralizing Human H7N9 Influenza Vaccine-Induced Monoclonal Antibodies Confer Protection. Cell Host Microbe 19, 800-813 (2016).
47. DiLillo, D. J., Tan, G. S., Palese, P. & Ravetch, J. V. Broadly neutralizing hemagglutinin stalk-specific antibodies require FcγR interactions for protection against influenza virus in vivo. Nat. Med. 20, 143-151 (2014).
48. He, W. et al. Alveolar macrophages are critical for broadly-reactive antibody-mediated protection against influenza A virus in mice. Nat. Commun. 8, 846 (2017).
49. DiLillo, D. J., Palese, P., Wilson, P. C. & Ravetch, J. V. Broadly neutralizing anti-influenza antibodies require Fc receptor engagement for in vivo protection. J. Clin. Invest. (2016). doi: 10.1172/JCI84428
50. Bosch, I. et al. Rapid antigen tests for dengue virus serotypes and Zika virus in patient serum. Sci. Transl. Med. 9, (2017).
51. Balmaseda, A. et al. Antibody-based assay discriminates Zika virus infection from other flaviviruses. Proc. Natl. Acad. Sci. U.S.A 114, 8384-8389 (2017).
52. Tsai, W.-Y. et al. Distinguishing Secondary Dengue Virus Infection From Zika Virus Infection With Previous Dengue by a Combination of 3 Simple Serological Tests. Clin. Infect. Dis. Off. Publ. Infect. Dis. Soc. Am. 65, 1829-1836 (2017).
53. Gao, X. et al. Delayed and highly specific antibody response to nonstructural protein 1 (NS1) revealed during natural human ZIKV infection by NS1-based capture ELISA. BMC Infect. Dis. 18, 275 (2018).
54. Dreyfus, C. et al. Highly conserved protective epitopes on influenza B viruses. Science 337, 1343-1348 (2012).
55. Duehr, J. et al. Tick-Borne Encephalitis Virus Vaccine-Induced Human Antibodies Mediate Negligible Enhancement of Zika Virus Infection In Vitro and in a Mouse Model. mSphere 3, e00011-18 (2018).

### 6.2 Example 2: Antibodies Elicited by an NS1-Based Vaccine Protect Mice Against Zika Virus

### 6.2.1 Introduction

Zika virus (ZIKV), a flavivirus related to dengue virus (DENV), has caused an epidemic that spread rapidly across the globe in the past decade¹. ZIKV infection can cause severe disease in humans, including microcephaly in newborns and Guillain-Barré syndrome in adults²⁻⁴. Although primarily spread by infected Aedes species mosquitoes, ZIKV can also be transmitted sexually or from mother to fetus^{5,6}. Ongoing transmission in the Americas and India suggest ZIKV is now endemic and much of the world's population is at continued risk of infection^{7,8} Due to the rapid spread of ZIKV and the particularly severe disease exhibited in developing human fetuses, effective vaccines and treatments are critically needed.

A number of studies in mice and non-human primates have shown the efficacy of multiple vaccine platforms⁹. DNA, mRNA, adenovirus, and purified inactivated virus platforms all have shown promising results in both preclinical and phase I studies¹⁰⁻¹⁴. Many of these vaccines are designed to protect against ZIKV infection by eliciting neutralizing antibodies that target the surface envelope glycoprotein E. These envelope-specific antibodies can be potently neutralizing and provide sterilizing immunity^{15,16}. However, an ongoing concern in the field of flavivirus vaccinology, and in dengue virus in particular, is the potential development of antibody dependent enhancement (ADE) of disease¹⁷. ADE occurs when antibodies bound to virions fail to neutralize the virus but facilitate virion internalization via the Fc receptors of innate immune cells. Increased viral internalization and subsequent replication leads to more severe disease outcomes. At present, there is no human epidemiologic evidence that prior immunity to ZIKV enhances dengue disease or vice-versa. However, in vitro and in vivo evidence suggests that enhancement of ZIKV or dengue virus can occur in experimental settings^{18,19}. As such, a vaccine approach targeting non-envelope viral proteins would minimize the potential for ADE of disease.

The immune response to acute flavivirus virus infection targets not only the E protein, but also the non-structural proteins including NS1. The flaviviral NS1 protein has been implicated in immune evasion and viral replication and has both intracellular and extracellular functions²⁰. Intracellularly, the NS1 protein localizes to sites of viral RNA synthesis and is critical for genome replication²¹. The NS1 protein is also trafficked to the plasma membrane where it binds the surface of infected cells by a putative glycosylphosphatidylinositol linker. The secreted form exists as a hexamer and accumulates to high levels in sera and tissues. The extracellular form of the NS1 protein is highly antigenic and is thought to modulate the humoral immune response. The extracellular form of the dengue virus NS1 protein can also activate complement pathways potentially leading to vascular leakage²². In the context of ZIKV, the NS1 protein contributes to evasion of the host antiviral response and has been found to enhance uptake of virus by mosquitoes^{23,24}. A potent immune response to the NS1 protein may have multifaceted beneficial effects including decreased transmission by halting the urban transmission cycle as well as reducing disease burden in humans by clearance of virally infected cells²⁵. Antibodies to the NS1 protein do not provide sterilizing immunity as they are non-neutralizing. However, NS1-specific antibodies are known to activate Fc-mediated effector functions such as antibody-dependent cell-mediated cytotoxicity (ADCC), antibody-dependent cell-mediated phagocytosis (ADCP) and antibody-dependent complement-mediated lysis²⁶⁻²⁹. Additionally, recent studies suggest the antibody response towards the ZIKV NS1 protein is highly specific and can be used for diagnostic purposes^{30,31}. As shown in Example 1, human antibodies that target the ZIKV NS1 protein can provide protection in mice against lethal challenge by ZIKV in an Fc-dependent manner³². A decrease in viral titer as well as reduction in morbidity and mortality in infected mice passively transferred with human anti-NS1 antibodies was shown. Therefore, NS1 may prove a key component of an effective ZIKV vaccine.

The NS1 protein of flaviviruses was considered a potential component in vaccine preparations. Vaccination with yellow fever virus NS1 protein prevented encephalitis in mice and lethality in macaques upon viral challenge^{33,34}. More recently, vaccination with the dengue virus NS1 protein prevented vascular leakage and disruption of endothelial barriers in mice³⁵. However, the dengue virus NS1 protein may also induce auto-antibodies that cross-react with host proteins present on endothelial cells and platelets, which may result in endothelial damage³⁶⁻³⁹. These phenomena, however, have not yet been reported for antibodies targeting the Zika NS1 protein. Today, a few groups have studied the role that NS1-mediated immunity may play in protection against ZIKV. Brault et al. have shown a ZIKV NS1 protein in a Modified Vaccinia Ankara vector protects mice from intracranial viral challenge⁴⁰. Two additional groups have combined NS1 with premembrane/membrane (prM/M) and E proteins and showed increased protection provided by NS1-prM/M-E as compared to prM/M-E alone^{41,42}. Notably, since none of these studies included passive transfer experiments, it is unclear whether the antibody or cell-mediated immune response contributed most to protection against disease.

This example provides a vaccination regimen consisting of a DNA prime and two NS1 protein boosts elicited high titers of antibodies to the ZIKV NS1 protein in wildtype mice. This example demonstrates that passive transfer of sera is sufficient to protect STAT2^{-/-} mice from lethal challenge, which suggests that the antibody-mediated immune response is critical to protect against disease. Sera from vaccinated mice engaged the FcγR in an in vitro Fc-FcγR reporter assay.

This example also demonstrates that NS1-mediated immunity is robust and long-lasting in humans by analyzing serum samples from acute and convalescent ZIKV infected patients. These antibodies generated against NS1 by natural viral infection are functionally active as measured by the same reporter assay. Notably, this example demonstrates that while polyclonal cross-reactive envelope antibodies elicited the Fc-dependent ADE of infection in vitro, these cross-reactive antibodies did not activate Fc-FcγR effector functions against ZIKV infected cells. This example indicates that the NS1-specific antibody response allows for robust Fc-dependent cell-mediated immunity, which has broad implications in the design of effective flavivirus vaccines.

### 6.2.2 Materials and Methods

Cells and viruses. Human embryonic kidney 293T (HEK 293T) cells (American Type Culture Collection [ATCC] catalog number CRL-1573) and African green monkey kidney (Vero) cells (ATCC) were grown in Dulbecco's modified Eagle medium (DMEM; Gibco) supplemented with 10% fetal bovine serum (FBS) (HyClone) and antibiotics (100 units/ml penicillin-100 μg/ml streptomycin [Pen-Strep]; Gibco). Human embryonic kidney Expi293F cells (Gibco) were grown in Expi293 expression media. The ZIKV PRVABC59 virus (2015/Puerto Rico, BEI NR-50684) and ZIKV MR766 virus (Rhesus/1947/Uganda, BEI NR-50065) were obtained from BEI Resources. Zika viruses were propagated in Vero cells in 1× minimum essential medium (MEM); after 72 h postinfection (hpi), cell culture supernatants were harvested, aliquoted, and stored at −80° C. until use.

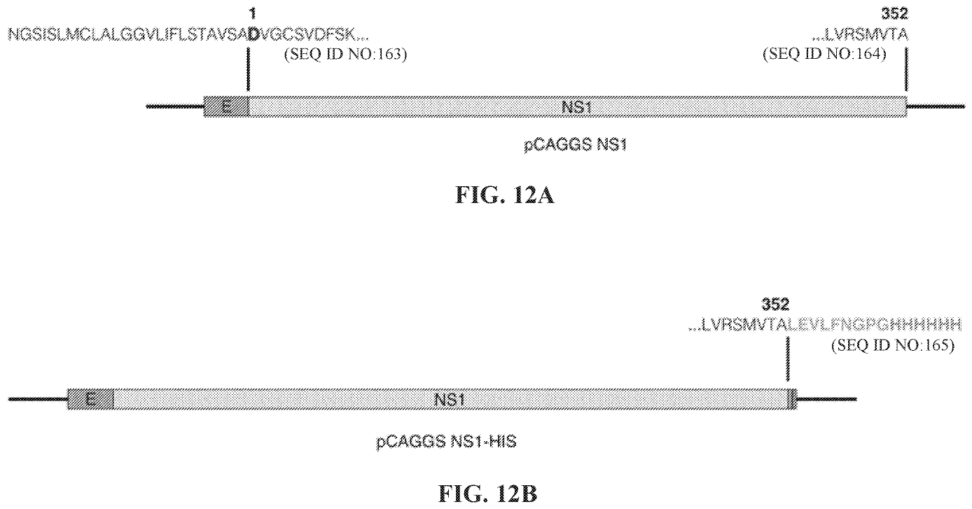

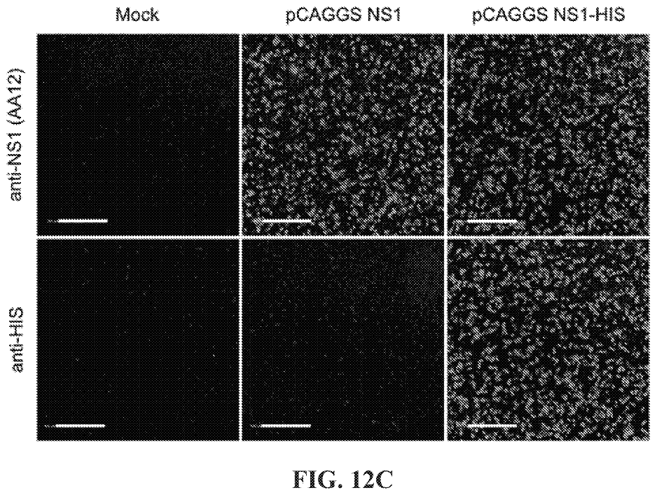

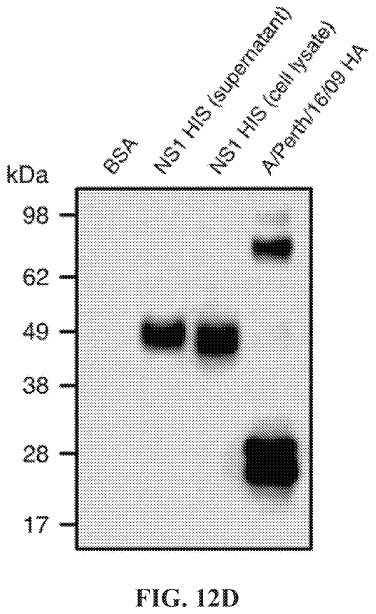

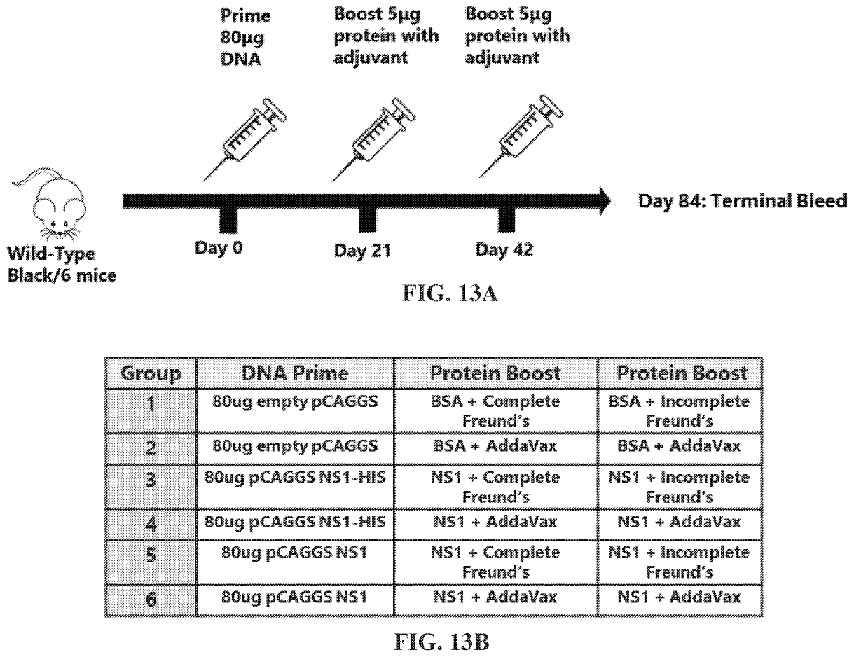

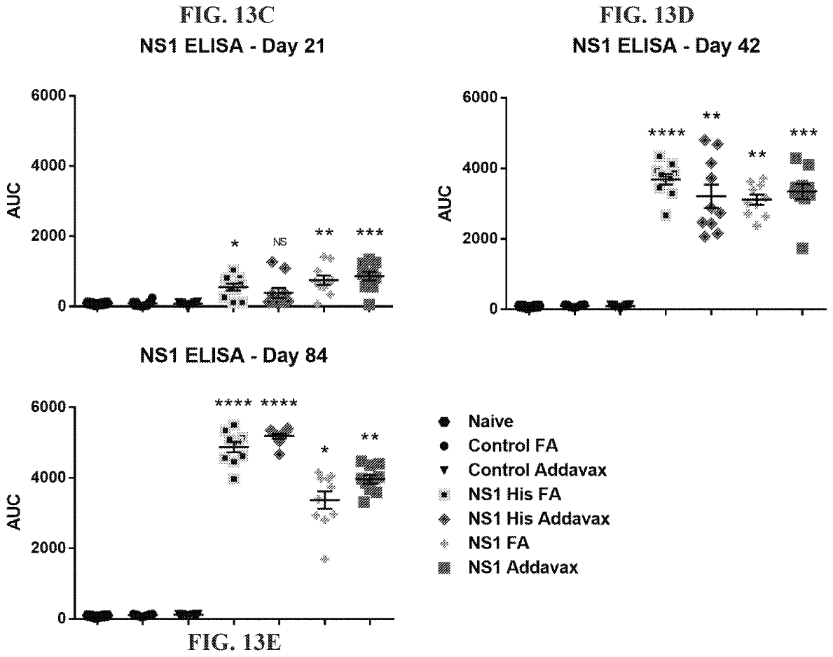

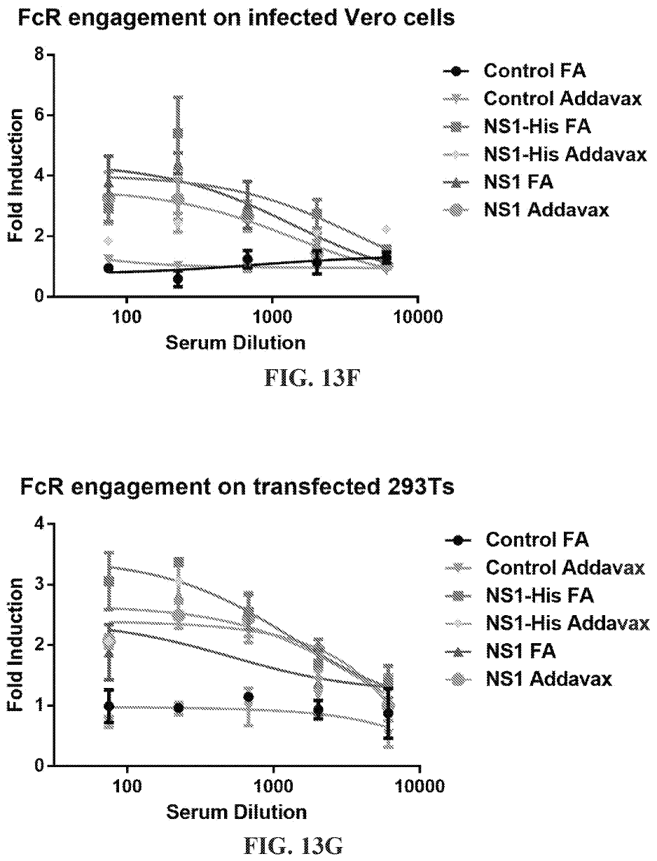

Recombinant Zika virus NS1. Two mammalian expression plasmids expressing NS1 of ZIKV PRV-ABC59 (2015/Puerto Rico; GenBank accession number KU501215, SEQ ID NO: 130) were generated by incorporating the last 24 amino acids of ZIKV envelope (NGSISLMCLALGGVLIFLSTAVSA, SEQ ID NO: 131) to the amino terminus of the NS1 coding region; the entire sequence was human-codon optimized using the Integrated DNA Technologies Codon Optimization tool. The first construct contained only the partial envelope and whole NS1 coding regions by inserting the synthetic gene insert into pCAGGS digested with NotI and XhoI (New England Biosciences), resulting in pCAGGS NS1 (FIG. 12A). Another construct, a PreScission Protease cleavage site (LEVLFNGPG, SEQ ID NO: 132) and a hexahistidine motif (HHHHHH, SEQ ID NO: 133) were added to the carboxy terminus of the NS1 coding region, resulting in pCAGGS NS1-His (FIG. 12B). Both constructs were generated using homologous recombination (In-Fusion; TaKaRa). To generate recombinant NS1 proteins, 30 ml of Expi293 cells were transfected with 30 μg of pCAGGS-NS1-His plasmids and 81 μl of ExpiFectamine transfection reagent (Gibco) as per the manufacturer's instructions. After 120 h, cells were pelleted by low-speed centrifu-gation and sonicated. Sonicated cells were pelleted again by centrifugation, and the supernatant was removed and incubated with Ni-NTA resin overnight at 4° C. The resin-supernatant mixture was then passed over 10-ml polypropylene columns (Qiagen). The retained resin was washed four times with 15 ml of washing buffer (50 mM Na2HCO3, 300 mM NaCl, 20 mM imidazole, pH 8), and protein was eluted with elution buffer (50 mM Na2HCO3, 300 mM NaCl, 300 mM imidazole, pH 8). The eluate was concentrated using Amicon Ultracel (Millipore) centrifugation units with a cutoff of 10 kDa, and buffer was exchanged with phosphate-buffered saline (PBS) at pH 7.4. Protein concentration was quantified using a Pierce bicinchoninic acid protein assay kit (Thermo Scientific) with a BSA standard curve. Purified soluble NS1 proteins were resolved in a reducing and denatured SDS-PAGE gel (in monomeric forms of around 45 kDa and in homodimeric forms of around 90 kDa) and visualized using SimplyBlue SafeStain (ThermoFisher, Inc.).

ELISA. Immulon 4 HBX ELISA plates (Thermo Scientific) were coated with recombinant ZIKV PRV-ABC59 NS1 protein (produced in-house) or recombinant envelope protein (MyBioSource accession number MBS319787) at 2 μg/ml in pH 9.41 carbonate buffer overnight at 4° C. Plates were washed three times with PBS between each step. After being blocked with 5% nonfat (NF) milk for 1 h, mouse sera were incubated at a starting concentration of 1:50, serially diluted 4-fold, and incubated for 2 h at room temperature. For experiments using human sera, a starting concentration of 1:40 was used. Horseradish peroxidase (HRP)-conjugated goat anti-human IgG antibody (AP504P; Millipore Sigma) or anti-mouse IgG antibody (AP503P; Millipore Sigma) was used to detect binding of IgG antibodies, followed by devel-opment with the HRP substrate (SigmaFast OPD; Sigma-Aldrich). Reactions were stopped by the addition of 3 M HCl, and absorbance was measured at 490 nm on a microplate spectrophotometer (BioRad). Experiments were performed in duplicate. A nonparametric multiple-comparison Kruskal-Wallis test was utilized to examine significance between groups. GraphPad Prism 6 was used to calculate area under the curve (AUC) values.

Immunofluorescence. A 24-well plate was treated with 30 μg/ml of poly-D-lysine (Millipore) for 1 h, followed by three washes with 1×PBS and a final wash with complete cell culture medium. HEK 293T cells (2×105 cells/well) were transfected in suspension with 0.5 μg of plasmid DNA (pCAGGS NS1 or pCAGGS NS1-His) and 2 μl of Lipofectamine 2000 (Invitrogen). Twenty-four hours posttransfection, cells were fixed with 0.5% paraformaldehyde (PFA)-1×PBS for 30 min. Cells were blocked with 5% nonfat milk for 30 min at room temperature, followed with incubation of 10 μg/ml of a human monoclonal antibody AA12 (32) or rabbit polyclonal antihistidine antibody (ThermoFisher) diluted in 1% nonfat milk-1×PBS for 1 h. Anti-human or anti-rabbit antibody conjugated to Alex Fluor 488 (Invitrogen) diluted in 1% nonfat milk-1×PBS at 1:1,000 were used as secondary antibodies. Stained cells were visualized using a Celigo imaging cytometer. Vero cells were infected with Zika virus PRVABC59 at a multiplicity of infection (MOI) of 0.5. After 24 h postinfection, the monolayer of Vero cells was fixed with 0.5% PFA-1×PBS. Cells were blocked with 5% nonfat milk for 30 min at room temperature. Blocking buffer was then discarded, and sera were added at a dilution of 1:100 in nonfat milk for 2 h at room temperature. Plates were washed three times with PBS between each step. After the cells were washed, an anti-mouse IgG secondary antibody conjugated to Alexa Fluor 488 (ThermoFisher) diluted 1:500 in nonfat milk was added to the monolayer, and plates were incubated in the dark for 1 h at room temperature. The cells were washed with PBS, and the monolayer was visualized using an Advanced Microscopy Group (AMG) Evos microscope (ThermoFisher).

Antibody-dependent effector functions. For experiments involving infected cells, Vero cells were seeded on 96-well, flat, white-bottom plates (Corning) and infected after 24 h with Zika virus PRVABC59 at an MOI of 0.01. For experiments involving transfected cells, HEK 293T cells were seeded onto 96-well, poly-D-lysine-coated, flat, white-bottom plates (Corning). After 24 h, the cells were transfected with 100 ng per well of pCAGGS-NS1 without the hexahistidine tag. At 16 h posttransfection or 40 h postinfection, the medium was removed and 25 μl of assay buffer (RPMI 1640 with 4% low-IgG FBS) was added to each well. Then, sera were added in a volume of 25 μl at a starting dilution of 1:75 and serially diluted 3-fold in assay buffer in duplicate. The sera were then incubated with the transfected or infected cells for 30 min at 37° C. Genetically modified Jurkat cells expressing either mouse FcγR IV or human FcγR IIIa with a luciferase reporter gene under the transcriptional control of nuclear-factor-activated T cell (NFAT) promoter were added at 7.5×104 cells in 25 μl per well, which is approximately a 1:2 ratio of target cells to effector cells (Promega). Cells were then incubated for another 6 h at 37° C. Bio-Glo Luciferase assay reagent was added, and luminescence was quantified using a microplate reader. Fold induction was measured in relative light units and calculated by subtracting the background signal from wells without effector cells and then dividing values for wells with antibody by values for those with no antibody added. Specifically, fold induction was calculated as follows: (RLUinduced−RLUbackground)/(RLUuninduced−RLUbackground). The mean values and standard errors of the means (SEM) were reported, and a nonlinear regression curve was generated using GraphPad Prism 6.

Mouse vaccination. All animal experiments were performed in an animal biosafety level 2 plus facility in accordance with the Icahn School of Medicine at Mount Sinai Institutional Animal Care and Use Committees (IACUC). Groups of 10 female STAT2−/− mice were vaccinated with 80 μg pCAGGS NS1-His, pCAGGS-NS1, or an empty vector in 40 μl of double-distilled H2O. DNA vaccines were delivered via intramuscular electroporation in the left posterior thigh muscles via a TriGrid electroporation device (Ichor Medical Systems). Protein-based vaccines (recombinant NS1 protein from PRVABC59 or BSA) were administered at a dose of 5 μg/mouse adjuvanted with either AddaVax (InvivoGen) intramuscularly at days 21 and 42 or Freund's complete adjuvant subcutaneously at day 21 (Sigma-Aldrich) and Freund's incomplete adjuvant subcutaneously at day 42 (Sigma-Aldrich). Six weeks after the last vaccination (day 84), animals were anesthetized with a ketamine-xylazine cocktail (0.15 mg of ketamine/kg of body weight and 0.03 mg of xylazine/kg per mouse), and serum was obtained via cardiac vein puncture.

Passive-transfer studies. Groups of 4 to 5 male and female B6.129-STAT2−/− mice (kindly provided by Christian Schindler; Columbia University) were passively transferred intraperitoneally with 200 μl pooled sera from vaccinated mice. Control mice received 200 μl pooled sera from mice vaccinated with DNA with an empty vector and BSA. Mice were challenged intradermally with 1,000 PFU of Zika virus PRVABC59 or 10 LD50s Zika (158 PFU) virus MR766 and evaluated for 14 days. Mice were monitored daily for weight and clinical signs. Clinical scoring was conducted using the predefined criteria, with a maximum possible score of 7: impact on walking, unresponsiveness, left hind leg paralyzed, right hind leg paralyzed, left front leg paralyzed, and right front leg paralyzed. Deceased animals were given a score of 7. Animals that showed more than 25% weight loss or full paralysis were humanely euthanized. Experiments were conducted with a balanced amount of male and female mice and with an even distribution of mice from different litters whenever possible. To determine statistical significance, the Mantel-Cox and Gehan-Breslow-Wilcoxon tests were used for survival curves, and a multiple t test and the Holm-Sidak method were utilized to analyze the weight curve and clinical scores. An asterisk(s) on a graph indicates the statistical significance (*, P<0.05) of a treatment group compared to the control group.

Donor samples. Deidentified TBEV-vaccinated donor serum samples were provided by the Vienna Blood Center in Austria as described previously (49). Deidentified Zika virus-infected blood donor plasma samples were obtained through the Global Virus Network Zika Serum Bank or Biodefense and Emerging Infections Research Resources Repository (BEI Resources).

Study approval. All studies conducted were considered by the Icahn School of Medicine at Mount Sinai's Institutional Review Board as not human subject research (NHSR).

Statistical analysis. Results from multiple experiments are presented as means±SEM. Multiple t tests were used to test for statistical differences between mean values. Data were analyzed with GraphPad Prism 6 software, and P values of <0.05 were considered statistically significant.

### 6.2.3 Results

Vaccination with a DNA plasmid and NS1 protein elicits high titers of anti-NS1 IgG in mice. Two ZIKV vaccine constructs were generated by introducing human codon-optimized sequences encoding the full-length NS1 protein from the Asian-lineage ZIKV PRVABC59 strain into a pCAGGS mammalian expression vector. The first construct, pCAGGS NS1, encodes the last 24 amino acids of the ZIKV envelope protein at the amino terminus, allowing for proper folding and anchoring of the NS1 protein to the lipid bilayer (43) followed by the complete coding region of the NS1 protein of ZIKV PRVABC59 (an aspartic acid marks the first residue of the NS1) (FIG. 12A). We also designed an expression plasmid designed to produce soluble NS1 protein. The pCAGGS NS1 expression plasmid was modified by adding a PreScission Protease cleavage site and a hexahistidine motif at the carboxy terminus, and the resulting plasmid was named pCAGGS NS1-His (FIG. 12B). To determine if these plasmids generated properly folded ZIKV NS1 protein, we transfected HEK 293T cells with pCAGGS NS1 or pCAGGS NS1-His. Mock-transfected cells served as a negative control. At 24 h posttransfection, expression of ZIKV NS1 on HEK 293T cells was confirmed using immunofluorescence by a human monoclonal antibody, AA12, or a polyclonal antihistidine antibody (32) (FIG. 12C). As expected, an antihistidine antibody detected only pCAGGS NS1-His. Mock-transfected cells were not detected by AA12 or polyclonal antihistidine antibody. Using the pCAGGS NS1-His plasmid, we expressed the NS1 protein in human embryonic kidney (HEK) Expi293 cells and purified the protein using a nickel-nitrilotriacetic acid (Ni-NTA) resin. Purified soluble NS1 protein was resolved by sodium dodecyl sulfate-polyacrylamide gel electrophoresis (SDS-PAGE) under both denaturing and reducing conditions. Western blot analysis using a polyclonal anti-His antibody demonstrated that the soluble His-tagged NS1 proteins purified from both the cell culture supernatant and lysates resolved at approximately 50 kDa as monomers (FIG. 12D). A soluble histidine-tagged hemagglutinin of influenza A virus strain A/Perth/16/09 (H3N2) was used as a positive control, while bovine serum albumin (BSA) served as a negative control. We then vaccinated groups of 10 mice as outlined by the vaccination strategy in FIGS. 13A and 13B. At day 0, wild-type C57BL/6 mice were primed with either 80 μg of pCAGGS NS1-His or pCAGGS NS1 via intramuscular electroporation. Next, mice were immunized intramuscularly with 5 g of adjuvanted NS1 protein at days 21 and 42. Mice receiving soluble NS1 protein with Freund's adjuvant received complete adjuvant at day 21 and incomplete adjuvant at day 42. Mice receiving soluble NS1 protein with Adda Vax received the same adjuvanted protein on both days 21 and 42. As a control, mice were vaccinated with an empty pCAGGS plasmid and boosted twice with BSA supplemented with either Freund's adjuvant or Adda Vax. Prior to administration of each vaccine component, serum samples were obtained by facial vein puncture. At day 84, the mice were anesthetized and terminally bled by cardiac puncture, and sera were collected for further analysis and passive-transfer studies.

An NS1-specific enzyme-linked immunosorbent assay (ELISA) was performed, and all NS1-vaccinated mice demonstrated a robust reactive antibody response after the DNA prime immunization followed by two protein boosts (FIGS. 13C to 13E). Significant differences from the naive group were observed in all vaccine groups on day 42 and day 84. On day 21, there was no significant difference between the NS1-His AddaVax group and the naive group. However, this group had sufficiently high titers by days 42 and 84. No differences were observed between the naive group and either of the two control groups. Additionally, no differences were observed within each vaccination group. A trend toward higher titers in the groups vaccinated with the His-tagged construct was observed by day 84. This is likely due to elicited anti-His antibodies recognizing His-tagged NS1 proteins used in our ELISAs.

Immunofluorescence studies demonstrated reactivity to Vero cells infected with the Asian-lineage ZIKV PRVABC59 in all treatment groups (see FIG. 18). As the NS1 protein is not present on the Zika virion itself but is expressed on the surfaces of infected cells, NS1-mediated immunity is unlikely to be sterilizing. Rather, NS1-specific antibodies are likely to protect via antibody-dependent cell-mediated effector functions. To determine whether sera from vaccinated mice are functionally active against infected cells, the ability of the NS1-vaccinated mouse sera to engage FcyRs was tested. A well-established in vitro assay previously used to assess the functional activities of monoclonal antibodies targeting the influenza virus hemagglutinin and the Zika virus NS1 protein (32, 44) was used. In this assay, engagement of the murine FcyR IV expressed on genetically modified effector (Jurkat) cells results in a quantifiable luminescent signal. Vero cells were infected with the ZIKV PRVABC59 strain and pooled (n=10) sera from vaccinated mice were added. Consistently with the ELISA results, sera from all NS1-vaccinated groups induced effector functions on ZIKV PRVABC59-infected cells (FIG. 13F), while sera from the control group were unable to engage FcyRs. To confirm that Fc-mediated effector functions are NS1 specific, 293T cells were transfected with the pCAGGS NS1 plasmid. As with the infected cells, all NS1-vaccinated groups induced effector functions but that the control groups did not (FIG. 13G), indicating that Fc-mediated activity was indeed NS1 specific.

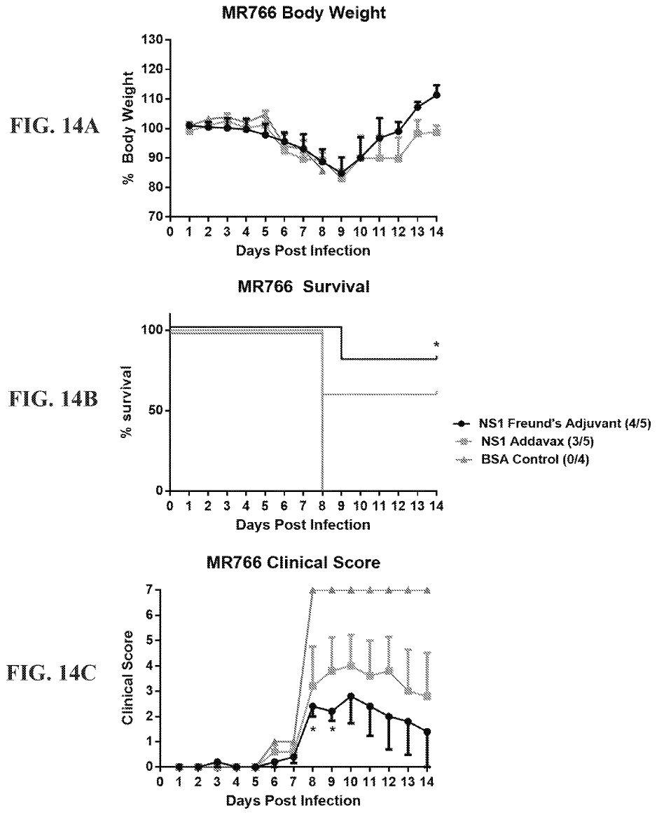

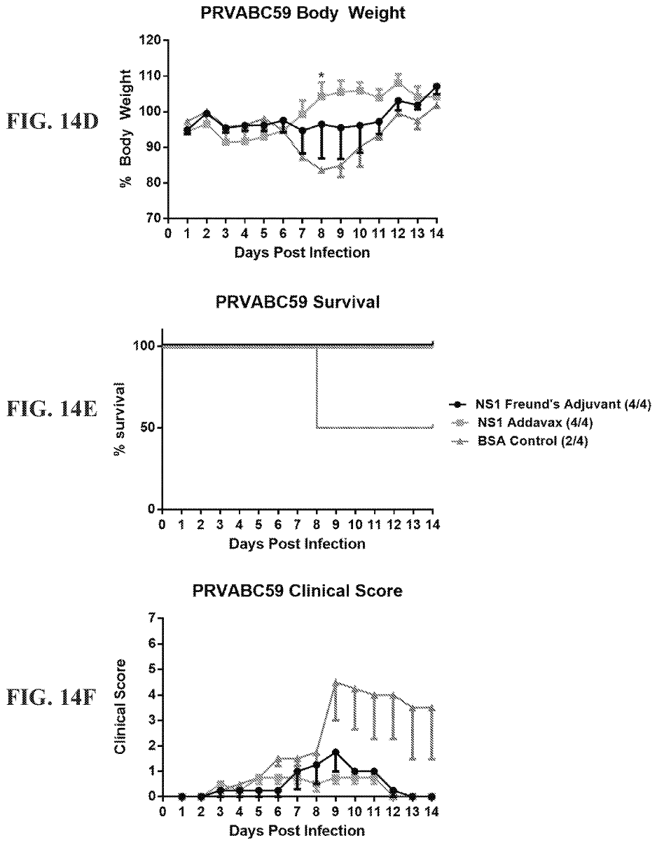

Passive transfer of immune sera protects STAT2−/− mice from lethal challenge. To determine whether antibodies elicited by our vaccine regimen are protective against ZIKV, 200 μl of pooled sera were passively transferred intraperitoneally into STAT2−/− mice, which are permissive to ZIKV infection and can display clinical signs of disease (45). Two hours after administration of sera, the mice were challenged intradermally with 10 50% lethal doses (LD50) of the African-lineage ZIKV MR766 strain. Mice were monitored daily for weight loss and scored for signs of disease, including difficulty walking, limb paralysis, and unresponsiveness. Animals exhibiting a clinical score of 5 or higher were euthanized and scored as succumbing to disease. Sera from mice given an NS1 booster and Freund's adjuvant provided the highest degree of protection, with 80% of the mice surviving the challenge, compared to 60% in the Adda Vax group and 0% in the BSA control group (FIGS. 14A to 14C). Next, protection against the homologous Asian-lineage strain PRVABC59, which is more closely related to contem-porary strains of ZIKV, was tested. 1,000 PFU of the PRVABC59 virus were administered, as a proper LD50 could not be administered due to a lack of virulence at the highest doses tested. 100% of mice given sera from NS1-vaccinated mice survived the challenge with PRVABC59, compared to 50% of mice treated with control sera (FIGS. 14D to 14F). As with the results of the MR766 challenge study, all mice displayed clinical signs of infection. No differences in lethality between male or female mice were observed. Though it is well established that ZIKV PRVABC59 displays less pathogenicity than MR766 in mice, significant differences in weight loss between the NS1-vaccinated mice and control mice were still able to be detected (45).

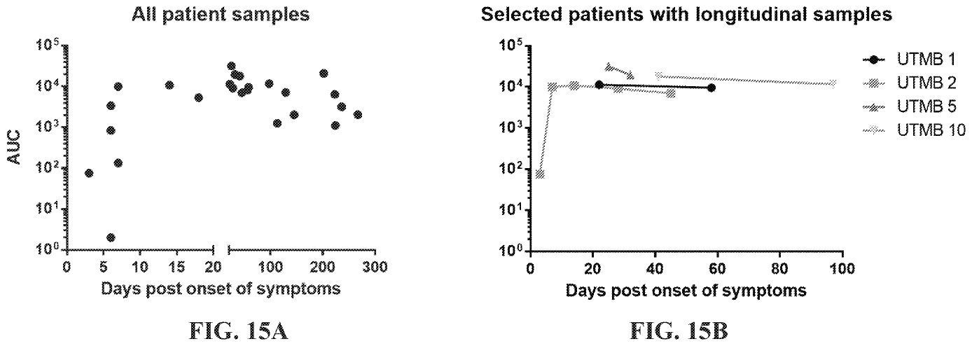

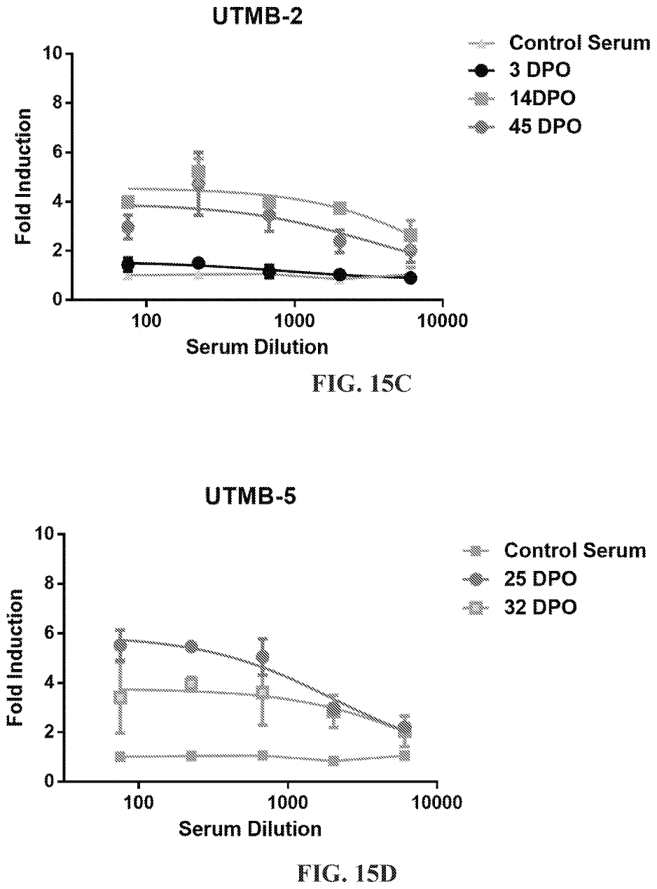

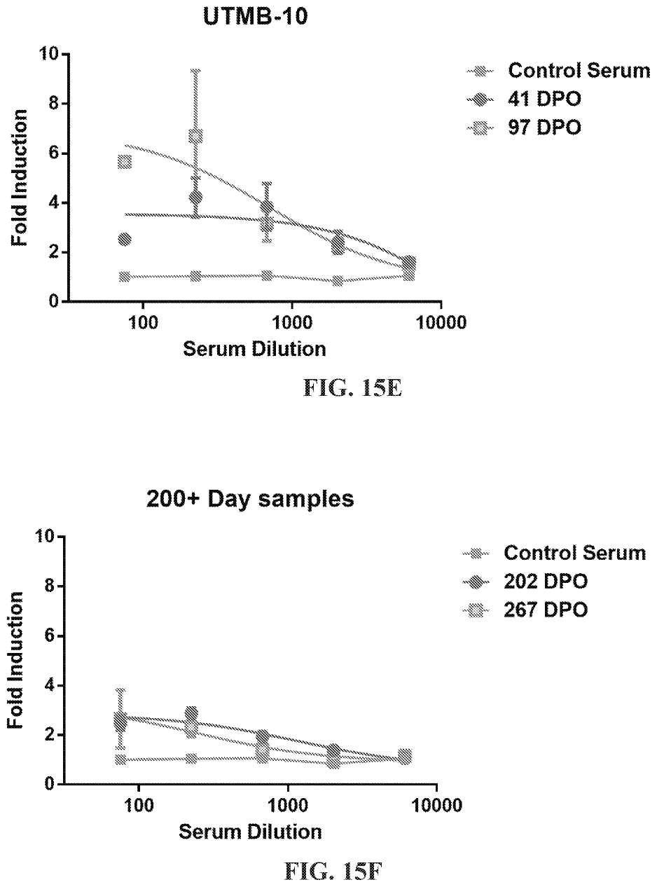

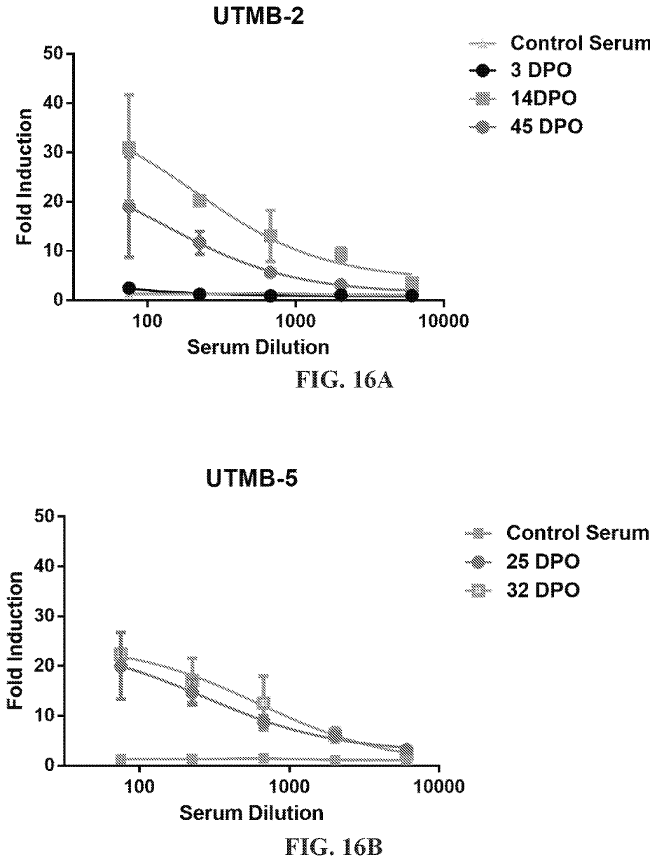

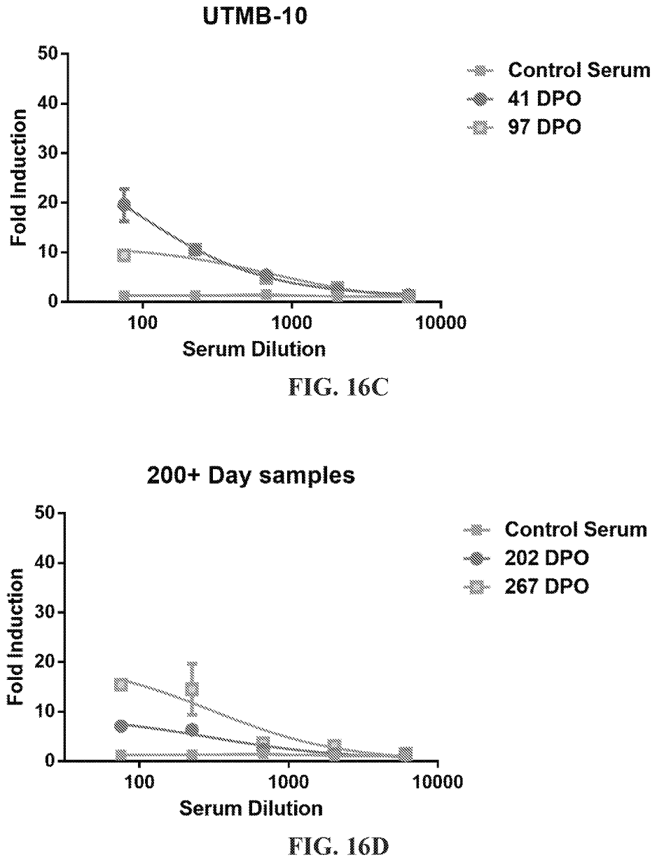

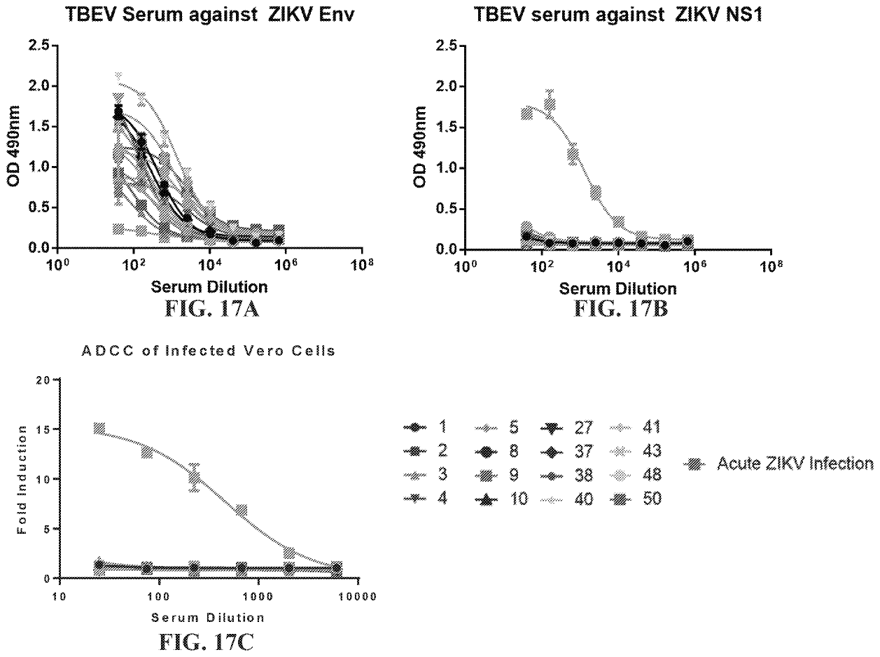

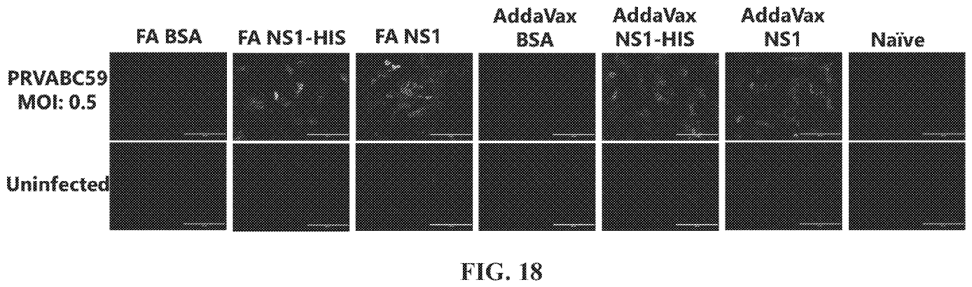

NS1-mediated immunity is long-lasting in humans and mediates Fc effector functions. The neutralizing activity of envelope-specific antibodies elicited during ZIKV infection is well documented and characterized (19, 46, 47). However, there are a paucity of data on the duration and mechanisms of action of NS1-specific antibodies in humans infected by ZIKV or other flaviviruses. To determine whether NS1-mediated immunity is relevant and long-lived in humans, serum samples were obtained from patients infected by ZIKV. These samples were taken from patients ranging from acutely ill to fully recovered, from 3 to 267 days postonset of symptoms (Tables 11 and 12). The reactivity of these serum samples to NS1 protein was determined by ELISA. NS1-specific antibodies became detectable at approximately day 10 post onset of symptoms and remained elevated throughout day 267, with minimal waning over time (FIG. 15A). Serum samples from the same individuals were obtained at multiple time points to represent a longitudinal response. In these matched samples, the NS1 response waned slightly over time but did not return to baseline levels (FIG. 15B). Next, the ability of serum samples from these individuals to elicit Fc-FcyR-mediated effector functions was evaluated. In vitro assays, showed measurable correlations between reactivity to NS1 and the ability to engage FcyR (FIGS. 15C to 15F). For instance, patient UTMB-2 had a low antibody titer to NS1 at day 3 postinfection and likewise did not show effector function activity on ZIKV PRVABC59-infected cells at that time point. However, at days 14 and 45 postinfection, both the patient's sera were reactive to NS1 by ELISA and functionally active, as measured by the ADCC reporter assay. Additionally, two samples taken later than day 200 postinfection were tested and showed that these sera were still able to induce effector functions. Next, the questions of whether antibodies to NS1 specifically contributed to the Fc-FcyR-mediated immunity by transfecting HEK 293T cells with a plasmid expressing NS1 and using the same ADCC reporter assay was addressed. Individuals who had a positive antibody response to infected Vero cells were found to also react with transfected HEK 293T cells (FIGS. 16A to 16D). These data show that the NS1 response elicited by natural ZIKV infection is long-lasting and contributes to Fc-mediated immunity in humans.

Cross-reactive antibodies against the envelope protein do not elicit FcyR effector functions in humans. Though a significant number of antibodies are generated against the NS1 protein, a larger portion of the antibody response is directed against the ZIKV envelope protein. Envelope-specific antibodies predominantly con-tribute to a potent neutralizing response and provide sterilizing immunity. Cross-reactive envelope-specific antibodies, however, are also known to be potent mediators of antibody-dependent enhancement (ADE) of disease (15). These antibodies are known to bind conserved epitopes near the fusion loop of the envelope glycoprotein and can bind divergent flaviviruses (48). Notably, in Duehr et al., 28 of 50 serum samples from tick-borne encephalitis virus (TBEV)-vaccinated individuals bound to recombinant ZIKV envelope protein by ELISA, while 36 of 50 serum samples had enhanced ZIKV infectivity in vitro (49). Since ADE of infection is Fc mediated, it was determined whether these same cross-reactive antibodies are able to elicit potentially beneficial Fc-mediated effector functions in vitro on infected cells. A set of serum samples from individuals vaccinated against TBEV, a member of the flavivirus family (49), were analyzed. Though the amino acid sequences of the TBEV and the ZIKV envelope proteins are divergent, exhibiting approximately 40% identity at the amino acid level (49), cross-reactive antibodies against conserved epitopes near the fusion loop of domain II of the envelope protein are often generated (15). Sixteen of the highest ELISA- and ADE-reactive serum samples from the work of Duehr et al. were analyzed for binding to the recombinant ZIKV E protein by ELISA, and all showed a positive response (FIG. 17A). As a control, sera from an acute ZIKV infection known to have a strong NS1-specific response with low reactivity to recombinant ZIKV E (32) were used. Next, it was confirmed that the vaccinated samples did not have antibodies targeting the ZIKV NS1 protein. The TBEV vaccine, which was used to vaccinate the human subjects, uses inactivated TBEV virus. As this vaccine does not contain NS1, serum samples from TBEV patients did not react with ZIKV NS1, while the positive control, serum from an acutely infected individual, did react (FIG. 17B). It was then tested whether these serum samples can elicit Fc-mediated effector functions in ZIKV PRVABC59-infected Vero cells. Out of the 16 TBEV-vaccinated serum samples tested, none were able to elicit Fc-mediated effector activity on ZIKV-infected cells, but sera from an individual acutely infected with ZIKV did (FIG. 17C). These data suggest that while cross-reactive envelope-specific antibodies elicited by TBEV vaccination might cause ADE of infection to occur in vitro, they do not induce Fc-mediated effector functions on infected cells. A possible explanation is that a strong NS1 antibody response is important for the clearance of ZIKV-infected cells via Fc-dependent cell-mediated activity. Conversely, due to the low levels of the envelope glycoprotein expressed at the surfaces of infected cells, cross-reactive E antibodies are unable to target these cells for Fc-mediated clearance.

### 6.2.4 Discussion

Several ZIKV vaccines are currently under various phases of development (10-14, 50-52). The aim of most of these candidate vaccines is to elicit potent naturalizing antibody responses using the envelope glycoprotein E as the major target antigen. ZIKV E-specific antibodies can provide sterilizing immunity, and characterization of a number of neutralizing monoclonal antibodies targeting the E protein revealed high neutraliz-ing activity with half-maximal inhibitory concentrations in the nanogram-per-milliliter level (19, 46, 47, 53, 54). However, the ZIKV NS1 protein is an equally viable target for a vaccine. Previous studies have demonstrated antibodies against other flavivirus NS1 proteins from yellow fever virus (YFV), West Nile virus (WNV), or DENV can limit or prevent flavivirus disease (26-28, 33-35). Additionally, vaccines that elicit NS1-specific antibodies do not cause antibody-dependent enhancement of disease, as these anti-bodies do not bind to the virion itself. This is pertinent, as the vaccine Dengvaxia against the closely related dengue virus was shown to increase the frequency of severe disease in dengue virus-naive children (55, 56). Thus, NS1 may be an overlooked component of a safe and effective ZIKV vaccine.

Recently, several groups have reported the effectiveness of NS1-based vaccines against ZIKV infection. In a paper by Brault et al., an NS1 vaccine incorporated into a modified vaccinia virus Ankara (MVA) vector was shown to protect wild-type mice against intracerebral challenge (40). As the same mice were vaccinated and subse-quently challenged, it is unclear whether cell-mediated or humoral immunity or both contributed to protection. Studies by Liu et al. and Li et al. incorporated NS1 in addition to PrM/M and E in either an adenovirus 2 (Ad2)- and a recombinant vesicular stomatitis virus (rVSV)-based vaccine, respectively (41, 42). Both studies showed that the inclusion of NS1 into the vaccine construct provides additional protection compared to PrM/M and E alone. Though the contribution of NS1 antibodies to protection is clearly shown, an Ad2-NS1 construct alone was not tested. However, an rVSV-NS1 construct without any structural protein components was shown to reduce viral titers compared to those in the unvaccinated control mice.

This study demonstrates that a vaccination strategy based solely on the ZIKV NS1 protein can elicit a strong antibody response that significantly protects mice against lethal challenge (FIGS. 13A-13G and 14A-14F). This strategy involved priming vaccinated mice with a DNA plasmid, followed by two protein boosts with either Freund's adjuvant or Add-aVax, which is an oil-in-water emulsion similar to MF59 found in a human seasonal influenza virus vaccine (57). Antibodies elicited by this vaccine bound potently to soluble NS1 protein by ELISA and recognized ZIKV-infected Vero cells, as measured by immunofluorescence. Pooled sera from vaccine groups also activated Fc-FcyR-mediated effector functions against infected Vero cells or NS1-transfected 293T cells in an ADCC reporter assay.

This study used a passive-transfer model in which sera from vaccinated mice were passively transferred to STAT2−/− mice. The mice then underwent a lethal challenge via intradermal infection of two ZIKV strains from different lineages (FIGS. 14A-14F). Though the ZIKV sequences are highly conserved, the two different strains were isolated 68 years apart and display different disease phenotypes in STAT2−/− mice (45). The efficacy of our NS1 vaccination strategy against the ZIKV MR766 strain, which due to its high lethality in mice represents a stringent challenge, was tested. The high lethality of MR766 was likely due to extensive passaging in the brains of mice. In this case, four of five mice receiving serum from the NS1 Freund's adjuvant group and three of five mice from the NS1 Adda Vax group survived. In contrast, none of the mice receiving serum from control vaccinated mice survived infection. The ZIKV PRVABC59 strain isolated in 2015 represents the modern circulating strain, was not mouse adapted, and is less pathogenic in mice. In this challenge model, all mice receiving serum from NS1-vaccinated mice survived, while two of four mice receiving serum from control-vaccinated mice succumbed to infection. Notably, none of the vaccinated mice were completely protected from ZIKV disease, as measured by weight loss or clinical score, suggesting that sterilizing immunity is not achieved. However, this is the first demonstration of NS1 antisera providing protection against lethal ZIKV challenge in a passive-transfer model, underscoring the importance of NS1-specific antibodies in mediating immunity to ZIKV. Though the exact mechanism of NS1-specific immunity needs to be further studied, we speculate that Fc-mediated viral clearance plays an important role in the prevention of disease progression by clearing virus-infected cells. Furthermore, both the Adda Vax and Freund's adjuvant treatment groups elicit high titers of antibodies and protection in passive-transfer studies that do not significantly differ from each other. Therefore, a minimally protective NS1-specific titer could not be quantified. Future studies might perform dose titrations of vaccine to determine the minimal NS1-specific antibody titer required for protection.

To determine whether NS1-mediated immunity is relevant and long-lived in humans, 31 serum samples from 16 different patients who were infected by ZIKV were obtained. These samples ranged from day 3 to day 267 postonset of symptoms and represent both the acute and the convalescent phase of illness. Binding to recombinant NS1 was tested by ELISA and showed that the antibodies become detectible by day 10 and last beyond day 267 (FIGS. 15A-15F). Based on these results, the NS1 protein of ZIKV is potently immunogenic. These results are to be expected, as the NS1 response in other flaviviruses has been well studied. To determine whether antibodies elicited by natural infection were functionally active, an assay to measure Fc-mediated effector functions was performed. All serum samples that were positive by ELISA were also positive in the reporter assay. In contrast, negative-control sera and sera from day 3 postonset of symptoms were unable to elicit Fc-mediated effector functions on in-fected cells. Additionally, all serum samples that were active against infected Vero cells were also active against NS1-transfected 293T cells (FIGS. 16A-16D). This suggests that the predominant Fc-mediated antibody response against Zika virus targets the NS1 protein. Additionally, the NS1 protein was shown to be sufficient to activate Fc-mediated effector functions on infected cells by human sera. Human monoclonal antibodies that target ZIKV NS1 are protective. However, future studies will look at purified polyclonal NS1 antibodies isolated from human sera to determine if passive transfer of these antibodies will protect mice against lethal challenge.

Fc-dependent responses mediated by virus-specific antibodies can generally be divided into two categories: responses that target viral particles and responses that mediate killing of virus-infected cells. In the first scenario, antibodies can facilitate the internalization of virions via Fc-mediated endocytosis into innate immune cells, where either degradation or replication can occur (58, 59). In the context of Zika virus and other flaviviruses, antibody-mediated uptake of virus increases the sites of virus repli-cation and can potentially enhance disease (18). Alternatively, antibodies can direct the killing of virus-infected cells by activating innate immune cells, such as natural killer cells, macrophages, and neutrophils, via Fc-FcyR interactions (60).

In contrast to the NS1 protein, the ZIKV envelope protein is not expressed at the cell surface (15). Nascent flaviviral particles bud internally from the Golgi apparatus, and structural proteins are not readily accessible on the surfaces of infected cells. It is possible that while envelope-specific antibodies can bind intact virion to elicit ADE, these antibodies are unable to bind infected cells and, thus, cannot evoke protective Fc-mediated effector functions as measured by our in vitro reporter assay. Data presented in this Example demonstrate that TBEV-vaccinated individuals can elicit cross-reactive anti-bodies toward the Zika virus E protein. The cross-reactivity of antibodies between TBEV vaccinees and ZIKV is not surprising given that many flaviviruses have common epitopes on the surface of the E protein. The TBEV vaccine preparation uses inactivated TBEV virus. Therefore, the NS1 component is not part of the TBEV vaccine, and there is no measurable antibody response to the ZIKV NS1 protein. These cross-reactive envelope antibodies could not engage FcyRs in reporter assay when tested on virus-infected cells (FIGS. 17A-17C). ADE of infection occurs when measured in vitro, however, because virions prominently display conserved fusion loop epitopes contrib-uting to enhanced viral uptake. It is possible that while E-specific antibodies are superior in providing sterilizing immunity against Zika virus infection, their protective efficacy can be limited by the possibility of ADE. Additionally, E-specific antibodies are not efficient in the clearance of virus-infected cells because of the lack of envelope protein displayed at the cell surface. In contrast, NS1-specific antibodies can direct the clearance of virally infected cells, as shown previously (32).

Overall, the work presented herein further establishes the importance of NS1 as a component of a safe and effective Zika virus vaccine. These data may explain how the incorporation of an NS1 component can enhance the effectiveness of a candidate ZIKV vaccine containing structural components only (42). The design of a safe and effective ZIKV vaccine will likely benefit from the incorporation of an NS1 immunogen to induce potent Fc-mediated immunity that clears virus-infected cells. Antibodies elicited by an NS1-based vaccine can protect in a lethal-challenge model and are functionally active as measured by a surrogate ADCC assay. Furthermore, NS1-specific antibodies are robust and long-lasting in humans and, based on mouse experiments, can provide protection against ZIKV disease via Fc-FcγR interactions.

### 6.2.5 References Cited in Example 2

1. Petersen, L. R., Jamieson, D. J. & Honein, M. A. Zika Virus. N. Engl. J. Med. 375, 294-295 (2016).
2. Brasil, P. et al. Guillain-Barré syndrome associated with Zika virus infection. The Lancet 387, 1482 (2016).
3. Brasil, P. et al. Zika Virus Infection in Pregnant Women in Rio de Janeiro. N. Engl. J. Med. 375, 2321-2334 (2016).
4. Honein, M. A. et al. Birth Defects Among Fetuses and Infants of US Women With Evidence of Possible Zika Virus Infection During Pregnancy. JAMA 317, 59-68 (2017).
5. Moreira, J., Peixoto, T. M., Siqueira, A. M. & Lamas, C. C. Sexually acquired Zika virus: a systematic review. Clin. Microbiol. Infect. Off. Publ. Eur. Soc. Clin. Microbiol. Infect. Dis. 23, 296-305 (2017).
6. Hajra, A. et al. Zika and pregnancy: A comprehensive review. Am. J. Reprod. Immunol. N. Y. N 1989 77, (2017).
7. Weaver, S. C. Prediction and prevention of urban arbovirus epidemics: A challenge for the global virology community. Antiviral Res. 156, 80-84 (2018).
8. Watts, A. G. et al. Potential Zika virus spread within and beyond India. J. Travel Med. (2018). doi: 10.1093/jtm/tay 132
9. Barouch, D. H., Thomas, S. J. & Michael, N. L. Prospects for a Zika Virus Vaccine. Immunity 46, 176-182 (2017).
10. Abbink, P. et al. Protective efficacy of multiple vaccine platforms against Zika virus challenge in rhesus monkeys. Science 353, 1129-1132 (2016).
11. Gaudinski, M. R. et al. Safety, tolerability, and immunogenicity of two Zika virus DNA vaccine candidates in healthy adults: randomised, open-label, phase 1 clinical trials. The Lancet 391, 552-562 (2018).
12. Richner, J. M. et al. Modified mRNA Vaccines Protect against Zika Virus Infection. Cell 168, 1114-1125.e10 (2017).
13. Shan, C. et al. A live-attenuated Zika virus vaccine candidate induces sterilizing immunity in mouse models. Nat. Med. 23, 763-767 (2017).
14. Modjarrad, K. et al. Preliminary aggregate safety and immunogenicity results from three trials of a purified inactivated Zika virus vaccine candidate: phase 1, randomised, double-blind, placebo-controlled clinical trials. The Lancet 391, 563-571 (2018).
15. Heinz, F. X. & Stiasny, K. The Antigenic Structure of Zika Virus and Its Relation to Other Flaviviruses: Implications for Infection and Immunoprophylaxis. Microbiol. Mol. Biol. Rev. MMBR 81, (2017).
16. Barba-Spaeth, G. et al. Structural basis of potent Zika-dengue virus antibody cross-neutralization. Nature 536, 48-53 (2016).
17. Whitehead, S. S., Blaney, J. E., Durbin, A. P. & Murphy, B. R. Prospects for a dengue virus vaccine. Nat. Rev. Microbiol. 5, 518-528 (2007).
18. Bardina, S. V. et al. Enhancement of Zika virus pathogenesis by preexisting antiflavivirus immunity. Science 356, 175-180 (2017).
19. Stettler, K. et al. Specificity, cross-reactivity, and function of antibodies elicited by Zika virus infection. Science 353, 823-826 (2016).
20. Rastogi, M., Sharma, N. & Singh, S. K. Flavivirus NS1: a multifaceted enigmatic viral protein. Virol. J. 13, 131 (2016).
21. Mackenzie, J. M., Jones, M. K. & Young, P. R. Immunolocalization of the Dengue Virus Nonstructural Glycoprotein NS1 Suggests a Role in Viral RNA Replication. Virology 220, 232-240 (1996).
22. Avirutnan, P. et al. Vascular leakage in severe dengue virus infections: a potential role for the nonstructural viral protein NS1 and complement. J. Infect. Dis. 193, 1078-1088 (2006).
23. Xia, H. et al. An evolutionary NS1 mutation enhances Zika virus evasion of host interferon induction. Nat. Commun. 9, 414 (2018).
24. Liu, J. et al. Flavivirus NS1 protein in infected host sera enhances viral acquisition by mosquitoes. Nat. Microbiol. 1, 16087 (2016).
25. Liu, Y. et al. Evolutionary enhancement of Zika virus infectivity in Aedes aegypti mosquitoes. Nature 545, 482-486 (2017).
26. Schlesinger, J. J., Foltzer, M. & Chapman, S. The Fc Portion of Antibody to Yellow Fever Virus NS1 Is a Determinant of Protection against YF Encephalitis in Mice. Virology 192, 132-141 (1993).
27. Chung, K. M., Thompson, B. S., Fremont, D. H. & Diamond, M. S. Antibody recognition of cell surface-associated NS1 triggers Fc-gamma receptor-mediated phagocytosis and clearance of West Nile Virus-infected cells. J. Virol. 81, 9551-9555 (2007).
28. Chung, K. M. et al. West Nile virus nonstructural protein NS1 inhibits complement activation by binding the regulatory protein factor H. Proc. Natl. Acad. Sci. 103, 19111-19116 (2006).
29. Wan, S.-W. et al. Therapeutic Effects of Monoclonal Antibody against Dengue Virus NS1 in a STAT1 Knockout Mouse Model of Dengue Infection. J. Immunol. Baltim. Md 1950 199, 2834-2844 (2017).
30. Lustig, Y. et al. Sensitivity and Kinetics of an NS1-Based Zika Virus Enzyme-Linked Immunosorbent Assay in Zika Virus-Infected Travelers from Israel, the Czech Republic, Italy, Belgium, Germany, and Chile. J. Clin. Microbiol. 55, 1894-1901 (2017).
31. Gao, X. et al. Delayed and highly specific antibody response to nonstructural protein 1 (NS1) revealed during natural human ZIKV infection by NS1-based capture ELISA. BMC Infect. Dis. 18, 275 (2018).
32. Bailey, M. J. et al. Human antibodies targeting Zika virus NS1 provide protection against disease in a mouse model. Nat. Commun. 9, 4560 (2018).
33. Schlesinger, J. J., Brandriss, M. W. & Walsh, E. E. Protection against 17D yellow fever encephalitis in mice by passive transfer of monoclonal antibodies to the nonstructural glycoprotein gp48 and by active immunization with gp48. J. Immunol. Baltim. Md 1950 135, 2805-2809 (1985).
34. Schlesinger, J. J., Brandriss, M. W., Cropp, C. B. & Monath, T. P. Protection against yellow fever in monkeys by immunization with yellow fever virus nonstructural protein NS1. J. Virol. 60, 1153-1155 (1986).
35. Beatty, P. R. et al. Dengue virus NS1 triggers endothelial permeability and vascular leak that is prevented by NS1 vaccination. Sci. Transl. Med. 7, 304ra141-304ra141 (2015).
36. Watterson, D., Modhiran, N. & Young, P. R. The many faces of the flavivirus NS1 protein offer a multitude of options for inhibitor design. Antiviral Res. 130, 7-18 (2016).
37. Chuang, Y.-C. et al. Dengue virus-induced autoantibodies bind to plasminogen and enhance its activation. J. Immunol. Baltim. Md 1950 187, 6483-6490 (2011).
38. Chuang, Y.-C., Lin, Y.-S., Liu, H.-S. & Yeh, T.-M. Molecular Mimicry between Dengue Virus and Coagulation Factors Induces Antibodies To Inhibit Thrombin Activity and Enhance Fibrinolysis. J. Virol. 88, 13759-13768 (2014).
39. Wang, T. T. et al. IgG antibodies to dengue enhanced for FcγRIIIA binding determine disease severity. Science 355, 395-398 (2017).
40. Brault, A. C. et al. A Zika Vaccine Targeting NS1 Protein Protects Immunocompetent Adult Mice in a Lethal Challenge Model. Sci. Rep. 7, 14769 (2017).
41. Li, A. et al. A Zika virus vaccine expressing premembrane-envelope-NS1 polyprotein. Nat. Commun. 9, 3067 (2018).
42. Liu, X. et al. Incorporation of NS1 and prM/M are important to confer effective protection of adenovirus-vectored Zika virus vaccine carrying E protein. Npj Vaccines 3, 29 (2018).
43. Somnuke, P., Hauhart, R. E., Atkinson, J. P., Diamond, M. S. & Avirutnan, P. N-linked glycosylation of dengue virus NS1 protein modulates secretion, cell-surface expression, hexamer stability, and interactions with human complement. Virology 413, 253-264 (2011).
44. He, W. et al. Epitope specificity plays a critical role in regulating antibody-dependent cell-mediated cytotoxicity against influenza A virus. Proc. Natl. Acad. Sci. 201609316 (2016). doi: 10.1073/pnas. 1609316113
45. Tripathi, S. et al. A novel Zika virus mouse model reveals strain specific differences in virus pathogenesis and host inflammatory immune responses. PLOS Pathog. 13, e1006258 (2017).
46. Robbiani, D. F. et al. Recurrent Potent Human Neutralizing Antibodies to Zika Virus in Brazil and Mexico. Cell 169, 597-609.e11 (2017).
47. Zhao, H. Structural basis of Zika virus-specific antibody protection. Cell 166, 1016-1027 (2016).
48. Beltramello, M. et al. The Human Immune Response to Dengue Virus Is Dominated by Highly Cross-Reactive Antibodies Endowed with Neutralizing and Enhancing Activity. Cell Host Microbe 8, 271-283 (2010).
49. Duehr, J. et al. Tick-Borne Encephalitis Virus Vaccine-Induced Human Antibodies Mediate Negligible Enhancement of Zika Virus Infection In Vitro and in a Mouse Model. mSphere 3, e00011-18 (2018).
50. Xu, K. et al. Recombinant Chimpanzee Adenovirus Vaccine AdC7-M/E Protects against Zika Virus Infection and Testis Damage. J. Virol. 92, (2018).
51. To, A. et al. Recombinant Zika Virus Subunits Are Immunogenic and Efficacious in Mice. mSphere 3, e00576-17 (2018).
52. Dowd, K. A. et al. Rapid development of a DNA vaccine for Zika virus. Science 354, 237-240 (2016).
53. Wang, Q. et al. Molecular determinants of human neutralizing antibodies isolated from a patient infected with Zika virus. Sci. Transl. Med. 8, 369ra179-369ra179 (2016).
54. Sapparapu, G. et al. Neutralizing human antibodies prevent Zika virus replication and fetal disease in mice. Nature 540, 443-447 (2016).
55. Ferguson, N. M. et al. Benefits and risks of the Sanofi-Pasteur dengue vaccine: Modeling optimal deployment. Science 353, 1033-1036 (2016).
56. Focus on dengue-Sanofi Pasteur. Available at: https://www.sanofipasteur.com/en/media-room/focus-on-diseases/dengue. (Accessed: 24 Sep. 2018)
57. Garçon, N., Chomez, P. & Mechelen, M. V. GlaxoSmithKline Adjuvant Systems in vaccines: concepts, achievements and perspectives. Expert Rev. Vaccines 6, 723-739 (2007).
58. Vargas-Inchaustegui, D. A. & Robert-Guroff, M. Fc Receptor-Mediated Immune Responses: New Tools But Increased Complexity in HIV Prevention. Curr. HIV Res. 11, 407-420 (2013).
59. Dugast, A.-S. et al. Decreased Fc receptor expression on innate immune cells is associated with impaired antibody-mediated cellular phagocytic activity in chronically HIV-1 infected individuals. Virology 415, 160-167 (2011).
60. Bournazos, S., DiLillo, D. J. & Ravetch, J. V. The role of Fc-FcγR interactions in IgG-mediated microbial neutralization. J. Exp. Med. 212, 1361-1369 (2015).

TABLE 9

| Antibody | Viral Strain | Kₒₙ1 (1/Ms) | K_{dis}1 (1/s) | K_{D}1 (M) | Kₒₙ2 (1/Ms) | K_{dis}2 (1/s) | K_{D}2 (M) | X² | R² |
|---|---|---|---|---|---|---|---|---|---|
| AA12 | MR766 | 1.10 × 10⁴ | 6.11 × 10⁻⁴ | 5.54 × 10⁻⁸ | 1.63 × 10⁵ | 1.62 × 10⁻² | 9.98 × 10⁻⁸ | 0.6389 | 0.9989 |
| AA12 | PRVABC59 | 6.03 × 10³ | 4.59 × 10⁻⁴ | 7.61 × 10⁻⁸ | 1.45 × 10⁵ | 2.13 × 10⁻² | 1.47 × 10⁻⁷ | 0.2677 | 0.9986 |
| FC12 | MR766 | No binding | No binding | No binding | No binding | No binding | No binding | No binding | No binding |
| FC12 | PRVABC59 | 7.98 × 10³ | 5.70 × 10⁻⁴ | 5.33 × 10⁻⁸ | 3.18 × 10⁵ | 1.70 × 10⁻² | 7.15 × 10⁻⁸ | 0.0658 | 0.9977 |
| EB9 | MR766 | 2.24 × 10⁴ | 6.52 × 10⁻⁴ | 2.92 × 10⁻⁸ | 1.15 × 10⁵ | 9.45 × 10⁻³ | 8.22 × 10⁻⁸ | 1.7281 | 0.9992 |
| EB9 | PRVABC59 | 2.23 × 10⁴ | 6.29 × 10⁻⁴ | 2.81 × 10⁻⁸ | 1.36 × 10⁵ | 9.93 × 10⁻³ | 7.31 × 10⁻⁸ | 1.0758 | 0.9996 |
| GB5 | MR766 | 2.22 × 10³ | 7.18 × 10⁻⁴ | 2.71 × 10⁻⁷ | 8.30 × 10⁴ | 3.55 × 10⁻⁴ | 3.23 × 10⁻⁷ | 0.0267 | 0.9967 |
| GB5 | PRVABC59 | 9.69 × 10³ | 7.45 × 10⁻⁴ | 7.69 × 10⁻⁸ | 1.73 × 10⁵ | 1.84 × 10⁻² | 1.06 × 10⁻⁷ | 0.0756 | 0.9985 |

TABLE 10

| | V- | J- | | % | | V- | J- | |
|---|---|---|---|---|---|---|---|---|
| Antibody | GENE | GENE | CDR3 | Identity | Isotype | GENE | GENE | CDR3 |
| AA12 | VH3- | JH3- | CARDRRGFDYW | 99% | IgG1 | VK1- | JK4- | CQQTYSTPLTF |
| | 53 | 02 | (SEQ ID NO: 155) | | | 39 | 01 | (SEQ ID NO: 159) |
| FC12 | VH3- | JH3- | CARGPVQLERRPLGAFDIW | 99% | IgG1 | VL3- | JL2- | CQAWDSSTVVF |
| | 53 | 02 | (SEQ ID NO: 156) | | | 1 | 01 | (SEQ ID NO: 160) |
| EB9 | VH3- | JH3- | CARWGGKRGGAFDIW | 100% | IgG1 | VK1- | JL2- | CQQSYSTPYTF |
| | 53 | 02 | (SEQ ID NO: 157) | | | 39 | 01 | (SEQ ID NO: 161) |
| GB5 | VH3- | JH3- | CARLIAAAGDYW | 99% | IgG1 | VK1- | JK1- | CQQSYSTPWTF |
| | 53 | 01 | (SEQ ID NO: 158) | | | 39 | 01 | (SEQ ID NO: 162) |

| | | | Reactivity | | |
|---|---|---|---|---|---|
| | | | to | Reactivity | |
| | % | | PRVABC59 | to MR766 | Neutralization |
| Antibody | Identity | Isotype | NS1 | NS1 | Activity (IC₅₀) |
| AA12 | 98% | kappa | Yes | Yes | None detected |
| FC12 | 100% | lambda | Yes | No | None detected |
| EB9 | 98% | kappa | Yes | Yes | None detected |
| GB5 | 98% | kappa | Yes | Yes | None detected |

Antibody Characteristics. VJ assignments, CDR3 sequences, % identity, and isotype for the antibody clones. IMGT/V-QUEST software was used to assign the germline reference for IGHV and IGLV and determine % identity to germline. ELISAs were done to determine reactivity and microneutralization assays were performed to determine neutralization activity at concentrations up to 100 μg/mL.

TABLE 11

| Patient | | | Country of | Days post |
|---|---|---|---|---|
| code | Age | Gender | exposure | illness onset |
| UTMB1 | n/a | F | Dominican Republic | 22 |
| UTMB1 | n/a | F | Dominican Republic | 58 |
| UTMB2 | 32 | F | Honduras | 3 |
| UTMB2 | 32 | F | Honduras | 7 |
| UTMB2 | 32 | F | Honduras | 14 |
| UTMB2 | 32 | F | Honduras | 28 |
| UTMB2 | 32 | F | Honduras | 45 |
| UTMB4 | 26 | F | Caribbean Islands | 18 |
| UTMB5 | 28 | F | Jamaica | 25 |
| UTMB5 | 29 | F | Jamaica | 32 |
| UTMB7 | 15 | F | Colombia | 129 |
| UTMB8 | 28 | F | Colombia | 113 |
| UTMB9 | 43 | M | El Salvador, Guatemala | 145 |
| UTMB10 | 33 | M | Haiti | 41 |
| UTMB10 | 33 | M | Haiti | 97 |
| UTMB11 | n/a | n/a | Haiti | 56 |

Serum samples obtained from Zika virus infected patients. Serum samples obtained via the Global Virus Network. Patient code, age, gender, country of exposure and date of illness onset are shown.

TABLE 12

| Patient | Days post |
|---|---|
| code | illness onset |
| NR-50611 | 233 |
| NR-50613 | 236 |
| NR-50615 | 224 |
| NR-50617 | 267 |
| NR-50619 | 202 |
| NR-50621 | 202 |
| NR-51058 | 6 |
| NR-51079 | 6 |
| NR-51118 | 7 |
| NR-50808 | “Acute” |
| NR-50809 | “Acute” |
| NR-50810 | “Acute” |
| NR-50818 | “Acute” |
| NR-50819 | “Acute” |
| NR-50820 | “Acute” |

Serum samples obtained from Zika virus infected patients. Serum samples obtained via BEI resources. Patient code and date of illness onset are shown.

### 7. EQUIVALENTS

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A recombinant monoclonal antibody that specifically binds to a Zika virus NS1, wherein the antibody comprises:
I. (a) (1) a variable heavy chain region (VH) complementarity determining region (CDR)1 comprising the amino acid sequence GFTVSSNY (SEQ ID NO:143), (2) a VH CDR2 comprising the amino acid sequence IYSGGST (SEQ ID NO:144), (3) a VH CDR3 comprising the amino acid sequence ARDRRGFDY (SEQ ID NO:145), ARWGGKRGGAFDI (SEQ ID NO: 146), ARLIAAAGDY (SEQ ID NO:147), or ARGPVQLERRPLGAFDI (SEQ ID NO:148), (4) a variable light chain region (VL) CDR1 comprising the amino acid sequence QSISSX, X is Y or H (SEQ ID NO:134), (5) a VL CDR2 comprising the amino acid sequence X1X2S, X1 is A or Q, X2 is A or D (SEQ ID NO:135), and (6) a VL CDR3 comprising the amino acid sequence QQX1YSTPX2T, X1 is T or S, X2 is L, Y, or W (SEQ ID NO:136); or
(b) (1) a VH antibody binding region (ABR)1 comprising the amino acid sequence FTVSSNYMS (SEQ ID NO:149), (2) a VH ABR2 comprising the amino acid sequence WVSVIYSGGSTYYA (SEQ ID NO:150), (3) a VH ABR3 comprising the amino acid sequence ARDRRGFDY (SEQ ID NO:151), ARWGGKRGGAFDI (SEQ ID NO:152), ARLIAAAGDY (SEQ ID NO:153), or ARGPVQLERRPLGAFDI (SEQ ID NO:154), (4) a VL ABR1 comprising the amino acid sequence QSISSXILN, X1 is Y or H (SEQ ID NO: 137), (5) a VL ABR2 comprising the amino acid sequence XILIYAASSLQS, X1 is F or L (SEQ ID NO:138), and (6) a VL ABR3 comprising the amino acid sequence QQX1YSTPX2, X1 is T or S, X2 is L, Y or W (SEQ ID NO:139);
II. (a) (1) a VH CDR1 comprising the amino acid sequence GFTVSSNY (SEQ ID NO: 17), (2) a VH CDR2 comprising the amino acid sequence IYSGGST (SEQ ID NO: 18), (3) a VH CDR3 comprising the amino acid sequence ARDRRGFDY (SEQ ID NO: 19), (4) a VL CDR1 comprising the amino acid sequence QSISSY (SEQ ID NO: 20), (5) a VL CDR2 comprising the amino acid sequence AAS (SEQ ID NO: 21), and (6) a VL CDR3 comprising the amino acid sequence QQTYSTPLT (SEQ ID NO:22);
(b) (1) a VH CDR1 comprising the amino acid sequence GFTVSSNY (SEQ ID NO: 45), (2) a VH CDR2 comprising the amino acid sequence IYSGGST (SEQ ID NO: 46), (3) a VH CDR3 comprising the amino acid sequence ARWGGKRGGAFDI (SEQ ID NO:47), (4) a VL CDR1 comprising the amino acid sequence QSISSH (SEQ ID NO: 48), (5) a VL CDR 2 comprising the amino acid sequence AAS (SEQ ID NO:49), and (6) a VL CDR3 comprising the amino acid sequence QQSYSTPYT (SEQ ID NO: 50);
(c) (1) a VH CDR1 comprising the amino acid sequence GFTVSSNY (SEQ ID NO: 73), (2) a VH CDR2 comprising the amino acid sequence IYSGGST (SEQ ID NO: 74), (3) a VH CDR3 comprising the amino acid sequence ARLIAAAGDY (SEQ ID NO: 75), (4) a VL CDR1 comprising the amino acid sequence QSISSY (SEQ ID NO:76), (5) a VL CDR2 comprising the amino acid sequence AAS (SEQ ID NO:77), and (6) a VL CDR3 comprising the amino acid sequence QQSYSTPWT (SEQ ID NO:78);
(d) (1) a VH CDR1 comprising the amino acid sequence GFTVSSNY (SEQ ID NO: 101), (2) a VH CDR2 comprising the amino acid sequence IYSGGST (SEQ ID NO: 102), (3) a VH CDR3 comprising the amino acid sequence ARGPVQLERRPLGAFDI (SEQ ID NO:103), (4) a VL CDR1 comprising the amino acid sequence KLGDKY (SEQ ID NO: 104), (5) a VL CDR2 comprising the amino acid sequence QDS (SEQ ID NO: 105), and (6) a VL CDR3 comprising the amino acid sequence QAWDSSTVV (SEQ ID NO:106);
III (a) (1) a VH antibody binding region (ABR)1 comprising the amino acid sequence FTVSSNYMS (SEQ ID NO:31), (2) a VH ABR2 comprising the amino acid sequence WVSVIYSGGSTYYA (SEQ ID NO: 32), (3) a VH ABR3 comprising the amino acid sequence ARDRRGFDY (SEQ ID NO:33), (4) a VL ABR1 comprising the amino acid sequence QSISSYLN (SEQ ID NO:34), (5) a VL ABR2 comprising the amino acid sequence LLIYAASSLQS (SEQ ID NO: 35), and (6) a VL ABR3 comprising the amino acid sequence QQTYSTPL (SEQ ID NO: 36);
(b) (1) a VH ABR1 comprising the amino acid sequence FTVSSNYMS (SEQ ID NO: 59), (2) a VH ABR2 comprising the amino acid sequence WVSVIYSGGSTYYA (SEQ ID NO: 60), (3) a VH ABR3 comprising the amino acid sequence ARWGGKRGGAFDI (SEQ ID NO:61), (4) a VL ABR1 comprising the amino acid sequence QSISSHLN (SEQ ID NO: 62), (5) a VL ABR2 comprising the amino acid sequence FLIYAASSLQS (SEQ ID NO: 63), and (6) a VL ABR3 comprising the amino acid sequence QQSYSTPY (SEQ ID NO:64);
(c) (1) a VH ABR1 comprising the amino acid sequence FTVSSNYMS (SEQ ID NO: 87), (2) a VH ABR2 comprising the amino acid sequence WVSVIYSGGSTYYA (SEQ ID NO:88), (3) a VH ABR3 comprising the amino acid sequence ARLIAAAGDY (SEQ ID NO:89), (4) a VL ABR1 comprising the amino acid sequence QSISSYLN (SEQ ID NO: 90), (5) a VL ABR2 comprising the amino acid sequence LLIYAASSLQS (SEQ ID NO: 91), and (6) a VL ABR3 comprising the amino acid sequence QQSYSTPW (SEQ ID NO: 92); or
(d) (1) a VH ABR1 comprising the amino acid sequence FTVSSNYMS (SEQ ID NO:115), (2) a VH ABR2 comprising the amino acid sequence WVSVIYSGGSTYYA (SEQ ID NO:116), (3) a VH ABR3 comprising the amino acid sequence ARGPVQLERRPLGAFDI (SEQ ID NO:117), (4) a VL ABR1 comprising the amino acid sequence KLGDKYAC (SEQ ID NO:118), (5) a VL ABR2 comprising the amino acid sequence LVIYQDSKRPS (SEQ ID NO:119), and (6) a VL ABR3 comprising the amino acid sequence QAWDSSTV (SEQ ID NO:120); or
IV. (a) a variable heavy chain region comprising the amino acid sequence of SEQ ID NO: 9 and a variable light chain region comprising the amino acid sequence of SEQ ID NO: 10;
(b) a variable heavy chain region comprising the amino acid sequence of SEQ ID NO: 11 and a variable light chain region comprising the amino acid sequence of SEQ ID NO: 12;
(c) a variable heavy chain region comprising the amino acid sequence of SEQ ID NO: 13 and a variable light chain region comprising the amino acid sequence of SEQ ID NO: 14;
(d) a variable heavy chain region comprising the amino acid sequence of SEQ ID NO: 15 and a variable light chain region comprising the amino acid sequence of SEQ ID NO: 16;
(e) a VH that is at least 95% identical to the amino acid sequence of SEQ ID NO: 9 and a VL that is at least 95% identical to the amino acid sequence of SEQ ID NO: 10;
(f) a VH that is at least 95% identical to the amino acid sequence of SEQ ID NO: 11 and a VL that is at least 95% identical to the amino acid sequence of SEQ ID NO: 12;
(g) a VH that is at least 95% identical to the amino acid sequence of SEQ ID NO: 13 and a VL that is at least 95% identical to the amino acid sequence of SEQ ID NO: 14; or
(h) a VH is at least 95% identical to the amino acid sequence of SEQ ID NO: 15 and a VL is at least 95% identical to the amino acid sequence of SEQ ID NO: 16.

2.-14. (canceled)

15. A pharmaceutical composition comprising an effective amount of the recombinant monoclonal antibody of claim 1 in an admixture with a pharmaceutically acceptable carrier.

16. A method for detecting a Zika virus infection in a biological sample, comprising contacting the recombinant monoclonal antibody of claim 1 with the biological sample and detecting the binding of the antibody to a Zika virus NS1.

17. A method for diagnosing a Zika virus infection in a subject, comprising contacting the recombinant monoclonal antibody of claim 1 with a biological sample from the subject and detecting the binding of the antibody to a Zika virus NS1, wherein an increase in the detection of the binding of the antibody in the biological sample relative to the detection of binding of the antibody to a negative control sample indicates that the subject has a Zika virus infection.

18. (canceled)

19. (canceled)

20. A method for preventing or treating a Zika virus infection in a subject, comprising administering to the subject the pharmaceutical composition of claim 15.

21. (canceled)

22. (canceled)

23. An isolated nucleic acid sequence comprising a nucleotide sequence encoding the recombinant monoclonal antibody of claim 1.

24. A host cell comprising the nucleic acid sequence of claim 23.

25. A recombinant NS1 polypeptide comprising the amino acid sequence of a Zika virus NS1 and the amino acid sequence of a fragment of a Zika virus envelope protein, wherein the amino acid sequence of the fragment of the Zika virus envelope protein is at the N-terminus of the amino acid sequence of the Zika virus NS1.

26. The recombinant NS1 polypeptide of claim 25, wherein:
(a) the fragment of the Zika virus envelope protein comprises the last 20 to 50 carboxy-terminal amino acid residues of the Zika virus envelope protein; or
(b) the fragment comprises the amino acid sequence NGSISLMCLALGGVLIFLSTAVSA (SEQ ID NO: 131).

27.-33. (canceled)

34. An isolated nucleic acid sequence comprising a nucleotide sequence encoding the recombinant NS1 protein of claim 25.

35. (canceled)

36. An expression vector comprising the nucleic acid sequence of claim 34.

37. (canceled)

38. (canceled)

39. A host cell engineered to express a recombinant NS1 polypeptide encoded by the nucleic acid sequence of claim 34.

40. (canceled)

41. A pharmaceutical composition comprising the recombinant NS1 polypeptide of claim 25, or a nucleic acid sequence comprising a nucleotide sequence encoding the recombinant NS1 polypeptide of claim 25 in an admixture with a pharmaceutically acceptable carrier.

42. A method for immunizing a subject against Zika virus, or preventing a Zika virus-mediated disease in a subject, or inducing an immune response against a Zika virus NS1, comprising administering to the subject a dose of the pharmaceutical composition of claim 41.

43.-45. (canceled)

46. A method for immunizing a subject against Zika virus, comprising: (a) administering to the subject a first pharmaceutical composition comprising a nucleic acid sequence comprising a nucleotide sequence encoding the recombinant NS1 polypeptide of claim 25; and (b) after a first certain period of time administering to the subject a second pharmaceutical composition comprising the recombinant NS1 polypeptide of claim 25.

47.-49. (canceled)

50. A recombinant monoclonal antibody that specifically binds to a Zika virus NS1, wherein the antibody comprises:
(i) (a) a variable heavy chain region (VH) and human IgG1 constant regions encoded by the pFUESss-CHIg-hIgG1 vector, wherein the VH comprises the amino acid sequence encoded by the cDNA sequence of SEQ ID NO: 1; and
(b) a variable light chain region (VL) and human kappa constant regions encoded by the pFUESss-CLIg-hK vector, wherein the VL comprises the amino acid sequence encoded by the cDNA sequence of SEQ ID NO: 2;
(ii) (a) a variable heavy chain region (VH) and human IgG1 constant regions encoded by the pFUESss-CHIg-hIgG1 vector, wherein the VH comprises the amino acid sequence encoded by the cDNA sequence of SEQ ID NO: 3; and
(b) a variable light chain region (VL) and human kappa constant regions encoded by the pFUESss-CLIg-hl2 vector, wherein the VL comprises the amino acid sequence encoded by the cDNA sequence of SEQ ID NO: 4;
(iii) (a) a variable heavy chain region (VH) and human IgG1 constant regions encoded by the pFUESss-CHIg-hIgG1 vector, wherein the VH comprises the amino acid sequence encoded by the cDNA sequence of SEQ ID NO: 5; and
(b) a variable light chain region (VL) and human kappa constant regions encoded by the pFUESss-CLIg-hK vector, wherein the VL comprises the amino acid sequence encoded by the cDNA sequence of SEQ ID NO: 6; or
(iv) (a) a variable heavy chain region (VH) and human IgG1 constant regions encoded by the pFUESss-CHIg-hIgG1 vector, wherein the VH comprises the amino acid sequence encoded by the cDNA sequence of SEQ ID NO: 7; and
(b) a variable light chain region (VL) and human lambda constant regions encoded by the pFUESss-CLIg-hK vector, wherein the VL comprises the amino acid sequence encoded by the cDNA sequence of SEQ ID NO: 8.

51. A method for reducing the risk of severe disease caused by a Zika virus infection in flavivirus naïve patient, comprising administering to the subject the recombinant monoclonal antibody of claim 50 before infection with the Zika virus, wherein the Zika virus is Rhesus/1947/Uganda Bei NR-50065 or H/PAN/2015/CDC-259359.

52. An expression vector comprising:
(a) pFUESss-CHIg-hIgG1 vector containing the cDNA sequence of SEQ ID NO: 1;
(b) pFUESss-CLIg-hK vector containing the cDNA sequence of SEQ ID NO: 2;
(c) pFUESss-CHIg-hIgG1 vector containing the cDNA sequence of SEQ ID NO: 3;
(d) pFUESss-CLIg-hl2 vector containing the cDNA sequence of SEQ ID NO: 4;
(e) pFUESss-CHIg-hIgG1 vector containing the cDNA sequence of SEQ ID NO: 5;
(f) pFUESss-CLIg-hK vector containing the cDNA sequence of SEQ ID NO: 6;
(g) pFUESss-CHIg-hIgG1 vector containing the cDNA sequence of SEQ ID NO: 7; or
(h) pFUESss-CLIg-hK vector containing the cDNA sequence of SEQ ID NO: 8.

53. HEK 293F cells expressing:
(i) (a) pFUESss-CHIg-hIgG1 vector containing the cDNA sequence of SEQ ID NO: 1; and
(b) pFUESss-CLIg-hK vector containing the cDNA sequence of SEQ ID NO: 2;
(ii) (a) pFUESss-CHIg-hIgG1 vector containing the cDNA sequence of SEQ ID NO: 3; and
(b) pFUESss-CLIg-hl2 vector containing the cDNA sequence of SEQ ID NO: 4;
(iii) (a) pFUESss-CHIg-hIgG1 vector containing the cDNA sequence of SEQ ID NO: 5; and
(b) pFUESss-CLIg-hK vector containing the cDNA sequence of SEQ ID NO: 6; or
(iv) (a) pFUESss-CHIg-hIgG1 vector containing the cDNA sequence of SEQ ID NO: 7; and
(b) pFUESss-CLIg-hK vector containing the cDNA sequence of SEQ ID NO: 8.

54. A method for producing a recombinant monoclonal antibody that specifically binds to a Zika virus NS1, comprising:
(a) culturing the HEK 293F cells of claim 53; and
(b) purifying the monoclonal antibody.

55. The recombinant monoclonal antibody of claim 50, wherein the Zika virus NS1 is the NS1 of Rhesus/1947/Uganda Bei NR-50065 or H/PAN/2015/CDC-259359.

56. The method of claim 54, wherein the Zika virus NS1 is the NS1 of Rhesus/1947/Uganda Bei NR-50065 or H/PAN/2015/CDC-259359.
